(19) United States
(12) Patent Application Publication
(10) Pub. No.: US 2026/0297188 A1
(43) Pub. Date: Oct. 1, 2026

(54) ANTIBODIES AND USES THEREOF

(71) Applicant: LTZ Therapeutics Inc., Redwood City, CA (US)

(72) Inventors: Jinze Li, Danville, CA (US); Jianhui Zhou, Fremont, CA (US); Vivekananda Kedage, Foster City, CA (US); Jie Hu, Mountain View, CA (US); Zhenfei Ning, Yantai, Shandong (CN); Jintao Feng, Shenzhen, Shandong (CN); Martin Treder, Reilingen (DE); Zhentao Sheng, Sunnyvale, CA (US); Su Huang, Rochester, MN (US)

(21) Appl. No.: 19/477,883

(22) PCT Filed: May 17, 2024
(86) PCT No.: PCT/US2024/029992

§ 371 Date: Oct. 23, 2025

(30) Foreign Application Priority Data
May 19, 2023 (WO) PCT/CN2023/095161
May 19, 2023 (WO) PCT/CN2023/095163
Dec. 8, 2023 (WO) PCT/CN2023/137346
Dec. 8, 2023 (WO) PCT/CN2023/137348

(51) Int. Cl. C07K 16/28; A61K 45/06; A61P 35/00; C07K 16/30

(52) U.S. Cl. CPC C07K 16/2803; A61K 45/06; A61P 35/00; C07K 16/2863; C07K 16/2866; C07K 16/2875; C07K 16/2878; C07K 16/30; C07K 16/3069; C07K 16/3092; C07K 2317/14; C07K 2317/24; C07K 2317/31; C07K 2317/33; C07K 2317/40; C07K 2317/522; C07K 2317/565; C07K 2317/622

(57) ABSTRACT

The disclosure relates to anti-NKp46 antibodies, antigen-binding fragments, and the uses thereof. The disclosure also relates to an antibody or antigen-binding fragment thereof, comprising a first antigen-binding domain that specifically binds to a tumor associated antigen (TAA) and a second antigen-binding domain that specifically binds to an NK cell surface antigen, and an Fc region with optimized Fc-effector function.

### CLAIM OF PRIORITY

This application claims the benefit of Application Nos PCT/CN2023/095161, filed May 19, 2023; PCT/CN2023/137348, filed Dec. 8, 2023; PCT/CN2023/095163, filed May 19, 2023; and PCT/CN2023/137346, filed Dec. 8, 2023. The entire contents of the foregoing applications are hereby incorporated by reference.

### TECHNICAL FIELD

This disclosure relates to anti-NKp46 antibodies, multispecific antibodies (e.g., bispecific antibodies or antigen-binding fragments thereof), and antibody drug conjugates derived therefrom.

### BACKGROUND

Cancer is currently one of the diseases that have the highest human mortality. According to the World Health Organization statistical data, in 2012, the number of global cancer incidence and death cases reached 14 million and 8.2 million, respectively. In China, the newly diagnosed cancer cases are 3.07 million, and the death toll is 2.2 million. Recent clinical and commercial success of anticancer antibodies has created great interest in antibody-based therapeutics.

A bispecific antibody is an artificial protein that can simultaneously bind to two different types of antigens or two different epitopes. This dual specificity opens up a wide range of applications, including redirecting T cells to tumor cells, dual targeting of different disease mediators, and delivering payloads to targeted sites. The approval of catumaxomab (anti-EpCAM and anti-CD3) and blinatumomab (anti-CD19 and anti-CD3) has become a major milestone in the development of bispecific antibodies.

There is a need to develop antibodies for use in various antibody-based therapeutics to treat cancers. As bispecific antibodies have various applications, there is also a need to continue to develop various therapeutics based on bispecific antibodies.

### SUMMARY

This disclosure relates to anti-NKp46 antibodies, antigen-binding fragment thereof, and the uses thereof.

This disclosure also relates to multispecific (e.g., bispecific) antibodies or antigen-binding fragments thereof, wherein the antibodies or antigen-binding fragments thereof specifically bind to a tumor associated antigen (TAA) and an NK cell surface antigen. In some embodiments, the antibodies or antigen-binding fragments thereof have an optimized Fc. In some embodiments, the antibody or antigen-binding fragment thereof has an increased binding affinity to the FcγRIIa receptor and/or the FcγRIIIa receptor.

In one aspect, provided herein are antibodies or antigen-binding fragments thereof that binds to NKp46, comprising: a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence, wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 5, 7, 9, respectively, and the selected VL CDRs 1, 2, 3 amino acid sequences are set forth in SEQ ID NOs: 10, 11, 12, respectively;
(2) the selected VH CDRs 1, 2, 3 amino acid sequences are set forth in SEQ ID NOs: 6, 8, 9, respectively, and the selected VL CDRs 1, 2, 3 amino acid sequences are set forth in SEQ ID NOs: 10, 11, 12, respectively;
(3) the selected VH CDRs 1, 2, 3 amino acid sequences are set forth in SEQ ID NOs: 15, 17, 19, respectively, and the selected VL CDRs 1, 2, 3 amino acid sequences are set forth in SEQ ID NOs: 20, 21, 22, respectively;
(4) the selected VH CDRs 1, 2, 3 amino acid sequences are set forth in SEQ ID NOs: 16, 18, 19, respectively, and the selected VL CDRs 1, 2, 3 amino acid sequences are set forth in SEQ ID NOs: 20, 21, 22, respectively,
(5) the selected VH CDRs 1, 2, 3 amino acid sequences are set forth in SEQ ID NOs: 25, 27, 29, respectively, and the selected VL CDRs 1, 2, 3 amino acid sequences are set forth in SEQ ID NOs: 30, 31, 32, respectively;
(6) the selected VH CDRs 1, 2, 3 amino acid sequences are set forth in SEQ ID NOs: 26, 28, 29, respectively, and the selected VL CDRs 1, 2, 3 amino acid sequences are set forth in SEQ ID NOs: 30, 31, 32, respectively; and

In some embodiments, the VH comprises CDRs 1, 2, 3 with the amino acid sequences set forth in SEQ ID NOs: 5, 7, and 9, respectively, and the VL comprises CDRs 1, 2, 3 with the amino acid sequences set forth in SEQ ID NOs: 10, 11, and 12, respectively, according to Kabat definition.

In some embodiments, the VH comprises CDRs 1, 2, 3 with the amino acid sequences set forth in SEQ ID NOs: 6, 8, and 9, respectively, and the VL comprises CDRs 1, 2, 3 with the amino acid sequences set forth in SEQ ID NOs: 10, 11, and 12, respectively, according to Chothia definition.

In some embodiments, the VH comprises CDRs 1, 2, 3 with the amino acid sequences set forth in SEQ ID NOs: 15, 17, and 19, respectively, and the VL comprises CDRs 1, 2, 3 with the amino acid sequences set forth in SEQ ID NOs: 20, 21, and 22, respectively, according to Kabat definition.

In some embodiments, the VH comprises CDRs 1, 2, 3 with the amino acid sequences set forth in SEQ ID NOs: 16, 18, and 19, respectively, and the VL comprises CDRs 1, 2, 3 with the amino acid sequences set forth in SEQ ID NOs: 20, 21, and 22, respectively, according to Chothia definition.

In some embodiments, the VH comprises CDRs 1, 2, 3 with the amino acid sequences set forth in SEQ ID NOs: 25, 27 and 29, respectively, and the VL comprises CDRs 1, 2, 3 with the amino acid sequences set forth in SEQ ID NOs: 30, 31 and 32, respectively, according to Kabat definition.

In some embodiments, the VH comprises CDRs 1, 2, 3 with the amino acid sequences set forth in SEQ ID NOs: 26, 28 and 29, respectively, and the VL comprises CDRs 1, 2, 3 with the amino acid sequences set forth in SEQ ID NOs: 30, 31 and 32, respectively, according to Chothia definition.

In some embodiments, the antibody or antigen-binding fragment specifically binds to human NKp46 or monkey NKp46.

In some embodiments, the antibody or antigen-binding fragment is a human or humanized antibody or antigen-binding fragment thereof.

In some embodiments, the antibody or antigen-binding fragment is a single-chain variable fragment (scFv) or a multi-specific antibody (e.g., a bispecific antibody).

In one aspect, provided herein are nucleic acids comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 5, 7, and 9, respectively, and wherein the VH, when paired with a light chain variable region (VL) comprising the amino acid sequence set forth in SEQ ID NO: 4 binds to NKp46;
(2) an immunoglobulin light chain or a fragment thereof comprising a VL comprising CDRs 1, 2, and 3 comprising the amino acid sequences set forth in SEQ ID NOs: 10, 11 and 12, respectively, and wherein the VL, when paired with a VH comprising the amino acid sequence set forth in SEQ ID NO: 3 binds to NKp46;
(3) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 6, 8 and 9, respectively, and wherein the VH, when paired with a light chain variable region (VL) comprising the amino acid sequence set forth in SEQ ID NO: 4 binds to NKp46;
(4) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 15, 17 and 19, respectively, and wherein the VH, when paired with a light chain variable region (VL) comprising the amino acid sequence set forth in SEQ ID NO: 14 binds to NKp46;
(5) an immunoglobulin light chain or a fragment thereof comprising a VL comprising CDRs 1, 2, and 3 comprising the amino acid sequences set forth in SEQ ID NOs: 20, 21 and 22, respectively, and wherein the VL, when paired with a VH comprising the amino acid sequence set forth in SEQ ID NO: 13 binds to NKp46;
(6) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 16, 18 and 19, respectively, and wherein the VH, when paired with a light chain variable region (VL) comprising the amino acid sequence set forth in SEQ ID NO: 14 binds to NKp46;
(7) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 25, 27 and 29, respectively, and wherein the VH, when paired with a light chain variable region (VL) comprising the amino acid sequence set forth in SEQ ID NO: 24 binds to NKp46;
(8) an immunoglobulin light chain or a fragment thereof comprising a VL comprising CDRs 1, 2, and 3 comprising the amino acid sequences set forth in SEQ ID NOs: 30, 31 and 32, respectively, and wherein the VL, when paired with a VH comprising the amino acid sequence set forth in SEQ ID NO: 23 binds to NKp46;
(9) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 26, 28 and 29, respectively, and wherein the VH, when paired with a light chain variable region (VL) comprising the amino acid sequence set forth in SEQ ID NO: 24 binds to NKp46;
(10) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 5, 7, and 9, respectively, and wherein the VH, when paired with a light chain variable region (VL) comprising the amino acid sequence set forth in SEQ ID NO: 36 binds to NKp46;
(11) an immunoglobulin light chain or a fragment thereof comprising a VL comprising CDRs 1, 2, and 3 comprising the amino acid sequences set forth in SEQ ID NOs: 10, 11 and 12, respectively, and wherein the VL, when paired with a VH comprising the amino acid sequence set forth in SEQ ID NO: 35 binds to NKp46;
(12) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 6, 8 and 9, respectively, and wherein the VH, when paired with a light chain variable region (VL) comprising the amino acid sequence set forth in SEQ ID NO: 36 binds to NKp46;
(13) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 15, 17 and 19, respectively, and wherein the VH, when paired with a light chain variable region (VL) comprising the amino acid sequence set forth in SEQ ID NO: 38 binds to NKp46;
(14) an immunoglobulin light chain or a fragment thereof comprising a VL comprising CDRs 1, 2, and 3 comprising the amino acid sequences set forth in SEQ ID NOs: 20, 21 and 22, respectively, and wherein the VL, when paired with a VH comprising the amino acid sequence set forth in SEQ ID NO: 37 binds to NKp46;
(15) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 16, 18 and 19, respectively, and wherein the VH, when paired with a light chain variable region (VL) comprising the amino acid sequence set forth in SEQ ID NO: 38 binds to NKp46;
(16) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 25, 27 and 29, respectively, and wherein the VH, when paired with a light chain variable region (VL) comprising the amino acid sequence set forth in SEQ ID NO: 34 binds to NKp46;
(17) an immunoglobulin light chain or a fragment thereof comprising a VL comprising CDRs 1, 2, and 3 comprising the amino acid sequences set forth in SEQ ID NOs: 30, 31 and 32, respectively, and wherein the VL, when paired with a VH comprising the amino acid sequence set forth in SEQ ID NO: 33 binds to NKp46;
(18) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 26, 28 and 29, respectively, and wherein the VH, when paired with a light chain variable region (VL) comprising the amino acid sequence set forth in SEQ ID NO: 34 binds to NKp46;

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 5, 7, and 9, respectively, or SEQ ID NOs: 6, 8 and 9, respectively.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 10, 11 and 12, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 15, 17 and 19, respectively, or SEQ ID NOs: 16, 18 and 19, respectively.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 20, 21 and 22, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 25, 27 and 29, respectively, or SEQ ID NOs: 26, 28 and 29, respectively.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 30, 31 and 32, respectively.

In some embodiments, the VH when paired with a VL specifically binds to human NKp46 or monkey NKp46, or the VL when paired with a VH specifically binds to human NKp46 or monkey NKp46.

In some embodiments, the immunoglobulin heavy chain or the fragment thereof is a human or humanized immunoglobulin heavy chain or a fragment thereof (e.g., a human IgG1 heavy chain or a fragment thereof), and the immunoglobulin light chain or the fragment thereof is a human or humanized immunoglobulin light chain or a fragment thereof.

In some embodiments, the nucleic acid encodes a single-chain variable fragment (scFv), a multi-specific antibody (e.g., a bispecific antibody) or a chimeric antigen receptor (CAR).

In some embodiments, the nucleic acid is cDNA.

In one aspect, provided herein are vectors comprising one or more of the nucleic acids described herein.

In one aspect, provided herein are vectors comprising two of the nucleic acids described herein, wherein the vector encodes the VH region and the VL region that together bind to NKp46.

In one aspect, provided herein are pairs of vectors, wherein each vector comprises one of the nucleic acids described herein, wherein together the pair of vectors encodes the VH region and the VL region that together bind to NKp46.

In one aspect, provided herein are cells comprising any one of the vectors described herein, or any of the pair of vectors described herein.

In some embodiments, the cell is a CHO cell.

In one aspect, provided herein are cells comprising one or more of the nucleic acids of described herein.

In one aspect, provided herein are cells comprising two of the nucleic acids described herein.

In some embodiments, the two nucleic acids together encode the VH region and the VL region that together bind to NKp46.

In one aspect, provided herein are methods of producing an antibody or an antigen-binding fragment thereof, the method comprising
(a) culturing any one of the cells described herein under conditions sufficient for the cell to produce the antibody or the antigen-binding fragment; and
(b) collecting the antibody or the antigen-binding fragment produced by the cell.

In one aspect, provided herein are antibodies or antigen-binding fragments thereof that binds to NKp46 comprising a heavy chain variable region (VH) comprising an amino acid sequence that is at least 80% identical to a selected VH sequence, and a light chain variable region (VL) comprising an amino acid sequence that is at least 80% identical to a selected VL sequence, wherein the selected VH sequence and the selected VL sequence are one of the following:
(1) the selected VH sequence is SEQ ID NO: 3, and the selected VL sequence is SEQ ID NO: 4;
(2) the selected VH sequence is SEQ ID NO: 13, and the selected VL sequence is SEQ ID NO: 14;
(3) the selected VH sequence is SEQ ID NO: 23, and the selected VL sequence is SEQ ID NO: 24;
(4) the selected VH sequence is SEQ ID NO: 33, and the selected VL sequence is SEQ ID NO: 34;
(5) the selected VH sequence is SEQ ID NO: 35, and the selected VL sequence is SEQ ID NO: 36; and
(6) the selected VH sequence is SEQ ID NO: 37, and the selected VL sequence is SEQ ID NO: 38.

In some embodiments, the VH comprises the sequence of SEQ ID NO: 3 and the VL comprises the sequence of SEQ ID NO: 4.

In some embodiments, the VH comprises the sequence of SEQ ID NO: 13 and the VL comprises the sequence of SEQ ID NO: 14.

In some embodiments, the VH comprises the sequence of SEQ ID NO: 23 and the VL comprises the sequence of SEQ ID NO: 24.

In some embodiments, the VH comprises the sequence of SEQ ID NO: 33 and the VL comprises the sequence of SEQ ID NO: 34.

In some embodiments, the VH comprises the sequence of SEQ ID NO: 35 and the VL comprises the sequence of SEQ ID NO: 36.

In some embodiments, the VH comprises the sequence of SEQ ID NO: 37 and the VL comprises the sequence of SEQ ID NO: 38.

In some embodiments, the antibody or antigen-binding fragment specifically binds to human NKp46 or monkey NKp46.

In some embodiments, the antibody or antigen-binding fragment is a human or humanized antibody or antigen-binding fragment thereof.

In some embodiments, the antibody or antigen-binding fragment is a single-chain variable fragment (scFv) or a multi-specific antibody (e.g., a bispecific antibody).

In one aspect, provided herein are antibodies or antigen-binding fragments thereof that cross-competes with any one of the antibodies or antigen-binding fragments thereof described herein.

In one aspect, provided herein are antibodies or antigen-binding fragments thereof that binds to NKp46 comprising a heavy chain variable region (VH) comprising VH CDR1, VH CDR2, and VH CDR3 that are identical to VH CDR1, VH CDR2, and VH CDR3 of a selected VH sequence, and a light chain variable region (VL) comprising VL CDR1, VL CDR2, and VL CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 3, and the selected VL sequence is SEQ ID NO: 4;
(2) the selected VH sequence is SEQ ID NO: 13, and the selected VL sequence is SEQ ID NO: 14;
(3) the selected VH sequence is SEQ ID NO: 23, and the selected VL sequence is SEQ ID NO: 24;
(4) the selected VH sequence is SEQ ID NO: 33, and the selected VL sequence is SEQ ID NO: 34;
(5) the selected VH sequence is SEQ ID NO: 35, and the selected VL sequence is SEQ ID NO: 36; and
(6) the selected VH sequence is SEQ ID NO: 37, and the selected VL sequence is SEQ ID NO: 38.

In one aspect, provided herein are methods of treating a subject having cancer or an autoimmune disease, the method comprising administering a therapeutically effective amount of a composition comprising any one of the antibodies or antigen-binding fragments thereof to the subject.

In some embodiments, the antibody or antigen-binding fragment thereof is a bispecific antibody that targets both an NK cell and a tumor cell expressing a tumor antigen.

In some embodiments, the method further comprises administering a therapeutically effective amount of an anti-OX40 antibody, an anti-PD1 antibody, an anti-PDL1 antibody, an anti-PDL2 antibody, an anti-LAG-3 antibody, an anti-TIGIT antibody, an anti-CTLA-4 antibody, an anti-GITR antibody, an anti-TIM-3 antibody, an anti-4-1BB antibody, and/or an anti-CD40 antibody, to the subject.

In one aspect, provided herein are methods of decreasing the rate of tumor growth, the method comprising contacting a tumor cell with an effective amount of a composition comprising any one of the antibodies or antigen-binding fragments thereof described herein.

In one aspect, provided herein are methods of killing a tumor cell, the method comprising contacting a tumor cell with an effective amount of a composition comprising any one of the antibodies or antigen-binding fragments thereof described herein.

In one aspect, provided herein are pharmaceutical compositions comprising any one of the antibodies or antigen-binding fragments thereof described herein, and a pharmaceutically acceptable carrier.

Also provided herein are antibodies or antigen-binding fragments thereof, comprising: i) a first antigen-binding domain that specifically binds to a first antigen, wherein the first antigen is a tumor associated antigen (TAA); ii) a second antigen-binding domain that specifically binds to a second antigen, wherein the second antigen is an NK cell surface antigen; iii) an Fc region.

In some embodiments, the first antigen-binding domain comprises a first heavy chain variable region (VH1) and a first light chain variable region (VL1); and the second antigen-binding domain comprises a second heavy chain variable region (VH2) and a second light chain variable region (VL2).

In some embodiments, the second antigen-binding domain is a single-chain fragment variable (scFv) domain comprising a light chain variable domain (VL2) and a heavy chain variable domain (VH2) linked by a first linker.

In some embodiments, the second antigen-binding domain is connected to the C-terminus of the light chain of the first antigen-binding domain through a second linker.

In some embodiments, the second antigen-binding domain is linked to the C-terminus of the Fc region.

In some embodiments, the VH1 is linked to a CH1 domain, and the VL1 is linked to a CL domain.

In some embodiments, the Fc region is an Fc region of human IgG1, IgG2, IgG3, or IgG4.

In some embodiments, the Fc region is an Fc region of human IgG1.

In some embodiments, the antibody or antigen-binding fragment thereof is a bispecific antibody or antigen-binding fragment thereof.

In some embodiments, the Fc region comprises one or more of the following (all numbering is according to EU numbering):
a Glutamic acid I at position 332;
a Leucine (L) at position 330;
an Alanine (A) at position 236; and/or
an Aspartic acid (D) at position 293.

In some embodiments, the Fc region comprises one of the following (all numbering is according to EU numbering):
i) an Alanine (A) at position 236, a Leucine (L) at position 330, and a Glutamic acid I at position 332;
ii) an Alanine (A) at position 236, an Aspartic acid (D) at position 293, a Leucine (L) at position 330, and a Glutamic acid I at position 332;
iii) an Alanine (A) at position 236.
iv) an Alanine (A) at position 236, an Aspartic acid (D) at position 293, and a Glutamic acid I at position 332;
v) an Aspartic acid (D) at position 293 and a Glutamic acid I at position 332; and
vi) an Aspartic acid (D) at position 293, a Leucine (L) at position 330, and a Glutamic acid I at position 332.

In some embodiments, the Fc region is afucosylated.

In some embodiments, the Fc region is an Fc region of human IgG1, wherein the Fc region comprises an Alanine (A) at position 236, a Leucine (L) at position 330, and a Glutamic acid I at position 332, wherein the Fc region comprises an amino acid sequence that is at least 80% identical to SEQ ID NO: 43 (the numbering is according to EU numbering).

In some embodiments, the NK cell surface antigen is selected from NKp46, NKp30, CD16, NKG2D and NKG2A.

In some embodiments, the NK cell surface antigen is NKp46.

In some embodiments, the tumor associated antigen (TAA) is CD79b, EGFR, EpCAM, B-cell maturation antigen (BCMA), DLL3, PSMA, STEAP1, MUC1, c-MET, LRRC15, CCR8, OX40, perform, granzyme B, Fas ligand (FasL), CDId, Membrane glycolipids, globotriaosyl-ceramide (Gb3Cer/CD77), gangliosides (GD2. GD3, and GM2), CD34, CD45, human leukocyte antigen-DR (HLA-DR), CD123, CD38, CLL1, CD105, CD71, SSC, MAGE, MUC16, CD19, WT-1, B7H3, TEM8, CD22, LI-CAM, ROR-1, CEA, 4-1BB, ETA, 5T4, adenocarcinoma antigen, alpha-fetoprotein (AFP), BAFF, B-lymphoma cell, C242 antigen. CA-125, carbonic anhydrase 9 (CA-IX), CCR4, CD133, CD152, CD20, CD125, CD200, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, IL-5, IL-13, IL-6, IL-15, insulin-like growth factor I receptor, integrin a5b1, integrin anb3, MORAb-009, MS4A1, mucin CanAg, N-glycolylneuraminic acid, NPC-1C, PD-1, PD-L1, PDGF-R a, PDL192, phosphatidylserine, prostatic carcinoma cells, RANKL, RON, SCH 900105, SDC1, SLAMF7, TAG-72, tenascin C, TGF beta 2, TGF-b, TRAIL-R1, TRAIL-R2, tumor antigen CTAA16.88, VEGF-A, VEGFR-1, VEGFR2, or vimentin.

In some embodiments, the first antigen is CD79b; and the second antigen is NKp46.

In some embodiments, the first heavy chain variable region (VH1) comprises complementarity determining regions (CDRs) 1, 2, and 3, wherein the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and the first light chain variable region (VL1) comprises CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence, wherein the selected VH1 CDRs 1, 2, and 3 amino acid sequences, and the selected VL1 CDRs 1, 2, and 3 amino acid sequences are one of the following:
(1) the selected VH1 CDRs 1, 2, 3 amino acid sequences are set forth in SEQ ID NOs: 49, 51 and 53, respectively, and the selected VL1 CDRs 1, 2, 3 amino acid sequences are set forth in SEQ ID NOs: 54, 55 and 56, respectively; and
(2) the selected VH1 CDRs 1, 2, 3 amino acid sequences are set forth in SEQ ID NOs: 50, 52 and 53, respectively, and the selected VL1 CDRs 1, 2, 3 amino acid sequences are set forth in SEQ ID NOs: 54, 55 and 56, respectively, and
(3) the selected VH1 CDRs 1, 2, 3 amino acid sequences and the selected VL1 CDRs 1, 2, 3 amino acid sequences are the corresponding VH CDRs 1, 2, 3 and VL CDRs 1, 2, 3 amino acid sequences of Polatuzumab (Genentech Inc.). PRV-3279 (MacroGenics Inc) or SHR-A1912 (Jiangsu Hengrui Pharmaceuticals Co., Ltd.).

In some embodiments, the second heavy chain variable region (VH2) comprises CDRs 1, 2, and 3, wherein the VH2 CDR1 region comprises an amino acid sequence that is at least 80% identical to a selected VH2 CDR1 amino acid sequence, the VH2 CDR2 region comprises an amino acid sequence that is at least 80% identical to a selected VH2 CDR2 amino acid sequence, and the VH2 CDR3 region comprises an amino acid sequence that is at least 80% identical to a selected VH2 CDR3 amino acid sequence; and the second light chain variable region (VL2) comprises CDRs 1, 2, and 3, wherein the VL2 CDR1 region comprises an amino acid sequence that is at least 80% identical to a selected VL2 CDR1 amino acid sequence, the VL2 CDR2 region comprises an amino acid sequence that is at least 80% identical to a selected VL2 CDR2 amino acid sequence, and the VL2 CDR3 region comprises an amino acid sequence that is at least 80% identical to a selected VL2 CDR3 amino acid sequence, wherein the selected VH2 CDRs 1, 2, and 3 amino acid sequences, and the selected VL2 CDRs 1, 2, and 3 amino acid sequences are one of the following:
(1) the selected VH2 CDRs 1, 2, 3 amino acid sequences are set forth in SEQ ID NOs: 5, 7, and 9, respectively, and the selected VL2 CDRs 1, 2, 3 amino acid sequences are set forth in SEQ ID NOs: 10, 11, and 12, respectively;
(2) the selected VH2 CDRs 1, 2, 3 amino acid sequences are set forth in SEQ ID NOs: 6, 8, and 9, respectively, and the selected VL2 CDRs 1, 2, 3 amino acid sequences are set forth in SEQ ID NOs: 10, 11 and 12, respectively;
(3) the selected VH2 CDRs 1, 2, 3 amino acid sequences are set forth in SEQ ID NOs: 15, 17, and 19, respectively, and the selected VL2 CDRs 1, 2, 3 amino acid sequences are set forth in SEQ ID NOs: 20, 21, and 22, respectively;
(4) the selected VH2 CDRs 1, 2, 3 amino acid sequences are set forth in SEQ ID NOs: 16, 18, and 19, respectively, and the selected VL2 CDRs 1, 2, 3 amino acid sequences are set forth in SEQ ID NOs: 20, 21 and 22, respectively;
(5) the selected VH2 CDRs 1, 2, 3 amino acid sequences are set forth in SEQ ID NOs: 25, 27, and 29, respectively, and the selected VL2 CDRs 1, 2, 3 amino acid sequences are set forth in SEQ ID NOs: 30, 31, and 32, respectively; and
(6) the selected VH2 CDRs 1, 2, 3 amino acid sequences are set forth in SEQ ID NOs: 26, 28, and 29, respectively, and the selected VL2 CDRs 1, 2, 3 amino acid sequences are set forth in SEQ ID NOs: 30, 31 and 32, respectively.

In some embodiments, (1) the selected VH1 CDRs 1, 2, 3 amino acid sequences are set forth in SEQ ID NOs: 49, 51, and 53, respectively, and the selected VL1 CDRs 1, 2, 3 amino acid sequences are set forth in SEQ ID NOs: 54, 55, and 56, respectively; and the selected VH2 CDRs 1, 2, 3 amino acid sequences are set forth in SEQ ID NOs: 5, 7, and 9, respectively, and the selected VL2 CDRs 1, 2, 3 amino acid sequences are set forth in SEQ ID NOs: 10, 11, and 12, respectively;
(2) the selected VH1 CDRs 1, 2, 3 amino acid sequences are set forth in SEQ ID NOs: 50, 52, and 53, respectively, and the selected VL1 CDRs 1, 2, 3 amino acid sequences are set forth in SEQ ID NOs: 54, 55, and 56, respectively; and the selected VH2 CDRs 1, 2, 3 amino acid sequences are set forth in SEQ ID NOs: 6, 8, and 9, respectively, and the selected VL2 CDRs 1, 2, 3 amino acid sequences are set forth in SEQ ID NOs: 10, 11 and 12, respectively;
(3) the selected VH1 CDRs 1, 2, 3 amino acid sequences are set forth in SEQ ID NOs: 49, 51, and 53, respectively, and the selected VL1 CDRs 1, 2, 3 amino acid sequences are set forth in SEQ ID NOs: 54, 55, and 56, respectively; and the selected VH2 CDRs 1, 2, 3 amino acid sequences are set forth in SEQ ID NOs: 15, 17, and 19, respectively, and the selected VL2 CDRs 1, 2, 3 amino acid sequences are set forth in SEQ ID NOs: 20, 21, and 22, respectively;
(4) the selected VH1 CDRs 1, 2, 3 amino acid sequences are set forth in SEQ ID NOs: 50, 52, and 53, respectively, and the selected VL1 CDRs 1, 2, 3 amino acid sequences are set forth in SEQ ID NOs: 54, 55, and 56, respectively; and the selected VH2 CDRs 1, 2, 3 amino acid sequences are set forth in SEQ ID NOs: 16, 18, and 19, respectively, and the selected VL2 CDRs 1, 2, 3 amino acid sequences are set forth in SEQ ID NOs: 20, 21 and 22, respectively;
(5) the selected VH1 CDRs 1, 2, 3 amino acid sequences are set forth in SEQ ID NOs: 49, 51, and 53, respectively, and the selected VL1 CDRs 1, 2, 3 amino acid sequences are set forth in SEQ ID NOs: 54, 55, and 56, respectively; and the selected VH2 CDRs 1, 2, 3 amino acid sequences are set forth in SEQ ID NOs: 25, 27, and 29, respectively, and the selected VL2 CDRs 1, 2, 3 amino acid sequences are set forth in SEQ ID NOs: 30, 31, and 32, respectively; or
(6) the selected VH1 CDRs 1, 2, 3 amino acid sequences are set forth in SEQ ID NOs: 50, 52, and 53, respectively, and the selected VL1 CDRs 1, 2, 3 amino acid sequences are set forth in SEQ ID NOs: 54, 55, and 56, respectively; and the selected VH2 CDRs 1, 2, 3 amino acid sequences are set forth in SEQ ID NOs: 26, 28, and 29, respectively, and the selected VL2 CDRs 1, 2, 3 amino acid sequences are set forth in SEQ ID NOs: 30, 31 and 32, respectively.

In some embodiments, the first antigen is BCMA; and the second antigen is NKp46.

In some embodiments, the first heavy chain variable region (VH1) comprises complementarity determining regions (CDRs) 1, 2, and 3, wherein the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and
the first light chain variable region (VL1) comprises CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence, wherein the selected VH1 CDRs 1, 2, and 3 amino acid sequences, and the selected VL1 CDRs 1, 2, and 3 amino acid sequences are as following:
   (1) the selected VH1 CDRs 1, 2, 3 amino acid sequences are set forth in SEQ ID NOs: 88, 89 and 90, respectively, and the selected VL1 CDRs 1, 2, 3 amino acid sequences are set forth in SEQ ID NOs: 91, 92 and 93, respectively.
   (2) the selected VH1 CDRs 1, 2, 3 amino acid sequences and the selected VL1 CDRs 1, 2, 3 amino acid sequences are from the corresponding VH CDRs 1, 2, 3 and VL CDRs 1, 2, 3 sequences of Zevorcabtagene Autoleucel (CARsgen Therapeutics Co. Ltd.), Elranatamab (Pfizer Inc.), Teclistamab (Janssen Pharmaceutica NV), Equecabtagene Autoleuce (Nanjing IASO Biotherapeutics Co., Ltd.), Ciltacabtagene autoleucel (Nanjing Legend Biotech Co., Ltd.), Idecabtagene Vicleucel (Bluebird bio Inc.), Belantamab (GSK Plc), Linvoseltamab (Regeneron Pharmaceuticals Inc.), Alnuctamab (Celgene Corp.), TNB-383B (TeneoBio Inc.) or SEA-BCMA (Seagen Inc.).

In some embodiments, the second heavy chain variable region (VH2) comprises CDRs 1, 2, and 3, wherein the VH2 CDR1 region comprises an amino acid sequence that is at least 80% identical to a selected VH2 CDR1 amino acid sequence, the VH2 CDR2 region comprises an amino acid sequence that is at least 80% identical to a selected VH2 CDR2 amino acid sequence, and the VH2 CDR3 region comprises an amino acid sequence that is at least 80% identical to a selected VH2 CDR3 amino acid sequence; and
the second light chain variable region (VL2) comprises CDRs 1, 2, and 3, wherein the VL2 CDR1 region comprises an amino acid sequence that is at least 80% identical to a selected VL2 CDR1 amino acid sequence, the VL2 CDR2 region comprises an amino acid sequence that is at least 80% identical to a selected VL2 CDR2 amino acid sequence, and the VL2 CDR3 region comprises an amino acid sequence that is at least 80% identical to a selected VL2 CDR3 amino acid sequence, wherein the selected VH2 CDRs 1, 2, and 3 amino acid sequences, and the selected VL2 CDRs 1, 2, and 3 amino acid sequences are as following:
the selected VH2 CDRs 1, 2, 3 amino acid sequences are set forth in SEQ ID NOs: 25, 27 and 29, respectively, and the selected VL2 CDRs 1, 2, 3 amino acid sequences are set forth in SEQ ID NOs: 30, 31, and 32, respectively.

In some embodiments, the selected VH1 CDRs 1, 2, 3 amino acid sequences are set forth in SEQ ID NOs: 88, 89 and 90, respectively, and the selected VL1 CDRs 1, 2, 3 amino acid sequences are set forth in SEQ ID NOs: 91, 92 and 93, respectively; and the selected VH2 CDRs 1, 2, 3 amino acid sequences are set forth in SEQ ID NOs: 25, 27 and 29, respectively, and the selected VL2 CDRs 1, 2, 3 amino acid sequences are set forth in SEQ ID NOs: 30, 31, and 32, respectively.

In some embodiments, the first antigen is EGFR; and the second antigen is NKp46.

In some embodiments, the first heavy chain variable region (VH1) comprises complementarity determining regions (CDRs) 1, 2, and 3, wherein the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and
the first light chain variable region (VL1) comprises CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence,
wherein the selected VH1 CDRs 1, 2, and 3 amino acid sequences, and the selected VL1 CDRs 1, 2, and 3 amino acid sequences are as following:
   (1) the selected VH1 CDRs 1, 2, 3 amino acid sequences are set forth in SEQ ID NOs: 94, 95 and 96, respectively, and the selected VL1 CDRs 1, 2, 3 amino acid sequences are set forth in SEQ ID NOs: 97, 98 and 99, respectively.
   (2) the selected VH1 CDRs 1, 2, 3 amino acid sequences and the selected VL1 CDRs 1, 2, 3 amino acid sequences are from the corresponding VH CDRs 1, 2, 3 and VL CDRs 1, 2, 3 amino acid sequences of Amivantamab (Janssen Biotech Inc.), Cetuximab (Merck Serono SA), Necitumumab (Eli Lilly & Co.), Panitumumab (Amgen Inc.), Nimotuzumab (Center For Molecular Immunology), BL-B01D1 (Sichuan Baili Pharmaceuticals Co., Ltd), Demupitamab (Sinocelltech Group Ltd.), Depatuxizumab (Ludwig Institute for Cancer Research Ltd.), Futuximab (Merck KGaA), Modotuximab (Merck KGaA), JMT-101 (Shanghai JMT Biological Technology Co Ltd), MRG-003 (Shanghai Miracogen Inc.), Pimurutamab (Shanghai Henlius Biotech Inc.), QL1203 (Qilu Pharmaceutical Co., Ltd.), SI-B001 (Sichuan Baili Pharmaceuticals Co., Ltd) or Ametumumab (Academy of Military Medical Sciences).

In some embodiments, the second heavy chain variable region (VH2) comprises CDRs 1, 2, and 3, wherein the VH2 CDR1 region comprises an amino acid sequence that is at least 80% identical to a selected VH2 CDR1 amino acid sequence, the VH2 CDR2 region comprises an amino acid sequence that is at least 80% identical to a selected VH2 CDR2 amino acid sequence, and the VH2 CDR3 region comprises an amino acid sequence that is at least 80% identical to a selected VH2 CDR3 amino acid sequence; and the second light chain variable region (VL2) comprises CDRs 1, 2, and 3, wherein the VL2 CDR1 region comprises an amino acid sequence that is at least 80% identical to a selected VL2 CDR1 amino acid sequence, the VL2 CDR2 region comprises an amino acid sequence that is at least 80% identical to a selected VL2 CDR2 amino acid sequence, and the VL2 CDR3 region comprises an amino acid sequence that is at least 80% identical to a selected VL2 CDR3 amino acid sequence,
wherein the selected VH2 CDRs 1, 2, and 3 amino acid sequences, and the selected VL2 CDRs 1, 2, and 3 amino acid sequences are as following:
the selected VH2 CDRs 1, 2, 3 amino acid sequences are set forth in SEQ ID NOs: 25, 27 and 29, respectively, and the selected VL2 CDRs 1, 2, 3 amino acid sequences are set forth in SEQ ID NOs: 30, 31, and 32, respectively.

In some embodiments, the selected VH1 CDRs 1, 2, 3 amino acid sequences are set forth in SEQ ID NOs: 94, 95 and 96, respectively, and the selected VL1 CDRs 1, 2, 3 amino acid sequences are set forth in SEQ ID NOs: 97, 98 and 99, respectively; and the selected VH2 CDRs 1, 2, 3 amino acid sequences are set forth in SEQ ID NOs: 25, 27 and 29, respectively, and the selected VL2 CDRs 1, 2, 3 amino acid sequences are set forth in SEQ ID NOs: 30, 31, and 32, respectively.

In some embodiments, the first antigen is EpCAM; and the second antigen is NKp46.

In some embodiments, the first heavy chain variable region (VH1) comprises complementarity determining regions (CDRs) 1, 2, and 3, wherein the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and
the first light chain variable region (VL1) comprises CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence,
wherein the selected VH1 CDRs 1, 2, and 3 amino acid sequences, and the selected VL1 CDRs 1, 2, and 3 amino acid sequences are as following:
   (1) the selected VH1 CDRs 1, 2, 3 amino acid sequences are set forth in SEQ ID NOs: 100, 101 and 102, respectively, and the selected VL1 CDRs 1, 2, 3 amino acid sequences are set forth in SEQ ID NOs: 103, 104 and 105, respectively;
   (2) the selected VH1 CDRs 1, 2, 3 amino acid sequences and the selected VL1 CDRs 1, 2, 3 amino acid sequences are the corresponding VH CDRs 1, 2, 3 and VL CDRs 1, 2, 3 amino acid sequences of Catumaxomab (Neovii Biotech GmbH), M-701 (Wuhan YZY Biopharma Co., Ltd.), Adecatumumab (Amgen Inc.), Edrecolomab (GSK Plc), Tefibazumab (Bristol Myers Squibb Co.), VB1-008 (Sesen Bio Inc.) or VB1-050 (Sesen Bio Inc.).

In some embodiments, the second heavy chain variable region (VH2) comprises CDRs 1, 2, and 3, wherein the VH2 CDR1 region comprises an amino acid sequence that is at least 80% identical to a selected VH2 CDR1 amino acid sequence, the VH2 CDR2 region comprises an amino acid sequence that is at least 80% identical to a selected VH2 CDR2 amino acid sequence, and the VH2 CDR3 region comprises an amino acid sequence that is at least 80% identical to a selected VH2 CDR3 amino acid sequence; and
the second light chain variable region (VL2) comprises CDRs 1, 2, and 3, wherein the VL2 CDR1 region comprises an amino acid sequence that is at least 80% identical to a selected VL2 CDR1 amino acid sequence, the VL2 CDR2 region comprises an amino acid sequence that is at least 80% identical to a selected VL2 CDR2 amino acid sequence, and the VL2 CDR3 region comprises an amino acid sequence that is at least 80% identical to a selected VL2 CDR3 amino acid sequence, wherein the selected VH2 CDRs 1, 2, and 3 amino acid sequences, and the selected VL2 CDRs 1, 2, and 3 amino acid sequences are as following:
the selected VH2 CDRs 1, 2, 3 amino acid sequences are set forth in SEQ ID NOs: 25, 27 and 29, respectively, and the selected VL2 CDRs 1, 2, 3 amino acid sequences are set forth in SEQ ID NOs: 30, 31, and 32, respectively.

In some embodiments, the selected VH1 CDRs 1, 2, 3 amino acid sequences are set forth in SEQ ID NOs: 100, 101 and 102, respectively, and the selected VL1 CDRs 1, 2, 3 amino acid sequences are set forth in SEQ ID NOs: 103, 104 and 105, respectively; and the selected VH2 CDRs 1, 2, 3 amino acid sequences are set forth in SEQ ID NOs: 25, 27 and 29, respectively, and the selected VL2 CDRs 1, 2, 3 amino acid sequences are set forth in SEQ ID NOs: 30, 31, and 32, respectively.

In some embodiments, the first antigen is DLL3; and the second antigen is NKp46.

In some embodiments, the first heavy chain variable region (VH1) comprises complementarity determining regions (CDRs) 1, 2, and 3, wherein the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and
the first light chain variable region (VL1) comprises CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence,
wherein the selected VH1 CDRs 1, 2, and 3 amino acid sequences, and the selected VL1 CDRs 1, 2, and 3 amino acid sequences are the corresponding VH CDRs 1, 2, 3 and VL CDRs 1, 2, 3 amino acid sequences of Rovalpituzumab tesirine (AbbVie Inc.), Tarlatamab (Amgen Inc.), HPN328 (Harpoon Therapeutics Inc.) or BI 764532 (Boehringer Ingelheim GmbH).

In some embodiments, the first antigen is PSMA; and the second antigen is NKp46.

In some embodiments, the first heavy chain variable region (VH1) comprises complementarity determining regions (CDRs) 1, 2, and 3, wherein the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and
the first light chain variable region (VL1) comprises CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence,
wherein the selected VH1 CDRs 1, 2, and 3 amino acid sequences, and the selected VL1 CDRs 1, 2, and 3 amino acid sequences are the corresponding VH CDRs 1, 2, 3 and VL CDRs 1, 2, 3 amino acid sequences of HPN-424 (Harpoon Therapeutics Inc.), LAVA-1207 (LAVA Therapeutics NV), REGN-5678 (Regeneron Pharmaceuticals Inc.), REGN4336 (Regeneron Pharmaceuticals Inc.), Acapatamab (Amgen Inc.) or AMG-340 (Amgen Inc.).

In some embodiments, the first antigen is STEAP1; and the second antigen is NKp46.

In some embodiments, the first heavy chain variable region (VH1) comprises complementarity determining regions (CDRs) 1, 2, and 3, wherein the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and
the first light chain variable region (VL1) comprises CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence,
wherein the selected VH1 CDRs 1, 2, and 3 amino acid sequences, and the selected VL1 CDRs 1, 2, and 3 amino acid sequences are the corresponding VH CDRs 1, 2, 3 and VL CDRs 1, 2, 3 amino acid sequences of Xaluritamig (Amgen Inc.), ABBV-969 (AbbVie Inc.) or Vandortuzumab vedotin (Genentech Inc.).

In some embodiments, the first antigen is MUC1; and the second antigen is NKp46.

In some embodiments, the first heavy chain variable region (VH1) comprises complementarity determining regions (CDRs) 1, 2, and 3, wherein the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and
the first light chain variable region (VL1) comprises CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence,
wherein the selected VH1 CDRs 1, 2, and 3 amino acid sequences, and the selected VL1 CDRs 1, 2, and 3 amino acid sequences are the corresponding VH CDRs 1, 2, 3 and VL CDRs 1, 2, 3 amino acid sequences of MAB-AR20.5 (Canariabio Inc.) or DS-3939 (Glycotope GmbH).

In some embodiments, the first antigen is c-MET; and the second antigen is NKp46.

In some embodiments, the first heavy chain variable region (VH1) comprises complementarity determining regions (CDRs) 1, 2, and 3, wherein the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and
the first light chain variable region (VL1) comprises CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence,
   wherein the selected VH1 CDRs 1, 2, and 3 amino acid sequences, and the selected VL1 CDRs 1, 2, and 3 amino acid sequences are the corresponding VH CDRs 1, 2, 3 and VL CDRs 1, 2, 3 amino acid sequences of Amivantamab-VMJM (Janssen Biotech Inc.), Telisotuzumab vedotin (AbbVie Inc.), ABBV-400 (AbbVie Inc.), Bafisontamab (EpimAb Biotherapeutics Inc.), CKD-702 (Chong Kun Dang Pharmaceutical Corp.), Emibetuzumab (Eli Lilly & Co.), RC-108 (RemeGen Co., Ltd.), MCLA-129 (Merus NV), REGN-5093 (Regeneron Pharmaceuticals Inc.) or REGN5093-M114 (Regeneron Pharmaceuticals Inc.).

In some embodiments, the first antigen is LRRC15; and the second antigen is NKp46.

In some embodiments, the first heavy chain variable region (VH1) comprises complementarity determining regions (CDRs) 1, 2, and 3, wherein the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80)% identical to a selected VH1 CDR3 amino acid sequence; and
the first light chain variable region (VL1) comprises CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence,
wherein the selected VH1 CDRs 1, 2, and 3 amino acid sequences, and the selected VL1 CDRs 1, 2, and 3 amino acid sequences are the corresponding VH CDRs 1, 2, 3 and VL CDRs 1, 2, 3 amino acid sequences of QL-315 (Qilu Pharmaceutical Co., Ltd.), RAD-502 (Radiopharm Theranostics Ltd.) or Samrotamab (AbbVie Inc.).

In some embodiments, the first antigen is CCR8; and the second antigen is NKp46.

In some embodiments, the first heavy chain variable region (VH1) comprises complementarity determining regions (CDRs) 1, 2, and 3, wherein the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence, and
the first light chain variable region (VL1) comprises CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence,
wherein the selected VH1 CDRs 1, 2, and 3 amino acid sequences, and the selected VL1 CDRs 1, 2, and 3 amino acid sequences are the corresponding VH CDRs 1, 2, 3 and VL CDRs 1, 2, 3 amino acid sequences of BMS-986340 (Bristol Myers Squibb Co.), LM-108 (LaNova Medicines Ltd.), S-531011 (Shionogi & Co., Ltd.), AMG-355 (Amgen Inc.), BAY3375968 (Bayer AG), BGB-A3055 (BeiGene Ltd.), CM369 (Keymed Biomedical Technology (Chengdu) Co., Ltd.), HBM-1022 (Harbour BioMed (Shanghai) Co., Ltd.), PSB-114 (Qilu Puget Sound Biotherapeutics Corp.) or SRF-114 (Surface Oncology Inc.).

In some embodiments, the first antigen is OX40; and the second antigen is NKp46.

In some embodiments, the first heavy chain variable region (VH1) comprises complementarity determining regions (CDRs) 1, 2, and 3, wherein the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and
the first light chain variable region (VL1) comprises CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence,
wherein the selected VH1 CDRs 1, 2, and 3 amino acid sequences, and the selected VL1 CDRs 1, 2, and 3 amino acid sequences are the corresponding VH CDRs 1, 2, 3 and VL CDRs 1, 2, 3 amino acid sequences of Amlitelimab (Kymab Ltd.), Rocatinlimab (Kyowa Kirin Co., Ltd.), INBRX-106 (Tnhibrx Inc.), BGB-A445 (BeiGene Ltd.), INCAGN-1949, Ivuxolimab (Pfizer Inc.), MEDI-6469 (Providence Cancer Center), BAT-6026 (Bio-Thera Solutions, Ltd.), IMG-007 (HUTCHMED (China) Ltd.), ABBV-368 (AbbVie Inc.) or BMS-986178 (Bristol Myers Squibb Co.).

In some embodiments, the Fc region has increased complement-dependent cytotoxicity (CDC), increased antibody-dependent cellular cytotoxicity (ADCC) or increased half-life.

In one aspect, provided herein are antibodies or antigen-binding fragments thereof that cross-competes with any one of the antibodies or antigen-binding fragments thereof.

In one aspect, provided herein are methods of treating a subject having cancer, the method comprising administering a therapeutically effective amount of a composition comprising any one of the antibodies or antigen-binding fragments thereof described herein to the subject.

In some embodiments, the cancer is a solid tumor or a blood tumor.

In some embodiments, the cancer is the cancer is breast cancer, lung cancer, stomach cancer, colorectal cancer, prostate cancer, ovarian cancer, colon cancer, esophageal cancer, tracheal cancer, gastric cancer bladder cancer, uterine cancer, rectal cancer, cancer of the small intestine, pancreatic cancer and liver cancer.

In some embodiments, the cancer is multiple myeloma, B-cell lymphoma, diffuse Large B-Cell Lymphoma, acute B-cell leukemia, chronic lymphocytic leukemia, B-cell prelymphocytic leukemia, spleen with villous lymphocytes Lymphoma, hairy cell leukemia, follicular lymphoma, and mantle cell lymphoma

In some embodiments, the subject is further treated with an effective amount of an anti-4-1BB antibody, an anti-OX40 antibody, an anti-PD-1 antibody, an anti-CTLA4 antibody, an anti-CD40 antibody, or an anti-PD-L1 antibody.

In one aspect, provided herein are methods of decreasing the rate of tumor growth, the method comprising contacting a tumor cell with an effective amount of a composition comprising an antibody or antigen-binding fragment thereof described herein.

In one aspect, provided herein are methods of killing a tumor cell, the method comprising contacting a tumor cell with an effective amount of a composition comprising any one of the antibodies or antigen-binding fragments thereof described herein.

In one aspect, provided herein are methods of increasing immune response in a subject, the method comprising administering to the subject an effective amount of a composition comprising any one of the antibodies or antigen-binding fragments thereof described herein.

In one aspect, provided herein are methods of treating a subject having autoimmune disease, the method comprising administering a therapeutically effective amount of a composition comprising the antibody or antigen-binding fragment thereof described herein.

In one aspect, the autoimmune disease is selected from rheumatoid arthritis, psoriasis, multiple sclerosis, immune thrombocytopenic purpura, myasthenia gravis, neuromyelitis optica, IgG4-related diseases, systemic Lupus Erythematosus, lupus nephritis, giant cell arteritis, takayasu disease, cold agglutinin disease, warm autoimmune hemolytic anemia, and anti-neutrophil cytoplasmic antibody (ANCA) associated vasculitides including for example, tranulomatosis with polyangiitis (GPA) (Wegener's Granulomatosis) or Microscopic Polyangiitis (MPA).

In one aspect, the autoimmune disease is multiple sclerosis, systemic lupus erythematosus or rheumatoid arthritis.

In one aspect, provided herein are pharmaceutical compositions comprising any one of the antibodies or antigen-binding fragments thereof described herein, and a pharmaceutically acceptable carrier.

As used herein, the term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as head and neck, respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, glioma and cancer of the small intestine. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. The term “hematopoietic neoplastic disorders” includes diseases involving hyperplastic/neoplastic cells of hematopoietic origin. A hematopoietic neoplastic disorder can arise from myeloid, lymphoid or erythroid lineages, or precursor cells thereof. A hematologic cancer is a cancer that begins in blood-forming tissue, such as the bone marrow, or in the cells of the immune system. Examples of hematologic cancer include e.g., leukemia, lymphoma, and multiple myeloma etc.

As used herein, a “autoimmune disease” is a condition arising from an abnormal immune response to a functioning body part. Symptoms of autoimmune diseases are diverse and transient, ranging from mild to severe, and generally include low grade fever and fatigue. There are various causes of autoimmune diseases. Some autoimmune diseases such as lupus run in families, and certain cases may be triggered by infections or other environmental factors. Some common diseases that are generally considered autoimmune include celiac disease, diabetes mellitus type 1, graves' disease, inflammatory bowel disease, multiple sclerosis, alopecia areata, addison's disease, pernicious anemia, psoriasis, rheumatoid arthritis, and systemic lupus erythematosus.

As used herein, the term “antigen-binding domain” refers to one or more protein domain(s) (e.g., formed from amino acids from a single polypeptide or formed from amino acids from two or more polypeptides (e.g., the same or different polypeptides) that is capable of specifically binding to one or more different antigen(s) (e.g., an effector antigen or tumor antigen). In some examples, an antigen-binding domain can bind to an antigen or epitope with specificity and affinity similar to that of naturally-occurring antibodies. In some embodiments, the antigen-binding domain can be an antibody or a fragment thereof. One example of an antigen-binding domain is an antigen-binding domain formed by VH-VL. In some embodiments, an antigen-binding domain can include an alternative scaffold. In some embodiments, the antigen-binding domain is a VHH. Non-limiting examples of antigen-binding domains are described herein. Additional examples of antigen-binding domains are known in the art. In some examples, an antigen-binding domain can bind to a single antigen (e.g., one of an effector antigen and a tumor antigen).

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an antigen. Non-limiting examples of antibodies include, monoclonal antibodies, polyclonal antibodies, multi-specific antibodies (e.g., bi-specific antibodies), single-chain antibodies, chimeric antibodies, human antibodies, and humanized antibodies. In some embodiments, an antibody can contain an Fc region of a human antibody. The term antibody also includes derivatives, e.g., bi-specific antibodies, single-chain antibodies, diabodies, linear antibodies, and multi-specific antibodies formed from antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain or a variable domain of light chain). Non-limiting examples of antibody fragments include, e.g., Fab, Fab′, F(ab′)2, and Fv fragments.

As used herein, the term “multispecific antibody” is an antibody that includes two or more different antigen-binding domains that collectively specifically bind two or more different epitopes. The two or more different epitopes may be epitopes on the same antigen (e.g., a single polypeptide present on the surface of a cell) or on different antigens (e.g., different proteins present on the surface of the same cell or present on the surface of different cells). In some aspects, a multispecific antibody binds two different epitopes (i.e., a “bispecific antibody”). In some aspects, a multispecific antibody binds three different epitopes (i.e., a “trispecific antibody”). In some aspects, a multispecific antibody binds four different epitopes (i.e., a “quadspecific antibody”). In some aspects, a multispecific antibody binds five different epitopes (i.e., a “quintspecific antibody”). Each binding specificity may be present in any suitable valency. Non-limiting examples of multispecific antibodies are described herein.

As used herein, the term “bispecific antibody” refers to an antibody that binds to two different epitopes. The epitopes can be on the same antigen or on different antigens.

As used herein, the term “chimeric antibody” refers to an antibody that contains a sequence present in at least two different species (e.g., antibodies from two different mammalian species such as a human and a mouse antibody or a human and a rabbit antibody). A non-limiting example of a chimeric antibody is an antibody containing the variable domain sequences (e.g., all or part of a light chain and/or heavy chain variable domain sequence) of a non-human (e.g., mouse or rabbit) antibody and the constant domains of a human antibody. Additional examples of chimeric antibodies are described herein and are known in the art.

As used herein, the term “humanized antibody” refers to a non-human antibody which contains minimal sequence derived from a non-human (e.g., mouse, or rabbit) immunoglobulin and contains sequences derived from a human immunoglobulin. In non-limiting examples, humanized antibodies are human antibodies (recipient antibody) in which hypervariable (e.g., CDR) region residues of the recipient antibody are replaced by hypervariable (e.g., CDR) region residues from a non-human antibody (e.g., a donor antibody), e.g., a mouse, rat, or rabbit antibody, having the desired specificity, affinity, and capacity. In some embodiments, the Fv framework residues of the human immunoglobulin are replaced by corresponding non-human (e.g., mouse or rabbit) immunoglobulin residues. In some embodiments, humanized antibodies may contain residues which are not found in the recipient antibody or in the donor antibody. These modifications can be made to further refine antibody performance. In some embodiments, the humanized antibody contains substantially all of at least one, and typically two, variable domains, in which all or substantially all of the hypervariable loops (CDRs) correspond to those of a non-human (e.g., mouse or rabbit) immunoglobulin and all or substantially all of the framework regions are those of a human immunoglobulin. The humanized antibody can also contain at least a portion of an immunoglobulin constant region (Fc), typically, that of a human immunoglobulin. Humanized antibodies can be produced using molecular biology methods known in the art. Non-limiting examples of methods for generating humanized antibodies are described herein.

As used herein, the term “single-chain antibody” refers to a single polypeptide that contains at least two immunoglobulin variable domains (e.g., a variable domain of a mammalian immunoglobulin heavy chain or light chain) that is capable of specifically binding to an antigen. Non-limiting examples of single-chain antibodies are described herein.

As used herein, the terms “subject” and “patient” are used interchangeably throughout the specification and describe an animal, human or non-human, to whom treatment according to the methods of the present invention is provided. Veterinary and non-veterinary applications are contemplated in the present disclosure. Human patients can be adult humans or juvenile humans (e.g., humans below the age of 18 years old). In addition to humans, patients include but are not limited to mice, rats, hamsters, guinea pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, when referring to an antibody, the phrases “specifically binding” and “specifically binds” mean that the antibody interacts with its target molecule (e.g., NKp46) preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to a NKp46 molecule may be referred to as a NKp46-specific antibody or an anti-NKp46 antibody.

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

As used herein, the term “NK cell surface antigen” refers to a molecule, e.g., a surface receptor on an NK cell, that is involved in the regulation and/or induction of NK cell functions. In some embodiments, an NK cell surface antigen is an NK cell activating receptor.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 shows the affinity of anti-NKp46 antibodies to human NKp46.

FIGS. 2A-2C show ELISA binding of chimeric and humanized anti-NKp46 antibodies.

FIGS. 3A-3C show BLI binding of chimeric and humanized anti-NKp46 antibodies.

FIGS. 4A-4B are schematic illustrations of examples of bispecific antibodies described herein.

FIG. 5 shows the activation of NK cells by CD79b/NKp46 bispecific antibodies.

FIG. 6 shows the binding abilities of CD79b/NKp46 bispecific antibodies with silent Fc to effector NK cells.

FIG. 7 shows the binding abilities of CD79b/NKp46 bispecific antibodies with silent Fc to target Ramos cells.

FIG. 8 shows CD79b/NKp46 bispecific antibodies with silenced Fc mediate target cancer B cell killing by NK cells.

FIG. 9 shows CD79b/NKp46 bispecific antibodies with wild type Fc mediate Ramos cell killing by NK cells.

FIG. 10 shows CD79b/NKp46 bispecific antibodies with wild type Fc mediate Daudi cell killing by NK cells.

FIGS. 11A-11D show CD79b/NKp46 bispecific antibodies with wild type Fc mediate endogenous B cell killing by PBMC.

FIGS. 12A-12B show the activation of NK cells by CD79b/NKp46 bispecific antibodies with optimized Fc.

FIG. 13 shows the activation of NK cells by CD79b/NKp46 bispecific antibodies with optimized Fc and CD79b antibodies with optimized Fc.

FIGS. 14A-14B show CD79b/NKp46 bispecific antibodies with optimized Fc mediate Ramos cell killing by NK cells.

FIG. 15 shows CD79b/NKp46 bispecific antibodies with optimized Fc mediate Ramos cell killing by PBMC.

FIG. 16 shows CD79b/NKp46 bispecific antibodies with optimized Fc mediate Nalm-6 cell killing by PBMC.

FIGS. 17A-17B show NK cell engagers with optimized Fc mediate Ramos cell killing by PBMC.

FIGS. 18A-18C show cytokine release activity of NK cell engagers with optimized Fc.

FIG. 19 shows NK cell engagers with optimized Fc mediated Nalm-6 cell killing by PBMC.

FIG. 20 shows the numbers of NK cell during target cell killing by CD79b/NKp46 bispecific antibodies with optimized Fc.

FIG. 21 shows the numbers of monocytes during target cell killing by CD79b/NKp46 bispecific antibodies with optimized Fc.

FIGS. 22A-22B show NK Cell engagers with optimized Fc mediate endogenous B cell killing by PBMC (test 1).

FIG. 23 shows NK Cell engagers with optimized Fc mediate endogenous B cell killing by PBMC (test 2).

FIGS. 24A-24B show cytokine release activity of NK cell engagers with optimized Fc.

FIGS. 25A-25D show NK cell engagers mediated target cancer B cell killing by monocytes and NK Cells.

FIG. 26 shows binding abilities of humanized NK cell engagers to NKp46 expression cells.

FIG. 27 shows binding abilities of humanized NK cell engagers to CD79b expression cells.

FIGS. 28A-28B show humanized NK cell engagers mediated target cancer B cell killing by PBMC (test 1).

FIGS. 29A-29B show humanized NK cell engagers mediated target cancer B cell killing by PBMC (test 2).

FIG. 30 shows plasma spiked NK cell engagers mediated target cancer B cell killing by PBMC.

FIG. 31 shows quantification of BCMA antigen density on multiple myeloma tumor cell and normal B cell surface.

FIG. 32 shows BCMA/NKp46 bispecific antibodies mediate target multiple myeloma cell killing by PBMC.

FIG. 33 shows some of the amino acid sequences described in the present disclosure.

### DETAILED DESCRIPTION

Natural killer (NK) cells play an important role in first-line defense against tumor and virus-infected cells. The activity of NK cells is tightly regulated by a repertoire of cell surface expressed inhibitory and activating receptors. NKp46 is a major NK cell-activating receptor that is involved in the elimination of target cells. NK cells form different types of synapses that result in distinct functional outcomes, cytotoxic, inhibitory, and regulatory. Recent studies revealed that complex integration of NK receptor signaling controls cytoskeletal rearrangement and other immune synapse-related events. Thus, anti-NKp46 antibodies can be potentially used as cancer therapies and treatment of other diseases such as autoimmune diseases.

The present disclosure provides examples of antibodies, antigen-binding fragment thereof, that bind to NKp46.

A multispecific antibody or antigen-binding fragment thereof is an artificial protein that can simultaneously bind to two or more than two different epitopes (e.g., on two different antigens). In some embodiments, the multispecific antibody is a bispecific antibody. A bispecific antibody or antigen-binding fragment thereof can have two arms. Each arm can have one heavy chain variable region and one light chain variable region, forming an antigen-binding domain (or an antigen-binding region). The two arms can bind to two different antigens. In some embodiments, additional antigen-binding domains can be added to a monoclonal antibody (e.g., to the C terminus of the light chain or the heavy chain).

This disclosure relates to multispecific (e.g., bispecific) antibodies or antigen-binding fragments thereof, comprising a first antigen-binding domain that specifically binds to a tumor associated antigen (TAA) and a second antigen-binding domain that specifically binds to an NK cell surface antigen.

### NKp46

Natural killer (NK) cells are granular lymphocytes that were initially recognized for their capacity to efficiently eliminate tumor cells without prior sensitization. Viral infections can also induce NK cytotoxicity. Indeed, NK cells are an important component of the innate immune response against viruses, as evidenced by the abundant infections in patients with primary NK cells deficiencies and mice lacking NK cells. Control of NK cell function is mainly regulated by its recognition of self MHC class I molecules through a set of inhibitory receptors—the killer-cell immunoglobulin-like receptors (KIRs) in humans and the Ly49 receptor family in mice. NK cells do not express variable antigen specific receptors. Instead, their activation is controlled by a repertoire of germline encoded activating receptors that recognize different ligands on the surface of susceptible target cells. Important NK activating receptors include NKG2D and three members of the natural cytotoxicity receptors group (NCR): NKp46, NKp44, and NKp30.

Distinct among the NCRs, NKp46 (NCR1) is evolutionary conserved between mice and humans, and NKp46 activity has been studied in several mouse models. NKp46 is uniquely expressed on all NK cell subsets and has been suggested as a possible target for NK cell ablation and as a pan NK cell marker. NKp46 is a 46 kDa type 1 transmembrane glycoprotein characterized by a 30-aa intracellular tail, 20-aa transmembrane domain, and two extracellular Ig-like domains that are contacted through a 25-aa short peptide. The transmembrane domain contains an Arg residue that can bind the signal adapter proteins FcϵRI and CD3ζ. Two NKp46 extracellular (D1-D2) crystal structures were resolved and showed similarity to the extracellular domains of different leukocyte immunoglobulin-like receptors (LIRs) and KIR receptors. The membrane-proximal domain (D2) was identified as the ligand-binding domain of NKp46 receptor.

To date, few pathogen-associated ligands and cellular co-ligands for NKp46 have been documented. Viral hemagglutinin (HA) of influenza and Sendai viruses, and Newcastle disease HA-neuraminidase can bind to NKp46 and induce NK activation. The intermediate filament protein Vimentin, expressed on Mycobacterium tuberculosis-infected monocytes, is involved in binding to the NKp46 receptor. Target cell-expressed heparan sulfate (HS) can bind with NKp46 and other NK receptors and serves as an accessory molecule for the recognition of cellular ligand(s) by NKp46. On the other hand, NK cell membrane-expressed HS proteoglycans may regulate the function and fate of NKp46 and other HS-binding NK receptors.

In addition to the role of NKp46 in recognition of cancer cells, several studies suggest the involvement of NKp46 in other pathological conditions. NKp46 was shown to be crucial for development of type 1 diabetes through recognition of an unknown ligand on pancreatic β-cells. It is also demonstrated that NKp46 contributes to clearance of Streptococcus pneumoniae by interacting with infected alveolar macrophages. NKp46 also mediates the killing of stellate cells in attenuated liver fibrosis and of HCV-infected hepatocytes. In a murine model of random mutation (Ncr1Noé/Noé), the lack of NKp46 surface expression resulted in hyper-responsiveness of NK cells following MCMV infection. NKp46 also regulates graft-versus-host disease and allergic response.

Although early studies of NK cells clearly showed that cytolytic activity is contact dependent, it was not until two and a half decades later that the intercellular complex between NK and target cells was thoroughly characterized, and the term NK cell immune synapse was coined. NK cell immune synapse formation and function is composed of several highly regulated stages. The role of human NKp46 in NK immune synapse formation and function was studied. It was reported that following the initiation of an NK-target cell interaction, NKp46 clusters at the cell membrane, specifically at the immune synapse. At the immune synapse, NKp46 mediates cytoskeletal rearrangement and cellular polarization.

More information related to NKp46 can be found e.g., in Hadad et al., Front Immunol. 2015; 6: 495; Sivori et al., J Exp Med (1997) 186:1129; Pessino et al., J Exp Med (1998) 188:953; and Westgaard et al., J Leukoc Biol (2004) 76:1200-6, each of which is incorporated herein by reference in the entirety.

### Anti-NKp46 Antibodies and Antigen-Binding Fragments

The disclosure provides antibodies and antigen-binding fragments thereof that specifically bind to NKp46 (e.g., human NKp46). The antibodies and antigen-binding fragments described herein are capable of binding to NKp46. In some embodiments, these antibodies activate NK cells and increases NK cell-mediated target cell (e.g., tumor cell) killing. In some embodiments, these antibodies can initiate complement-dependent cytotoxicity (CDC) or antibody-dependent cellular cytotoxicity (ADCC). In some embodiments, these antibodies bind to cells (e.g., NK cells) expressing NKp46.

The disclosure provides, e.g., anti-NKp46 antibodies ch3-7 (also referred to as “3-7”), ch5-13 (also referred to as “5-13”), and ch6-1 (also referred to as “6-1”), the modified antibodies thereof, including, e.g., chimeric antibodies, humanized antibodies, and human antibodies.

The disclosure also provides humanized anti-NKp46 antibodies h3-7 (humanized antibody of ch3-7), h5-13 (humanized antibody of ch5-13) and h6-1 (humanized antibody of ch6-1).

The CDR sequences for ch3-7, and ch3-7 derived antibodies (e.g., humanized antibodies such as h3-7) include CDRs of the heavy chain variable domain, SEQ ID NOs: 5, 7, and 9, and CDRs of the light chain variable domain, SEQ ID NOs: 10, 11 and 12, as defined by Kabat definition.

The CDRs can also be defined by Chothia system. Under the Chothia definition, the CDR sequences of the heavy chain variable domain are set forth in SEQ ID NOs: 6, 8, and 9, and CDR sequences of the light chain variable domain are set forth in SEQ ID NOs: 10, 11 and 12.

The CDR sequences for ch5-13, and ch5-13 derived antibodies (e.g., humanized antibodies such as h5-13) include CDRs of the heavy chain variable domain, SEQ ID NOs: 15, 17, and 19, and CDRs of the light chain variable domain, SEQ ID NOs: 20, 21, and 22, as defined by Kabat definition. Under Chothia definition, the CDR sequences of the heavy chain variable domain are set forth in SEQ ID NOs: 16, 18 and 19, and CDRs of the light chain variable domain are set forth in SEQ ID NOs: 20, 21, and 22.

The CDR sequences for ch6-1, and ch6-1 derived antibodies (e.g., humanized antibodies such as h6-1) include CDRs of the heavy chain variable domain, SEQ ID NOs: 25, 27, and 29, and CDRs of the light chain variable domain, SEQ ID NOs: 30, 31, and 32, as defined by Kabat definition. Under Chothia definition, the CDR sequences of the heavy chain variable domain are set forth in SEQ ID NOs: 26, 28, and 29, and CDRs of the light chain variable domain are set forth in SEQ ID NOs: 30, 31, and 32.

The amino acid sequence for the heavy chain variable region of ch3-7 antibody is set forth in SEQ ID NO: 3. The amino acid sequence for the light chain variable region of ch3-7 antibody is set forth in SEQ ID NO: 4.

The amino acid sequence for the heavy chain variable region of ch5-13 antibody is set forth in SEQ ID NO: 13. The amino acid sequence for the light chain variable region of ch5-13 antibody is set forth in SEQ ID NO: 14.

The amino acid sequence for the heavy chain variable region of ch6-1 antibody is set forth in SEQ ID NO: 23. The amino acid sequence for the light chain variable region of ch6-1 antibody is set forth in SEQ ID NO: 24.

The amino acid sequence for the heavy chain variable region of h3-7 antibody is set forth in SEQ ID NO: 35. The amino acid sequence for the light chain variable region of h3-7 antibody is set forth in SEQ ID NO: 36.

The amino acid sequence for the heavy chain variable region of h5-13 antibody is set forth in SEQ ID NO: 37. The amino acid sequence for the light chain variable region of h5-13 antibody is set forth in SEQ ID NO: 38.

The amino acid sequence for the heavy chain variable region of h6-1 antibody is set forth in SEQ ID NO: 33. The amino acid sequence for the light chain variable region of h6-1 antibody is set forth in SEQ ID NO: 34.

The amino acid sequences for heavy chain variable regions and light variable regions of the modified antibodies are also provided. In some embodiments, the heavy chain variable region is at least 80%, 85%, 90%, 91%, 92%, 93%, 94%, 95%, 96%, 97%, 98%, or 99% identical to SEQ ID NO: 3, 13, 23, 33, 35 or 37. In some embodiments, the light chain variable region is at least 80%, 85%, 90%, 91%, 92%, 93%, 94%, 95%, 96%, 97%, 98%, or 99% identical to SEQ ID NO: 4, 14, 24, 34, 36 or 38. The heavy chain variable region sequence can be paired with the corresponding light chain variable region sequence, and together they bind to NKp46.

In some embodiments, the variable regions are fully human, e.g., derived from human heavy chain immunoglobulin locus sequences (e.g., recombination of human IGHV, IGHD, and IGHJ genes), and/or human kappa chain immunoglobulin locus sequences (e.g., recombination of human IGKV and IGKJ genes).

Furthermore, in some embodiments, the antibodies or antigen-binding fragments thereof described herein can also contain one, two, or three heavy chain variable region CDRs selected from the group of SEQ ID NOs: 5, 7, and 9, SEQ ID NOs: 6, 8, and 9, SEQ ID NOs: 15, 17 and 19; SEQ ID NOs: 16, 18 and 19; SEQ ID NOs: 25, 27 and 29; and SEQ ID NOs: 26, 28 and 29; and/or one, two, or three light chain variable region CDRs selected from the group of SEQ ID NOs: 10, 11 and 12, SEQ ID NOs: 20, 21 and 22, and SEQ ID NOs: 30, 31 and 32.

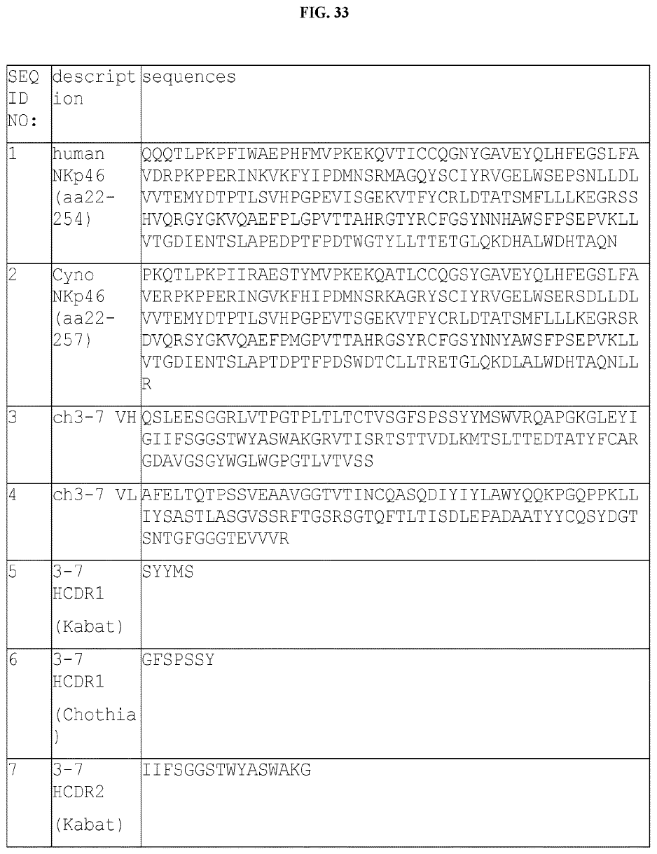

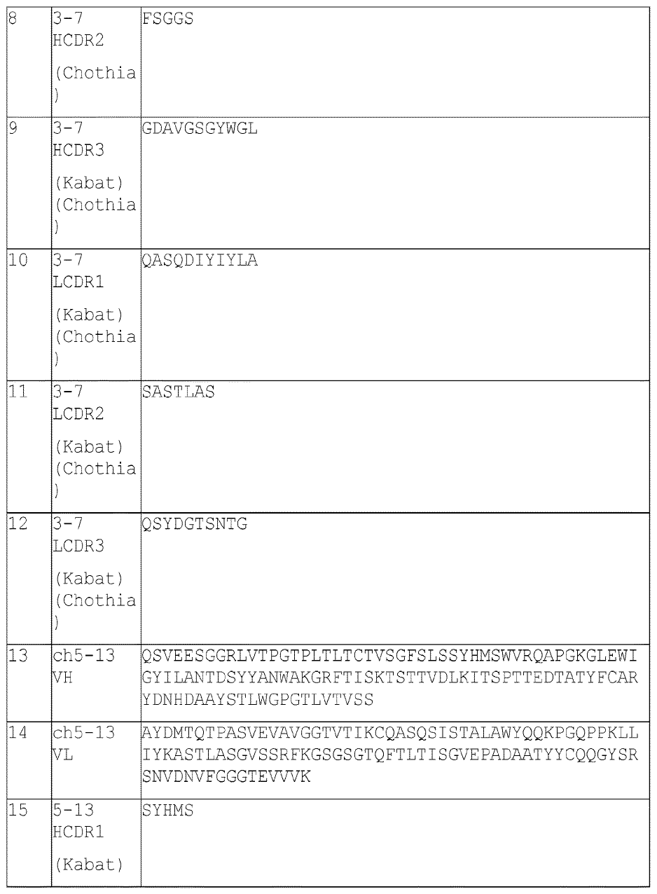

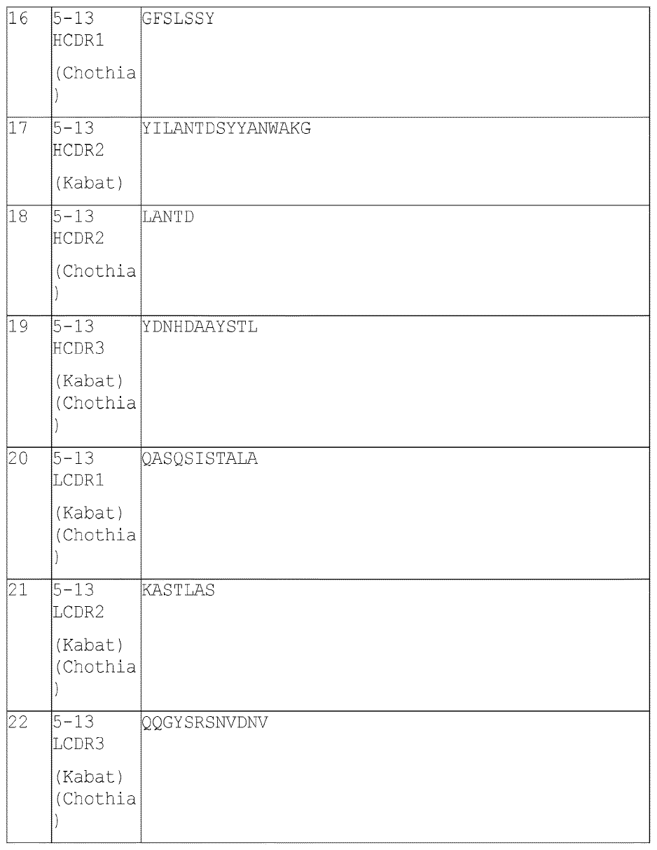

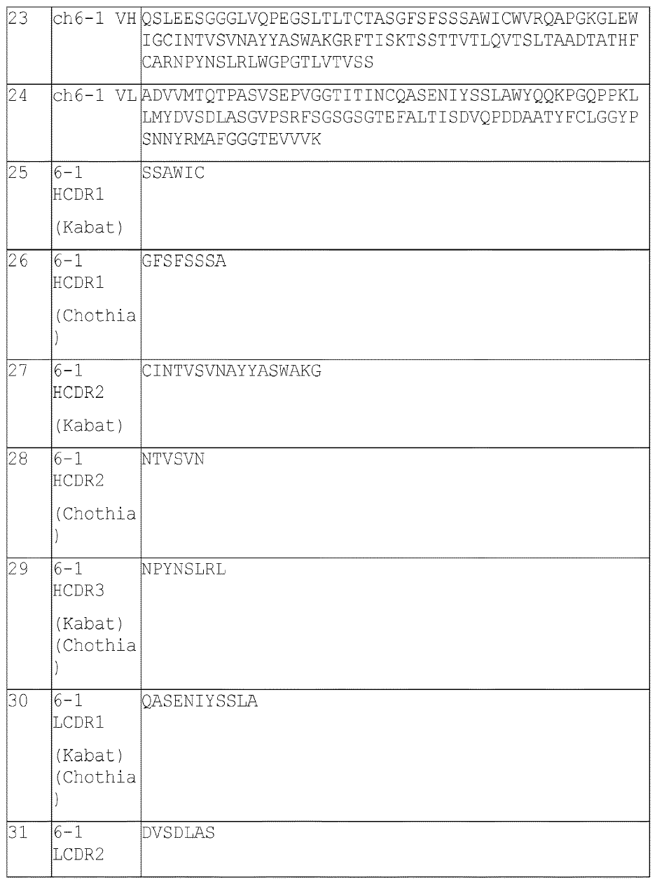

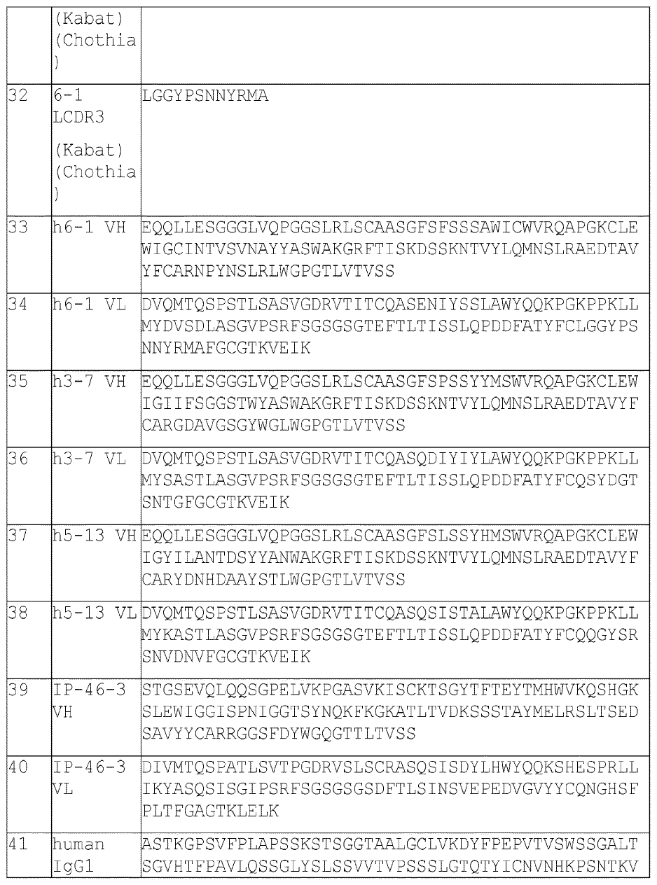

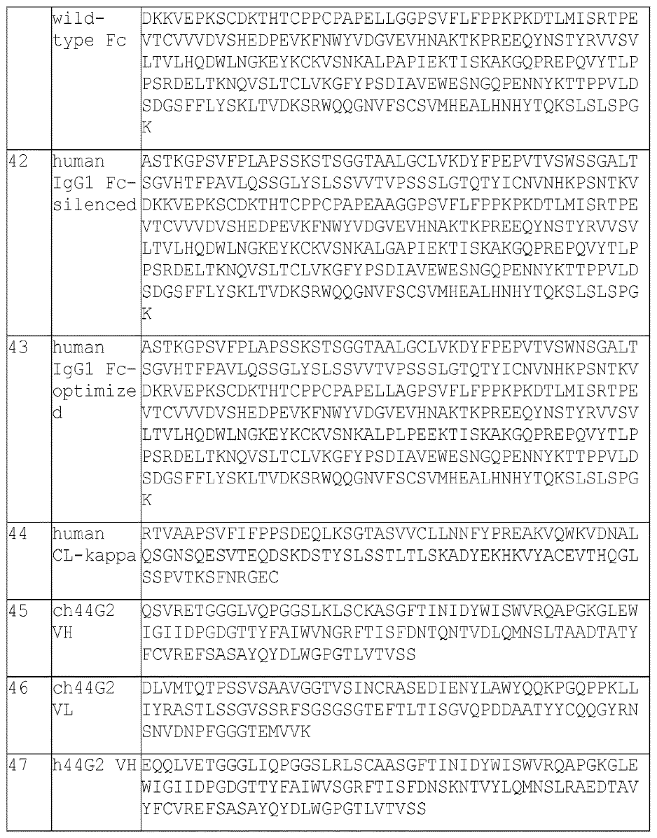

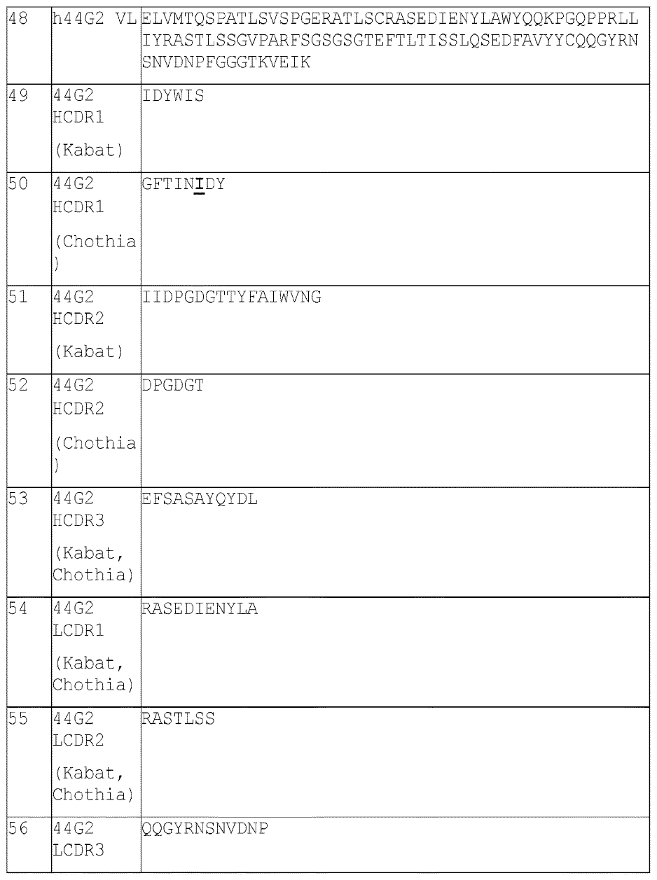

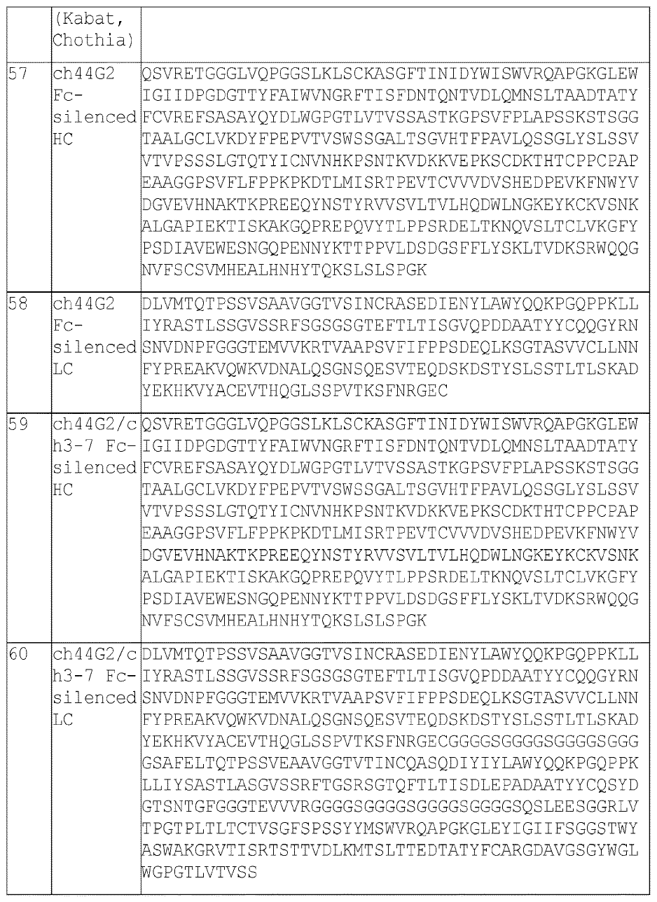

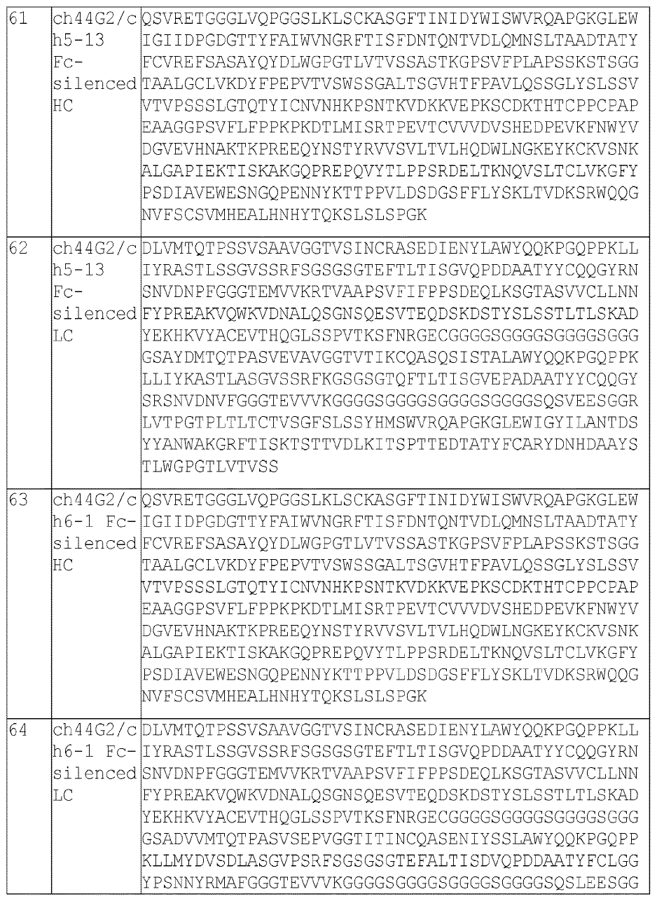

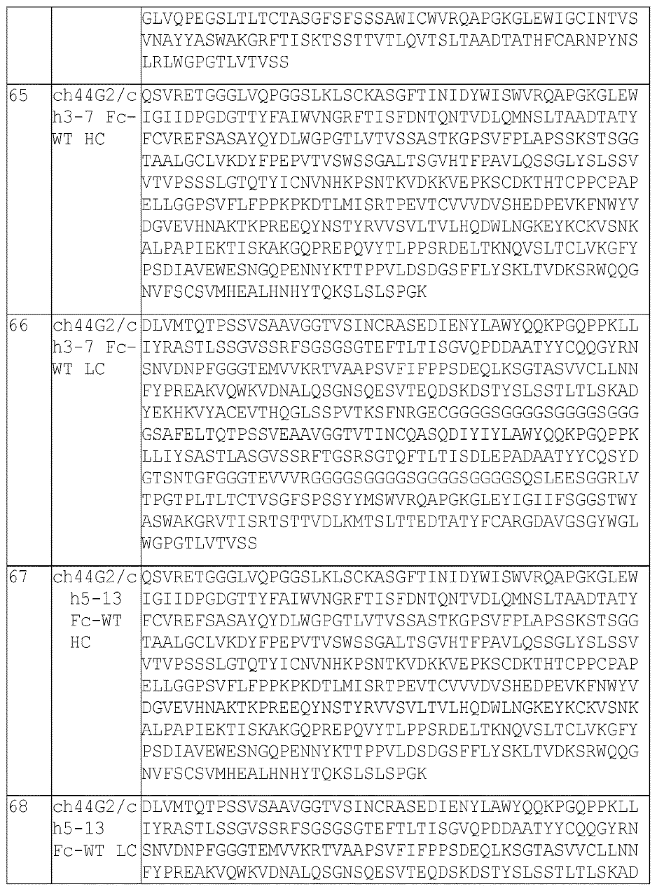

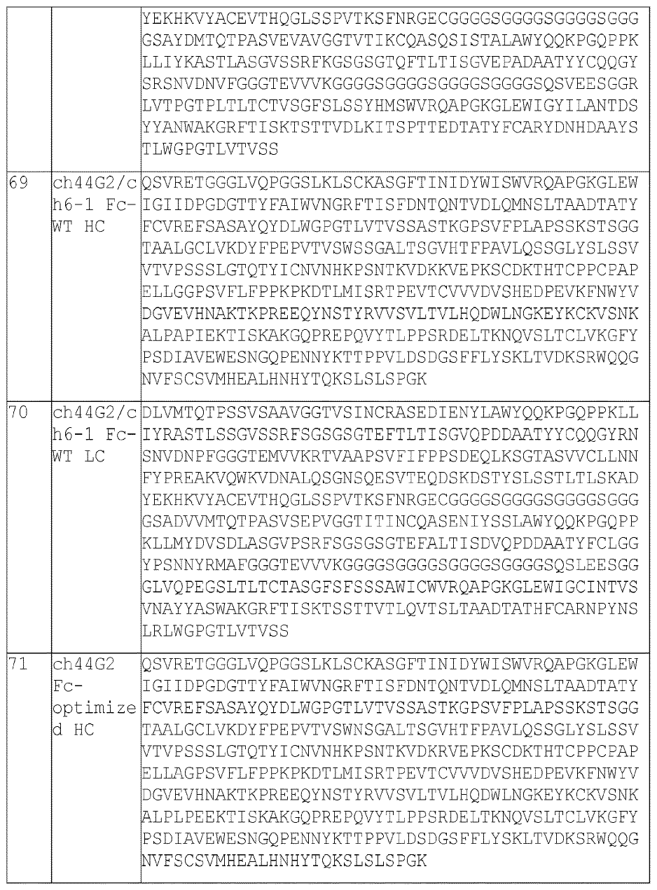

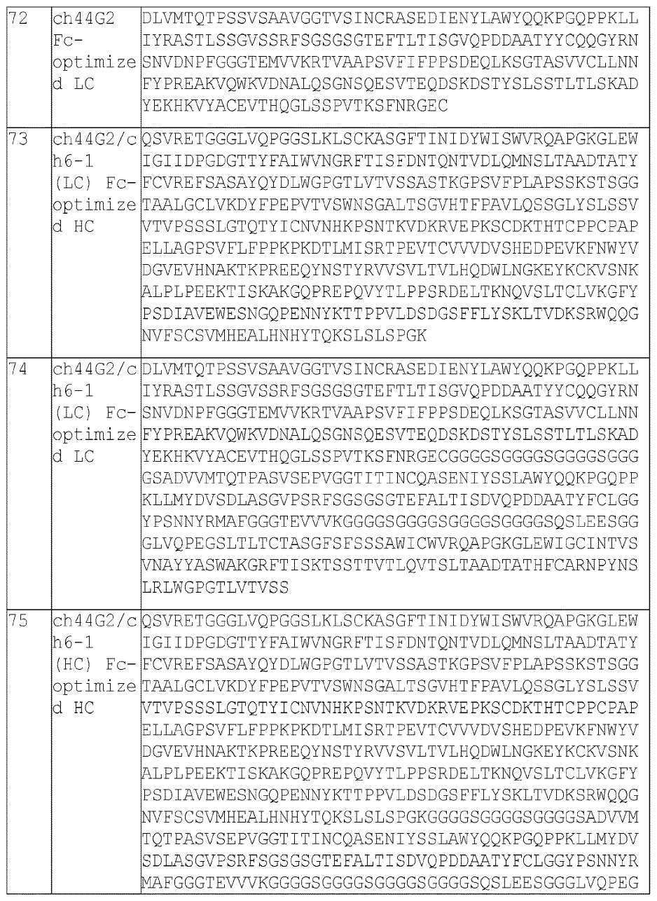

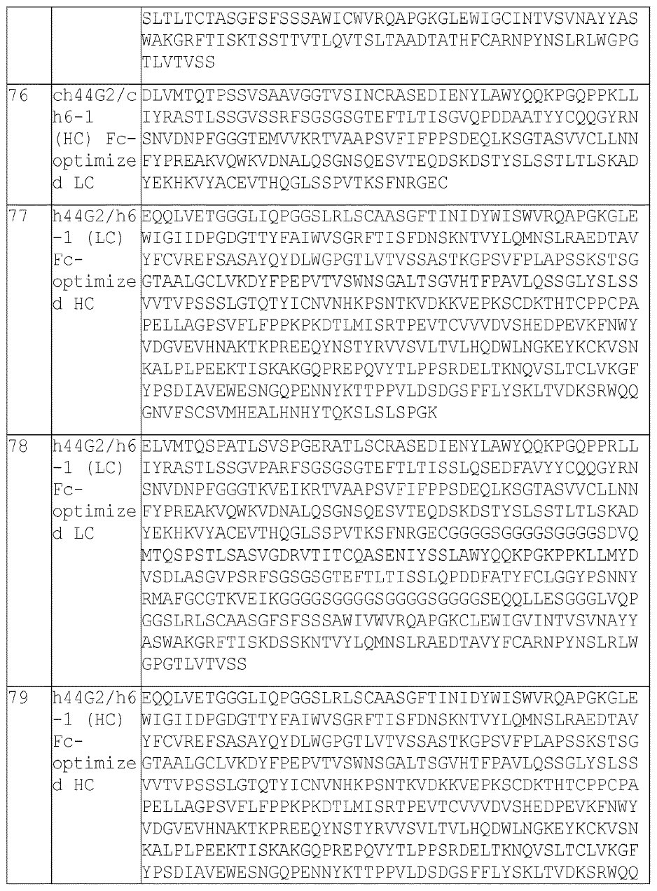

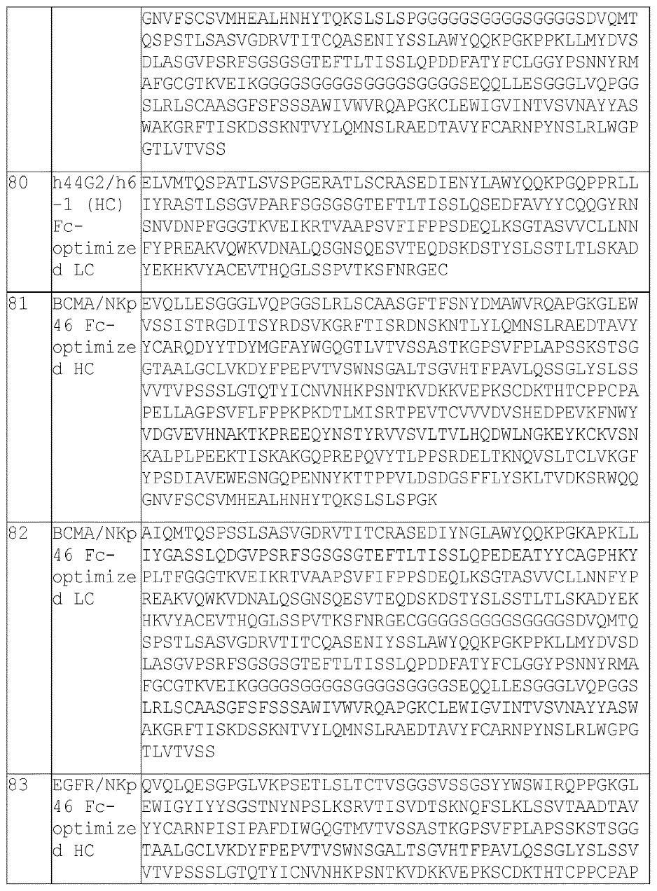

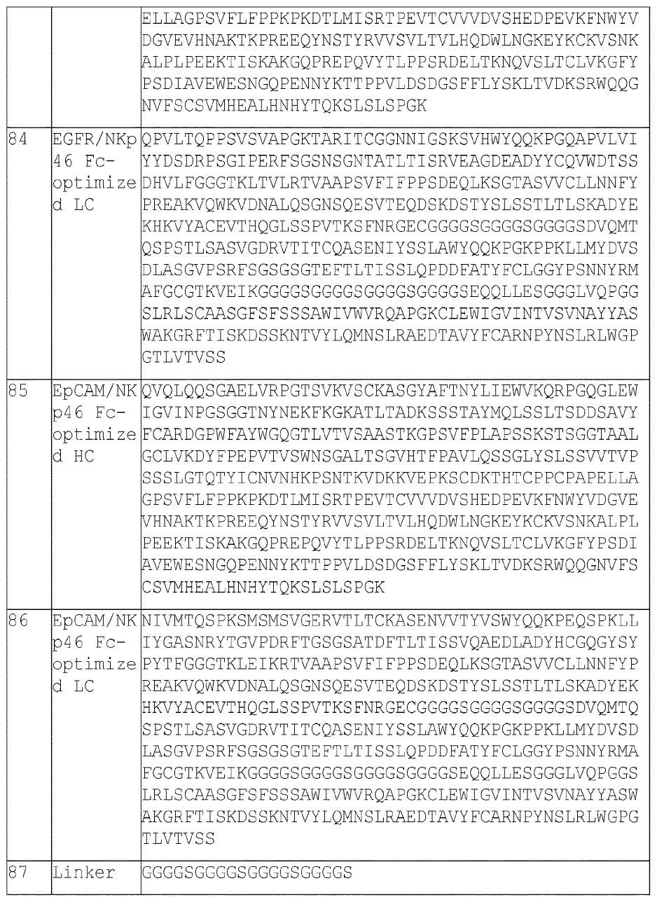

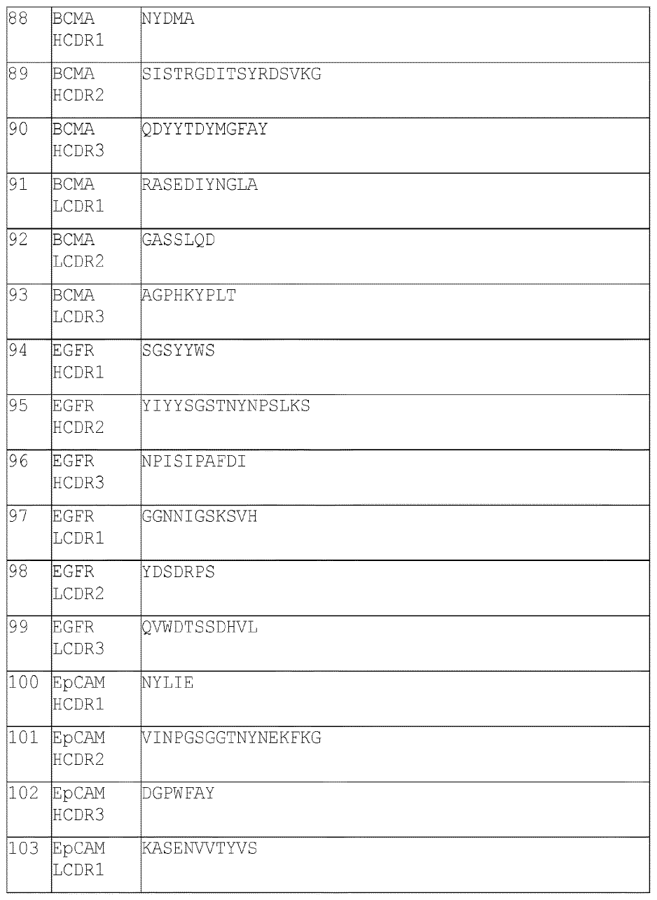

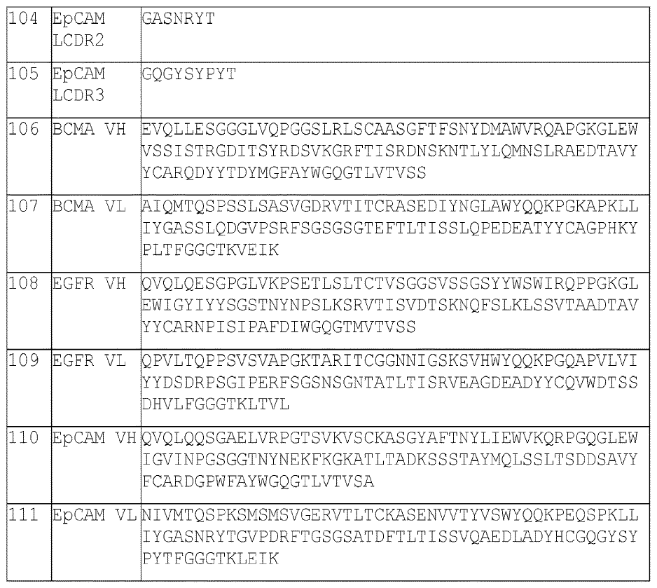

In some embodiments, the antibodies can have a heavy chain variable region (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence. In some embodiments, the antibodies can have a light chain variable region (VL) comprising CDRs 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence. The selected VH and VH CDRs 1, 2, 3 amino acid sequences and the selected VL and VL CDRs, 1, 2, 3 amino acid sequences are shown in FIG. 33.

In some embodiments, the antibody or an antigen-binding fragment described herein can contain a heavy chain variable domain containing one, two, or three of the CDRs of SEQ ID NO: 1 with zero, one or two amino acid insertions, deletions, or substitutions; SEQ ID NO: 2 with zero, one or two amino acid insertions, deletions, or substitutions; SEQ ID NO: 3 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 5 with zero, one or two amino acid insertions, deletions, or substitutions; SEQ ID NO: 7 with zero, one or two amino acid insertions, deletions, or substitutions; SEQ ID NO: 9 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 6 with zero, one or two amino acid insertions, deletions, or substitutions; SEQ ID NO: 8 with zero, one or two amino acid insertions, deletions, or substitutions; SEQ ID NO: 9 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 15 with zero, one or two amino acid insertions, deletions, or substitutions; SEQ ID NO: 17 with zero, one or two amino acid insertions, deletions, or substitutions; SEQ ID NO: 19 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 16 with zero, one or two amino acid insertions, deletions, or substitutions; SEQ ID NO: 18 with zero, one or two amino acid insertions, deletions, or substitutions; SEQ ID NO: 19 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 25 with zero, one or two amino acid insertions, deletions, or substitutions; SEQ ID NO: 27 with zero, one or two amino acid insertions, deletions, or substitutions; SEQ ID NO: 29 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 26 with zero, one or two amino acid insertions, deletions, or substitutions; SEQ ID NO: 28 with zero, one or two amino acid insertions, deletions, or substitutions; SEQ ID NO: 29 with zero, one or two amino acid insertions, deletions, or substitutions.

In some embodiments, the antibody or an antigen-binding fragment described herein can contain a light chain variable domain containing one, two, or three of the CDRs of SEQ ID NO: 10 with zero, one or two amino acid insertions, deletions, or substitutions; SEQ ID NO: 11 with zero, one or two amino acid insertions, deletions, or substitutions; SEQ ID NO: 12 with zero, one or two amino acid insertions, deletions, or substitutions.

In some embodiments, the antibody or an antigen-binding fragment described herein can contain a light chain variable domain containing one, two, or three of the CDRs of SEQ ID NO: 20 with zero, one or two amino acid insertions, deletions, or substitutions; SEQ ID NO: 21 with zero, one or two amino acid insertions, deletions, or substitutions; SEQ ID NO: 22 with zero, one or two amino acid insertions, deletions, or substitutions.

In some embodiments, the antibody or an antigen-binding fragment described herein can contain a light chain variable domain containing one, two, or three of the CDRs of SEQ ID NO: 30 with zero, one or two amino acid insertions, deletions, or substitutions; SEQ ID NO: 31 with zero, one or two amino acid insertions, deletions, or substitutions; SEQ ID NO: 32 with zero, one or two amino acid insertions, deletions, or substitutions.

The insertions, deletions, and substitutions can be within the CDR sequence, or at one or both terminal ends of the CDR sequence. In some embodiments, the CDR is determined based on Kabat definition scheme. In some embodiments, the CDR is determined based on Chothia definition scheme. In some embodiments, the CDR is determined based on a combination of Kabat and Chothia definition scheme. In some embodiments, the CDR is determined based on IMGT definition. In some embodiments, the CDR is determined based on contact definition.

The disclosure also provides antibodies or antigen-binding fragments thereof that bind to NKp46. The antibodies or antigen-binding fragments thereof contain a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence is SEQ ID NO: 3, and the selected VL sequence is SEQ ID NO: 4. In some embodiments, the selected VH sequence is SEQ ID NO: 13 and the selected VL sequence is SEQ ID NO: 14. In some embodiments, the selected VH sequence is SEQ ID NO: 23 and the selected VL sequence is SEQ ID NO: 24. In some embodiments, the selected VH sequence is SEQ ID NO: 33 and the selected VL sequence is SEQ ID NO: 34. In some embodiments, the selected VH sequence is SEQ ID NO: 35 and the selected VL sequence is SEQ ID NO: 36. In some embodiments, the selected VH sequence is SEQ ID NO. 37 and the selected VL sequence is SEQ ID NO: 38.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90%, 95%, or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

The disclosure also provides nucleic acid comprising a polynucleotide encoding a polypeptide comprising an immunoglobulin heavy chain or an immunoglobulin light chain. When the polypeptides are paired with corresponding polypeptide (e.g., a corresponding heavy chain variable region or a corresponding light chain variable region), the paired polypeptides bind to NKp46.

The anti-NKp46 antibodies and antigen-binding fragments can also be antibody variants (including derivatives and conjugates) of antibodies or antibody fragments and multi-specific (e.g., bi-specific) antibodies or antibody fragments. Additional antibodies provided herein are polyclonal, monoclonal, multimeric, multi-specific (e.g., bi-specific), human antibodies, chimeric antibodies (e.g., human-mouse chimera or human-rabbit chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG1, IgG2, IgG3, IgG4, IgA1, and IgA2), or subclass. In some embodiments, the antibody or antigen-binding fragment thereof is an IgG antibody or antigen-binding fragment thereof.

Fragments of antibodies are suitable for use in the methods provided so long as they retain the desired affinity and specificity of the full-length antibody. Thus, a fragment of an antibody that binds to NKp46 will retain an ability to bind to NKp46. An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site. Single-chain Fv or (scFv) antibody fragments comprise the VH and VL domains (or regions) of antibody, wherein these domains are present in a single polypeptide chain. Generally, the scFv polypeptide further comprises a polypeptide linker between the VH and VL domains, which enables the scFv to form the desired structure for antigen binding.

The present disclosure also provides an antibody or antigen-binding fragment thereof that cross-competes with any antibody or antigen-binding fragment as described herein. The cross-competing assay is known in the art, and is described e.g., in Moore et al., “Antibody cross-competition analysis of the human immunodeficiency virus type 1 gp120 exterior envelope glycoprotein.” Journal of Virology 70.3 (1996): 1863-1872, which is incorporated herein reference in its entirety. In one aspect, the present disclosure also provides an antibody or antigen-binding fragment thereof that binds to the same epitope or region as any antibody or antigen-binding fragment as described herein. The epitope binning assay is known in the art, and is described e.g., in Estep et al. “High throughput solution-based measurement of antibody-antigen affinity and epitope binning.” MAbs. Vol. 5. No. 2. Taylor & Francis, 2013, which is incorporated herein reference in its entirety.

### Methods of Making Anti-NKp46 Antibodies

An isolated fragment of human NKp46 (e.g., extracellular region) can be used as an immunogen to generate antibodies using standard techniques for polyclonal and monoclonal antibody preparation. Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to an agent that is immunogenic in the species to be immunized. Animals can be injected with the antigenic peptide or protein more than one time (e.g., twice, three times, or four times).

The full-length polypeptide or protein can be used or, alternatively, antigenic peptide fragments thereof can be used as immunogens. The antigenic peptide of a protein comprises at least 8 (e.g., at least 10, 15, 20, or 30) amino acid residues of the amino acid sequence of NKp46 and encompasses an epitope of the protein such that an antibody raised against the peptide forms a specific immune complex with the protein. As described above, the full length sequence of human NKp46 is known in the art (amino acids 22-254 of human NKp46 is shown in SEQ ID NO: 1). The full length sequence of monkey NKp46 is known in the art (amino acids 22-257 of monkey NKp46 is shown in SEQ ID NO: 2). In some embodiments, a His-tagged human NKp46 protein is used as the immunogen.

An immunogen typically is used to prepare antibodies by immunizing a suitable subject (e.g., human or transgenic animal expressing at least one human immunoglobulin locus). An appropriate immunogenic preparation can contain, for example, a recombinantly-expressed or a chemically-synthesized polypeptide (e.g., a fragment of human NKp46). The preparation can further include an adjuvant, such as Freund's complete or incomplete adjuvant, or a similar immunostimulatory agent.

Polyclonal antibodies can be prepared as described above by immunizing a suitable subject with a NKp46 polypeptide, or an antigenic peptide thereof (e.g., part of NKp46, such as the extracellular region) as an immunogen. The antibody titer in the immunized subject can be monitored over time by standard techniques, such as with an enzyme-linked immunosorbent assay (ELISA) using the immobilized NKp46 polypeptide or peptide. If desired, the antibody molecules can be isolated from the mammal (e.g., from the blood) and further purified by well-known techniques, such as protein A or protein G chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler et al. (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985), or trioma techniques. The technology for producing hybridomas is well known (see, generally, Current Protocols in Immunology, 1994, Coligan et al. (Eds.), John Wiley & Sons, Inc., New York, NY). Hybridoma cells producing a monoclonal antibody are detected by screening the hybridoma culture supernatants for antibodies that bind the polypeptide or epitope of interest, e.g., using a standard ELISA assay.

Variants of the antibodies or antigen-binding fragments described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antigen-binding fragments will have increased affinity for the target protein, e.g., NKp46. Any combination of deletions, insertions, and/or combinations can be made to arrive at an antibody or antigen-binding fragment thereof that has increased binding affinity for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Antibodies disclosed herein can be derived from any species of animal, including mammals. Non-limiting examples of native antibodies include antibodies derived from humans, primates, e.g., monkeys and apes, cows, pigs, horses, sheep, camelids (e.g., camels and llamas), chicken, goats, and rodents (e.g., rats, mice, hamsters and rabbits), including transgenic rodents genetically engineered to produce human antibodies.

Human and humanized antibodies include antibodies having variable and constant regions derived from (or having the same amino acid sequence as those derived from) human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs.

A humanized antibody, typically has a human framework (FR) grafted with non-human CDRs. Thus, a humanized antibody has one or more amino acid sequence introduced into it from a source which is non-human. These non-human amino acid residues are often referred to as “import” residues, which are typically taken from an “import” variable domain. Humanization can be essentially performed by e.g., substituting rodent CDRs or CDR sequences for the corresponding sequences of a human antibody. These methods are described in e.g., Jones et al. “Replacing the complementarity-determining regions in a human antibody with those from a mouse.” Nature 321.6069 (1986); 522; Riechmann et al. “Reshaping human antibodies for therapy.” Nature 332.6162 (1988): 323; Dall'Acqua et al. “Antibody humanization by framework shuffling.” Methods 36.1 (2005): 43-60; each of which is incorporated by reference herein in its entirety. Accordingly, “humanized” antibodies are chimeric antibodies wherein substantially less than an intact human V domain has been substituted by the corresponding sequence from a non-human species. In practice, humanized antibodies are typically non-human (e.g., mouse or rabbit) antibodies in which some CDR residues and some FR residues are substituted by residues from analogous sites in human antibodies.

It is further important that antibodies be humanized with retention of high specificity and affinity for the antigen and other favorable biological properties. To achieve this goal, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved.

Ordinarily, amino acid sequence variants of the human, humanized, or chimeric anti-NKp46 antibody will contain an amino acid sequence having at least 75%, 80%, 85%, 90%, 95%, 96%, 97%, 98%, or 99% percent identity with a sequence present in the light or heavy chain of the original antibody.

Identity or homology with respect to an original sequence is usually the percentage of amino acid residues present within the candidate sequence that are identical with a sequence present within the human, humanized, or chimeric anti-NKp46 antibody or fragment, after aligning the sequences and introducing gaps, if necessary, to achieve the maximum percent sequence identity, and not considering any conservative substitutions as part of the sequence identity.

Additional modifications to the anti-NKp46 antibodies or antigen-binding fragments can be made. For example, a cysteine residue(s) can be introduced into the Fc region, thereby allowing interchain disulfide bond formation in this region. The homodimeric antibody thus generated may have any increased half-life in vitro and/or in vivo. Homodimeric antibodies with increased half-life in vitro and/or in vivo can also be prepared using heterobifunctional cross-linkers as described, for example, in Wolff et al. Wolff et al. (“Monoclonal antibody homodimers: enhanced antitumor activity in nude mice.” Cancer research 53.11 (1993): 2560-2565). Alternatively, an antibody can be engineered which has dual Fc regions.

In some embodiments, a covalent modification can be made to the anti-NKp46 antibody or antigen-binding fragment thereof. These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatization agent that is capable of reacting with selected side chains or the N- or C-terminal residues.

In some embodiments, antibody variants are provided having a carbohydrate structure that lacks fucose attached (directly or indirectly) to an Fc region. For example, the amount of fucose in such antibody composition may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; or position 314 in Kabat numbering); however, Asn297 may also be located about +3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the antibody can be further engineered to replace the Asparagine at position 297 with Alanine (N297A).

In some embodiments, to facilitate production efficiency by avoiding Fab-arm exchange, the Fc region of the antibodies was further engineered to replace the serine at position 228 (EU numbering) of IgG4 with proline (S228P). A detailed description regarding S228 mutation is described, e.g., in Silva et al. “The S228P mutation prevents in vivo and in vitro IgG4 Fab-arm exchange as demonstrated using a combination of novel quantitative immunoassays and physiological matrix preparation.” Journal of Biological Chemistry 290.9 (2015); 5462-5469, which is incorporated by reference in its entirety.

### Tumor Associated Antigens

A tumor associated antigen (TAA) is an antigenic substance produced in tumor cells. It can trigger an immune response in the host. Tumor antigens are useful tumor markers in identifying tumor cells with diagnostic tests and are potential candidates for use in cancer therapy. Numerous tumor-associated antigens are known in the art (see, e.g., Yu et al., Cancers (Basel). 2023 April; 15(8): 2323; and Tong et al., Mol Cancer, 2022 Nov. 1; 21(1):206, each of which is incorporated herein by reference in its entirety).

In some embodiments, the TAA is selected from the group consisting of: CD79b, EpCAM, B-cell maturation antigen (BCMA), C-MET, LRRC15, MUC1, OX40, STEAP1, PSMA, DLL3, EGFR, perforin, granzyme B, Fas ligand (FasL), CD1d, Membrane glycolipids, globotriaosyl-ceramide (Gb3Cer/CD77), gangliosides (GD2, GD3, and GM2), CD34, CD45, human leukocyte antigen-DR (HLA-DR), CD123, CD38, CLL1, CD105, CD71, SSC, MAGE, MUC16, CD19, WT-1, B7H3, TEM8, CD22, LI-CAM, ROR-1, CEA, 4-1BB, ETA, 5T4, adenocarcinoma antigen, alpha-fetoprotein (AFP), BAFF, B-lymphoma cell, C242 antigen, CA-125, carbonic anhydrase 9 (CA-IX), CCR4, CD133, CDI 52, CD20, CD125, CD200, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, IL-5, IL-13, IL-6, IL-15, insulin-like growth factor I receptor, integrin a5b1, integrin anb3, MORAb-009, MS4A1, MUC1, mucin CanAg, N-glycolylneuraminic acid, NPC-1C, PD-1, PD-L1, PDGF-R a, PDL192, phosphatidylserine, prostatic carcinoma cells, RANKL, RON, SCH 900105, SDC1, SLAMF7, TAG-72, tenascin C, TGF beta 2, TGF-b, TRAIL-RI, TRAIL-R2, tumor antigen CTAA16.88, VEGF-A, VEGFR-1, VEGFR2, and vimentin.

In some embodiments, the multispecific (e.g., bispecific) antibodies or antigen-binding fragments thereof, comprises a first antigen-binding domain that specifically binds to a tumor associated antigen (TAA) and a second antigen-binding domain that specifically binds to an NK cell surface antigen (e.g., NKp46). In some embodiments, the first antigen-binding domain specifically binds to CD79b. In some embodiments, the first antigen-binding domain specifically binds to BCMA. In some embodiments, the first antigen-binding domain specifically binds to EGFR. In some embodiments, the first antigen-binding domain specifically binds to EpCAM. In some embodiments, the first antigen-binding domain specifically binds to DLL3. In some embodiments, the first antigen-binding domain specifically binds to PSMA. In some embodiments, the first antigen-binding domain specifically binds to STEAP1. In some embodiments, the first antigen-binding domain specifically binds to MUC1. In some embodiments, the first antigen-binding domain specifically binds to c-MET. In some embodiments, the first antigen-binding domain specifically binds to LRRC15. In some embodiments, the first antigen-binding domain specifically binds to OX40.

In some embodiments, the first antigen-binding domain comprises a heavy chain variable region (VH1) that comprises complementarity determining regions (CDRs) 1, 2, and 3, wherein the VH1 CDR1 region comprises an amino acid sequence that is about or at least 80%, 85%, 90%, 95% or 100% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and the first antigen-binding domain comprises a light chain variable region (VL1) that comprises CDRs 1, 2, and 3, wherein the VL1 CDR1 region comprises an amino acid sequence that is about or at least 80%, 85%, 90%, 95% or 100% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence.

In some embodiments, the first antigen-binding domain comprises a heavy chain variable region (VH1) that comprises an amino acid sequence that is about or at least 80%, 85%, 90%, 95% or 100% identical to a selected VH1 amino acid sequence, and the first antigen-binding domain comprises a light chain variable region (VL1) that comprises an amino acid sequence that is about or at least 80%, 85%, 90%, 95% or 100% identical to a selected VL1 amino acid sequence.

In some embodiments, the first antigen-binding domain specifically binds to DLL3; and the second antigen-binding domain that specifically binds to NKp46.

In some embodiments, the selected CDRs of the DLL3 antigen-binding fragment are the corresponding VH CDRs 1, 2, 3 and VL CDRs 1, 2, 3 amino acid sequences of Rovalpituzumab tesirine (AbbVie Inc.), Tarlatamab (Amgen Inc.), HPN328 (Harpoon Therapeutics Inc.) or BI 764532 (Boehringer Ingelheim GmbH). In some embodiments, the selected VH1 or VL1 sequences of the DLL3 antigen binding fragment are the corresponding VH or VL amino acid sequences of Rovalpituzumab tesirine (AbbVie Inc.), Tarlatamab (Amgen Inc.), HPN328 (Harpoon Therapeutics Inc.) or BI 764532 (Boehringer Ingelheim GmbH).

In some embodiments, the first antigen-binding domain specifically binds to PSMA; and the second antigen-binding domain that specifically binds to NKp46.

In some embodiments, the selected CDRs of the PSMA antigen-binding fragment are the corresponding VH CDRs 1, 2, 3 and VL CDRs 1, 2, 3 amino acid sequences of HPN-424 (Harpoon Therapeutics Inc.), LAVA-1207 (LAVA Therapeutics NV), REGN-5678 (Regeneron Pharmaceuticals Inc.), REGN4336 (Regeneron Pharmaceuticals Inc.), Acapatamab (Amgen Inc.) or AMG-340 (Amgen Inc.). In some embodiments, the selected VH1 or VL1 sequences of the PSMA antigen binding fragment are the corresponding VH or VL amino acid sequences of HPN-424 (Harpoon Therapeutics Inc.), LAVA-1207 (LAVA Therapeutics NV), REGN-5678 (Regeneron Pharmaceuticals Inc.), REGN4336 (Regeneron Pharmaceuticals Inc.), Acapatamab (Amgen Inc.) or AMG-340 (Amgen Inc.).

In some embodiments, the first antigen-binding domain specifically binds to is STEAP1; and second antigen-binding domain that specifically binds to NKp46.

In some embodiments, the selected CDRs of the STEAP1 antigen-binding fragment are the corresponding VH CDRs 1, 2, 3 and VL CDRs 1, 2, 3 amino acid sequences of Xaluritamig (Amgen Inc.), ABBV-969 (AbbVie Inc.) or Vandortuzumab vedotin (Genentech Inc.). In some embodiments, the selected VH1 or VL1 sequences of the STEAP antigen binding fragment are the corresponding VH or VL amino acid sequences of Xaluritamig (Amgen Inc.), ABBV-969 (AbbVie Inc.) or Vandortuzumab vedotin (Genentech Inc.).

In some embodiments, the first antigen-binding domain specifically binds to MUC1; and the second antigen-binding domain that specifically binds to NKp46.

In some embodiments, the selected CDRs of the MUC1 antigen-binding fragment are the corresponding VH CDRs 1, 2, 3 and VL CDRs 1, 2, 3 amino acid sequences of MAB-AR20.5 (Canariabio Inc.) or DS-3939 (Glycotope GmbH). In some embodiments, the selected VH1 or VL1 sequences of the MUC1 antigen binding fragment are the corresponding VH or VL amino acid sequences of MAB-AR20.5 (Canariabio Inc.) or DS-3939 (Glycotope GmbH).

In some embodiments, the first antigen-binding domain specifically binds to c-MET; and the second antigen-binding domain that specifically binds to NKp46.

In some embodiments, the selected CDRs of the c-MET antigen-binding fragment are the corresponding VH CDRs 1, 2, 3 and VL CDRs 1, 2, 3 amino acid sequences of Amivantamab-VMJM (Janssen Biotech Inc.), Telisotuzumab vedotin (AbbVie Inc.), ABBV-400 (AbbVie Inc.), Bafisontamab (EpimAb Biotherapeutics Inc.), CKD-702 (Chong Kun Dang Pharmaceutical Corp.), Emibetuzumab (Eli Lilly & Co.), RC-108 (RemeGen Co., Ltd.), MCLA-129 (Merus NV), REGN-5093 (Regeneron Pharmaceuticals Inc.) or REGN5093-M114 (Regeneron Pharmaceuticals Inc.). In some embodiments, the selected VH1 or VL1 sequences of the c-MET antigen binding fragment are the corresponding VH or VL amino acid sequences of Amivantamab-VMJM (Janssen Biotech Inc.), Telisotuzumab vedotin (AbbVie Inc.), ABBV-400 (AbbVie Inc.), Bafisontamab (EpimAb Biotherapeutics Inc.), CKD-702 (Chong Kun Dang Pharmaceutical Corp.), Emibetuzumab (Eli Lilly & Co.), RC-108 (RemeGen Co., Ltd.), MCLA-129 (Merus NV), REGN-5093 (Regeneron Pharmaceuticals Inc.) or REGN5093-M114 (Regeneron Pharmaceuticals Inc.).

In some embodiments, the first antigen-binding domain specifically binds to LRRC15; and the second antigen-binding domain that specifically binds to NKp46.

In some embodiments, the selected CDRs of the LRRC15 antigen-binding fragment are the corresponding VH CDRs 1, 2, 3 and VL CDRs 1, 2, 3 amino acid sequences of QL-315 (Qilu Pharmaceutical Co., Ltd.), RAD-502 (Radiopharm Theranostics Ltd.) or Samrotamab (AbbVie Inc.). In some embodiments, the selected VH1 or VL1 sequences of the LRRC5 antigen binding fragment are the corresponding VH or VL amino acid sequences of QL-315 (Qilu Pharmaceutical Co., Ltd.), RAD-502 (Radiopharm Theranostics Ltd.) or Samrotamab (AbbVie Inc.).

In some embodiments, the first antigen-binding domain specifically binds to CCR8; and the second antigen-binding domain that specifically binds to NKp46.

In some embodiments, the selected CDRs of the CCR8 antigen-binding fragment are the corresponding VH CDRs 1, 2, 3 and VL CDRs 1, 2, 3 amino acid sequences of BMS-986340 (Bristol Myers Squibb Co.), LM-108 (LaNova Medicines Ltd.), S-531011 (Shionogi & Co., Ltd.), AMG-355 (Amgen Inc.), BAY3375968 (Bayer AG), BGB-A3055 (BeiGene Ltd.), CM369 (Keymed Biomedical Technology (Chengdu) Co., Ltd.), HBM-1022 (Harbour BioMed (Shanghai) Co., Ltd.), PSB-114 (Qilu Puget Sound Biotherapeutics Corp.) or SRF-114 (Surface Oncology Inc.). In some embodiments, the selected VH1 or VL1 sequences of the CCR8 antigen binding fragment are the corresponding VH or VL amino acid sequences of BMS-986340 (Bristol Myers Squibb Co.), LM-108 (LaNova Medicines Ltd.), S-531011 (Shionogi & Co., Ltd.), AMG-355 (Amgen Inc.), BAY3375968 (Bayer AG), BGB-A3055 (BeiGene Ltd.), CM369 (Keymed Biomedical Technology (Chengdu) Co., Ltd.), HBM-1022 (Harbour BioMed (Shanghai) Co., Ltd.), PSB-114 (Qilu Puget Sound Biotherapeutics Corp.) or SRF-114 (Surface Oncology Inc.).

In some embodiments, the first antigen-binding domain specifically binds to OX40; and the second antigen-binding domain that specifically binds to NKp46.

In some embodiments, the selected CDRs of the OX40 antigen-binding fragment are the corresponding VH CDRs 1, 2, 3 and VL CDRs 1, 2, 3 amino acid sequences of Amlitelimab (Kymab Ltd.), Rocatinlimab (Kyowa Kirin Co., Ltd.), INBRX-106 (Tnhibrx Inc.), BGB-A445 (BeiGene Ltd.), INCAGN-1949, Ivuxolimab (Pfizer Inc.), MEDI-6469 (Providence Cancer Center), BAT-6026 (Bio-Thera Solutions, Ltd.), IMG-007 (HUTCHMED (China) Ltd.), ABBV-368 (AbbVie Inc.) or BMS-986178 (Bristol Myers Squibb Co.). In some embodiments, the selected VH1 or VL1 sequences of the OX40 antigen binding fragment are the corresponding VH or VL amino acid sequences of Amlitelimab (Kymab Ltd.), Rocatinlimab (Kyowa Kirin Co., Ltd.), INBRX-106 (Inhibrx Inc.), BGB-A445 (BeiGene Ltd.), INCAGN-1949, Ivuxolimab (Pfizer Inc.), MEDI-6469 (Providence Cancer Center), BAT-6026 (Bio-Thera Solutions, Ltd.), IMG-007 (HUTCHMED (China) Ltd.), ABBV-368 (AbbVie Inc.) or BMS-986178 (Bristol Myers Squibb Co.).

The sequences of the commercially available antibodies are known in the art.

### NK Cell Surface Antigen

The “NK cell surface antigen” refers to a molecule (e.g., a surface receptor) on an NK cell, that is involved in the regulation and/or induction of NK cell functions.

Natural killer (NK) cells play important roles in innate defenses against viruses and in the control of tumor growth and metastasis. The regulation/induction of NK cell function is mediated by an array of activating or inhibitory surface receptors. In humans, major activating receptors involved in target cell killing are the natural cytotoxicity receptors (NCRs) and NKG2D. Activating receptors recognize ligands that are overexpressed or expressed de novo upon cell stress, viral infection, or tumor transformation. The HLA-class 1-specific inhibitory receptors, including KIRs recognizing HLA-class I allotypic determinants and CD94/NKG2A recognizing the class-Ib HLA-E, constitute a fail-safe mechanism to avoid unwanted NK-mediated damage to healthy cells. Other receptors such as PD-1, primarily expressed by activated T lymphocytes, are important inhibitory checkpoints of immune responses that ensure T-cell tolerance. PD-1 also may be expressed by NK cells in cancer patients. Since PD-1 ligand (PD-L1) may be expressed by different tumors, PD-1/PD-L1 interactions inactivate both T and NK cells. Thus, the reliable evaluation of PD-L1 expression in tumors has become a major issue to select patients who may benefit from therapy with mAbs disrupting PD-1/PD-L1 interactions. Recently, NKG2A was revealed to be an important checkpoint controlling both NK and T-cell activation.

Human NK cells express several receptors that can trigger their function upon interaction with specific ligands on the surface of transformed, virus-infected, or stressed cells. The NCRs are among the major activating NK receptors and consist of three elements, called NKp46 (also referred to as NCR1/CD335) NKp44 (also referred to as NCR2/CD33623), and NKp30 (also referred to as NCR3/CD337). These molecules were classically described as germline-encoded receptors and are important for inducing NK cell cytotoxic function against tumors and infected cells. NKp46 and NKp30 are expressed on nearly all resting human NK cells, upregulated on activated NK cells and downregulated on “adaptive” NK cells that are found in CMV+ individuals. Unlike NKp46 and NKp30, NKp44 is constitutively expressed only on CD56bright NK cells, but it is acquired by essentially all NK cells after activation by cytokines. NCRs are type I transmembrane molecules belonging to the immunoglobulin-like family. These receptors were named in accordance with their molecular weights according to sodium dodecyl sulfate polyacrylamide gel electrophoresis (NKp46, NKp30, and NKp44). Their transmembrane domains contain a positively charged amino acid that allows their association with the transmembrane regions of the TCR-ζ and/or FcεRI-γ (for NKp30 and NKp46) or KARAP/DAP1266 (for NKp44) adaptor proteins. The physical association between NKp44 and KARAP/DAP12 is essential for NKp44 surface expression and that the decreased surface density of NKp30 and NKp46 on “adaptive” NK cells is associated with the lack of FcεRI-γ expression in these cells. Another important activating NK receptor is NKG2D, a type II transmembrane and C-type lectin-like receptor, which may be expressed on cytotoxic T cells. NKG2D ligands are represented by ULBPs and MICA/B, 83 which are HLA-cl I structural homologs that are upregulated in infected, stressed, and tumor cells. Notably, shedding of NKG2D ligands by tumor cells may represent a mechanism of tumor escape.

Human NK cells express two different classes of HLA-class I-specific inhibitory receptors: members of the KIR/CD158 family and the CD94/NKG2A (CD94/CD159a) heterodimer. KIRs are type I transmembrane receptors specific for polymorphic HLA-A, B and C molecules, whereas NKG2A is a type II transmembrane receptor that recognizes HLA-E, a non-classical HLA molecule characterized by limited polymorphism. To transduce inhibitory signals, both types of inhibitory receptors contain ITIM motifs in their cytoplasmic tail. In addition, activating forms of KIRs have been identified. Different from inhibitory KIRs, activating KIRs lack ITIM motifs in their cytoplasmic tail and have a transmembrane domain carrying a charged amino acid residue that mediates the association with the ITAM-bearing molecule KARAP/DAP12. The role of activating KIRs in the immune response is only partially known. Specificity for HLA-cl I molecules has been demonstrated for only a few of them.

A detailed review of the NK cell surface antigens can be found, e.g., in Sivori et al., Cellular & Molecular Immunology, volume 16, pages 430-441 (2019), the entire content of which is incorporated herein by reference.

In some embodiments, an NK cell surface antigen is an NK cell activating receptor.

In some embodiments, the NK cell surface antigen is selected from NKp46, NKp30, CD16, NKG2D and NKG2A.

### Multispecific Antibodies or Antigen-Binding Fragments Thereof

In one aspect, provided herein are antibodies or antigen-binding fragments thereof, comprising: i) a first antigen-binding domain that specifically binds to a first antigen, wherein the first antigen is a tumor associated antigen (TAA); ii) a second antigen-binding domain that specifically binds to a second antigen, wherein the second antigen is an NK cell surface antigen. In some embodiments, the antibody comprises a fragment crystallizable region (Fc region). In some embodiments, the antibody is a bispecific antibody.

In some embodiments, the bispecific antibodies or antigen-binding fragments thereof (e.g., anti-CD79b/NKp46 antibodies) specifically bind to a tumor associated antigen (e.g., CD79b) and an NK cell surface antigen (e.g., NKp46), and such bispecific antibodies have optimized Fc regions.

In some embodiments, the first antigen-binding domain comprises a first heavy chain variable region (VH1) and a first light chain variable region (VL1); and the second antigen-binding domain comprises a second heavy chain variable region (VH2) and a second light chain variable region (VL2).

In some embodiments, the first antigen-binding domain is a human or humanized antigen-binding domain; and/or the second antigen-binding domain is a human or humanized antigen-binding domain.

In some embodiments, the first antigen-binding domain is a single-chain variable fragment (scFV); and/or the second antigen-binding domain is a scFv.

In some embodiments, the multispecific antibody described herein is designed to have the Fc region that comprises one of the following (all numbering is according to EU numbering):
i) an Alanine (A) at position 236, a Leucine (L) at position 330, and a Glutamic acid I at position 332;
ii) an Alanine (A) at position 236, an Aspartic acid (D) at position 293, a Leucine (L) at position 330, and a Glutamic acI(E) at position 332;
iii) an Alanine (A) at position 236;
iv) an Alanine (A) at position 236, an Aspartic acid (D) at position 293, and a GlutamicIid (E) at position 332;
v) an Aspartic acid (D) at position 293 and a GlutaI acid (E) at position 332; and
vi) an Aspartic acid (D) at position 293, a Leucine (L) at position 330, and a GlImic acid (E) at position 332.

In some embodiments, the antibody or antigen-binding fragment thereof comprises a fragment crystallizable region (Fc region). In some embodiments, the multispecific antibody described herein is designed to have an IgG1 subtype structure with the optimized mutations (G236A, A330L, and 1332E mutations in EU numbering). In some embodiments, the multispecific antibody described herein is designed to have an IgG1 Fc region having an Alanine (A) at position 236, a Leucine (L) at position 330, and aIutamic acid (E) at position 332 in EU numbering. In some embodiments, The Fc region comprises an amino acid sequence that is about or at least 60%, 65%, 70%, 75%, 80%, 85%, 90%, 95%, or 100% identical to SEQ ID NO: 43.

In some embodiments, the multispecific antibody described herein is designed to have an IgG1 subtype structure with the GASDALIE mutations (G236A, S293D, A330L, and 1332E mutations in EU numbering). In some embodiments, the multispecific antibody described herein is designed to have an IgG1 Fc region having an Alanine (A) at position 236, an Aspartic acid (D) at position 293, a Leucine (L) at position 330, anI Glutamic acid (E) at position 332 in EU numbering.

In some embodiments, the multispecific antibody described herein is designed to have an IgG1 subtype structure with the G236A mutation in EU numbering. In some embodiments, the multispecific antibody described herein is designed to have an IgG1 Fc region having an Alanine (A) at position 236 in EU numbering.

In some embodiments, the multispecific antibody described herein is designed to have an IgG1 subtype structure with the G236A, S293D, and T332E mutations in EU numbering. In some embodiments, the multispecific antibody described herein is designed to have an IgG1 Fc region having an Alanine (A) at position 236, an Aspartic acid (D) at position 293, Id a Glutamic acid (E) at position 332 in EU numbering.

In some embodiments, the multispecific antibody described herein is designed to have an IgG1 subtype structure with the S293D, and 1332E mutations in EU numbering. In some embodiments, the multispecific antibody described herein is designed to have an IgG1 Fc region having an Aspartic acid (D) at position 293, and a Glutamic acid (E) at position 332 in EU numbering.

In some embodiments, the multispecific antibody described herein is designed to have an IgG1 subtype structure with the S293D, A330L, and 1332E mutations in EU numbering. In some embodiments, the multispecific antibody described herein is designed to have an IgG1 Fc region having an Aspartic acid (D) at position 293, a Leucine (L) at position 330, and a Glutamic acid (E) at position 332 in EU numbering.

In some embodiments, the multispecific antibody described herein can be designed to have an IgG1 subtype structure with knobs-into-holes (KIH) mutations, which can promote heterodimerization and avoid mispairing between the two heavy chains. In some embodiments, the multispecific antibody has a higher endocytosis rate than the corresponding monoclonal antibodies or the control multispecific antibodies. In some embodiments, the antibody or antigen-binding fragment thereof has an increased binding affinity to the FcγRIIa receptor and/or the FcγRIIIa receptor.

The first antigen-binding fragment and the second antigen-binding fragment of the bi-specific antibodies or antigen-binding fragments described herein can be in any suitable configurations. In some embodiments, wherein the second antigen-binding domain is a single-chain fragment variable (scFv) domain comprising a light chain variable domain (VL) and a heavy chain variable domain (VH) linked by a first linker.

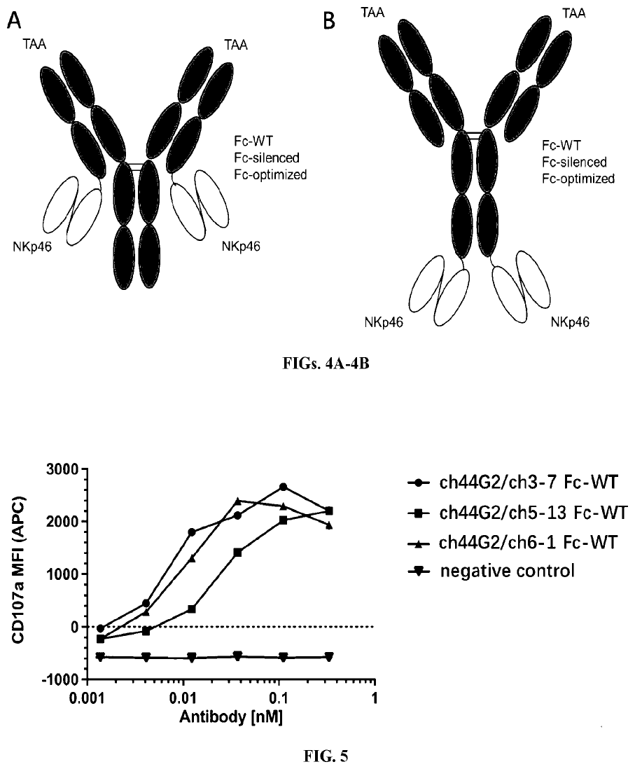

In some embodiments, the second antigen-binding domain is linked to the C-terminus of the light chain of the first antigen-binding domain through a second linker. In some embodiments, the second antigen-binding domain is connected to the C-terminus of the light chain of the first antigen-binding domain through a second linker. In some embodiments, the heavy chain variable domain of the first antigen-binding domain is linked to the Fc region. In some embodiments, the VH1 is linked to a CHI domain, and the VL1 is linked to a CL domain. A schematic illustration of this configuration is shown in FIGS. 4A-4B.

In some embodiments, the second antigen-binding domain is linked to the C-terminus of the Fc region. In some embodiments, the heavy chain variable domain of the first antigen-binding domain is linked to the Fc region. A schematic illustration of this configuration is shown in FIG. 4B. In some embodiments, the heavy chain variable domain of the first antigen-binding domain is linked to the Fc region via a third linker.

In some embodiments, the multispecific antibody has a heavy chain sequence comprising a wild-type IgG1 Fc region (SEQ ID NO: 41). In some embodiments, the multispecific antibody has a heavy chain sequence comprising an Fc region that is about or at least 80%, 85%, 86%, 87%, 88%, 89%, 90%, 91%, 92%, 93%, 94%, 95%, 96%, 97%, 98%, 99% E or 100% identical to SEQ ID NO: 41.

In some embodiments, the multispecific antibody has a heavy chain sequence comprising an IgG1 Fc region with the optimized mutations (SEQ ID NO: 43). In some embodiments, the multispecific antibody has a heavy chain sequence comprising an Fc region that is about or at least 80%, 85%, 86%, 87%, 88%, 89%, 90%, 91%, 92%, 93%, 94%, 95%, 96%, 97%, 98%, 99% or 100% identical to SEQ ID NO: 43.

In some embodiments, the Fc region of the multispecific antibody is afucosylated.

The linkers described herein can be any suitable linkers known in the art. In some embodiments, the linker includes an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 87

### Anti-CD79b/NKp46 Antibodies and Antigen-binding Fragments Thereof

CD79b is a cell-surface antigen expressed on mature B cells but absent on plasma cells. The immunoglobulin (Ig) beta protein (CD79b) dimerizes with Ig alpha (CD79a) to form the signaling component of the B-cell antigen receptor complex (BCR). Given the critical role of Ig beta in BCR signaling, it has been suggested that its variable expression in CLL may induce either cell proliferation or apoptosis. High CD79b expression has been associated with atypical morphology, strong expression of surface Ig, advanced clinical stage and short overall survival in B-CLL. Moreover, high CD38 expression was correlated with high CD40, CD69 and CD79b which are consistent with an activation phenotype. Furthermore, it has been demonstrated that the ZAP-70+ CLL subgroup shows a rapid disease progression and an inferior overall survival. Since it has been described that BCR engagement has significant effects on B-CLL cell survival, activation and cell cycle progression, CD79b is an important prognostic parameter and could be used to early identify and stratify B-CLL progressive patients.

A majority of mature malignancies of B-cell origin, including non-Hodgkin lymphoma (NHL) and chronic lymphocytic leukemia, express CD79b and demonstrate rapid internalization of anti-CD79b antibodies. Studies with polatuzumab vedotin, an antibody-drug conjugate (ADC) targeting CD79b for delivery of the microtubule-disrupting agent—monomethyl auristatin E (MMAE)—have clinically validated CD79b as a useful target for transporting cytotoxic agents to the malignant cell.

A detailed review of CD79b and its functions can be found in Del Poeta et al., Blood (2008) 112 (11): 1054; Herrera et al., Clin Cancer Res. 2022 Apr. 1; 28(7): 1294-1301; and Huang et al., Pathol Int 2011; 61:122-9, each of which is incorporated by reference in its entirety.

Natural killer (NK) cells are granular lymphocytes that were initially recognized for their capacity to efficiently eliminate tumor cells without prior sensitization. Viral infections can also induce NK cytotoxicity. Indeed, NK cells are an important component of the innate immune response against viruses, as evidenced by the abundant infections in patients with primary NK cells deficiencies and mice lacking NK cells. In healthy human adults. NK cells compromise 5-15% of peripheral blood lymphocytes. The majority of NK cells (~90%) are CD56dim CD16bright, and can induce a strong cytolytic response, while 10% are CD56bright-CD16null/dim and are capable of rapid cytokine secretion. Control of NK cell function is mainly regulated by its recognition of self MHC class I molecules through a set of inhibitory receptors—the killer-cell immunoglobulin-like receptors (KIRs) in humans and the Ly49 receptor family in mice. NK cells do not express variable antigen specific receptors. Instead, their activation is controlled by a repertoire of germline encoded activating receptors that recognize different ligands on the surface of susceptible target cells. Important NK activating receptors include NKG2D and three members of the natural cytotoxicity receptors group (NCR): NKp46, NKp44, and NKp30.

Distinct among the NCRs, NKp46 (NCR1) is evolutionary conserved between mice and humans, and NKp46 activity has been studied in several mouse models. NKp46 is uniquely expressed on all NK cell subsets and has been suggested as a possible target for NK cell ablation and as a pan NK cell marker. NKp46 is a 46 kDa type 1 transmembrane glycoprotein characterized by a 30-aa intracellular tail, 20-aa transmembrane domain, and two extracellular Ig-like domains that are contacted through a 25-aa short peptide. The transmembrane domain contains an Arg residue that can bind the signal adapter proteins FcϵRI and CD3ζ. Two NKp46 extracellular (D1-D2) crystal structures were resolved and showed similarity to the extracellular domains of different leukocyte immunoglobulin-like receptors (LIRs) and KIR receptors. The membrane-proximal domain (D2) was identified as the ligand-binding domain of NKp46 receptor.

In addition to the role of NKp46 in recognition of cancer cells, several studies suggest the involvement of NKp46 in other pathological conditions. NKp46 was shown to be crucial for development of type 1 diabetes through recognition of an unknown ligand on pancreatic β-cells. It is also demonstrated that NKp46 contributes to clearance of Streptococcus pneumoniae by interacting with infected alveolar macrophages. NKp46 also mediates the killing of stellate cells in attenuated liver fibrosis and of HCV-infected hepatocytes. In a murine model of random mutation (Ncr1Noé/Noé), the lack of NKp46 surface expression resulted in hyper-responsiveness of NK cells following MCMV infection. NKp46 also regulates graft-versus-host disease and allergic response.

More information related to NKp46 can be found e.g., in Hadad et al., Front Immunol. 2015; 6: 495; Sivori et al., J Exp Med (1997) 186:1129; Pessino et al., J Exp Med (1998) 188:953; and Westgaard et al., J Leukoc Biol (2004) 76:1200-6, each of which is incorporated herein by reference in the entirety.

In some embodiments, the multispecific (e.g., bispecific) anti-CD79b/NKp46 antibody described herein has an agonistic activity on the activation of NK cells. In some embodiments, the activation of NK cells is increased by about or at least 5%, 10%, 15%, 200%, 25%, 30%, 35%, 40%, 45%, 50%, 55%, 60%, 65%, 70%, 75%, 80%, 85%, 90%, 95%, or 100% upon contact with the anti-CD79b/NKp46 antibody described herein, compared to the activation of NK cells without contact with such antibody. In some embodiments, the activation of NK cells is measured by the expression of activation marker such as CD56. In some embodiments, the anti-CD79b/NKp46 antibody described herein binds to NK cells with the optimized mutations on the Fc region (SEQ ID NO: 43).

In some embodiments, the multispecific (e.g., bispecific) anti-CD79b/NKp46 antibody described herein binds to NK cells with silent mutations (e.g., LALA-PG mutations) on the Fc region. In some embodiments, the anti-CD79b/NKp46 antibody described herein binds to target cells (e.g., Ramos cells) with silent mutations (e.g., LALA-PG mutations) on the Fc region.

In some embodiments, the multispecific (e.g., bispecific) anti-CD79b/NKp46 antibody described herein activates NK cells (e.g., through the engagement of NKp46), which mediate the killing of target cells (e.g., B cells or B cell lines such as Ramos cells or Daudi cells). In some embodiments, the anti-CD79b/NKp46 antibody described herein activates NK cells with silent mutations (e.g., LALA-PG mutations) on the Fc region. In some embodiments, the anti-CD79b/NKp46 antibody described herein includes a wild-type human IgG1 Fc region. In some embodiments, the anti-CD79b/NKp46 antibody described herein binds to NK cells with the optimized mutations (SEQ ID NO: 43) on the Fc region.

In some embodiments, the multispecific (e.g., bispecific) anti-CD79b/NKp46 antibody described herein activates NK cells (e.g., through the engagement of NKp46) in a peripheral blood mononuclear cell (PBMC) population. In some embodiments, the PBMCs mediate the killing of target cells (e.g., B cells). In some embodiments, about 5%, 10%, 15%, 20%, 25%, 30%, 35%, 40%, 45%, 50%, 55%, 60%, 65%, 70%, 75%, 80%, 85%, 90%, 95% or more of the PBMCs are NK cells.

In some embodiments, the multispecific (e.g., bispecific) anti-CD79b/NKp46 antibody described herein promotes monocyte-mediated killing of target cells (e.g., B cells or B cell lines such as Ramos cells). In some embodiments, the anti-CD79b/NKp46 antibody described herein includes a wild-type human IgG1 Fc region. In some embodiments, the anti-CD79b/NKp46 antibody described herein binds to NK cells with the optimized mutations (SEQ ID NO: 43) on the Fc region. In some embodiments, the target cell is a CD79+ cancer cell. In some embodiments, the target cells is Ramos cell. In some embodiments, the killing of the target cells is by eith”r or ‘both of phagocytosis and trogocytosis type of cancer cell killing mechanism. In some embodiments, about 5%, 10%, 15%, 20%, 25%, 30%, 35%, 40%, 45%, 50%, 55%, 60%, 65%, 70%, 75%, 80%, 85%, 90%, 95% or more of the target cells are killed.

In some embodiments, there is no significant change of cell numbers (e.g., NK cell numbers or monocyte cell numbers) after the killing of the target cells.

The disclosure provides multispecific (e.g., bispecific) antibodies and antigen-binding fragments thereof that specifically bind to CD79b/NKp46 (e.g., human CD79b/NKp46). In one aspect, the disclosure provides an anti-CD79b/NKp46 multispecific (e.g., bispecific) antibody or antigen-binding fragment thereof, comprising: a first antigen-binding domain that specifically binds to CD79b; and a second antigen-binding domain that specifically binds to NKp46.

In some embodiments, the first antigen-binding domain comprises a first heavy chain variable region (VH1) and a first light chain variable region (VL1), and the second antigen-binding domain comprises a second heavy chain variable region (VH2) and a second light chain variable region (VL2).

In some embodiments, the first heavy chain variable region (VH1) comprises complementarity determining regions (CDRs) 1, 2, and 3, wherein the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and the first light chain variable region (VL1) comprises CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence, wherein the selected VH1 CDRs 1, 2, and 3 amino acid sequences, and the selected VL1 CDRs 1, 2, and 3 amino acid sequences are one of the following:
(1) the selected VH1 CDRs 1, 2, 3 amino acid sequences are set forth in SEQ ID NOs: 49, 51 and 53, respectively, and the selected VL1 CDRs 1, 2, 3 amino acid sequences are set forth in SEQ ID NOs: 54, 55 and 56, respectively; and
(2) the selected VH1 CDRs 1, 2, 3 amino acid sequences are set forth in SEQ ID NOs: 50, 52 and 53, respectively, and the selected VL1 CDRs 1, 2, 3 amino acid sequences are set forth in SEQ ID NOs: 54, 55 and 56, respectively, and
(3) the selected VH1 CDRs 1, 2, 3 amino acid sequences and the selected VL1 CDRs 1, 2, 3 amino acid sequences are the corresponding VH CDRs 1, 2, 3 and VL CDRs, 1, 2, 3 of Polatuzumab, PRV-3279 and SHR-A1912.

In some embodiments, the second heavy chain variable region (VH2) comprises CDRs 1, 2, and 3, wherein the VH2 CDR1 region comprises an amino acid sequence that is at least 80% identical to a selected VH2 CDR1 amino acid sequence, the VH2 CDR2 region comprises an amino acid sequence that is at least 80% identical to a selected VH2 CDR2 amino acid sequence, and the VH2 CDR3 region comprises an amino acid sequence that is at least 80% identical to a selected VH2 CDR3 amino acid sequence; and
the second light chain variable region (VL2) comprises CDRs 1, 2, and 3, wherein the VL2 CDR1 region comprises an amino acid sequence that is at least 80% identical to a selected VL2 CDR1 amino acid sequence, the VL2 CDR2 region comprises an amino acid sequence that is at least 80% identical to a selected VL2 CDR2 amino acid sequence, and the VL2 CDR3 region comprises an amino acid sequence that is at least 80% identical to a selected VL2 CDR3 amino acid sequence, wherein the selected VH2 CDRs 1, 2, and 3 amino acid sequences, and the selected VL2 CDRs 1, 2, and 3 amino acid sequences are one of the following:
   (1) the selected VH2 CDRs 1, 2, 3 amino acid sequences are set forth in SEQ ID NOs: 5, 7, and 9, respectively, and the selected VL2 CDRs 1, 2, 3 amino acid sequences are set forth in SEQ ID NOs: 10, 11, and 12, respectively;
   (2) the selected VH2 CDRs 1, 2, 3 amino acid sequences are set forth in SEQ ID NOs: 6, 8, and 9, respectively, and the selected VL2 CDRs 1, 2, 3 amino acid sequences are set forth in SEQ ID NOs: 10, 11 and 12, respectively;
   (3) the selected VH2 CDRs 1, 2, 3 amino acid sequences are set forth in SEQ ID NOs: 15, 17, and 19, respectively, and the selected VL2 CDRs 1, 2, 3 amino acid sequences are set forth in SEQ ID NOs: 20, 21, and 22, respectively;
   (4) the selected VH2 CDRs 1, 2, 3 amino acid sequences are set forth in SEQ ID NOs: 16, 18, and 19, respectively, and the selected VL2 CDRs 1, 2, 3 amino acid sequences are set forth in SEQ ID NOs: 20, 21 and 22, respectively;
   (5) the selected VH2 CDRs 1, 2, 3 amino acid sequences are set forth in SEQ ID NOs: 25, 27, and 29, respectively, and the selected VL2 CDRs 1, 2, 3 amino acid sequences are set forth in SEQ ID NOs: 30, 31, and 32, respectively; and
   (6) the selected VH2 CDRs 1, 2, 3 amino acid sequences are set forth in SEQ ID NOs: 26, 28, and 29, respectively, and the selected VL2 CDRs 1, 2, 3 amino acid sequences are set forth in SEQ ID NOs: 30, 31 and 32, respectively.

In some embodiments, the selected VH1 CDRs 1, 2, 3 amino acid sequences are set forth in SEQ ID NOs: 49, 51 and 53, respectively, and the selected VL1 CDRs 1, 2, 3 amino acid sequences are set forth in SEQ ID NOs: 54, 55 and 56, respectively; and the selected VH2 CDRs 1, 2, 3 amino acid sequences are set forth in SEQ ID NOs: 5, 7, and 9, respectively, and the selected VL2 CDRs 1, 2, 3 amino acid sequences are set forth in SEQ ID NOs: 10, 11, and 12, respectively;

In some embodiments, the selected VH1 CDRs 1, 2, 3 amino acid sequences are set forth in SEQ ID NOs: 50, 52 and 53, respectively, and the selected VL1 CDRs 1, 2, 3 amino acid sequences are set forth in SEQ ID NOs: 54, 55 and 56, respectively; and the selected VH2 CDRs 1, 2, 3 amino acid sequences are set forth in SEQ ID NOs: 6, 8, and 9, respectively, and the selected VL2 CDRs 1, 2, 3 amino acid sequences are set forth in SEQ ID NOs: 10, 11 and 12, respectively;

In some embodiments, the selected VH1 CDRs 1, 2, 3 amino acid sequences are set forth in SEQ ID NOs: 49, 51 and 53, respectively, and the selected VL1 CDRs 1, 2, 3 amino acid sequences are set forth in SEQ ID NOs: 54, 55 and 56, respectively; and the selected VH2 CDRs 1, 2, 3 amino acid sequences are set forth in SEQ ID NOs: 15, 17, and 19, respectively, and the selected VL2 CDRs 1, 2, 3 amino acid sequences are set forth in SEQ ID NOs: 20, 21, and 22, respectively;

In some embodiments, the selected VH1 CDRs 1, 2, 3 amino acid sequences are set forth in SEQ ID NOs: 50, 52 and 53, respectively, and the selected VL1 CDRs 1, 2, 3 amino acid sequences are set forth in SEQ ID NOs: 54, 55 and 56, respectively; and the selected VH2 CDRs 1, 2, 3 amino acid sequences are set forth in SEQ ID NOs: 16, 18, and 19, respectively, and the selected VL2 CDRs 1, 2, 3 amino acid sequences are set forth in SEQ ID NOs: 20, 21 and 22, respectively;

In some embodiments, the selected VH1 CDRs 1, 2, 3 amino acid sequences are set forth in SEQ ID NOs: 49, 51 and 53, respectively, and the selected VL1 CDRs 1, 2, 3 amino acid sequences are set forth in SEQ ID NOs: 54, 55 and 56, respectively; and the selected VH2 CDRs 1, 2, 3 amino acid sequences are set forth in SEQ ID NOs: 25, 27, and 29, respectively, and the selected VL2 CDRs 1, 2, 3 amino acid sequences are set forth in SEQ ID NOs: 30, 31, and 32, respectively;

In some embodiments, the selected VH1 CDRs 1, 2, 3 amino acid sequences are set forth in SEQ ID NOs: 50, 52 and 53, respectively, and the selected VL1 CDRs 1, 2, 3 amino acid sequences are set forth in SEQ ID NOs: 54, 55 and 56, respectively; and the selected VH2 CDRs 1, 2, 3 amino acid sequences are set forth in SEQ ID NOs: 26, 28, and 29, respectively, and the selected VL2 CDRs 1, 2, 3 amino acid sequences are set forth in SEQ ID NOs: 30, 31 and 32, respectively.

In some embodiments, the first heavy chain variable region comprises a sequence that is at least 80%, 85%, 90%, 95%, 99%, or 100% identical to SEQ ID NO: 45, the first light chain variable region comprises a sequence that is at least 80%, 85%, 90%, 95%, 99%, or 100% identical to SEQ ID NO: 46, the second heavy chain variable region comprises a sequence that is at least 80%, 85%, 90%, 95%, 99%, or 100% identical to SEQ ID NO: 3, and the second light chain variable region comprises a sequence that is at least 80%, 85%, 90%, 95%, 99%, or 100% identical to SEQ ID NO: 4.

In some embodiments, the first heavy chain variable region comprises a sequence that is at least 80%, 85%, 90%, 95%, 99%, or 100% identical to SEQ ID NO: 45, the first light chain variable region comprises a sequence that is at least 80%, 85%, 90%, 95%, 99%, or 100% identical to SEQ ID NO: 46, the second heavy chain variable region comprises a sequence that is at least 80%, 85%, 90%, 95%, 99%, or 100% identical to SEQ ID NO: 13, and the second light chain variable region comprises a sequence that is at least 80%, 85%, 90%, 95%, 99%, or 100% identical to SEQ ID NO: 14.

In some embodiments, the first heavy chain variable region comprises a sequence that is at least 80%, 85%, 90%, 95%, 99%, or 100% identical to SEQ ID NO: 45, the first light chain variable region comprises a sequence that is at least 80%, 85%, 90%, 95%, 99%, or 100% identical to SEQ ID NO: 46, the second heavy chain variable region comprises a sequence that is at least 80%, 85%, 90%, 95%, 99%, or 100% identical to SEQ ID NO: 23, and the second light chain variable region comprises a sequence that is at least 800, 85%, 90%, 95%, 99%, or 100% identical to SEQ ID NO: 24.

In some embodiments, the first heavy chain variable region comprises a sequence that is at least 80%, 85%, 90%, 95%, 99%, or 100% identical to SEQ ID NO: 47, the first light chain variable region comprises a sequence that is at least 80%, 85%, 900%, 95%, 99%, or 100% identical to SEQ ID NO: 48, the second heavy chain variable region comprises a sequence that is at least 80%, 85%, 90%, 95%, 99%, or 100% identical to SEQ ID NO: 33, and the second light chain variable region comprises a sequence that is at least 80%, 85%, 90%, 95%, 99%, or 100% identical to SEQ ID NO: 34.

In some embodiments, the first heavy chain variable region comprises a sequence that is at least 80%, 85%, 90%, 95%, 99%, or 100% identical to SEQ ID NO: 47, the first light chain variable region comprises a sequence that is at least 80%, 85%, 90%, 95%, 99%, or 100% identical to SEQ ID NO: 48, the second heavy chain variable region comprises a sequence that is at least 80%, 85%, 90%, 95%, 99%, or 100% identical to SEQ ID NO: 35, and the second light chain variable region comprises a sequence that is at least 80%, 85%, 90%, 95%, 99%, or 100% identical to SEQ ID NO: 36.

In some embodiments, the first heavy chain variable region comprises a sequence that is at least 80%, 85%, 90%, 95%, 99%, or 100% identical to SEQ ID NO: 47, the first light chain variable region comprises a sequence that is at least 80%, 85%, 90%, 95%, 99%, or 100% identical to SEQ ID NO: 48, the second heavy chain variable region comprises a sequence that is at least 80%, 85%, 90%, 95%, 99%, or 100% identical to SEQ ID NO: 37, and the second light chain variable region comprises a sequence that is at least 80%, 85%, 90%, 95%, 99%, or 100% identical to SEQ ID NO: 38.

In some embodiments, the VH1 comprises an amino acid sequence that is at least 80%, 85%, 90%, 95%, 99%, or 100% identical to a selected VH sequence, and the VL1 comprises an amino acid sequence that is at least 80%, 85%, 90%, 95%, 99%, or 100% identical to a selected VL sequence, wherein the selected VH sequence is SEQ ID NO: 45, and the selected VL sequence is SEQ ID NO: 46.1n some embodiments, the VH2 comprises an amino acid sequence that is at least 80%, 85%, 90%, 95%, 99%, or 100% identical to a selected VH sequence, and the VL2 comprises an amino acid sequence that is at least 80%, 85%, 90%, 95%, 99%, or 100% identical to a selected VL sequence, wherein the selected VH sequence and the selected VL sequence are one of the following:
(1) the selected VH sequence is SEQ ID NO: 3, and the selected VL sequence is SEQ ID NO: 4;
(2) the selected VH sequence is SEQ ID NO: 13, and the selected VL sequence is SEQ ID NO: 14; and
(3) the selected VH sequence is SEQ ID NO: 23, and the selected VL sequence is SEQ ID NO: 24.

In some embodiments, the VH1 comprises VH1 CDR1, VH1 CDR2, and VH1 CDR3 that are identical to VH CDR1, VH CDR2, and VH CDR3 of a selected VH sequence; and the VL1 comprising VL1 CDR1, VL1 CDR2, and VL1 CDR3 that are identical to VL CDR1, VL CDR2, and VL CDR3 of a selected VL sequence, wherein the selected VH sequence is SEQ ID NO: 33, and the selected VL sequence is SEQ ID NO: 34.

In some embodiments, the VH2 comprises VH2 CDR1, VH2CDR2, and VH2 CDR3 that are identical to VH CDR1, VH CDR2, and VH CDR3 of a selected VH sequence; and the VL2 comprising VL2 CDR1, VL2 CDR2, and VL2 CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 3, and the selected VL sequence is SEQ ID NO: 4;
(2) the selected VH sequence is SEQ ID NO: 13, and the selected VL sequence is SEQ ID NO: 14; and
(3) the selected VH sequence is SEQ ID NO: 23, and the selected VL sequence is SEQ ID NO: 24.

In some embodiments, the VH1 comprises an amino acid sequence that is at least 80%, 85%, 90%, 95%, 99%, or 100% identical to a selected VH sequence, and the VL1 comprises an amino acid sequence that is at least 80%, 85%, 90%, 95%, 99%, or 100% identical to a selected VL sequence, wherein the selected VH sequence is SEQ ID NO: 47, and the selected VL sequence is SEQ ID NO: 48.

In some embodiments, the VH2 comprises an amino acid sequence that is at least 80%, 85%, 90%, 95%, 99%, or 100% identical to a selected VH sequence, and the VL2 comprises an amino acid sequence that is at least 80%, 85%, 90%, 95%, 99%, or 100% identical to a selected VL sequence, wherein the selected VH sequence and the selected VL sequence are one of the following:
(1) the selected VH sequence is SEQ ID NO: 33, and the selected VL sequence is SEQ ID NO: 34;
(2) the selected VH sequence is SEQ ID NO: 35, and the selected VL sequence is SEQ ID NO: 36; and
(3) the selected VH sequence is SEQ ID NO: 37, and the selected VL sequence is SEQ ID NO: 38.

In some embodiments, the VH1 comprises VH1 CDR1, VH1 CDR2, and VH1 CDR3 that are identical to VH CDR1, VH CDR2, and VH CDR3 of a selected VH sequence; and the VL1 comprising VL1 CDR1, VL1 CDR2, and VL1 CDR3 that are identical to VL CDR1, VL CDR2, and VL CDR3 of a selected VL sequence, wherein the selected VH sequence is SEQ ID NO: 47, and the selected VL sequence is SEQ ID NO: 48.

In some embodiments, the VH2 comprises VH2 CDR1, VH2CDR2, and VH2 CDR3 that are identical to VH CDR1, VH CDR2, and VH CDR3 of a selected VH sequence; and the VL2 comprising VL2 CDR1, VL2 CDR2, and VL2 CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 33, and the selected VL sequence is SEQ ID NO: 34;
(2) the selected VH sequence is SEQ ID NO: 35, and the selected VL sequence is SEQ ID NO: 36; and
(3) the selected VH sequence is SEQ ID NO: 37, and the selected VL sequence is SEQ ID NO: 38.

In some embodiments, the first antigen-binding domain specifically binds to human, mouse, monkey, or dog CD79b; and/or the second antigen-binding domain specifically binds to human, mouse, monkey, or dog NKp46.

In some embodiments, the first antigen-binding domain is a human or humanized antigen-binding domain; and/or the second antigen-binding domain is a human or humanized antigen-binding domain.

In some embodiments, the first antigen-binding domain is a single-chain variable fragment (scFV); and/or the second antigen-binding domain is a scFv.

In some embodiments, the antibody or antigen-binding fragment thereof comprises a fragment crystallizable region (Fc region). In some embodiments, the multispecific (e.g., bispecific) anti-CD79b/NKp46 antibody described herein can be designed to have an IgG1 subtype structure with the optimized mutations (G236A, A330L, and 1332E mutations in EU numbering). In some embodiments, the anti-CD79b/NKp46 antibody described herein can be designed to have an IgG1 Fc region having an Alanine (A) at position 236, a Leucine (L) at position 330, and a Glutamic acid (E) at position 332 in EU numbering. In some embodiments, The Fc region comprises an amino acid sequence that is about or at least 60%, 65%, 70%, 75%, 80%, 85%, 90%, 95%, or 100% identical to SEQ ID NO: 43.

In some embodiments, the multispecific (e.g., bispecific) anti-CD79b/NKp46 antibody described herein can be designed to have an IgG1 subtype structure with knobs-into-holes (KIH) mutations, which can promote heterodimerization and avoid mispairing between the two heavy chains. In some embodiments, the anti-CD79b/NKp46 antibody has a higher endocytosis rate than the corresponding monoclonal antibodies or the control bispecific antibodies.

The first antigen-binding fragment and the second antigen-binding fragment of the bi-specific antibodies or antigen-binding fragments described herein can be in any suitable configurations. In some embodiments, wherein the second antigen-binding domain is a single-chain fragment variable (scFv) domain comprising a light chain variable domain (VL) and a heavy chain variable domain (VH) linked by a first linker.

In some embodiments, the second antigen-binding domain is linked to the C-terminus of the light chain of the first antigen-binding domain through a second linker. In some embodiments, the heavy chain variable domain of the first antigen-binding domain is linked to the Fc region. A schematic illustration of this configuration is shown in FIGS. 4A-4B.

In some embodiments, the multispecific (e.g., bispecific) anti-CD79b/NKp46 antibody includes a heavy chain sequence of ch44G2. In some embodiments, the heavy chain of ch44G2 includes an IgG1 Fc region comprising the LALA-PG mutations (SEQ ID NO: 48). In some embodiments, the heavy chain of ch44G2 comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 58, 59, 61, or 63.

In some embodiments, the light chain of the multispecific (e.g., bispecific) anti-CD79b/NKp46 antibody (e.g., ch44G2/ch3-7 Fc-silenced light chain) comprises an amino acid sequence that is about or at least 80%, 85%, 86%, 87%, 88%, 89%, 90%, 91%, 92%, 93%, 94%, 95%, %%, 97%, 98%, 99% or 100% identical to SEQ ID NO: 42.

In some embodiments, the heavy chain of the multispecific (e.g., bispecific) anti-CD79b/NKp46 antibody (e.g., ch44G2/ch3-7 Fc-silenced heavy chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 59; and the light chain of the multispecific (e.g., bispecific) anti-CD79b/NKp46 antibody (e.g., ch44G2/ch3-7 Fc-silenced light chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 60.

In some embodiments, the light chain of the multispecific (e.g., bispecific) anti-CD79b/NKp46 antibody (e.g., ch44G2/ch5-13 Fc-silenced light chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 62.

In some embodiments, the heavy chain of the multispecific (e.g., bispecific) anti-CD79b/NKp46 antibody (e.g., ch44G2/ch5-13 Fc-silenced heavy chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 61; and the light chain of the multispecific (e.g., bispecific) anti-CD79b/NKp46 antibody (e.g., ch44G2/ch5-13 Fc-silenced light chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 62.

In some embodiments, the light chain of the multispecific (e.g., bispecific) anti-CD79b/NKp46 antibody (e.g., ch44G2/ch6-1 Fc-silenced light chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 64.

In some embodiments, the heavy chain of the multispecific (e.g., bispecific) anti-CD79b/NKp46 antibody (e.g., ch44G2/ch6-1 Fc-silenced heavy chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 63; and the light chain of the multispecific (e.g., bispecific) anti-CD79b/NKp46 antibody (e.g., ch44G2/ch6-1 Fc-silenced light chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 64.

In some embodiments, the multispecific (e.g., bispecific) anti-CD79b/NKp46 antibody includes a heavy chain sequence of ch44G2. In some embodiments, the heavy chain of ch44G2 includes a wild-type IgG1 Fc region (SEQ ID NO: 41). In some embodiments, the heavy chain of ch44G2 comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 65, 67 and 69.

In some embodiments, the light chain of the multispecific (e.g., bispecific) anti-CD79b/NKp46 antibody (e.g., ch44G2/ch3-7 Fc-WT light chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 66.

In some embodiments, the heavy chain of the multispecific (e.g., bispecific) anti-CD79b/NKp46 antibody (e.g., ch44G2/ch3-7 Fc-WT heavy chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 65; and the light chain of the multispecific (e.g., bispecific) anti-CD79b/NKp46 antibody (e.g., ch44G2/ch3-7 Fc-WT light chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 66.

In some embodiments, the light chain of the multispecific (e.g., bispecific) anti-CD79b/NKp46 antibody (e.g., ch44G2/ch5-13 Fc-WT light chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 68.

In some embodiments, the heavy chain of the multispecific (e.g., bispecific) anti-CD79b/NKp46 antibody (e.g., ch44G2/ch5-13 Fc-WT heavy chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 67; and the light chain of the multispecific (e.g., bispecific) anti-CD79b/NKp46 antibody (e.gch44G2/ch5-13 Fc-WT light chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 68.

In some embodiments, the light chain of the multispecific (e.g., bispecific) anti-CD79b/NKp46 antibody (e.g., ch44G2/ch6-1 Fc-WT light chain) comprises an amino acid sequence that is about or at least 80%, 85%, 86/c, 87%, 88%, 89%, 90%, 91%, 92%, 93%, 94%, 95%, 96%, 97%, 98%, 99% or 100% identical to SEQ ID NO: 70.

In some embodiments, the heavy chain of the multispecific (e.g., bispecific) anti-CD79b/NKp46 antibody (e.g., ch44G2/ch6-1 Fc-WT heavy chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 69; and the light chain of the multispecific (e.g., bispecific) anti-CD79b/NKp46 antibody (e.g., ch44G2/ch6-1 Fc-WT light chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 70.

In some embodiments, the multispecific (e.g., bispecific) anti-CD79b/NKp46 antibody includes a heavy chain sequence of ch44G2. In some embodiments, the heavy chain of ch44G2 includes an IgG1 Fc region comprising the optimized mutations (SEQ ID NO: 43). In some embodiments, the heavy chain of ch44G2 comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 75, 77, 79, 81, 83, or 85.

In some embodiments, the light chain of the multispecific (e.g., bispecific) anti-CD79b/NKp46 antibody (e.g., ch44G2/ch6-1 (LC) Fc-optimized light chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 74.

In some embodiments, the heavy chain of the multispecific (e.g., bispecific) anti-CD79b/NKp46 antibody (e.g., ch44G2/ch6-1 (LC) Fc-optimized heavy chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 73; and the light chain of the multispecific (e.g., bispecific) anti-CD79b/NKp46 antibody (e.g., ch44G2/ch6-1 (LC) Fc-optimized light chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 74.

In some embodiments, the light chain of the multispecific (e.g., bispecific) anti-CD79b/NKp46 antibody (e.g., ch44G2/ch6-1 (HC) Fc-optimized light chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 76.

In some embodiments, the heavy chain of the multispecific (e.g., bispecific) anti-CD79b/NKp46 antibody (e.g., ch44G2/ch6-1 (HC) Fc-optimized heavy chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 75; and the light chain of the multispecific (e.g., bispecific) anti-CD79b/NKp46 antibody (e.g., ch44G2/ch6-1 (HC) Fc-optimized light chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 76.

In some embodiments, the light chain of the multispecific (e.g., bispecific) anti-CD79b/NKp46 antibody (e.g., h44G2/h6-1 (LC) Fc-optimized light chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 78.

In some embodiments, the heavy chain of the multispecific (e.g., bispecific) anti-CD79b/NKp46 antibody (e.g., h44G2/h6-1 (LC) Fc-optimized heavy chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 77, and the light chain of the multispecific (e.g., bispecific) anti-CD79b/NKp46 antibody (e.g., h44G2/h6-1 (LC) Fc-optimized light chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 78.

In some embodiments, the light chain of the multispecific (e.g., bispecific) anti-CD79b/NKp46 antibody (e.g., h44G2/h6-1 (HC) Fc-optimized light chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 80.

In some embodiments, the heavy chain of the multispecific (e.g., bispecific) anti-CD79b/NKp46 antibody (e.g., h44G2/h6-1 (HC) Fc-optimized heavy chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 79; and the light chain of the multispecific (e.g., bispecific) anti-CD79b/NKp46 antibody (e.g., h44G2/h6-1 (HC) Fc-optimized light chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 80.

In some embodiments, the second antigen-binding domain is linked to the C-terminus of the Fc region. In some embodiments, the heavy chain variable domain of the first antigen-binding domain is linked to the Fc region. A schematic illustration of this configuration is shown in FIG. 4B. In some embodiments, the heavy chain variable domain of the first antigen-binding domain is linked to the Fc region via a third linker.

In some embodiments, the anti-CD79b/NKp46 antibody includes a light chain sequence of ch44G2. In some embodiments, the light chain of ch44G2 comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 58.

The linkers described herein can be any suitable linkers known in the art. In some embodiments, the linker includes an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 87.

In some embodiments, knobs-into-holes mutations were introduced in the Fc regions of the multispecific (e.g., bispecific) antibodies to reduce the chance of wrong pairing between the two heavy chains.

The disclosure provides, e.g., anti-CD79b/NKp46 antibodies ch44G2/ch3-7 (also referred to as “44G2/3-7” or “44G2/ch3-7”), ch44G2/ch5-13 (also referred to as “44G2/5-13” or “44G2/ch5-13”) and ch44G2/ch6-1 (also referred to as “44G2/6-1” or “44G2/ch6-1”), the modified antibodies thereof, including, e.g., chimeric antibodies, humanized antibodies, and human antibodies.

In some embodiments, the multispecific (e.g., bispecific) anti-CD79b/NKp46 antibodies, or antibody fragments thereof include the combinations of anti-CD79b and anti-NKp46 antigen-binding domains shown in FIG. 33.

In some embodiments, ch44G2/ch3-7 refers to a multispecific (e.g., bispecific) anti-CD79b/NKp46 antibody that contains a first anti-CD79b antigen-binding domain that is derived from ch44G2 and a second anti-NKp46 antigen-binding domain that is derived from ch3-7. In some embodiments, the first anti-CD79b antigen-binding domain comprises the CDRs of ch44G2. In some embodiments, the second anti-NKp46 antigen-binding domain comprises the CDRs of ch3-7. In some embodiments, the first anti-CD79b antigen-binding domain comprises the VH and VL of ch44G2. In some embodiments, the second anti-NKp46 antigen-binding domain comprises the VH and VL of ch3-7.

In some embodiments, ch44G2/ch5-13 refers to a multispecific (e.g., bispecific) anti-CD79b/NKp46 antibody that contains a first anti-CD79b antigen-binding domain that is derived from ch44G2 and a second anti-NKp46 antigen-binding domain that is derived from ch5-13. In some embodiments, the first anti-CD79b antigen-binding domain comprises the CDRs of ch44G2.

In some embodiments, the second anti-NKp46 antigen-binding domain comprises the CDRs of ch5-13. In some embodiments, the first anti-CD79b antigen-binding domain comprises the VH and VL of ch44G2. In some embodiments, the second anti-NKp46 antigen-binding domain comprises the VH and VL of ch5-13.

In some embodiments, ch44G2/ch6-1 refers to a multispecific (e.g., bispecific) anti-CD79b/NKp46 antibody that contains a first anti-CD79b antigen-binding domain that is derived from ch44G2 and a second anti-NKp46 antigen-binding domain that is derived from ch6-1. In some embodiments, the first anti-CD79b antigen-binding domain comprises the CDRs of ch44G2. In some embodiments, the second anti-NKp46 antigen-binding domain comprises the CDRs of ch6-1. In some embodiments, the first anti-CD79b antigen-binding domain comprises the VH and VL of ch44G2. In some embodiments, the second anti-NKp46 antigen-binding domain comprises the VH and VL of ch6-1.

In some embodiments, the anti-CD79b/NKp46 antibody is a bispecific antibody. Bispecific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

Any of the anti-CD79b/NKp46 antibodies or antigen-binding fragments thereof described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an anti-CD79b/NKp46 antibody or an antigen-binding fragment in vitro (e.g., in tissue culture or when stored as a pharmaceutical composition) or in vivo (e.g., in a human).

The disclosure also provides nucleic acid comprising a polynucleotide encoding an anti-CD79b/NKp46 antibody. The immunoglobulin heavy chain or immunoglobulin light chain in the anti-CD79b/NKp46 antibody comprises CDRs as shown in FIG. 33. When the polypeptides are paired with corresponding polypeptide (e.g., a corresponding heavy chain variable region or a corresponding light chain variable region), the paired polypeptides bind to NKp46 and/or CD79b.

The anti-CD79b/NKp46 antibodies can also be anti-CD79b/NKp46 antibody variants (including derivatives and conjugates) of anti-CD79b/NKp46 antibodies or antibody fragments. Additional anti-CD79b/NKp46 antibodies provided herein are polyclonal, monoclonal, multispecific (multimeric, e.g., bispecific), human antibodies, chimeric antibodies (e.g., human-mouse chimera), single-chain antibodies, intracellularly-made antibodies (i.e., intrabodies), and antigen-binding fragments thereof. The anti-CD79b/NKp46 antibodies can be of any type (e.g., IgG, IgE, IgM, IgD, IgA, and IgY), class (e.g., IgG1, IgG2, IgG3, IgG4, IgA1, and IgA2), or subclass. In some embodiments, the anti-CD79b/NKp46 antibody or antigen-binding fragment is an IgG (e.g., IgG1 Fc region is shown in SEQ ID NO: 41) antibody or antigen-binding fragment thereof.

Fragments of anti-CD79b/NKp46 antibodies are suitable for use in the methods provided so long as they retain the desired affinity and specificity to both NKp46 and CD79b. Thus, a fragment of an anti-CD79b/NKp46 antibody will retain an ability to bind to NKp46 and CD79b.

### Anti-BCMA/NKp46 Antibodies and Antigen-Binding Fragments Thereof

B cell maturation antigen (BCMA) is encoded by a 2.92-kb TNFRSF17 gene located on the short arm of chromosome 16 (16p13.13) and composed of 3 exons separated by 2 introns. BCMA is a 184 amino acid and 20.2-kDa type III transmembrane glycoprotein, with the extracellular N terminus containing a conserved motif of 6 cysteines. BCMA was found to be a member of tumor necrosis factor (TNF) receptor (TNFR) superfamily. There are four natural splice variants of human BCMA that present with different receptor binding affinities, membrane-anchoring ability, and intracellular domain signaling. BCMA is a novel treatment target for multiple myeloma (MM) due to its highly selective expression in malignant plasma cells (PCs).

A detailed review of BCMA and its functions can be found in Yu et al., J Hematol Oncol. 2020; 13: 125, which is incorporated by reference in its entirety.

In some embodiments, the multispecific (e.g., bispecific) anti-BCMA/NKp46 antibody described herein has an agonistic activity on the activation of NK cells. In some embodiments, the activation of NK cells is increased by about or at least 5%, 10%, 15%, 20%, 25%, 30%, 35%, 40%, 45%, 50%, 55%, 60%, 65%, 70%, 75%, 80%, 85%, 90%, 95%, or 100% upon contact with the anti-BCMA/NKp46 antibody described herein, compared to the activation of NK cells without contact with such antibody. In some embodiments, the activation of NK cells is measured by the expression of activation marker such as CD56. In some embodiments, the anti-BCMA/NKp46 antibody described herein binds to NK cells with the optimized mutations on the Fc region (SEQ ID NO: 43).

In some embodiments, the multispecific (e.g., bispecific) anti-BCMA/NKp46 antibody described herein binds to NK cells with silent mutations (e.g., LALA-PG mutations) on the Fc region. In some embodiments, the anti-BCMA/NKp46 antibody described herein binds to target cells (e.g., Ramos cells) with silent mutations (e.g., LALA-PG mutations) on the Fc region.

In some embodiments, the multispecific (e.g., bispecific) anti-BCMA/NKp46 antibody described herein activates NK cells (e.g., through the engagement of NKp46), which mediate the killing of target cells (e.g., B cells or B cell lines such as Ramos cells or Daudi cells). In some embodiments, the anti-BCMA/NKp46 antibody described herein activates NK cells with silent mutations (e.g., LALA-PG mutations) on the Fc region. In some embodiments, the anti-BCMA/NKp46 antibody described herein includes a wild-type human IgG1 Fc region. In some embodiments, the anti-BCMA/NKp46 antibody described herein binds to NK cells with the optimized mutations (SEQ ID NO: 42) on the Fc region.

In some embodiments, the multispecific (e.g., bispecific) anti-BCMA/NKp46 antibody described herein activates NK cells (e.g., through the engagement of NKp46) in a peripheral blood mononuclear cell (PBMC) population. In some embodiments, the PBMCs mediate the killing of target cells (e.g., B cells). In some embodiments, about 5%, 10%, 15%, 20%, 25%, 30%, 35%, 40%, 45%, 50%, 55%, 60%, 65%, 70%, 75%, 80%, 85%, 90%, 95% or more of the PBMCs are NK cells.

In some embodiments, the multispecific (e.g., bispecific) anti-BCMA/NKp46 antibody described herein promotes monocyte-mediated killing of target cells (e.g., B cells or B cell lines such as Ramos cells). In some embodiments, the anti-BCMA/NKp46 antibody described herein includes a wild-type human IgG1 Fc region. In some embodiments, the anti-BCMA/NKp46 antibody described herein binds to NK cells with the optimized mutations (SEQ ID NO: 43) on the Fc region. In some embodiments, the target cells is Ramos cell. In some embodiments, the killing of the target cells is by either or both of phagocytosis and trogocytosis type of cancer cell killing mechanism. In some embodiments, about 5%, 10%, 15%, 20%, 25%, 30%, 35%, 40%, 45%, 50%, 55%, 60%, 65%, 70%, 75%, 80%, 85%, 90%, 95% or more of the target cells are killed.

In some embodiments, there is no significant change of cell numbers (e.g., NK cell numbers or monocyte cell numbers) after the killing of the target cells.

The disclosure provides multispecific (e.g., bispecific) antibodies and antigen-binding fragments thereof that specifically bind to BCMA/NKp46 (e.g., human BCMA/NKp46). In one aspect, the disclosure provides an anti-BCMA/NKp46 multispecific (e.g., bispecific) antibody or antigen-binding fragment thereof, comprising: a first antigen-binding domain that specifically binds to BCMA; and a second antigen-binding domain that specifically binds to NKp46.

In some embodiments, the first antigen-binding domain comprises a first heavy chain variable region (VH1) and a first light chain variable region (VL1); and the second antigen-binding domain comprises a second heavy chain variable region (VH2) and a second light chain variable region (VL2).

In some embodiments, the first heavy chain variable region (VH1) comprises complementarity determining regions (CDRs) 1, 2, and 3, wherein the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and the first light chain variable region (VL1) comprises CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence, wherein the selected VH1 CDRs 1, 2, and 3 amino acid sequences, and the selected VL1 CDRs 1, 2, and 3 amino acid sequences are as following:
(1) the selected VH1 CDRs 1, 2, 3 amino acid sequences are set forth in SEQ ID NOs: 88, 89 and 90, respectively, and the selected VL1 CDRs 1, 2, 3 amino acid sequences are set forth in SEQ ID NOs: 91, 92 and 93, respectively; or
(2) the selected VH1 CDRs 1, 2, 3 amino acid sequences and the selected VL1 CDRs 1, 2, 3 amino acid sequences are the corresponding VH CDRs 1, 2, 3 and VL CDRs 1, 2, 3 of Zevorcabtagene Autoleucel, Elranatamab, Teclistamab, Equecabtagene Autoleuce, Ciltacabtagene autoleucel, Idecabtagene Vicleucel, Belantamab, Linvoseltamab, Alnuctamab, TNB-383B and SEA-BCMA.

In some embodiments, the second heavy chain variable region (VH2) comprises CDRs 1, 2, and 3, wherein the VH2 CDR1 region comprises an amino acid sequence that is at least 80% identical to a selected VH2 CDR1 amino acid sequence, the VH2 CDR2 region comprises an amino acid sequence that is at least 80% a identical to a selected VH2 CDR2 amino acid sequence, and the VH2 CDR3 region comprises an amino acid sequence that is at least 80% identical to a selected VH2 CDR3 amino acid sequence; and the second light chain variable region (VL2) comprises CDRs 1, 2, and 3, wherein the VL2 CDR1 region comprises an amino acid sequence that is at least 80% identical to a selected VL2 CDR1 amino acid sequence, the VL2 CDR2 region comprises an amino acid sequence that is at least 80% identical to a selected VL2 CDR2 amino acid sequence, and the VL2 CDR3 region comprises an amino acid sequence that is at least 80% identical to a selected VL2 CDR3 amino acid sequence, wherein the selected VH2 CDRs 1, 2, and 3 amino acid sequences, and the selected VL2 CDRs 1, 2, and 3 amino acid sequences are as following:
the selected VH2 CDRs 1, 2, 3 amino acid sequences are set forth in SEQ ID NOs: 25, 27 and 29, respectively, and the selected VL2 CDRs 1, 2, 3 amino acid sequences are set forth in SEQ ID NOs: 30, 31, and 32, respectively.

In some embodiments, the selected VH1 CDRs 1, 2, 3 amino acid sequences are set forth in SEQ ID NOs: 88, 89 and 90, respectively, and the selected VL1 CDRs 1, 2, 3 amino acid sequences are set forth in SEQ ID NOs: 91, 92 and 93, respectively; and the selected VH2 CDRs 1, 2, 3 amino acid sequences are set forth in SEQ ID NOs: 26, 28 and 29, respectively, and the selected VL2 CDRs 1, 2, 3 amino acid sequences are set forth in SEQ ID NOs: 30, 31, and 32, respectively.

In some embodiments, the first heavy chain variable region comprises a sequence that is at least 80%, 85%, 90%, 95%, 99%, or 100% identical to SEQ ID NO: 106, the first light chain variable region comprises a sequence that is at least 80%, 85%, 90%, 95%, 99%, or 100% identical to SEQ ID NO: 107, the second heavy chain variable region comprises a sequence that is at least 80%, 85%, 90%, 95%, 99%, or 100% identical to SEQ ID NO: 33, and the second light chain variable region comprises a sequence that is at least 80%, 85%, 90%, 95%, 99%, or 100% identical to SEQ ID NO: 34.

In some embodiments, the VH1 comprises VH1 CDR1, VH1 CDR2, and VH1 CDR3 that are identical to VH CDR1, VH CDR2, and VH CDR3 of a selected VH sequence; and the VL1 comprising VL1 CDR1, VL1 CDR2, and VL1 CDR3 that are identical to VL CDR1, VL CDR2, and VL CDR3 of a selected VL sequence, wherein the selected VH sequence is SEQ ID NO: 106, and the selected VL sequence is SEQ ID NO: 107.

In some embodiments, the VH2 comprises VH2 CDR1, VH2CDR2, and VH2 CDR3 that are identical to VH CDR1, VH CDR2, and VH CDR3 of a selected VH sequence; and the VL2 comprising VL2 CDR1, VL2 CDR2, and VL2 CDR3 that are identical to VL CDR1, VL CDR2, and VL CDR3 of a selected VL sequence, wherein the selected VH sequence is SEQ ID NO: 33, and the selected VL sequence is SEQ ID NO: 34.

In some embodiments, the first antigen-binding domain specifically binds to human, mouse, monkey, or dog BCMA; and/or the second antigen-binding domain specifically binds to human, mouse, monkey, or dog NKp46.

In some embodiments, the first antigen-binding domain is a human or humanized antigen-binding domain; and/or the second antigen-binding domain is a human or humanized antigen-binding domain.

In some embodiments, the first antigen-binding domain is a single-chain variable fragment (scFV); and/or the second antigen-binding domain is a scFv.

In some embodiments, the antibody or antigen-binding fragment thereof comprises a fragment crystallizable region (Fc region). In some embodiments, the multispecific (e.g., bispecific) anti-BCMA/NKp46 antibody described herein can be designed to have an IgG1 subtype structure with the optimized mutations (G236A, A330L, and 1332E mutations in EU numbering).

In some embodiments, the anti-BCMA/NKp46 antibody described herein can be designed to have an IgG1 Fc region having an Alanine (A) at position 236, a Leucine (L) at position 330, and a Glutamic acid (E) at position 332 in EU numbering. In some embodiments, The Fc region comprises an amino acid sequence that is about or at least 60%, 65%, 70%, 75%, 80%, 85%, 90%, 95%, or 100% identical to SEQ ID NO: 43.

In some embodiments, the multispecific (e.g., bispecific) anti-BCMA/NKp46 antibody described herein can be designed to have an IgG1 subtype structure with knobs-into-holes (KIH) mutations, which can promote heterodimerization and avoid mispairing between the two heavy chains. In some embodiments, the anti-BCMA/NKp46 antibody has a higher endocytosis rate than the corresponding monoclonal antibodies or the control bispecific antibodies.

The first antigen-binding fragment and the second antigen-binding fragment of the bi-specific antibodies or antigen-binding fragments described herein can be in any suitable configurations. In some embodiments, wherein the second antigen-binding domain is a single-chain fragment variable (scFv) domain comprising a light chain variable domain (VL) and a heavy chain variable domain (VH) linked by a first linker.

In some embodiments, the second antigen-binding domain is linked to the C-terminus of the light chain of the first antigen-binding domain through a second linker. In some embodiments, the heavy chain variable domain of the first antigen-binding domain is linked to the Fc region. A schematic illustration of this configuration is shown in FIGS. 4A-4B.

In some embodiments, the multispecific (e.g., bispecific) anti-BCMA/NKp46 antibody includes a heavy chain sequence of comprising an IgG1 Fc region comprising the optimized mutations (SEQ ID NO: 43).

In some embodiments, the heavy chain of the multispecific (e.g., bispecific) anti-BCMA/NKp46 antibody (e.g., BCMA/NKp46 Fc-optimized heavy chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 81.

In some embodiments, the light chain of the multispecific (e.g., bispecific) anti-BCMA/NKp46 antibody (e.g., BCMA/NKp46 Fc-optimized light chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 82.

In some embodiments, the heavy chain of the multispecific (e.g., bispecific) anti-BCMA/NKp46 antibody (e.g., BCMA/NKp46 Fc-optimized heavy chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 81; and the light chain of the multispecific (e.g., bispecific) anti-BCMA/NKp46 antibody (e.g., BCMA/NKp46 Fc-optimized light chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 82.

In some embodiments, the second antigen-binding domain is linked to the C-terminus of the Fc region. In some embodiments, the heavy chain variable domain of the first antigen-binding domain is linked to the Fc region. A schematic illustration of this configuration is shown in FIG. 4B. In some embodiments, the heavy chain variable domain of the first antigen-binding domain is linked to the Fc region via a third linker.

In some embodiments, knobs-into-holes mutations were introduced in the Fc regions of the multispecific (e.g., bispecific) antibodies to reduce the chance of wrong pairing between the two heavy chains.

The disclosure provides, e.g., anti-BCMA/NKp46 antibody BCMA/NKp46 Fc-optimized, the modified antibodies thereof, including, e.g., chimeric antibodies, humanized antibodies, and human antibodies.

In some embodiments, the multispecific (e.g., bispecific) anti-BCMA/NKp46 antibodies, or antibody fragments thereof include the combinations of anti-BCMA and anti-NKp46 antigen-binding domains shown in FIG. 33.

In some embodiments, BCMA/NKp46 refers to a multispecific (e.g., bispecific) anti-BCMA/NKp46 antibody that contains a first anti-BCMA antigen-binding domain that is derived from a BCMA antibody and a second anti-NKp46 antigen-binding domain that is derived from an NKp46 antibody. In some embodiments, the first anti-BCMA antigen-binding domain comprises the CDRs of the BCMA antibody. In some embodiments, the second anti-NKp46 antigen-binding domain comprises the CDRs of the NKp46 antibody. In some embodiments, the first anti-BCMA antigen-binding domain comprises the VH and VL of the BCMA antibody. In some embodiments, the second anti-NKp46 antigen-binding domain comprises the VH and VL of the NKp46 antibody.

In some embodiments, the anti-BCMA/NKp46 antibody is a bispecific antibody. Bispecific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan).

Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

Any of the anti-BCMA/NKp46 antibodies or antigen-binding fragments thereof described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an anti-BCMA/NKp46 antibody or an antigen-binding fragment in vitro (e.g., in tissue culture or when stored as a pharmaceutical composition) or in vivo (e.g., in a human).

The disclosure also provides nucleic acid comprising a polynucleotide encoding an anti-BCMA/NKp46 antibody. The immunoglobulin heavy chain or immunoglobulin light chain in the anti-BCMA/NKp46 antibody comprises CDRs as shown in FIG. 33. When the polypeptides are paired with corresponding polypeptide (e.g., a corresponding heavy chain variable region or a corresponding light chain variable region), the paired polypeptides bind to NKp46 and/or BCMA.

The anti-BCMA/NKp46 antibodies can also be anti-BCMA/NKp46 antibody variants (including derivatives and conjugates) of anti-BCMA/NKp46 antibodies or antibody fragments.

Additional anti-BCMA/NKp46 antibodies provided herein are polyclonal, monoclonal, multispecific (multimeric, e.g., bispecific), human antibodies, chimeric antibodies (e.g., human-mouse chimera), single-chain antibodies, intracellularly-made antibodies (i.e., intrabodies), and antigen-binding fragments thereof. The anti-BCMA/NKp46 antibodies can be of any type (e.g., IgG, IgE, IgM, IgD, IgA, and IgY), class (e.g., IgG1, IgG2, IgG3, IgG4, IgA1, and IgA2), or subclass. In some embodiments, the anti-BCMA/NKp46 antibody or antigen-binding fragment is an IgG (e.g., IgG1 Fc region is shown in SEQ ID NO: 41) antibody or antigen-binding fragment thereof.

Fragments of anti-BCMA/NKp46 antibodies are suitable for use in the methods provided so long as they retain the desired affinity and specificity to both NKp46 and BCMA. Thus, a fragment of an anti-BCMA/NKp46 antibody will retain an ability to bind to NKp46 and BCMA.

### Anti-EGFR/NKp46 Antibodies and Antigen-Binding Fragments Thereof

The epidermal growth factor receptor (EGFR) has served as the founding member of the large family of growth factor receptors harboring intrinsic tyrosine kinase function. High abundance of EGFR and large internal deletions are frequently observed in brain tumors, whereas point mutations and small insertions within the kinase domain are common in lung cancer. For these reasons EGFR and its preferred heterodimer partner, HER2/ERBB2, became popular targets of anti-cancer therapies. Nevertheless, EGFR research keeps revealing unexpected observations, which are reviewed herein. Once activated by a ligand, EGFR initiates a time-dependent series of molecular switches comprising downregulation of a large cohort of microRNAs, up-regulation of newly synthesized mRNAs, and covalent protein modifications, collectively controlling phenotype-determining genes. In addition to microRNAs, long non-coding RNAs and circular RNAs play critical roles in EGFR signaling. Along with driver mutations. EGFR drives metastasis in many ways. Paracrine loops comprising tumor and stromal cells enable EGFR to fuel invasion across tissue barriers, survival of clusters of circulating tumor cells, as well as colonization of distant organs.

The epidermal growth factor receptor (EGFR) is a rational target for cancer therapy because it is commonly expressed at a high level in a variety of solid tumors and it has been implicated in the control of cell survival, proliferation, metastasis and angiogenesis. However, despite evidence to suggest that EGFR expression is associated with a poor prognosis in some tumours (e.g. breast, head and neck carcinomas), the situation is by no means clear-cut.

A detailed review of EGFR and its functions can be found in Uribe et al, Cancers (Basel), 2021 Jun. 1; 13(11):2748, and Ciardiello et al., Eur J Cancer, 2003 July; 39(10):1348-54, the content of each of which is incorporated by reference in its entirety.

In some embodiments, the multispecific (e.g., bispecific) anti-EGFR/NKp46 antibody described herein has an agonistic activity on the activation of NK cells. In some embodiments, the activation of NK cells is increased by about or at least 5%, 10%, 15%, 20%, 25%, 30%, 35%, 40%, 45%, 50%, 55%, 60%, 65%, 70%, 75%, 80%, 85%, 90%, 95%, or 100% upon contact with the anti-EGFR/NKp46 antibody described herein, compared to the activation of NK cells without contact with such antibody. In some embodiments, the activation of NK cells is measured by the expression of activation marker such as CD56. In some embodiments, the anti-EGFR/NKp46 antibody described herein binds to NK cells with the optimized mutations on the Fc region (SEQ ID NO. 43).

In some embodiments, the multispecific (e.g., bispecific) anti-EGFR/NKp46 antibody described herein binds to NK cells with silent mutations (e.g., LALA-PG mutations) on the Fc region. In some embodiments, the anti-EGFR/NKp46 antibody described herein binds to target cells (e.g., Ramos cells) with silent mutations (e.g., LALA-PG mutations) on the Fc region.

In some embodiments, the multispecific (e.g., bispecific) anti-EGFR/NKp46 antibody described herein activates NK cells (e.g., through the engagement of NKp46), which mediate the killing of target cells (e.g., B cells or B cell lines such as Ramos cells or Daudi cells). In some embodiments, the anti-EGFR/NKp46 antibody described herein activates NK cells with silent mutations (e.g., LALA-PG mutations) on the Fc region. In some embodiments, the anti-EGFR/NKp46 antibody described herein includes a wild-type human IgG1 Fc region. In some embodiments, the anti-EGFR/NKp46 antibody described herein binds to NK cells with the optimized mutations (SEQ ID NO: 43) on the Fc region.

In some embodiments, the multispecific (e.g., bispecific) anti-EGFR/NKp46 antibody described herein activates NK cells (e.g., through the engagement of NKp46) in a peripheral blood mononuclear cell (PBMC) population. In some embodiments, the PBMCs mediate the killing of target cells (e.g., B cells). In some embodiments, about 5%, 100%, 15%, 20%, 25%, 30%, 35%, 40%, 45%, 50%, 55%, 60%, 65%, 70%, 75%, 80%, 85%, 90%, 95% or more of the PBMCs are NK cells.

In some embodiments, the multispecific (e.g., bispecific) anti-EGFR/NKp46 antibody described herein promotes monocyte-mediated killing of target cells (e.g., B cells or B cell lines such as Ramos cells). In some embodiments, the anti-EGFR/NKp46 antibody described herein includes a wild-type human IgG1 Fc region. In some embodiments, the anti-EGFR/NKp46 antibody described herein binds to NK cells with the optimized mutations (SEQ ID NO: 43) on the Fc region. In some embodiments, the target cells is Ramos cell. In some embodiments, the killing of the target cells is by eith”r or ‘both of phagocytosis and trogocytosis type of cancer cell killing mechanism. In some embodiments, about 5%, 10%, 15%, 20%, 25%, 30%, 35%, 40%, 45%, 50%, 55%, 60%, 65%, 70%, 75%, 80%, 85%, 90%, 95% or more of the target cells are killed.

In some embodiments, there is no significant change of cell numbers (e.g., NK cell numbers or monocyte cell numbers) after the killing of the target cells.

The disclosure provides multispecific (e.g., bispecific) antibodies and antigen-binding fragments thereof that specifically bind to EGFR/NKp46 (e.g., human EGFR/NKp46). In one aspect, the disclosure provides an anti-EGFR/NKp46 multispecific (e.g., bispecific) antibody or antigen-binding fragment thereof, comprising: a first antigen-binding domain that specifically binds to EGFR; and a second antigen-binding domain that specifically binds to NKp46.

In some embodiments, the first antigen-binding domain comprises a first heavy chain variable region (VH1) and a first light chain variable region (VL1); and the second antigen-binding domain comprises a second heavy chain variable region (VH2) and a second light chain variable region (VL2).

In some embodiments, the first heavy chain variable region (VH1) comprises complementarity determining regions (CDRs) 1, 2, and 3, wherein the VH1 CDR1 region comprises an amino acid sequence that is at least 80% a identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and the first light chain variable region (VL1) comprises CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence, wherein the selected VH1 CDRs 1, 2, and 3 amino acid sequences, and the selected VL1 CDRs 1, 2, and 3 amino acid sequences are as following:
(1) the selected VH1 CDRs 1, 2, 3 amino acid sequences are set forth in SEQ ID NOs: 94, 95 and 96, respectively, and the selected VL1 CDRs 1, 2, 3 amino acid sequences are set forth in SEQ ID NOs: 97, 98 and 99, respectively; or
(2) the selected VH1 CDRs 1, 2, 3 amino acid sequences and the selected VL1 CDRs 1, 2, 3 amino acid sequences are the corresponding VH CDRs 1, 2, 3, and VL CDRs 1, 2, 3 of Amivantamab, Cetuximab, Necitumumab, Panitumumab, Nimotuzumab, BL-B01D1, Demupitamab, Depatuxizumab, Futuximab, Modotuximab, JMT-101, MRG-003, Pimurutamab, QL1203, SI-BOO1 or Ametumumab.

In some embodiments, the second heavy chain variable region (VH2) comprises CDRs 1, 2, and 3, wherein the VH2 CDR1 region comprises an amino acid sequence that is at least 80% identical to a selected VH2 CDR1 amino acid sequence, the VH2 CDR2 region comprises an amino acid sequence that is at least 80% identical to a selected VH2 CDR2 amino acid sequence, and the VH2 CDR3 region comprises an amino acid sequence that is at least 80% identical to a selected VH2 CDR3 amino acid sequence; and
the second light chain variable region (VL2) comprises CDRs 1, 2, and 3, wherein the VL2 CDR1 region comprises an amino acid sequence that is at least 80% identical to a selected VL2 CDR1 amino acid sequence, the VL2 CDR2 region comprises an amino acid sequence that is at least 80% identical to a selected VL2 CDR2 amino acid sequence, and the VL2 CDR3 region comprises an amino acid sequence that is at least 80% identical to a selected VL2 CDR3 amino acid sequence,
wherein the selected VH2 CDRs 1, 2, and 3 amino acid sequences, and the selected VL2 CDRs 1, 2, and 3 amino acid sequences are as following: the selected VH2 CDRs 1, 2, 3 amino acid sequences are set forth in SEQ ID NOs: 25, 27 and 29, respectively, and the selected VL2 CDRs 1, 2, 3 amino acid sequences are set forth in SEQ ID NOs: 30, 31, and 32, respectively.

In some embodiments, the selected VH1 CDRs 1, 2, 3 amino acid sequences are set forth in SEQ ID NOs: 94, 95 and 96, respectively, and the selected VL1 CDRs 1, 2, 3 amino acid sequences are set forth in SEQ ID NOs: 97, 98 and 99, respectively; and the selected VH2 CDRs 1, 2, 3 amino acid sequences are set forth in SEQ ID NOs: 25, 27 and 29, respectively, and the selected VL2 CDRs 1, 2, 3 amino acid sequences are set forth in SEQ ID NOs: 30, 31, and 32, respectively;

In some embodiments, the first heavy chain variable region comprises a sequence that is at least 80%, 85%, 90%, 95%, 990%, or 100% identical to SEQ ID NO: 108, the first light chain variable region comprises a sequence that is at least 80%, 85%, 90%, 95%, 99%, or 100% identical to SEQ ID NO: 109, the second heavy chain variable region comprises a sequence that is at least 80%, 85%, 90%, 95%, 99%, or 100% identical to SEQ ID NO: 33, and the second light chain variable region comprises a sequence that is at least 80%, 85%, 90%, 95%, 99%, or 100% identical to SEQ ID NO: 34.

In some embodiments, the VH1 comprises VH1 CDR1, VH1 CDR2, and VH1 CDR3 that are identical to VH CDR1, VH CDR2, and VH CDR3 of a selected VH sequence; and the VL1 comprising VL1 CDR1, VL1 CDR2, and VL1 CDR3 that are identical to VL CDR1, VL CDR2, and VL CDR3 of a selected VL sequence, wherein the selected VH sequence is SEQ ID NO: 108, and the selected VL sequence is SEQ ID NO: 109.

In some embodiments, the VH2 comprises VH2 CDR1, VH2CDR2, and VH2 CDR3 that are identical to VH CDR1, VH CDR2, and VH CDR3 of a selected VH sequence; and the VL2 comprising VL2 CDR1, VL2 CDR2, and VL2 CDR3 that are identical to VL CDR1, VL CDR2, and VL CDR3 of a selected VL sequence, wherein the selected VH sequence is SEQ ID NO: 33, and the selected VL sequence is SEQ ID NO: 34.

In some embodiments, the first antigen-binding domain specifically binds to human, mouse, monkey, or dog EGFR; and/or the second antigen-binding domain specifically binds to human, mouse, monkey, or dog NKp46.

In some embodiments, the first antigen-binding domain is a human or humanized antigen-binding domain; and/or the second antigen-binding domain is a human or humanized antigen-binding domain.

In some embodiments, the first antigen-binding domain is a single-chain variable fragment (scFV); and/or the second antigen-binding domain is a scFv.

In some embodiments, the antibody or antigen-binding fragment thereof comprises a fragment crystallizable region (Fc region). In some embodiments, the multispecific (e.g., bispecific) anti-EGFR/NKp46 antibody described herein can be designed to have an IgG1 subtype structure with the optimized mutations (G236A, A330L, and 1332E mutations in EU numbering).

In some embodiments, the anti-EGFR/NKp46 antibody described herein can be designed to have an IgG1 Fc region having an Alanine (A) at position 236, a Leucine (L) at position 330, and a Glutamic acid (E) at position 332 in EU numbering. In some embodiments, The Fc region comprises an amino acid sequence that is about or at least 60%, 65%, 70%, 75%, 80%, 85%, 90%, 95%, or 100% identical to SEQ ID NO: 43.

In some embodiments, the multispecific (e.g., bispecific) anti-EGFR/NKp46 antibody described herein can be designed to have an IgG1 subtype structure with knobs-into-holes (KIH) mutations, which can promote heterodimerization and avoid mispairing between the two heavy chains. In some embodiments, the anti-EGFR/NKp46 antibody has a higher endocytosis rate than the corresponding monoclonal antibodies or the control bispecific antibodies.

The first antigen-binding fragment and the second antigen-binding fragment of the bi-specific antibodies or antigen-binding fragments described herein can be in any suitable configurations. In some embodiments, wherein the second antigen-binding domain is a single-chain fragment variable (scFv) domain comprising a light chain variable domain (VL) and a heavy chain variable domain (VH) linked by a first linker.

In some embodiments, the second antigen-binding domain is linked to the C-terminus of the light chain of the first antigen-binding domain through a second linker. In some embodiments, the heavy chain variable domain of the first antigen-binding domain is linked to the Fc region. A schematic illustration of this configuration is shown in FIGS. 4A-4B.

In some embodiments, the multispecific (e.g., bispecific) anti-EGFR/NKp46 antibody includes a heavy chain sequence of comprising an IgG1 Fc region comprising the optimized mutations (SEQ ID NO: 43).

In some embodiments, the heavy chain of the multispecific (e.g., bispecific) anti-EGFR/NKp46 antibody (e.g., EGFR/NKp46 Fc-optimized heavy chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 83.

In some embodiments, the light chain of the multispecific (e.g., bispecific) anti-EGFR/NKp46 antibody (e.g., EGFR/NKp46 Fc-optimized light chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 84.

In some embodiments, the heavy chain of the multispecific (e.g., bispecific) anti-EGFR/NKp46 antibody (e.g., EGFR/NKp46 Fc-optimized heavy chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 83; and the light chain of the multispecific (e.g., bispecific) anti-EGFR/NKp46 antibody (e.g., EGFR/NKp46 Fc-optimized light chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 84.

In some embodiments, the second antigen-binding domain is linked to the C-terminus of the Fc region. In some embodiments, the heavy chain variable domain of the first antigen-binding domain is linked to the Fc region. A schematic illustration of this configuration is shown in FIG. 4B. In some embodiments, the heavy chain variable domain of the first antigen-binding domain is linked to the Fc region via a third linker.

In some embodiments, knobs-into-holes mutations were introduced in the Fc regions of the multispecific (e.g., bispecific) antibodies to reduce the chance of wrong pairing between the two heavy chains.

The disclosure provides, e.g., anti-EGFR/NKp46 antibody EGFR/NKp46 Fc-optimized, the modified antibodies thereof, including, e.g., chimeric antibodies, humanized antibodies, and human antibodies.

In some embodiments, the multispecific (e.g., bispecific) anti-EGFR/NKp46 antibodies, or antibody fragments thereof include the combinations of anti-EGFR and anti-NKp46 antigen-binding domains shown in FIG. 33.

In some embodiments, EGFR/NKp46 refers to a multispecific (e.g., bispecific) anti-EGFR/NKp46 antibody that contains a first anti-EGFR antigen-binding domain that is derived from a EGFR antibody and a second anti-NKp46 antigen-binding domain that is derived from an NKp46 antibody. In some embodiments, the first anti-EGFR antigen-binding domain comprises the CDRs of the EGFR antibody. In some embodiments, the second anti-NKp46 antigen-binding domain comprises the CDRs of the NKp46 antibody. In some embodiments, the first anti-EGFR antigen-binding domain comprises the VH and VL of the EGFR antibody. In some embodiments, the second anti-NKp46 antigen-binding domain comprises the VH and VL of the NKp46 antibody.

In some embodiments, the anti-EGFR/NKp46 antibody is a bispecific antibody. Bispecific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

Any of the anti-EGFR/NKp46 antibodies or antigen-binding fragments thereof described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an anti-EGFR/NKp46 antibody or an antigen-binding fragment in vitro (e.g., in tissue culture or when stored as a pharmaceutical composition) or in vivo (e.g., in a human).

The disclosure also provides nucleic acid comprising a polynucleotide encoding an anti-EGFR/NKp46 antibody. The immunoglobulin heavy chain or immunoglobulin light chain in the anti-EGFR/NKp46 antibody comprises CDRs as shown in FIG. 33. When the polypeptides are paired with corresponding polypeptide (e.g., a corresponding heavy chain variable region or a corresponding light chain variable region), the paired polypeptides bind to NKp46 and/or EGFR

The anti-EGFR/NKp46 antibodies can also be anti-EGFR/NKp46 antibody variants (including derivatives and conjugates) of anti-EGFR/NKp46 antibodies or antibody fragments. Additional anti-EGFR/NKp46 antibodies provided herein are polyclonal, monoclonal, multispecific (multimeric, e.g., bispecific), human antibodies, chimeric antibodies (e.g., human-mouse chimera), single-chain antibodies, intracellularly-made antibodies (i.e., intrabodies), and antigen-binding fragments thereof. The anti-EGFR/NKp46 antibodies can be of any type (e.g., IgG, IgE, IgM, IgD, IgA, and IgY), class (e.g., IgG1, IgG2, IgG3, IgG4, IgA1, and IgA2), or subclass. In some embodiments, the anti-EGFR/NKp46 antibody or antigen-binding fragment is an IgG (e.g., IgG1 Fc region is shown in SEQ ID NO: 41) antibody or antigen-binding fragment thereof.

Fragments of anti-EGFR/NKp46 antibodies are suitable for use in the methods provided so long as they retain the desired affinity and specificity to both NKp46 and EGFR. Thus, a fragment of an anti-EGFR/NKp46 antibody will retain an ability to bind to NKp46 and EGFR.

### Anti-EPCAM/NKp46 Antibodies and Antigen-Binding Fragments Thereof

EpCAM is a homophilic type I transmembrane glycoprotein belonging to the small GA733 protein family. Previous studies demonstrated that EpCAM participated in cell adhesion. However, other reports revealed EpCAM as a negative regulator of classic cadherin-mediated adhesion. These inconsistent results implied the complexity of EpCAM in cancers. Apart from mediating cell adhesion, EpCAM extracellular domain (EpEX) and intracellular domain (EpICD), which can be released from membrane-bound EpCAM following TNF-α-converting enzyme (TACE)- and presenilin-2 (PS-2)-mediated cleavage, act as ligand for signal transduction receptor or as transcriptional cofactor. These molecular mechanisms further border the functions of EpCAM and complicate its role in cancer progression.

A detailed review of EPCAM and its functions can be found in Liu et al., Exp Hematol Oncol. 2022; 11: 97, the content of which is incorporated by reference in its entirety.

In some embodiments, the multispecific (e.g., bispecific) anti-EpCAM/NKp46 antibody described herein has an agonistic activity on the activation of NK cells. In some embodiments, the activation of NK cells is increased by about or at least 5%, 10%, 15%, 20%, 25%, 30%, 35%, 40%, 45%, 50%, 55%, 60%, 65%, 70%, 75%, 80%, 85%, 90%, 95%, or 100% upon contact with the anti-EpCAM/NKp46 antibody described herein, compared to the activation of NK cells without contact with such antibody. In some embodiments, the activation of NK cells is measured by the expression of activation marker such as CD56. In some embodiments, the anti-EpCAM/NKp46 antibody described herein binds to NK cells with the optimized mutations on the Fc region (SEQ ID NO: 43).

In some embodiments, the multispecific (e.g., bispecific) anti-EpCAM/NKp46 antibody described herein binds to NK cells with silent mutations (e.g., LALA-PG mutations) on the Fc region. In some embodiments, the anti-EpCAM/NKp46 antibody described herein binds to target cells (e.g., Ramos cells) with silent mutations (e.g., LALA-PG mutations) on the Fc region.

In some embodiments, the multispecific (e.g., bispecific) anti-EpCAM/NKp46 antibody described herein activates NK cells (e.g., through the engagement of NKp46), which mediate the killing of target cells (e.g., B cells or B cell lines such as Ramos cells or Daudi cells). In some embodiments, the anti-EpCAM/NKp46 antibody described herein activates NK cells with silent mutations (e.g., LALA-PG mutations) on the Fc region. In some embodiments, the anti-EpCAM/NKp46 antibody described herein includes a wild-type human IgG1 Fc region. In some embodiments, the anti-EpCAM/NKp46 antibody described herein binds to NK cells with the optimized mutations (SEQ ID NO: 43) on the Fc region.

In some embodiments, the multispecific (e.g., bispecific) anti-EpCAM/NKp46 antibody described herein activates NK cells (e.g., through the engagement of NKp46) in a peripheral blood mononuclear cell (PBMC) population. In some embodiments, the PBMCs mediate the killing of target cells (e.g., B cells). In some embodiments, about 5%, 10%, 15%, 20%, 25%, 30%, 35%, 40%, 45%, 50%, 55%, 60%, 65%, 70%, 75%, 80%, 85%, 90%, 95% or more of the PBMCs are NK cells.

In some embodiments, the multispecific (e.g., bispecific) anti-EpCAM/NKp46 antibody described herein promotes monocyte-mediated killing of target cells (e.g., B cells or B cell lines such as Ramos cells). In some embodiments, the anti-EpCAM/NKp46 antibody described herein includes a wild-type human IgG1 Fc region. In some embodiments, the anti-EpCAM-NKp46 antibody described herein binds to NK cells with the optimized mutations (SEQ ID NO: 43) on the Fc region. In some embodiments, the target cells is Ramos cell. In some embodiments, the killing of the target cells is by either or both of phagocytosis and trogocytosis type of cancer cell killing mechanism. In some embodiments, about 5%, 10%, 15%, 20%, 25%, 30%, 35%, 40%, 45%, 50%, 55%, 60%, 65%, 70%, 75%, 80%, 85%, 90%, 95% or more of the target cells are killed.

In some embodiments, there is no significant change of cell numbers (e.g., NK cell numbers or monocyte cell numbers) after the killing of the target cells.

The disclosure provides multispecific (e.g., bispecific) antibodies and antigen-binding fragments thereof that specifically bind to EpCAM/NKp46 (e.g., human EpCAM/NKp46). In one aspect, the disclosure provides an anti-EpCAM/NKp46 multispecific (e.g., bispecific) antibody or antigen-binding fragment thereof, comprising: a first antigen-binding domain that specifically binds to EpCAM; and a second antigen-binding domain that specifically binds to NKp46.

In some embodiments, the first antigen-binding domain comprises a first heavy chain variable region (VH1) and a first light chain variable region (VL1); and the second antigen-binding domain comprises a second heavy chain variable region (VH2) and a second light chain variable region (VL2).

In some embodiments, the first heavy chain variable region (VH1) comprises complementarity determining regions (CDRs) 1, 2, and 3, wherein the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and the first light chain variable region (VL1) comprises CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence, wherein the selected VH1 CDRs 1, 2, and 3 amino acid sequences, and the selected VL1 CDRs 1, 2, and 3 amino acid sequences are as following: the selected VH1 CDRs 1, 2, 3 amino acid sequences are set forth in SEQ ID NOs: 100, 101 and 102, respectively, and the selected VL1 CDRs 1, 2, 3 amino acid sequences are set forth in SEQ ID NOs: 103, 104 and 105, respectively, or
the selected VH1 CDRs 1, 2, 3 amino acid sequences and the selected VL1 CDRs 1, 2, 3 amino acid sequences are the corresponding VH CDRs 1, 2, 3 and VL CDRs 1, 2, 4 of Catumaxomab, M-701, Adecatumumab, Edrecolomab, Tefibazumab, VB1-008 and VB1-050.

In some embodiments, the second heavy chain variable region (VH2) comprises CDRs 1, 2, and 3, wherein the VH2 CDR1 region comprises an amino acid sequence that is at least 80% identical to a selected VH2 CDR1 amino acid sequence, the VH2 CDR2 region comprises an amino acid sequence that is at least 80% identical to a selected VH2 CDR2 amino acid sequence, and the VH2 CDR3 region comprises an amino acid sequence that is at least 80% identical to a selected VH2 CDR3 amino acid sequence; and
the second light chain variable region (VL2) comprises CDRs 1, 2, and 3, wherein the VL2 CDR1 region comprises an amino acid sequence that is at least 80% identical to a selected VL2 CDR1 amino acid sequence, the VL2 CDR2 region comprises an amino acid sequence that is at least 80% identical to a selected VL2 CDR2 amino acid sequence, and the VL2 CDR3 region comprises an amino acid sequence that is at least 80% identical to a selected VL2 CDR3 amino acid sequence,
wherein the selected VH2 CDRs 1, 2, and 3 amino acid sequences, and the selected VL2 CDRs 1, 2, and 3 amino acid sequences are as following: the selected VH2 CDRs 1, 2, 3 amino acid sequences are set forth in SEQ ID NOs: 25, 27 and 29, respectively, and the selected VL2 CDRs 1, 2, 3 amino acid sequences are set forth in SEQ ID NOs: 30, 31, and 32, respectively.

In some embodiments, the selected VH1 CDRs 1, 2, 3 amino acid sequences are set forth in SEQ ID NOs: 100, 101 and 102, respectively, and the selected VL1 CDRs 1, 2, 3 amino acid sequences are set forth in SEQ ID NOs: 103, 104 and 105, respectively; and the selected VH2 CDRs 1, 2, 3 amino acid sequences are set forth in SEQ ID NOs: 25, 27 and 29, respectively, and the selected VL2 CDRs 1, 2, 3 amino acid sequences are set forth in SEQ ID NOs: 30, 31, and 32, respectively;

In some embodiments, the first heavy chain variable region comprises a sequence that is at least 80%, 85%, 90%, 95%, 99%, or 100% identical to SEQ ID NO: 114, the first light chain variable region comprises a sequence that is at least 80%, 85%, 90%, 95%, 99%, or 100% identical to SEQ ID NO: 111, the second heavy chain variable region comprises a sequence that is at least 80%, 85%, 90%, 95%, 99%, or 100% identical to SEQ ID NO: 33, and the second light chain variable region comprises a sequence that is at least 80%, 85%, 90%, 95%, 99%, or 100% identical to SEQ ID NO: 34.

In some embodiments, the VH1 comprises VH1 CDR1, VH1 CDR2, and VH1 CDR3 that are identical to VH CDR1, VH CDR2, and VH CDR3 of a selected VH sequence; and the VL1 comprising VL1 CDR1, VL1 CDR2, and VL1 CDR3 that are identical to VL CDR1, VL CDR2, and VL CDR3 of a selected VL sequence, wherein the selected VH sequence is SEQ ID NO: 110, and the selected VL sequence is SEQ ID NO: 111.

In some embodiments, the VH2 comprises VH2 CDR1, VH2CDR2, and VH2 CDR3 that are identical to VH CDR1, VH CDR2, and VH CDR3 of a selected VH sequence; and the VL2 comprising VL2 CDR1, VL2 CDR2, and VL2 CDR3 that are identical to VL CDR1, VL CDR2, and VL CDR3 of a selected VL sequence, wherein the selected VH sequence is SEQ ID NO: 33, and the selected VL sequence is SEQ ID NO: 34.

In some embodiments, the first antigen-binding domain specifically binds to human, mouse, monkey, or dog EpCAM; and/or the second antigen-binding domain specifically binds to human, mouse, monkey, or dog NKp46.

In some embodiments, the first antigen-binding domain is a human or humanized antigen-binding domain; and/or the second antigen-binding domain is a human or humanized antigen-binding domain.

In some embodiments, the first antigen-binding domain is a single-chain variable fragment (scFV); and/or the second antigen-binding domain is a scFv.

In some embodiments, the antibody or antigen-binding fragment thereof comprises a fragment crystallizable region (Fc region). In some embodiments, the multispecific (e.g., bispecific) anti-EpCAM/NKp46 antibody described herein can be designed to have an IgG1 subtype structure with the optimized mutations (G236A, A330L, and 1332E mutations in EU numbering). In some embodiments, the anti-EpCAM/NKp46 antibody described herein can be designed to have an IgG1 Fc region having an Alanine (A) at position 236, a Leucine (L) at position 330, and a Glutamic acid (E) at position 332 in EU numbering. In some embodiments, The Fc region comprises an amino acid sequence that is about or at least 60%, 65%, 70%, 75%, 80%, 85%, 90%, 95%, or 100% identical to SEQ ID NO: 43.

In some embodiments, the multispecific (e.g., bispecific) anti-EpCAM/NKp46 antibody described herein can be designed to have an IgG1 subtype structure with knobs-into-holes (KIH) mutations, which can promote heterodimerization and avoid mispairing between the two heavy chains. In some embodiments, the anti-EpCAM/NKp46 antibody has a higher endocytosis rate than the corresponding monoclonal antibodies or the control bispecific antibodies.

The first antigen-binding fragment and the second antigen-binding fragment of the bi-specific antibodies or antigen-binding fragments described herein can be in any suitable configurations. In some embodiments, wherein the second antigen-binding domain is a single-chain fragment variable (scFv) domain comprising a light chain variable domain (VL) and a heavy chain variable domain (VH) linked by a first linker.

In some embodiments, the second antigen-binding domain is linked to the C-terminus of the light chain of the first antigen-binding domain through a second linker. In some embodiments, the heavy chain variable domain of the first antigen-binding domain is linked to the Fc region. A schematic illustration of this configuration is shown in FIGS. 4A-4B.

In some embodiments, the multispecific (e.g., bispecific) anti-EpCAM/NKp46 antibody includes a heavy chain sequence of comprising an IgG1 Fc region comprising the optimized mutations (SEQ ID NO: 43).

In some embodiments, the heavy chain of the multispecific (e.g., bispecific) anti-EpCAM/NKp46 antibody (e.g., EpCAM/NKp46 Fc-optimized heavy chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 85.

In some embodiments, the light chain of the multispecific (e.g., bispecific) anti-EpCAM/NKp46 antibody (e.g., EpCAM/NKp46 Fc-optimized light chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 86.

In some embodiments, the heavy chain of the multispecific (e.g., bispecific) anti-EpCAM/NKp46 antibody (e.g., EpCAM/NKp46 Fc-optimized heavy chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO: 85; and the light chain of the multispecific (e.g., bispecific) anti-EpCAM/NKp46 antibody (e.g., EpCAM/NKp46 Fc-optimized light chain) comprises an amino acid sequence that is about or at least 80%, 85%, 86%, 87%, 88%, 89%, 90%, 91%, 92%, 93%, 94%, 95%, 96%, 97%, 98%, 99% or 100% identical to SEQ ID NO. 86.

In some embodiments, the second antigen-binding domain is linked to the C-terminus of the Fc region. In some embodiments, the heavy chain variable domain of the first antigen-binding domain is linked to the Fc region. A schematic illustration of this configuration is shown in FIG. 4B. In some embodiments, the heavy chain variable domain of the first antigen-binding domain is linked to the Fc region via a third linker.

In some embodiments, knobs-into-holes mutations were introduced in the Fc regions of the multispecific (e.g., bispecific) antibodies to reduce the chance of wrong pairing between the two heavy chains.

The disclosure provides, e.g., anti-EpCAM/NKp46 antibody EpCAM/NKp46 Fc-optimized, the modified antibodies thereof, including, e.g., chimeric antibodies, humanized antibodies, and human antibodies.

In some embodiments, the multispecific (e.g., bispecific) anti-EpCAM/NKp46 antibodies, or antibody fragments thereof include the combinations of anti-EpCAM and anti-NKp46 antigen-binding domains shown in FIG. 33.

In some embodiments, EpCAM/NKp46 refers to a multispecific (e.g., bispecific) anti-EpCAM/NKp46 antibody that contains a first anti-EpCAM antigen-binding domain that is derived from a EpCAM antibody and a second anti-NKp46 antigen-binding domain that is derived from an NKp46 antibody. In some embodiments, the first anti-EpCAM antigen-binding domain comprises the CDRs of the EpCAM antibody. In some embodiments, the second anti-NKp46 antigen-binding domain comprises the CDRs of the NKp46 antibody. In some embodiments, the first anti-EpCAM antigen-binding domain comprises the VH and VL of the EpCAM antibody. In some embodiments, the second anti-NKp46 antigen-binding domain comprises the VH and VL of the NKp46 antibody.

In some embodiments, the anti-EpCAM/NKp46 antibody is a bispecific antibody. Bispecific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

Any of the anti-EpCAM/NKp46 antibodies or antigen-binding fragments thereof described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an anti-EpCAM/NKp46 antibody or an antigen-binding fragment in vitro (e.g., in tissue culture or when stored as a pharmaceutical composition) or in vivo (e.g., in a human).

The disclosure also provides nucleic acid comprising a polynucleotide encoding an anti-EpCAM/NKp46 antibody. The immunoglobulin heavy chain or immunoglobulin light chain in the anti-EpCAM/NKp46 antibody comprises CDRs as shown in FIG. 33. When the polypeptides are paired with corresponding polypeptide (e.g., a corresponding heavy chain variable region or a corresponding light chain variable region), the paired polypeptides bind to NKp46 and/or EpCAM.

The anti-EpCAM/NKp46 antibodies can also be anti-EpCAM/NKp46 antibody variants (including derivatives and conjugates) of anti-EpCAM/NKp46 antibodies or antibody fragments. Additional anti-EpCAM/NKp46 antibodies provided herein are polyclonal, monoclonal, multispecific (multimeric, e.g., bispecific), human antibodies, chimeric antibodies (e.g., human-mouse chimera), single-chain antibodies, intracellularly-made antibodies (i.e., intrabodies), and antigen-binding fragments thereof. The anti-EpCAM/NKp46 antibodies can be of any type (e.g., IgG, IgE, IgM, IgD, IgA, and IgY), class (e.g., IgG1, IgG2, IgG3, IgG4, IgA1, and IgA2), or subclass. In some embodiments, the anti-EpCAM/NKp46 antibody or antigen-binding fragment is an IgG (e.g., IgG1 Fc region is shown in SEQ ID NO. 41) antibody or antigen-binding fragment thereof.

Fragments of anti-EpCAM/NKp46 antibodies are suitable for use in the methods provided so long as they retain the desired affinity and specificity to both NKp46 and EpCAM. Thus, a fragment of an anti-EpCAM/NKp46 antibody will retain an ability to bind to NKp46 and EpCAM.

### Antibodies and Antigen Binding Fragments Thereof

In some embodiments, the antibodies described herein (monospecific antibodies and multispecific antibodies (e.g., bispecific antibodies)) and antigen-binding fragments thereof can have various forms.

In general, antibodies (also called immunoglobulins) can be made up of two classes of polypeptide chains, light chains and heavy chains. A non-limiting antibody (anti-NKp46, anti-CD79b/NKp46, anti-BCMA/NKp46, anti-EGFR/NKp46, or anti-EpCAM/NKp46 antibody) of the present disclosure can be an intact, four immunoglobulin chain antibody comprising two heavy chains and two light chains. The heavy chain of the antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain (the amino acid sequence of human kappa light chain is shown in SEQ ID NO: 44) or a lambda light chain.

The hypervariable regions, known as the complementary determining regions (CDRs), form loops that comprise the principle antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding domain.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin, “Protein sequence and structure analysis of antibody variable domains,” Antibody engineering, Springer Berlin Heidelberg, 2001. 422-“39, Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132; 211-250; Martin et al., Methods Enzymol. 203:121-53 (1991); Morea et al., Biophys Chem. 68(1-3):9-16 (October 1997); Morea et al., J Mol Biol. 275(2):269-94 (January 1998); Chothia et al., Nature 342(6252):877-83 (December 1989); Ponomarenko and Bourne, BMC Structural Biology 7:64 (2007); each of which is incorporated herein by reference in its entirety.

The CDRs are important for recognizing an epitope of an antigen. As used herein, an “epitope” is the smallest portion of a target molecule capable of being specifically bound by the antigen-binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.”., in Vidarsson, et al, “IgG subclasses and allotypes: from structure to effector functions.” Frontiers in immunology” 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody can also be an immunoglobulin molecule that is derived from any species (e.g., human, rodent, mouse, rat, or camelid). The antigen-binding domain or antigen binding fragment is a portion of an antibody that retains specific binding activity of the intact antibody, i.e., any portion of an antibody that is capable of specific binding to an epitope on the intact antibody's target molecule. It includes, e.g., Fab, Fab′, F(ab′)2, and variants of these fragments. Thus, in some embodiments, an antibody or antigen binding fragment thereof can comprise e.g., a scFv, a Fv, a Fd, a dAb, a bispecific antibody, a bispecific scFv, a diabody, a linear antibody, a single-chain antibody molecule, a multispecific antibody formed from antibody fragments, and any polypeptide that includes a binding domain which is, or is homologous to, an antibody binding domain. Non-limiting examples of antigen-binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the scFv in a multispecific (e.g., bispecific) antibody has two heavy chain variable domains, and two light chain variable domains. In some embodiments, the scFv has two antigen binding regions (Antigen binding regions: A and B), and the two antigen binding regions can bind to the respective target antigens with different affinities.

In some embodiments, the multispecific (e.g., bispecific) antibodies or antigen-binding fragments thereof can comprises one, two, or three heavy chain variable region CDRs selected from FIG. 33.

In some embodiments, the antibodies described herein can be conjugated to a therapeutic agent. The antibody-drug conjugate comprising the antibody or antigen-binding fragment thereof can covalently or non-covalently bind to a therapeutic agent. In some embodiments, the therapeutic agent is a cytotoxic or cytostatic agent (e.g., monomethyl auristatin E, monomethyl auristatin F, cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DMA, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs). In some embodiments, the therapeutic agent is MMAE or MMEAF. In some embodiments, the therapeutic agent is conjugated via a linker, e.g., a VC linker. Details of the linkers used for ADCs can be found” e.g., in Su, Z. et al. “Antibody-drug conjugates: Recent adva” ces in linker chemistry.” Acta Pharmaceutica Sinica B (2021), which is incorporated herein by reference in its entirety.

Multispecific (e.g., bispecific) antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

Any of the antibodies or antigen-binding fragments thereof described herein may be conjugated to a stabilizing molecule (e.g., a molecule that increases the half-life of the antibody or antigen-binding fragment thereof in a subject or in solution). Non-limiting examples of stabilizing molecules include, a polymer (e.g., a polyethylene glycol) or a protein (e.g., serum albumin, such as human serum albumin). The conjugation of a stabilizing molecule can increase the half-life or extend the biological activity of an anti-CD79b/NKp46 antibody or an antigen-binding fragment in vitro (e.g., in tissue culture or when stored as a pharmaceutical composition) or in vivo (e.g., in a human).

The antibodies or antigen-binding fragments thereof can also have various forms. Many different formats of antibodies or antigen-binding fragments thereof are known in the art, and are described e.g., in Suurs, et al. “A review of bispecific antibodies and antibody constructs in oncology and clinical challenges,” Pharmacology & therapeutics (2019), which is incorporated herein by reference in the entirety.

In some embodiments, the antibody is a BiTe, a (scFv)2, a nanobody, a nanobody-HSA, a DART, a TandAb, a scDiabody, a scDiabody-CH3, scFv-CH-CL-scFv, a HSAbody, scDiabody-HAS, or a tandem-scFv. In some embodiments, the anti-CD79b/NKp46 antibody is a VHH-scAb, a VHH-Fab, a Dual scFab, a F(ab′)2, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a κλx-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)—IgG, TgG(L)-V, V(L)-IgG, KIH TgG-scFab, 2scFv-TgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)2-scFv2, a scFv-KIH, a Fab-scFv-Fc, a tetravalent HCAb, a scDiabody-Fc, a Diabody-Fc, a tandem scFv-Fc, an Intrabody, a dock and lock, a ImmTAC, an TgG-IgG conjugate, a Cov-X-Body, or a scFv1-PEG-scFv2.

In some embodiments, the antibody can be a TrioMab. In a TrioMab, the two heavy chains are from different species, wherein different sequences restrict the heavy-light chain pairing.

In some embodiments, the antibody has two different heavy chains and one common light chain. Heterodimerization of heavy chains can be based on the knobs-into-holes or some other heavy chain pairing technique.

In some embodiments, CrossMAb technique can be used produce antibodies. CrossMAb technique can be used enforce correct light chain association in bispecific heterodimeric IgG antibodies, this technique allows the generation of various bispecific antibody formats, including bi-(1+1), tri-(2+1) and tetra-(2+2) valent bispecific antibodies, as well as non-Fc tandem antigen-binding fragment (Fab)-based antibodies. These formats can be derived from any existing antibody pair using domain crossover, without the need for the identification of common light chains, post-translational processing/in vitro chemical assembly or the introduction of a set of mutations enforcing correct light chain association. The method is described in Klein et al., “The use of CrossMAb technology for the generation of bi- and “ultispecific antibodies.” MAbs. Vol. 8. No. 6. Taylor & Francis, 2016, which is incorporated by reference in its entirety. In some embodiments, the CHI in the heavy chain and the CL domain in the light chain are swapped.

The bispecific antibody can be a Duobody. The Fab-exchange mechanism naturally occurring in IgG4 antibodies is mimicked in a controlled matter in IgG1 antibodies, a mechanism called controlled Fab exchange. This format can ensure specific pairing between the heavy-light chains.

In Dual-variable-domain antibody (DVD-Ig), additional VH and variable light chain (VL) domain are added to each N-terminus for bispecific targeting. This format resembles the IgG-scFv, but the added binding domains are bound individually to their respective N-termini instead of a scFv to each heavy chain N-terminus.

In scFv-IgG, the two scFv are connected to the C-terminus of the heavy chain (CH3). The scFv-IgG format has two different bivalent binding sites and is consequently also called tetravalent. There are no heavy-chain and light-chain pairing problem in the scFv-IgG.

In some embodiments, the bispecific antibody can have a IgG-TgG format. Two intact IgG antibodies are conjugated by chemically linking the C-terminals of the heavy chains.

The bispecific antibody can also have a Fab-scFv-Fc format. In Fab-scFv-Fc format, a light chain, heavy chain and a third chain containing the Fc region and the scFv are assembled. It can ensure efficient manufacturing and purification.

In some embodiments, the bispecific antibody can be a TF. Three Fab fragments are linked by disulfide bridges. Two fragments target the tumor associated antigen (TAA) and one fragment targets a hapten. The TF format does not have an Fc region.

ADAPTIR has two scFvs bound to each side of an Fc region. It abandons the intact IgG as a basis for its construct, but conserves the Fc region to extend the half-life and facilitate purification.

Dual affinity retargeting (DART) has two peptide chains connecting the opposite fragments, thus VLA with VHB and VLB with VHA, and a sulfur bond at their C-termini fusing them together. In DART, the sulfur bond can improve stability over BiTEs.

In DART-Fc, an Fc region is attached to the DART structure. It can be generated by assembling three chains, two via a disulfide bond, as with the DART. One chain contains half of the Fc region which will dimerize with the third chain, only expressing the Fc region. The addition of Fc region enhances half-life leading to longer effective concentrations, avoiding continuous IV.

In tetravalent DART, four peptide chains are assembled. Basically, two DART molecules are created with half an Fc region and will dimerize. This format has bivalent binding to both targets, thus it is a tetravalent molecule.

Tandem diabody (TandAb) comprises two diabodies. Each diabody consists of an VHA and VLB fragment and a VHA and VLB fragment that are covalently associated. The two diabodies are linked with a peptide chain. It can improve stability over the diabody consisting of two scFvs. It has two bivalent binding sites.

The ScFv-scFv-toxin includes toxin and two scFv with a stabilizing linker. It can be used for specific delivery of payload.

In some embodiments, the bispecific antibody in present disclosure is designed to be 1+1 (monovalent for each target) and has an IgG1 subtype structure. This can reduce the avidity to cells with low expression levels of the first and second epitope, and increase the avidity to cells that co-express the first and second epitope, to achieve enhanced targeting function.

In some embodiments, the bispecific antibodies include KIH mutations. In some embodiments, the bispecific antibody includes a first antigen-binding domain that specifically binds to the first epitope, and a second antigen-binding domain that specifically binds to the second epitope. In some embodiments, the first antigen-binding domain includes a heavy chain that including one or more knob mutations (a knob heavy chain), and the second antigen-binding domain includes a heavy chain including one or more hole mutations (a hole heavy chain). In some embodiments, the first antigen-binding domain includes a heavy chain that includes one or more hole mutations (a hole heavy chain), and the second antigen-binding domain that includes a heavy chain including one or more knob mutations (a knob heavy chain).

### Antibody Characteristics

In some embodiments, the antibodies, or antigen-binding fragments thereof can initiate NK cell mediated target cell (tumor cell) killing, CDC or ADCC.

The disclosure provides antibodies and antigen-binding fragments thereof that can specifically bind to NKp46. The antibodies or antigen-binding fragments thereof described herein can block the binding between NKp46 and its ligands.

The disclosure provides antibodies and antigen-binding fragments thereof that can specifically bind to a TAA and an NK cell engager. The antibodies or antigen-binding fragments thereof described herein can block the binding between the TAA and its ligands.

General techniques can be used to measure the affinity of an antibody for an antigen include, e.g., ELISA, RIA, and surface plasmon resonance (SPR). Affinities can be deduced from the quotient of the kinetic rate constants (KD=koff/kon). In some implementations, the antibodies or antigen-binding fragments thereof can bind to the TAA with a dissociation rate (koff) of less than 0.1 s-1, less than 0.01 s-1, less than 0.001 s-1, less than 0.0001 s-1, or less than 0.00001 s-1. In some embodiments, the dissociation rate (koff) is greater than 0.01 s-1, greater than 0.001 s-1, greater than 0.0001 s-1, greater than 0.00001 s-1, or greater than 0.000001 s-1.

In some embodiments, kinetic association rates (kon) is greater than 1×102/Ms, greater than 1×103/Ms, greater than 1×104/Ms, greater than 1×105/Ms, or greater than 1×106/Ms. In some embodiments, kinetic association rates (kon) is less than 1-105/Ms, less than 1×106/Ms, or less than 1×107/Ms.

In some embodiments, the antibodies or antigen-binding fragments thereof can bind to the TAA with a KD of less than 1×10-6 M, less than 1×10-7 M, less than 1×10-8 M, less than 1×10-9 M, or less than 1×10-10 M. In some embodiments, the KD is less than 50 nM, 40 nM, 30 nM, 20 nM, 10 nM, 9 nM, 8 nM, 7 nM, 6 nM, 5 nM, 4 nM, 3 nM, 2 nM, or 1 nM. In some embodiments, KD is greater than 1×10-7 M, greater than 1×10-8 M, greater than 1×10-9 M, or greater than 1×10-10 M.

The antibodies or antigen-binding fragments thereof can also include an antigen-binding domain that can specifically bind to an NK cell surface antigen. In some embodiments, the antibodies or antigen-binding fragments thereof described herein activates NK cells and enhances NK cell-mediated cell killing (e.g., tumor cell killing). In some embodiments, the antibody can reduce tumor volume in an animal. In some embodiments, the antibody can reduce or slow down the progression of an autoimmune disease.

In some embodiments, the antibody or antigen-binding fragment thereof described herein has an agonistic activity on NK cell activation. In some embodiments, the antibody or antigen-binding fragment thereof described herein increases the expression of an activation marker (e.g., CD69) on NK cells. In some embodiments, the expression of the activation marker (e.g., CD69) is increased by about or at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or 100%.

Thus, in some embodiments, the antibody described herein is a NK cell surface antigen agonist. In some embodiments, the antibody described herein is a NK cell surface antigen antagonist.

In some implementations, the antibodies or antigen-binding fragments thereof can bind to the NK cell surface antigen with a dissociation rate (koff) of less than 0.1 s-1, less than 0.01 s-1, less than 0.001 s-1, less than 0.0001 s-1, or less than 0.00001 s-1. In some embodiments, the dissociation rate (koff) is greater than 0.01 s-1, greater than 0.001 s-1, greater than 0.0001 s-1, greater than 0.00001 s-1, or greater than 0.000001 s-1.

In some embodiments, kinetic association rates (kon) is greater than 1×102/Ms, greater than 1×103/Ms, greater than 1×104/Ms, greater than 1×105/Ms, or greater than 1×106/Ms. In some embodiments, kinetic association rates (kon) is less than 1×105/Ms, less than 1×106/Ms, or less than 1×107/Ms.

Affinities can be deduced from the quotient of the kinetic rate constants (KD=koff/kon). In some embodiments, KD is less than 1×10-6 M, less than 1×10-7 M, less than 1×10-8 M, less than 1×10-9 M, or less than 1×10-10 M. In some embodiments, the KD is less than 50 nM, 40 nM, 30 nM, 20 nM, 15 nM, 10 nM, 9 nM, 8 nM, 7 nM, 6 nM, 5 nM, 4 nM, 3 nM, 2 nM, or 1 nM.

In some embodiments, KD is greater than 1×10-7 M, greater than 1×10-8 M, greater than 1×10-9 M, or greater than 1×10-10 M.

Because the multispecific antibody (e.g., bispecific antibody) binds to both the TAA and the NK cell surface antigen, for cells that express both these molecules, the antibody has a higher binding affinity to these cells. Avidity can be used to measure the binding affinity of an antibody to these cells. Avidity is the accumulated strength of multiple affinities of individual non-covalent binding interactions.

In some embodiments, the antibody, antigen-binding fragment, or ADC has a tumor growth inhibition rate or percentage (TGI %) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the anti-CD79b/NKp46 antibody, antigen-binding fragment, or ADC has a tumor growth inhibition percentage that is less than 60%, 70%, 80%, 90%, 100%, 110%, 120%, 130%, 140%, or 150%. The TGI (%) can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41 days after the treatment starts. As used herein, the tumor growth inhibition rate or percentage (TGI %) is calculated using the following formula:

$$TGI ( \% ) = [ 1 - ( Ti - T 0 ) / ( Vi - V 0 ) ] \times 100 \%$$

Ti is the average tumor volume in the treatment group on day i. T0 is the average tumor volume in the treatment group on day zero. Vi is the average tumor volume in the control group on day i. V0 is the average tumor volume in the control group on day zero.

In some embodiments, the antibody, antigen-binding fragment, or ADC has a functional Fc region. In some embodiments, effector function of a functional Fc region is antibody-dependent cell-mediated cytotoxicity (ADCC). In some embodiments, effector function of a functional Fc region is phagocytosis. In some embodiments, effector function of a functional Fc region is ADCC and phagocytosis. In some embodiments, the Fc region is human IgG1, human IgG2, human IgG3, or human IgG4.

In some embodiments, the antibody, antigen-binding fragment, or ADC does not have a functional Fc region. For example, the antibodies or antigen-binding fragments thereof are Fab, Fab′, F(ab′)2, and Fv fragments. In some embodiments, the anti-CD79b/NKp46 antibodies or antigen-binding fragments thereof as described herein have an Fc region without effector function.

In some embodiments, the Fc is a human IgG4 Fc. In some embodiments, the Fc does not have a functional Fc region. For example, the Fc region has LALA mutations (L234A and L235A mutations in EU numbering), or LALA-PG mutations (L234A, L235A, P329G mutations in EU numbering). The amino acid sequence of the LALA-PG mutant of human IgG1 is shown in SEQ ID NO: 42.

In some embodiments, the antibody or antigen-binding fragment thereof comprises a fragment crystallizable region (Fc region). In some embodiments, the antibody described herein can be designed to have an IgG1 subtype structure with the optimized mutations (G236A, A330L, and 1332E mutations in EU numbering). In some embodiments, the antibody described herein can be designed to have an IgG1 Fc region having an Alanine (A) at position 236, a Leucine (L) at position 330, and a Glutamic acid (E) at position 332 in EU numbering. In some embodiments, The Fc region comprises an amino acid sequence that is about or at least 60%, 65%, 70%, 75%, 80%, 85%, 90%, 95%, or 100% identical to SEQ ID NO: 43.

Some other modifications to the Fc region can be made. For example, a cysteine residue(s) can be introduced into the Fc region, thereby allowing interchain disulfide bond formation in this region. The homodimeric fusion protein thus generated may have any increased half-life in vitro and/or in vivo.

In some embodiments, the IgG4 has S228P mutation (EU numbering). The S228P mutation prevents in vivo and in vitro IgG4 Fab-arm exchange.

In some embodiments, the antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; or position 314 in Kabat numbering); however, Asn297 may also be located about 3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the antibody can be further engineered to replace the Asparagine at position 297 with Alanine (N297A).

### Recombinant Vectors

The present disclosure also provides recombinant vectors (e.g., expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of antibody polypeptides or fragments thereof by recombinant techniques.

In some embodiments, the antibody is an anti-NKp46, anti-CD79b/NKp46, anti-BCMA/NKp46, anti-EGFR/NKp46, or anti-EpCAM/NKp46 antibody. The full length sequence of human NKp46 is known in the art (amino acids 22-254 of human NKp46 is shown in SEQ ID NO: 1). The full-length sequence of monkey NKp46 is known in the art (amino acids 22-257 of monkey NKp46 is shown in SEQ ID NO: 2). In some embodiments, a His-tagged human NKp46 protein is used as the immunogen.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells. Suitable systems are disclosed, for example, in Fisher-Hoch et al., 1989, Proc. Natl. Acad. Sci. USA 86:317-321; Flexner et al., 1989, Ann. N.Y. Acad Sci. 569:86-103; Flexner et al., 1990, Vaccine, 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques, 6:616-627, 1988; Rosenfeld et al., 1991, Science, 252:431-434; Kolls et al., 1994, Proc. Natl. Acad. Sci. USA, 91:215-219; Kass-Eisler et al., 1993, Proc. Natl. Acad. Sci. USA, 90:11498-11502; Guzman et al., 1993, Circulation, 88:2838-2848; and Guzman et al., 1993, Cir. Res., 73:1202-1207. Techniques for incorporating DNA into such expression systems are well known to those of ordinary skill in the art. The DNA may also be “naked,” as described, for example, in Ulmer et al., 1993, Science, 259:1745-1749, and Cohen, 1993, Science. 259:1691-1692. The uptake of naked DNA may be increased by coating the DNA onto biodegradable beads that are efficiently transported into the cells.

For expression, the DNA insert comprising a polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH may be used. For reviews, see Ausubel et al. (1989) Current Protocols in Molecular Biology, John Wiley & Sons, New York, N.Y, and Grant et al., Methods Enzymol., 153: 516-544 (1997).

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986), which is incorporated herein by reference in its entirety.

Transcription of DNA encoding an antibody of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein.

The disclosure also provides a nucleic acid sequence that has a homology of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% to any nucleotide sequence as described herein, and an amino acid sequence that has a homology of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% to any amino acid sequence as described herein.

In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, 200, 250, 300, 350, or 400 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

The disclosure provides one or more nucleic acid encoding any of the polypeptides as described herein. In some embodiments, the nucleic acid (e.g., cDNA) includes a polynucleotide encoding a polypeptide of a heavy chain as described herein. In some embodiments, the nucleic acid includes a polynucleotide encoding a polypeptide of a light chain as described herein. In some embodiments, the nucleic acid includes a polynucleotide encoding a scFv polypeptide as described herein.

In some embodiments, the vector can have two of the nucleic acids as described herein, wherein the vector encodes the VL region and the VH region that together bind to the TAA (e.g., CD79b). In some embodiments, a pair of vectors is provided, wherein each vector comprises one of the nucleic acids as described herein, wherein together the pair of vectors encodes the VL region and the VH region that together bind to the TAA (e.g., CD79b, BCMA, EGFR, or EpCAM).

In some embodiments, the vector includes two of the nucleic acids as described herein, wherein the vector encodes the VL region and the VH region that together bind to the NK cell surface antigen (e.g., NKp46). In some embodiments, a pair of vectors is provided, wherein each vector comprises one of the nucleic acids as described herein, wherein together the pair of vectors encodes the VL region and the VH region that together bind to the NK cell surface antigen (e.g., NKp46).

### Methods of Treatment

The methods described herein include methods for the treatment of disorders associated with cancer. Generally, the methods include administering a therapeutically effective amount of the antibody (e.g., anti-NKp46, anti-CD79b/NKp46, anti-BCMA/NKp46, anti-EGFR/NKp46, or anti-EpCAM/NKp46 antibody) as described herein, to a subject who is in need of, or who has been determined to be in need of, such treatment.

As used in this context, to “treat” means to ameliorate at least one symptom of the disorder associated with cancer. Often, cancer results in death; thus, a treatment can result in an increased life expectancy (e.g., by at least 1, 2, 3, 4, 5, 6, 7, 8, 9, 10, 11, 12 months, or by at least 1, 2, 3, 4, 5, 6, 7, 8, 9, 10 years). Administration of a therapeutically effective amount of an agent described herein for the treatment of a condition associated with cancer will result in decreased number of cancer cells and/or alleviated symptoms.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In some embodiments, the cancer is a chemotherapy resistant cancer.

In one aspect, the disclosure also provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of the antibody (e.g., anti-NKp46, anti-CD79b/NKp46, anti-BCMA/NKp46, anti-EGFR/NKp46, anti-EpCAM/NKp46 antibody, or antibody-drug conjugates) as described herein to a subject in need thereof, e.g., a subject having, or identified or diagnosed as having, a cancer, e.g., B cell lymphoma, bladder cancer, breast cancer, cervical cancer, colorectal cancer, gastric cancer, non-small cell lung cancer (NSCLC), mesothelioma, ovarian cancer, pancreatic cancer, prostate cancer, oral cancer, or renal cancer. In some embodiments, the cancer is a solid tumor, lung cancer, head and neck cancer, thyroid cancer, central nervous system (CNS) cancer, liver cancer, or brain cancer.

As used herein, the terms “subject” and “patient” are used interchangeably throughout the specification and describe an animal, human or non-human, to whom treatment according to the methods of the present invention is provided. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

In some embodiments, the compositions and methods disclosed herein can be used for treatment of patients at risk for a cancer. Patients with cancer can be identified with various methods known in the art.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the antibody, antigen binding fragment, antibody-drug conjugates, antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of the antibody, or antigen binding fragment is an amount sufficient to ameliorate, stop, stabilize, reverse, inhibit, slow and/or delay progression of an autoimmune disease or a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro.

Effective amounts and schedules for administering the antibody, antigen binding fragment, antibody-drug conjugates, and/or compositions disclosed herein may be determined empirically, and making such determinations is within the skill in the art. Those skilled in the art will understand that the dosage that must be administered will vary depending on, for example, the mammal that will receive the antibody, antigen binding fragment, antibody-drug conjugates, and/or compositions disclosed herein, the route of administration, the particular type of the agent or compositions disclosed herein used and other drugs being administered to the mammal.

A typical daily dosage of an effective amount of the antibody, antigen binding fragment, antibody-drug conjugates (e.g., anti-NKp46, anti-CD79b/NKp46, anti-BCMA/NKp46, anti-EGFR/NKp46, anti-EpCAM/NKp46 antibody, or antibody-drug conjugates) is 0.01 mg/kg to 100 mg/kg. In some embodiments, the dosage can be less than 100 mg/kg, 30 mg/kg, 20 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about or at least 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In some embodiments, one or more additional therapeutic agents can be administered to the subject. The additional therapeutic agent can comprise one or more inhibitors selected from the group consisting of an inhibitor of B-Raf, an CD79b inhibitor, a BCMA inhibitor, an EGFR inhibitor, an EpCAM inhibitor, an inhibitor of a MEK, an inhibitor of ERK, an inhibitor of K-Ras, an inhibitor of c-Met, an inhibitor of NKp46, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDO1) (e.g., epacadostat).

In some embodiments, the additional therapeutic agent can comprise one or more inhibitors selected from the group consisting of an inhibitor of CD79b, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-PD-1 antibody, an anti-PD-L1 antibody, anti-PD-L2 antibody, an anti-LAG-3 antibody, an anti-TIGIT antibody, an anti-BTLA antibody, an anti-CTLA4 antibody, an anti-CD40 antibody, an anti-OX40 antibody, an anti-4-1BB antibody, an anti-TIM3 antibody, or an anti-GITR antibody.

### Pharmaceutical Compositions and Routes of Administration

Also provided herein are pharmaceutical compositions that contain at least one (e.g., one, two, three, or four) of the antibodies or antigen-binding fragments, or antibody-drug conjugates (e.g., anti-NKp46, anti-CD79b/NKp46, anti-BCMA/NKp46, anti-EGFR/NKp46, anti-EpCAM/NKp46 antibody, or antibody-drug conjugates) described herein. The pharmaceutical compositions may be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant.

Compositions containing one or more of any of the antibodies or antigen-binding fragments, or antibody-drug conjugates (anti-NKp46, anti-CD79b/NKp46, anti-BCMA/NKp46, anti-EGFR/NKp46, anti-EpCAM/NKp46 antibody-drug conjugates) described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Toxicity and therapeutic efficacy of compositions can be determined by standard pharmaceutical procedures in cell cultures or experimental animals (e.g., monkeys). One can determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the antibodies or antigen-binding fragments, or antibody-drug conjugates (anti-NKp46, anti-CD79b/NKp46, anti-BCMA/NKp46, anti-EGFR/NKp46, anti-EpCAM/NKp46 antibody-drug conjugates) will be an amount that treats the disease (e.g., kills cancer cells) in a subject (e.g., a human subject identified as having cancer), or a subject identified as being at risk of developing the disease (e.g., a subject who has previously developed cancer but now has been cured), decreases the severity, frequency, and/or duration of one or more symptoms of a disease in a subject (e.g., a human). The effectiveness and dosing of any of the antibodies or antigen-binding fragments, or antibody-drug conjugates (e.g., anti-NKp46, anti-CD79b/NKp46, anti-BCMA/NKp46, anti-EGFR/NKp46, anti-EpCAM/NKp46 antibody, or antibody-drug conjugates) described herein can be determined by a health care professional or veterinary professional using methods known in the art, as well as by the observation of one or more symptoms of disease in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the antibodies or antigen-binding fragments, or antibody-drug conjugates (e.g., anti-NKp46, anti-CD79b/NKp46, anti-BCMA/NKp46, anti-EGFR/NKp46, anti-EpCAM/NKp46 antibody, or antibody-drug conjugates) described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; or about 0.1 mg/kg to about 0.5 mg/kg). While these doses cover a broad range, one of ordinary skill in the art will understand that therapeutic agents vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the therapeutic agent in vivo.

The pharmaceutical compositions can be included in a container, pack, or dispenser together with instructions for administration. The disclosure also provides methods of manufacturing the antibodies or antigen-binding fragments, or antibody-drug conjugates (e.g., anti-NKp46, anti-CD79b/NKp46, anti-BCMA/NKp46, anti-EGFR/NKp46, anti-EpCAM/NKp46 antibody, or antibody-drug conjugates) for various uses as described herein.

### EXAMPLES

The invention is further described in the following examples, which do not limit the scope of the invention described in the claims.

### Example 1: Generation of Anti-NKp46 Antibodies

Rabbit Immunizations with NKp46 Antigen

To generate monoclonal antibodies against human NKp46, two New Zealand white hu rabbits were immunized with human NKp46 ECD antigen (SEQ ID NO: 1, aa. 22-254, his-tagged from AcroBiosystems), using either complete Freund's adjuvant or incomplete Freund's adjuvant. Serum titer was monitored by ELISA after the second injection.

### Screening of Single B Cells Producing Monoclonal Antibody to NKp46

After 4 immunizations at 200 μg/rabbit/injection, rabbits with good titer (>1:100,000) received a final booster shot and the spleens were harvested 7 days after the final injection.

Isolation of Splenocytes from Rabbits

All the following procedures (except for centrifugation) were performed in a biological safety cabinet. Rabbit spleens were harvested 7 days after the final booster shot. Splenocytes were prepared in a sterile cell strainer placed in the bottom of a 100 mm sterile petri dish containing 20 mL RPMI+1% of Penicillin-Streptomycin (P/S). Using sterile forceps, spleen tissues were transferred to a cell strainer. Specifically, while holding a spleen with forceps, the spleen was cut into small pieces, which were then pressed through the mesh of cell strainer. Tissue debris was washed with 10 mL RPMI+1% P/S. Spleen cells were transferred from the petri dish to a new 50 mL conical tube. RPMI+1% P/S was added to a final volume of 50 ml. Cells were centrifuged at 400×g for 5 min and supernatant was aspirated. 13 mL ACK buffer (Gibco cat #A1049201) was added to re-suspend the cells. The suspended cells were incubated at room temperature for 1 minute. RPMI+1% P/S was added to reach a final volume of 50 ml. Cells were centrifuged at 400×g for 5 min and supernatant was aspirated. The cell pellet was re-suspended in RPMI+10% FBS+1% P/S. The cells were centrifuged at 400×g for 5 min and supernatant was aspirated. The pellet was re-suspended in 15 ml RPMI+10% FBS+1% P/S. The cells were pipetted through 100 μm cell strainer into a 50 mL conical tube to remove cell clumps. Splenocytes were then seeded at desired density (e.g., 4E6 cells/ml) with proper medium for sorting. The remaining splenocytes were frozen in 900% serum+100% DMSO at 6×10⁷ cells/vial (~1.8 ml) at −80° C. overnight. The frozen cells were transferred into liquid nitrogen tank for long term storage.

### Antigen Specific B Cell Sorting

For B cell sorting, freshly isolated or thawed splenocytes (~2×10⁸ splenocytes) were cultured in B-cell culture media (RPMI-1640, 15% FBS, 1×HEPES, 1×2-ME (2-Mercaptoethanol), 1% Penicillin/Streptomycin) overnight before sorting. 96-well B cell feeding plates were prepared accordingly one day before the sorting. On the day of sorting, suspended and loosely attached splenocytes were collected by gently pipetting medium against the culturing surface of flask. The cells were then transferred to a conical tube and centrifuged at 400×g for 3 minutes. The cell pellet was washed with fluorescence-activated cell sorting (FACS) buffer (1×PBS+0.5% BSA) twice. Biotinylated antigen was added at 5 μg/ml (final concentration). The mixture was incubated at room temperature (RT) for 20 min. The staining mixture was then centrifuged at 400×g for 3 min and the cells were re-suspended in FACS buffer. The cells were transferred into a 1.5 ml amber Eppendorf tube. The staining antibody mixtures were then added to the cells. The staining mixture was incubated at 4° C. for 15-30 min, and then centrifuged at 400×g for 3 min. The cell pellet was washed twice with FACS buffer. The washed cell pellet was re-suspended at ~10⁷ cells/ml in 1×PBS+1% FBS. Using Fluorescence-activated cell sorting, antigen specific single B cells were sorted into 96-well plates (20 plates for each rabbit). 96-well B cell culture plates with sorted B cells were cultured in 37° C. with 5% CO₂ for 12 days.

### Screening of Single B Cell Culture

On day 8 post sorting, 15 μL of B cell culture supernatant was collected from each well for antigen specific ELISA. Briefly, the B cell culture supernatants were transferred to a NKp46 extracellular domain (ECD) coated, then blocked 384-well plate. The cells were incubated at room temperature for 1 hour and washed 3× with PBS plus 0.05% Tween-20. Antibody secreted were detected using goat anti-rabbit IgG HRP+TMB substrate. B cell supernatants that met the cut off OD450 (>0.5 or 3× higher than the pre-immune serum) were then selected. Using ELISA, the selected cells were screened against Cynomolgus NKp46 (SEQ ID NO: 2) for cross-species binding.

Initial ELISA screening of supernatants from 40 plates (96-well plate) sorted B cell were screened for NKp46 specific antibodies. Approximately 39 NKp46 antigen (both human and Cynomolgus) specific positive B cell clones were identified.

On day 12 post sorting, the B cell culture plates were centrifuged at 400×g for 3 min. Supernatants from positive clones (with OD greater than selected cutoff from antigen specific ELISA) were collected and the cell pellets were preserved in 100 μL DNA/RNA shield (Zymo Cat #R1100-250) in 250 μL PCR tubes. Collected supernatants were subjected to additional tests as described below.

Recovery of VH and V Gene Sequences from Selected Anti-NKp46 Clones

DNA fragments encoding the heavy chain variable domain (VH) and light chain variable domain (VL) from B cell clones showing high cell surface binding affinity to NKp46 were amplified via 5′ RACE (rapid amplification of cDNA ends), TOPO cloned, and sequenced.

Screening of NKp46 Specific Antibodies that Bind to Cell Surface Expressed NKp46.

NKp46 is a member of the natural cytotoxicity receptor (NCR) family. It is anticipated that not all antibody clones that bind to NKp46 in ELISA would also bind to cell surface expressed NKp46 due to epitope accessibility. The top 9 NKp46 specific clones identified by ELISA were expressed and purified as rabbit IgG. These rabbit antibodies were subjected to cell surface binding assay with primary NK cells purified from healthy human donors (Stemcell Technologies, #17955) by FACS. About 50,000 NK (purified using Stemcell Technologies kit #17955 from human PBMC donor) cells were incubated with serially diluted antibodies in FACS buffer (PBS+0.5% BSA) for 30 mins on ice. After incubation cells were washed twice and detected with anti-rabbit IgG FITC (Biolegend, #406403) secondary antibody. For analysis, the mean fluorescence intensity (MFI of FITC) of each antibody was determined and plotted using GraphPad prism software (Version 9.4.1; GraphPad Software Inc). All top 9 ELISA positive clones tested were shown to bind to NKp46 on cell surface in a dose dependent manner. 3 out of the 9 clones (3-7, 5-13 and 6-1) were sequenced.

### Example 2: Chimeric NKp46 Antibodies Constructions

To express chimeric rabbit/human IgG1 antibodies, constant regions of the heavy chain (CHI-3) of human IgG1 (SEQ ID NO.: 41) and the constant region of human light chain Kappa (CL-kappa) (SEQ ID NO.: 44) were synthesized and cloned into pcDNA3.4 respectively. pcDNA3.4 containing CHI-3 of human IgG1 (pcDNA3.4-huIgG1-Hc) was further digested with EcoRU/NheI for the cloning of VH sequences. And EcoRI/BsiWI were used to digest VL sequences receiving vector, pcDNA3.4 expressing human CL-kappa (pcDNA3.4-huKappa-Lc). VH and VL sequences of three selected rabbit anti-Nkp46 antibodies, ch3-7, ch5-13 and ch6-1, and that of the benchmark antibody IP-46-3, were synthesized (IDT) with designed overlapped sequences at both 5′ and 3′ ends which will anneal and assemble (NEB NEBuilder® HiFi DNA Assembly) with the corresponding ends of receiving vectors. The amino acid sequence of the heavy chain variable region (VH) of IP-46-3 is shown in SEQ ID NO: 39, and the amino acid sequence of the light chain variable region (VL) of IP-46-3 is shown in SEQ ID NO: 40.

### Example 3: Transfection and Purification of Chimeric NKp46 Antibodies

All antibody light chain and heavy chain coding sequences were generated by direct DNA synthesis and cloned into mammalian expression vector. The cloned sequences were verified by sanger sequencing.

Chinese hamster ovary (CHO) cells were grown in CHOgro medium (Mirus) complemented with 4 mM L-Glutamine and 0.33% Poloxamer-188 and diluted to 4×10⁶ cells/ml with fresh cell culture media at the time of transfection. Cells were transfected by lipofection with 1 μg of plasmid DNA per 4×10⁶ cells. Transfected cells were kept in incubator with shaking at 125 rpm in 5% CO₂ and 70% humidity environment at 32° C.

Protein expression titers were measured every 5 days. Cell supernatant was collected by centrifugation after 10-14 days post-transfection and filtered through a 0.22 μm PES membrane filter. Expressed antibodies were purified by Protein A chromatography (TOYOPEARL® AF-rProtein A HC-650M resin, Tosoh) and aggregates were removed by anion exchange resin (TOYOPEARL NH2-750F, Tosoh).

Purified antibodies were analyzed by HPLC (Dionex UltiMate® 3000-RS UHPLC, ThermoFisher) controlled by Chromeleon® software. A size exclusion column TSKgel UP-SW3000, 2 μm, 4.6 mm ID×15 cm (Tosoh) was used to resolve antibodies purity and aggregation.

All purified antibodies were 97% or greater monomer purity, sterilized through 0.22 μm PES membrane filter, and used for binding and functional assays.

### Example 4: SPR Affinity of Anti-NKp46 Antibodies

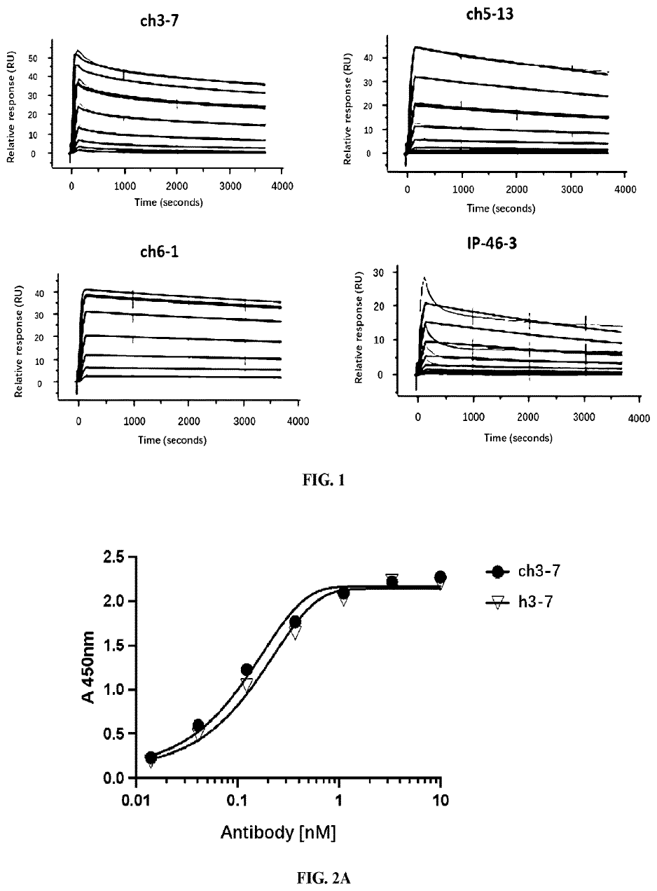

The absolute affinity of anti-NKp46 antibodies were determined using surface plasmon resonance (SPR). Cytiva Biacore (8K+) system and software was used for data collection and all assays were performed at 25° C. The binding and running buffer for assays was PBS-P+ (Cytiva, #28995084). The anti-NKp46 antibodies (ch3-7, ch5-13, ch6-1 and IP-46-3) at 0.5 ug/ml were captured on a Series S sensor chip protein A (Cytiva, #1033554302) using contact time of 60 s and at a flow rate of 10 μl/min. 7 startup cycles consisting of buffer flow at 30 μl/min with a contact time of 120 s and dissociation time of 30 s were performed before the antigen was injected. Recombinant human NKp46, His Tag (Acrobiosystems, #NC1-H52H4) was each serially diluted (concentration series was 44 nM, 22 nM, 1 nM, 5.5 nM, 2.75 nM, 1.375 nM, 0.6875 nM and 0.3437 nM). The contact time for antigen injections was 120 s at a flow rate of 30 μl/min, and the dissociation time was 3,600 s. The assay was performed in multiple cycle kinetics mode. Data were evaluated using Insight Evaluation Software and fitted using a 1:1 binding kinetics fit model. FIG. 1 and Table 1 show that the anti-NKp46 antibodies, ch3-7, ch5-13 and ch6-1 had much higher affinity to the target NKp46 protein than the benchmark antibody IP-46-3.

TABLE 1
SPR affinity of anti-NKp46 antibodies

| Antibody | kon(1/Ms) | kd(1/s) | KD(M) |
|---|---|---|---|
| ch3-7 | 2.04e+10 | 4.05e−01 | 1.99e−11 |
| ch5-13 | 4.25e+05 | 8.22e−05 | 1.94e−10 |
| ch6-1 | 2.00e+06 | 4.18e−05 | 2.09e−11 |
| IP-46-3 | 4.02e+05 | 1.45e−04 | 3.61e−10 |

### Example 5. Humanization of Anti-NKp46 Antibodies

Three rabbit anti-human NKp46 monoclonal antibody clones, 3-7, 5-13 and 6-1 were humanized by grafting the CDRs of the leading antibodies into selected human germline frameworks closest to the rabbit frameworks identified by IgBLAST (www.ncbi.nlm.nih.gov/igblast/) and/or IMGT/DomainGapAlig (www.imgt.org/3Dstructure-DB/cgil/DomainGapAlign.cgi) with. Human germline IGHV, IGKV, IGHJ and IGKJ were selected based on sequence similarity within both frameworks and CDRs. Certain human germline framework residues were back mutated to corresponding rabbit residues to maintain canonical loop structure and light chain/heavy chain interface (Padlan Mol. Immunol., 1994, 31:169; Foote and Winter JMB, 1992, 224:487; Padlan Mol. Immunol., 1994, 31:169). By humanization, h3-7, h5-13 and h6-1 humanized antibody were generated.

### Example 6: ELISA and BLI Binding of Chimeric and Humanized Anti-NKp46 Antibodies

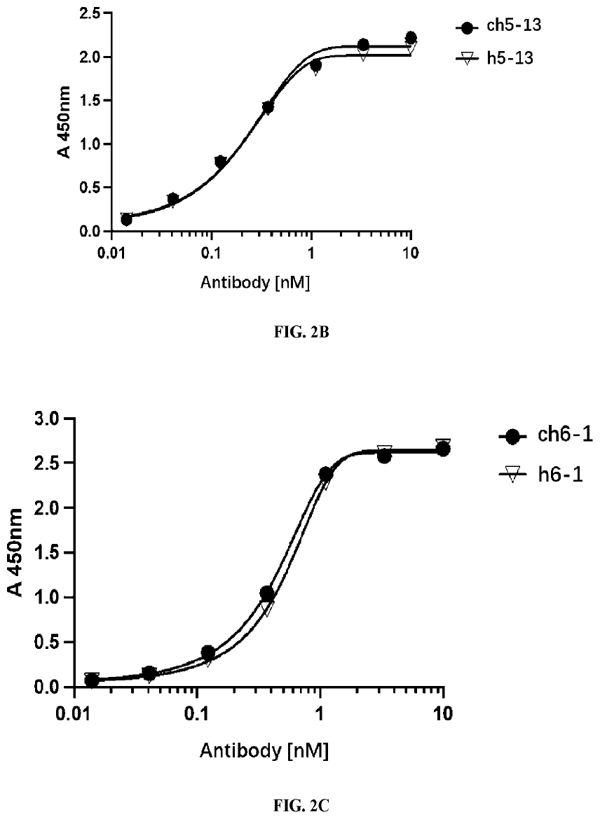

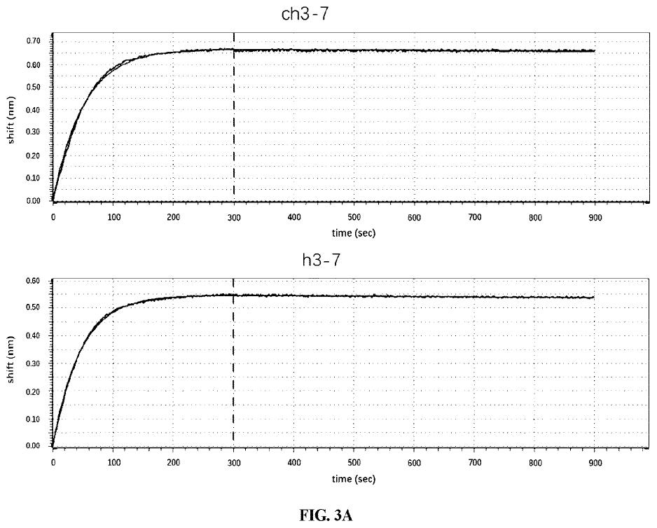

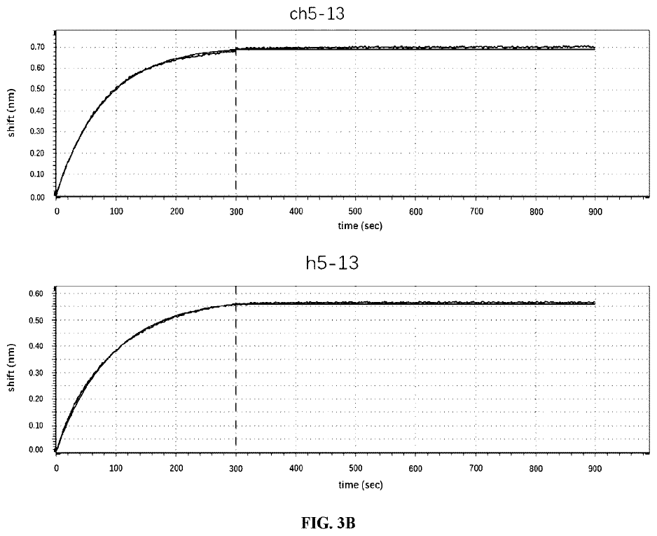

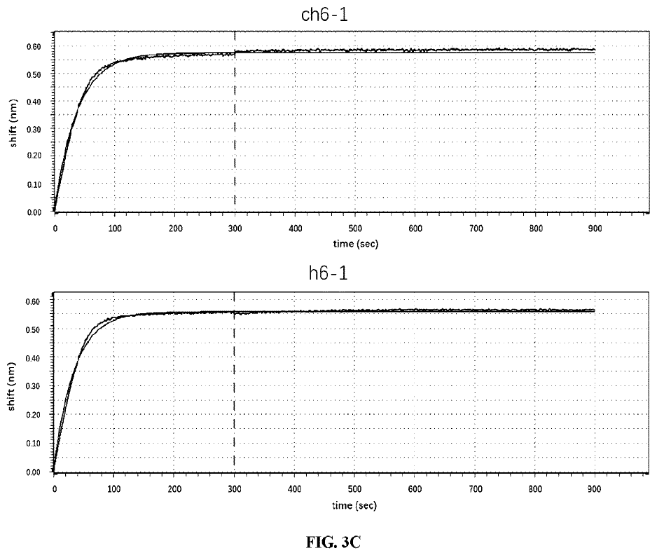

The NKp46 target affinities of chimeric and humanized forms of the NKp46 hIgG1 antibody were evaluated. For ELISA binding, 1 μg/ml (in PBS) of recombinant human NKp46, His Tag (Acro Biosystems, #NC1-H52H4) was coated on an ELISA plate overnight. ELISA plates were washed and blocked with blocking buffer (PBS+1% BSA), followed by incubation with serially diluted NKp46 primary antibodies. NKp46 antibody binding was quantified using an anti-human IgG HRP secondary antibody (Biolegend Catalog 410902) and HRP substrate. For BLI binding, 10 μg/ml of NKp46 antibodies were captured on an anti-human IgG Fc probe (Gator Bio Catalog 160024), followed by measuring association and dissociation with recombinant human NKp46, His Tag (Acrobiosystems, #NCI-H52H4) at a single concentration of 11 nM in PBS+0.05% Tween-20. Absolute KD was determined by applying a 1:1 binding model. Both ELISA and BLI binding assays showed that there was no significant difference in affinity between the chimeric and humanized forms of the NKp46 antibodies (FIGS. 2A-2C and FIGS. 3A-3C), suggesting that the humanization process did not result in a loss of NKp46 target affinity.

### Example 7: Molecule Constructions of TAA/NKp46 Bispecific Antibodies

To generate different TAA/NKp46 LC bispecific antibody (FIG. 4A), the scFv (single-chain fragment variable VL-GS-VH) of NKp46 antibody was connected to the C terminal of the TAA (tumor associated antigen) antibody LC (VL-Ck) through a GS linker, meanwhile the TAA VH was linked with wild-type Fc, silenced Fc (LALAPG) or optimized Fc. ch44G2/ch3-7 Fc-silenced, ch44G2/ch5-13 Fc-silenced, ch44G2/ch6-1 Fc-silenced, ch44G2/ch3-7 Fc-WT, ch44G2/ch5-13 Fc-WT, ch44G2/ch6-1 Fc-WT, ch44G2/ch6-1 (LC) Fc-optimized, h44G2/h6-1 (LC) Fc-optimized, BCMA/NKp46 Fc-optimized, EGFR/NKp46 Fc-optimized and EpCAM/NKp46 Fc-optimized are of this LC format.

ch44G2/ch6-1 (HC) Fc-optimized and h44G2/h6-1 (HC) Fc-optimized are of this HC format.

To generate different TAA/Nkp46 HC bispecific antibody (FIG. 4B), the scFv (single-chain fragment variable VL-GS-VH) of NKp46 antibody was connected to the C terminal of the TAA HC Fc, meanwhile the Fc wild-type Fc, silenced Fc (LALAPG) or optimized Fc. ch44G2/ch6-1 (HC) Fc-optimized and h44G2/h6-1 (HC) Fc-optimized are of this HC format.

Fragments were synthesized (IDT) and assembled (NEB NEBuilder® HiFi DNA Assembly) into pcDNA3.4 or pcDNA3.4 carrying desired fragments as described above. The assembled plasmid was transformed to Competent E. coli (NEB® 5-alpha) and clone with correct sequence, based on sequencing results (Elim Biopharm), was further cultured with LB containing Carbenicillin (100 ug/ml) for plasmid purification (QIAGEN Plasmid Plus Kits). Plasmids were eluted in Nuclease-free H2O (Sigma) and stored at −80° C.

### Example 8: Transfection and Purification of TAA/Nkp46 Bispecific Antibodies

All antibody light chain and heavy chain coding sequences were generated by direct DNA synthesis and cloned into mammalian expression vector. The cloned sequences were verified by sanger sequencing.

Chinese hamster ovary (CHO) cells were grown in CHOgro medium (Mirus) complemented with 4 mM L-Glutamine and 0.33% Poloxamer-188 and diluted to 4×10⁶ cells/ml with fresh cell culture media at the time of transfection. Cells were transfected by lipofection with 1 μg of plasmid DNA per 4×10⁶ cells. Transfected cells were kept in incubator with shaking at 125 rpm in 5% CO₂ and 70% humidity environment at 32° C.

Protein expression titers was measured every 5 days. Cell supernatant was collected by centrifugation after 10-14 days post-transfection and filtered through a 0.22 μm PES membrane filter. Expressed antibodies were purified by Protein A chromatography (TOYOPEARL® AF-rProtein A HC-650M resin, Tosoh) and aggregates were removed by anion exchange resin (TOYOPEARL NH2-750F, Tosoh).

Purified antibodies were analyzed by HPLC (Dionex UltiMate® 3000-RS UHPLC, ThermoFisher) controlled by Chromeleon® 9 software. A size exclusion column TSKgel UP-SW3000, 2 μm, 4.6 mm ID×15 cm (Tosoh) was used to resolve antibodies purity and aggregation.

All purified antibodies were 97% or greater monomer purity, sterilized through 0.22 μm PES membrane filter, and used for binding and functional assays.

### Example 9: Activation of NK Cells by Bispecific Antibodies Comprising NKp46-Binding Fragments

The ability of NKp46 antibodies to activate NK (NKp46+) cells in the presence of target Ramos (CD79b+) cells was evaluated. CD79b/NKp46 antibodies ch44G2/ch3-7 Fc-WT, ch44G2/ch5-13 Fc-WT and ch44G2/ch6-1 Fc-WT and negative isotype control were serially diluted (concentration series was 0.001 nM, 0.005 nM, 0.01 nM, 0.05 nM, 0.1 nM and 0.5 nM). CD56+NK cells were purified using negative selection (Stemcell Technologies, #17955). Purified NK cells were cultured overnight in complete RPMI media (with 10% heat inactivated FBS and 5% penicillin/streptomycin). About 100,000 NK cells were incubated with 20,000 Ramos (E:T ratio of 5:1) in the presence of serially diluted antibodies for 4 hours at 37° C. After incubation, cells were stained with a flow cytometry antibody cocktail containing CD56-FTTC (Biolegend, #362546), and CD107a-APC (Biolegend, #328620) in FACS buffer (PBS+0.5% BSA) on ice for 30 mins. After washing twice with FACS buffer, the cells were re-suspended in 100 μl of FACS buffer with Sytox blue live/dead stain (Thermofisher, #S34857) and analyzed with a Cytek cytometer. FACS analysis showed that CD56+NK cells had increased activation marker CD107a in the presence of CD79b/NKp46 antibodies compared to negative isotype control. Significantly, FIG. 5 shows that bispecific antibodies ch44G2/ch3-7 Fc-WT, ch44G2/ch5-13 Fc-WT and ch44G2/ch6-1 Fc-WT activated NK cells potently, indicating that they are effective agonists.

### Example 10: Binding Abilities of NK Cell Engagers with Silenced Fc to Effector NK Cells

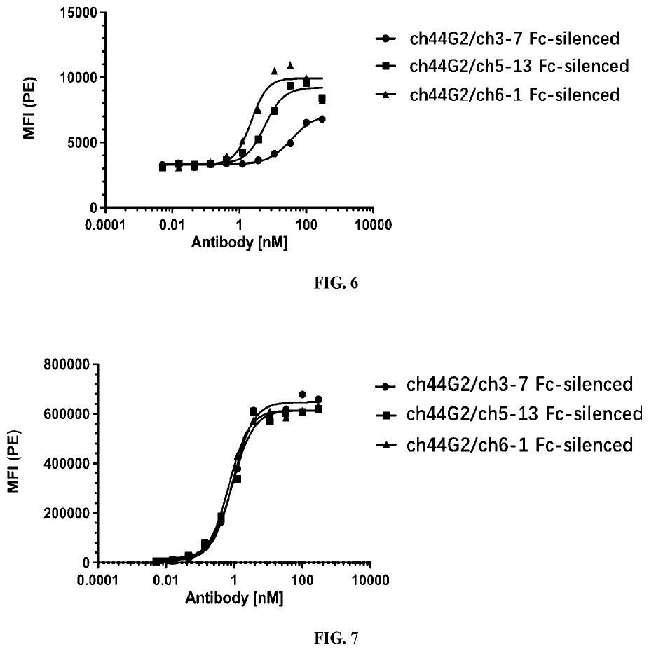

Binding abilities of NK cell engagers to effector NK cells were evaluated. The NK cell engagers (e.g., CD79b/NKp46 bispecific antibodies ch44G2/ch3-7 Fc-silenced, ch44G2/ch5-13 Fc-silenced and ch44G2/ch6-1 Fc-silenced) were serially diluted. About 50,000 CD56+NK cell (purified using Stemcell Technologies kit #17955 from human PBMC donor #474) and Ramos cells were incubated with serially diluted CD79b/NKp46 bispecific antibodies in FACS buffer (PBS+0.5% BSA) for 30 mins on ice. After incubation cells were washed twice and detected with anti-human IgG PE secondary antibody (Jackson Research, #109-116-170). For analysis, the mean fluorescence intensity (MFI of PE) of each antibody was determined and plotted using GraphPad prism software (Version 9.4.1; GraphPad Software Inc). FIG. 6 shows that all three CD79b/NKp46 bispecific antibodies can bind with NK cells.

### Example 11: Binding Abilities of NK Cell Engagers with Silenced Fc to Target Ramos Cells

Binding abilities of NK cell engagers (e.g., CD79b/NKp46 bispecific antibodies) to target Ramos cells were evaluated. CD79b/NKp46 bispecific antibodies (ch44G2/ch3-7 Fc-silenced, ch44G2/ch5-13 Fc-silenced and ch44G2/ch6-1 Fc-silenced) were serially diluted. About 50,000 CD56+NK cells (purified using Stemcell Technologies kit #17955 from human PBMC donor #474) and Ramos cells were incubated with serially diluted CD79b/NKp46 bispecific antibodies in FACS buffer (PBS+0.5% BSA) for 30 mins on ice. After incubation cells were washed twice and detected with anti-human IgG PE secondary antibody (Jackson Research, #109-116-170). For analysis, the mean fluorescence intensity (MFI of PE) of each antibody was determined and plotted using GraphPad prism software (Version 9.4.1; GraphPad Software Inc). FIG. 7 shows that all three CD79b/NKp46 bispecific antibodies can bind Ramos (CD79+) cells with similar affinity.

### Example 12: NK Cell Engagers with Silenced Fc Mediate Target Cancer B Cell Killing by NK Cells

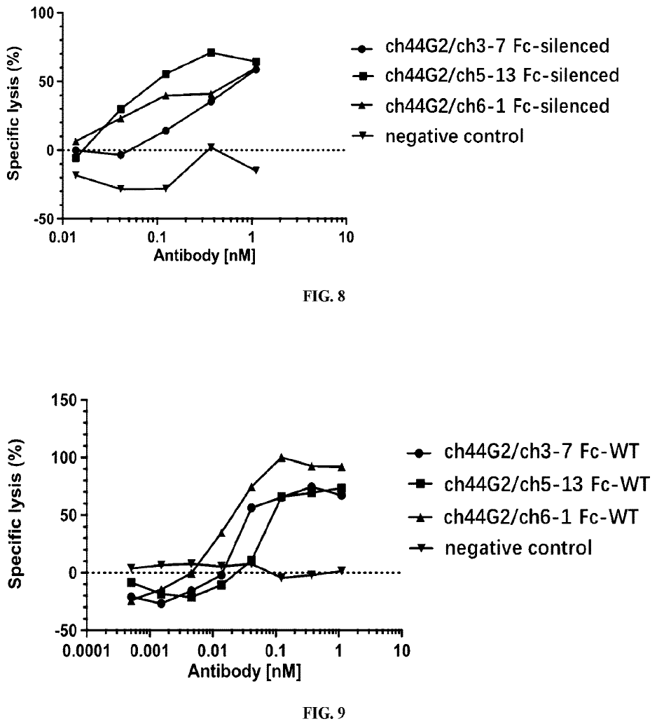

The ability of NK cell engagers with Fc-silenced mutations to kill target Daudi cell by effector NK cells was evaluated. The NK cell engagers (e.g., CD79b/NKp46 bispecific antibodies ch44G2/ch3-7 Fc-silenced, ch44G2/ch5-13 Fc-silenced and ch44G2/ch6-1 Fc-silenced) and negative control were serially diluted. CD56+NK cells were purified using negative selection (Stemcell Technologies, #17955) from indicated human PBMC donors. Daudi cells were labelled with CFSE (Thermofisher, #C34554). About 100,000 NK cells were incubated with 20,000 CFSE+ Ramos or Daudi (E:T ratio of 5:1) in the presence of serially diluted antibodies in complete RPMI media (with 10% heat inactivated FBS and 5% penicillin/streptomycin) for 24 hours at 37° C. After incubation, the cells were re-suspended in 100 μl of FACS buffer with Sytox blue live/dead stain (Thermofisher, #S34857) and analyzed with a Cytek cytometer. Live Daudi B cells were gated as Sytox-CFSE+ by FACS, and absolute cell count was obtained by collecting a fixed volume for all treatment conditions. Specific lysis of cell killing was calculated as (live target cancer cell number in nontreatment group−live target cancer number in treatment group)/(live target cancer number in treatment group with highest activity−live target cancer number in nontreatment group)×100. FIG. 8 shows that three CD79b/NKp46 antibodies with Fc-silenced mutations were able to potently kill target Daudi cells by NK cells, suggesting that antibodies can engage NKp46 receptor alone in NK cells and still able to potently kill cancer cells.

### Example 13: NK Cell Engagers with Wild Type Fc Mediate Target Cancer B Cell Killing by NK Cells

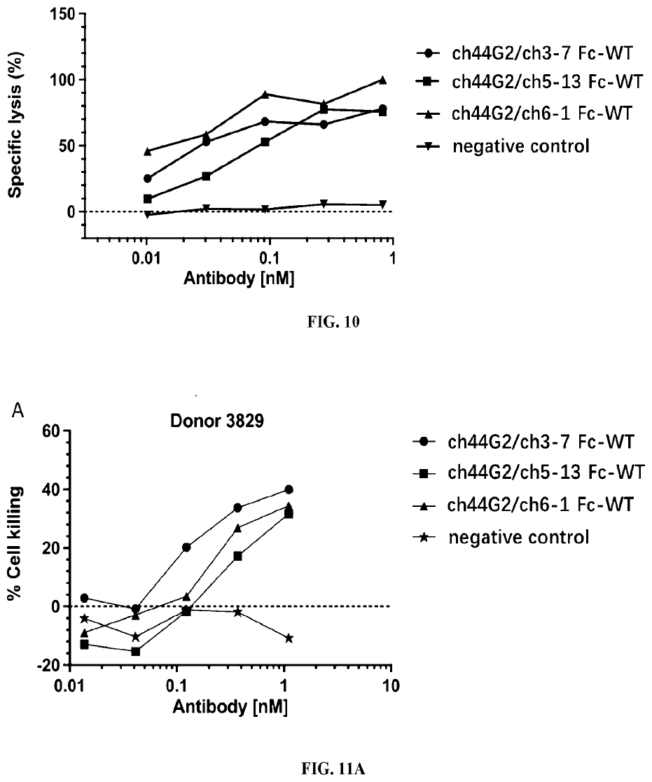

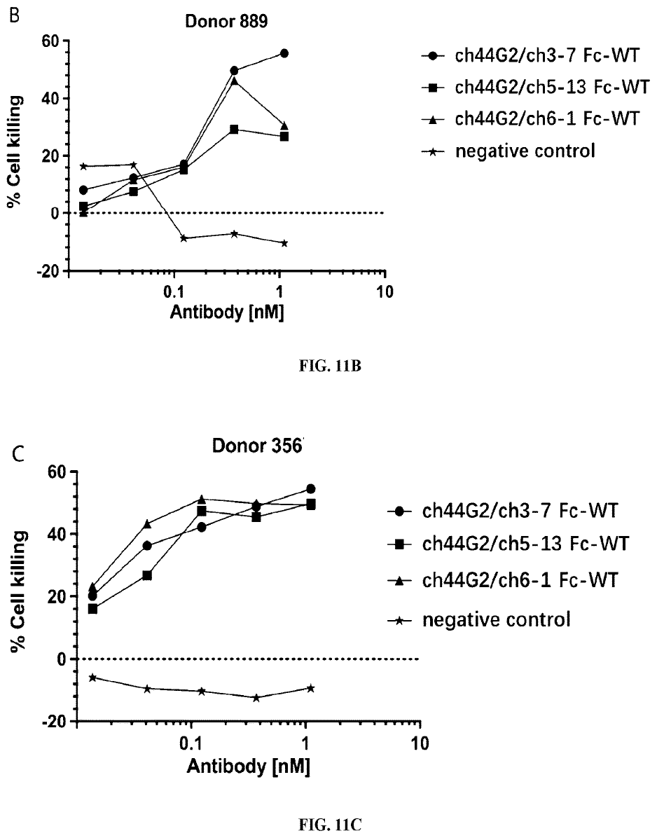

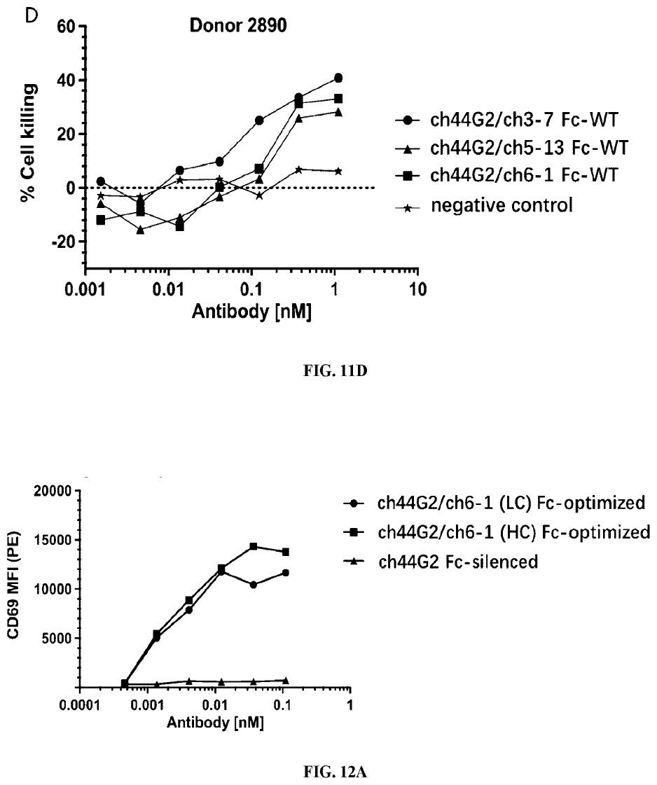

The ability of NK cell engagers with wild type Fc to kill target cancer B cells (CD79+ Ramos and Daudi cells) by effector NK cells was evaluated. The NK cell engagers CD79b/NKp46 bispecific antibodies (ch44G2/ch3-7 Fc-WT, ch44G2/ch5-13 Fc-WT and ch44G2/ch6-1 Fc-WT) and negative control were serially diluted. CD56+NK cells were purified using negative selection (Stemcell Technologies, #17955) from indicated human PBMC donors. Ramos and Daudi cells were labelled with CFSE (Thermofisher, #C34554). About 100,000 NK cells were incubated with 20,000 CFSE+ Ramos or Daudi (E:T ratio of 5:1) in the presence of serially diluted antibodies in complete RPMI media (with 10% heat inactivated FBS and 5% penicillin/streptomycin) for 24 hours at 37° C. After incubation, the cells were re-suspended in 100 μl of FACS buffer with Sytox blue live/dead stain (Thermofisher, #S34857) and analyzed with a Cytek cytometer. Live Ramos and Daudi B cells were gated as Sytox-CFSE+ by FACS, and absolute cell count was obtained by collecting a fixed volume for all treatment conditions. Specific lysis of cell killing was calculated as (live target cancer cell number in nontreatment group−live target cancer number in treatment group)/(live target cancer number in treatment group with highest activity−live target cancer number in nontreatment group)×100. The three CD79b/NKp46 bispecific antibodies efficiently killed both CD79+ cancer cells Ramos and Daudi (FIG. 9 and FIG. 10) compared to negative control, indicating that any tumor cell which expresses target antigen CD79b can be killed with the CD79b/NKp46 antibodies by recruiting NK cells.

### Example 14: NK Cell Engagers with Wild Type Fc Mediate Endogenous B Cell Killing by PBMC

The ability of NK cell engagers to kill endogenous B cells in PBMC donors with different NK cell percentage was tested. The NK cell engagers (e.g., CD79b/NKp46 bispecific antibodies ch44G2/ch3-7 Fc-WT, ch44G2/ch5-13 Fc-WT and ch44G2/ch6-1 Fc-WT) and negative control were serially diluted. About 100,000 PBMC (purified from different buffy coat of healthy donors using ficoll gradient) was combined with indicated serially diluted CD79b/NKp46 bispecific antibodies in complete RPMI media (with 10% heat inactivated FBS and 5% penicillin/streptomycin) and incubated for 24 hours at 37° C. After incubation, cells were stained with CD19-PE (Biolegend, #982402) in FACS buffer (PBS+0.5% BSA) on ice for 30 mins. After washing twice with FACS buffer, the cells were re-suspended in 100 μl of FACS buffer with Sytox blue live/dead stain (Thermofisher, #S34857) and analyzed with a Cytek cytometer. Live B cells were gated as Sytox-CD19+ B cells by FACS, and absolute cell count was obtained by collecting a fixed volume for all treatment conditions. Percentage of cell killing was calculated as (live B cell number in nontreatment group−live B cell number in treatment group)/(live B cell number in nontreatment group)×100. FIGS. 11A-11D and Table 2 show CD79b/NKp46 bispecific antibodies can efficiently kill endogenous B cells in all PBMC donors tested compared to negative isotype control. Significantly, even for donors (#889 and #2890) with a low NK cell percentage, CD79b/NKp46 bispecific antibodies could engage NK cells and potently kill B cells.

TABLE 2
NK cell percentage of different doners' PMBC

| Donor | NK cell % in PBMC |
|---|---|
| 889 | 6.5% |
| 356 | 20.5% |
| 2890 | 9% |
| 3829 | 11.5% |

### Example 15: Activation of NK Cells by NK Cell Engagers with Optimized Fc

The ability of NK cell engagers with optimized Fc to activate NK cells in the presence of target Ramos cells was evaluated. NK cell engagers CD79b/NKp46 bispecific antibodies with optimized Fc (ch44G2/ch6-1 (LC) Fc-optimized and ch44G2/ch6-1 (HC) Fc-optimized) and negative control (ch44G2 Fc-silenced) were serially diluted. CD56+NK cells were purified using negative selection (Stemcell Technologies, #17955) from human PBMC donor #393. Purified NK cells were cultured overnight in complete RPMI media (with 10% heat inactivated FBS and 5% penicillin/streptomycin). About 100,000 NK cells were incubated with 20,000 Ramos (E:T ratio of 5:1) in the presence of serially diluted CD79b/NKp46 bispecific antibodies for 4 hours at 37° C. After incubation, cells were stained with a flow cytometry antibody cocktail containing CD56-FITC (Biolegend, #362546), CD69-PE (Biolegend, #985202) and CD107a-APC (Biolegend, #328620) in FACS buffer (PBS+0.5% BSA) on ice for 30 mins. After washing twice with FACS buffer, the cells were re-suspended in 100 μl of FACS buffer with Sytox blue live/dead stain (Thermofisher, #S34857) and analyzed with a Cytek cytometer. FIGS. 12A-12B shows that CD56+NK cells had increased activation markers CD69 and CD107a in the presence of CD79b/NKp46 antibodies compared to negative isotype control.

The agonistic activity of NK cell engagers and monospecific antibody was assessed. The NK cell engagers CD79b/NKp46 bispecific antibody ch44G2/ch6-1 (LC) Fc-optimized and control (ch44G2 Fc-optimized) were serially diluted. CD56+NK cells were purified using negative selection (Stemcell Technologies, #17955) from human PBMC donor #301. Purified NK cells were cultured overnight in complete RPMI media (with 10% heat inactivated FBS and 5% penicillin/streptomycin). About 100,000 NK (NKp46+) cells were incubated with 20,000 Ramos (CD79b+) (E:T ratio of 5:1) in the presence of serially diluted antibodies for 4 hours at 37° C.

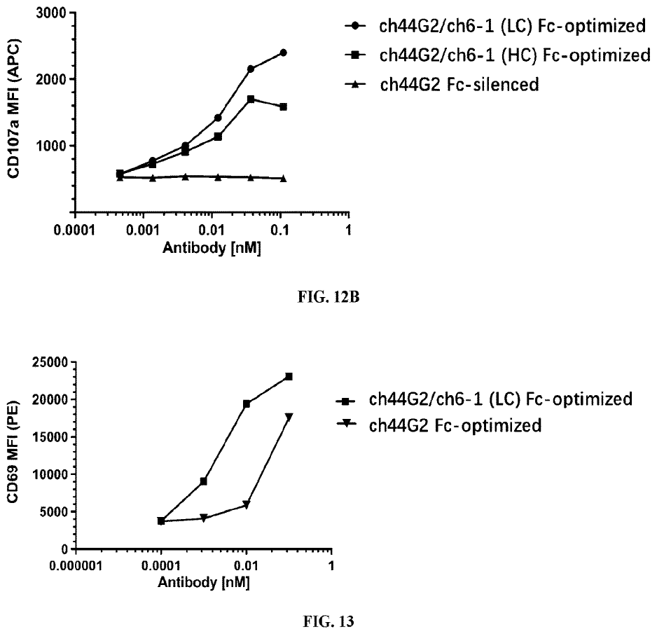

After incubation, cells were stained with a flow cytometry antibody cocktail containing CD56-FITC (Biolegend, #362546), and CD69-PE (Biolegend, #985202) in FACS buffer (PBS+0.5% BSA) on ice for 30 mins. After washing twice with FACS buffer, the cells were re-suspended in 100 μl of FACS buffer with Sytox blue live/dead stain (Thermofisher, #S34857) and analyzed with a Cytek cytometer. The analysis in FIG. 13 shows that CD56+NK cells had increased activation marker CD69 in the presence of CD79b/NKp46 antibody compared to CD79b antibody suggesting that NK cell activation can be further enhanced by adding NKp46 ScFv.

### Example 16: NK Cell Engagers with Optimized Fc Mediated Target Cancer B Cell Killing by NK Cells

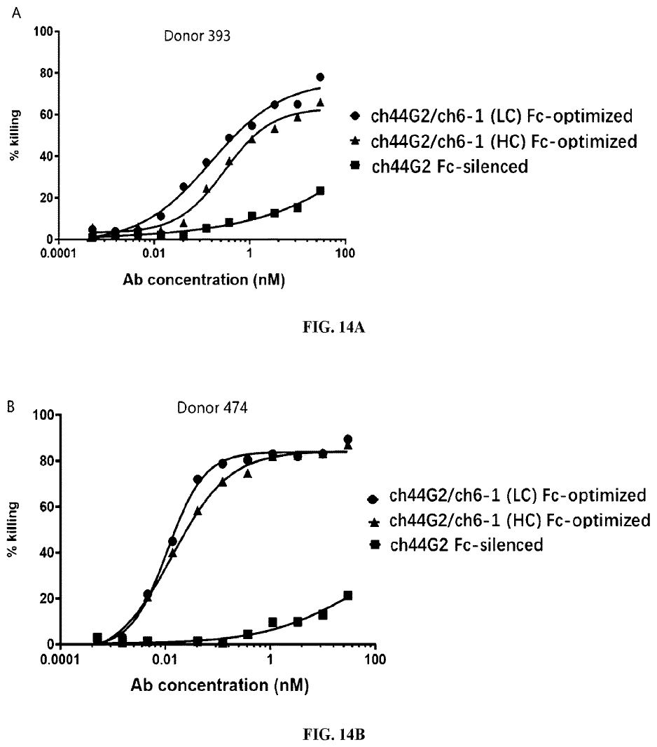

The ability of NK cell engagers to kill malignant B cell line Ramos cell by effector NK cells was evaluated. The NK cell engagers (e.g., CD79b/NKp46 bispecific antibodies ch44G2/ch6-1 (LC) Fc-optimized and ch44G2/ch6-1 (HC) Fc-optimized) and negative control (ch44G2 Fc-silenced) were serially diluted. Ramos as target cells were stained with CFSE first, then mixed with fresh isolated NK cells (E:T=5:1) in 96-well-round-bottom plate, further incubated with serial diluted CD79b/NKp46 bispecific antibodies (1 to 3 dilution from 30 nM) for 48 hrs, then the plate were spin down, medium were saved for cytokine releasing ELISA, cells were stained with live dead staining, then processed on Cytek flowcytometry, percent of cell killing was calculated as (live tumor cell number in nontreatment group−live tumor cell number in treatment group)/live tumor cell number in nontreatment group×100. Data of two donors were shown in FIGS. 14A-14B. The maximum killing percentage was about 70 to 80% for the two NK cell engagers, while for ch44G2 Fc-silenced, the maximum killing was around 20% for both donors.

### Example 17: NK Cell Engagers with Optimized Fc Mediated Target Cancer B Cell Killing by PBMC

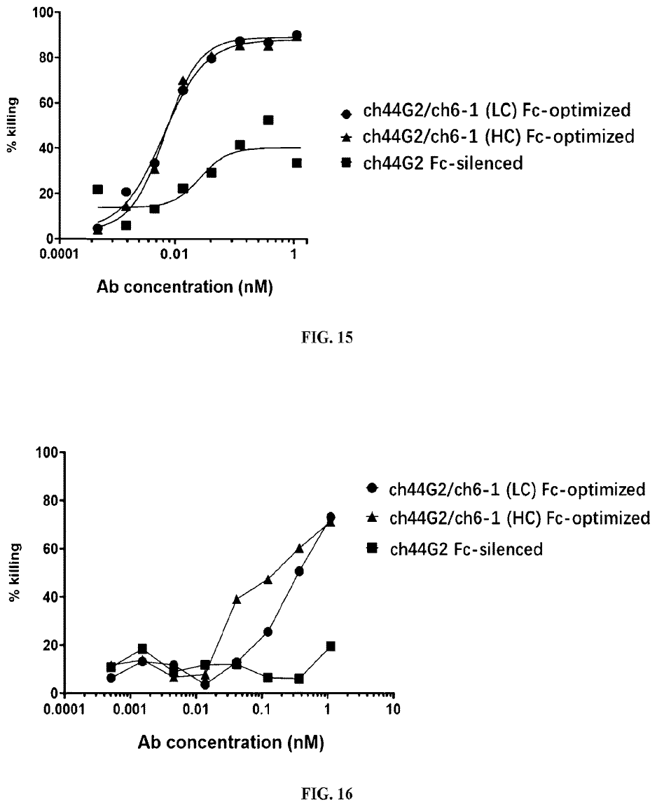

The NK cell engagers showed promising malignant B cell killing efficacy by purified NK cells (E:T=5:1), but their behavior in peripheral blood mononuclear cells (PBMCs) with less NK effector cells are still unknown. In purpose to answer this question, malignant B cells, and fresh prepared PBMC were incubated with the NK engagers to test their functions in PBMC. The NK cell engagers CD79b/NKp46 bispecific antibodies (ch44G2/ch6-1 (LC) Fc-optimized and ch44G2/ch6-1 (HC) Fc-optimized) and negative control (ch44G2 Fc-silenced) were serially diluted. Malignant B cells with different CD79b copies (Ramos and Nalm-6) were stained with CFSE first, then spiked into PBMC (E:T=10:1) in 96-well-round-bottom plate, further incubated with serial diluted CD79b/NKp46 bispecific antibodies (1 to 3 dilution from 30 nM), after 48 hrs incubation, the plate were spin down, medium were saved for cytokine releasing ELISA, cells were stained with live dead staining, then processed on Cytek flowcytometry, percent of cell killing was calculated as (live tumor cell number in nontreatment group−live tumor cell number in treatment group)/live tumor cell number in nontreatment group×100. As shown in FIG. 15 and FIG. 16, the NK engagers showed potent malignant B cell killing effect at very low concentration (less than 1 nM) in vitro, the maximum specific tumor cell killing was around 85% for Ramos at 48 hrs. For Nalm-6, the number was around 80%. The adjusted E:T=0.21:1 for Ramos cell killing; for Nalm-6 cells, the adjusted E:T=0.35:1, counted by NK cells to total B cells (CD19+) cells, indicating NK cell engagers can promote ADCC efficiently at very low E:T ratio of NK cells to target cells.

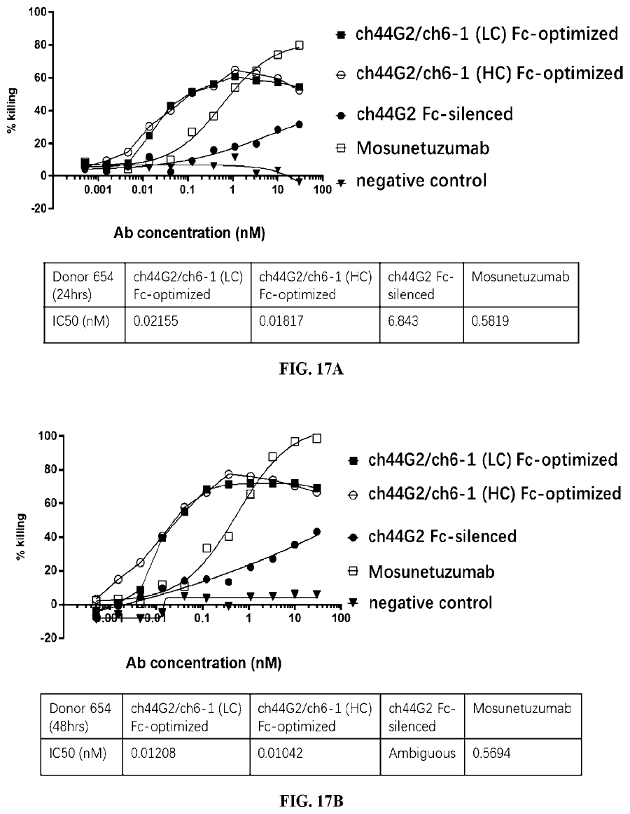

The cancer cell killing activity of NK cell engagers was evaluated. Malignant B cell line Ramos (20,000/well) were stained with CFSE (1:60,000 dilution from 25 mM) first, then spiked into fresh PBMC (2E5/well) (Donor 654 from Stanford blood center) (E:T=10:1) in 96-well-round-bottom plate, further incubated with our serial diluted CD79b/NKp46 antibody (1 to 3 dilution from 30 nM), after 24 hrs and 48 hrs incubation, the plate were spin down, medium were saved for cytokine releasing ELISA, the cells were stained with CD19-PE (Biolegend, #982402) in FACS buffer (PBS+0.5% BSA) on ice for 30 mins. After washing twice with FACS buffer, the cells were re-suspended in 100 μl of FACS buffer with Sytox blue live/dead staining (Thermofisher, #S34857) and analyzed with a Cytek cytometer. Percent of cell killing was calculated as (live tumor cell number in nontreatment group−live tumor cell number in treatment group)/live tumor cell number in nontreatment group×100. Mosunetuzumab (Mosun, Ichor Bio) was used as the bench-mark positive control. The adjusted E:T=1.21:1, counted by the total of NK cells to the total of B (CD19+) cells. As shown in FIGS. 17A-17B, the NK engagers showed potent Ramos cell killing effect at very low concentration (less than 1 nM) in vitro, the maximum specific tumor cell killing was around 60% at 24 hrs and 85% at 48 hrs. The IC50 of the two clones, ch44G2/ch6-1 (LC) Fc-optimized and ch44G2/ch6-1 (HC) Fc-optimized, were about 0.2 nM for 24 hrs and 0.1 nM for 48 hrs, which were lower than the Mosunetuzumab positive control treatment group.

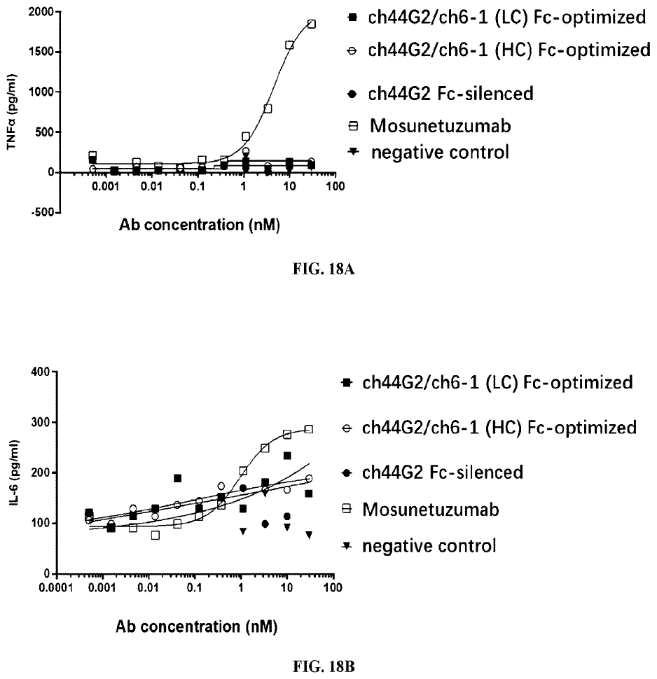

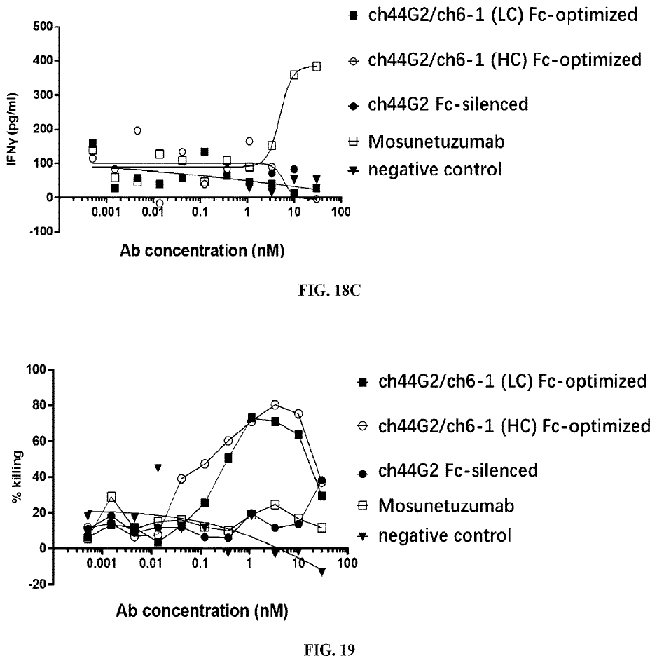

Cytokine release activity of NK cell engagers was evaluated. Supernatant of PBMC mediated Ramos killing assay were collected at 24 hrs and 20 ul of the supernatant were used for TNFα (R&D, DY210-05), IL-6 (R&D, DY206-05) and IFNγ (R&D,DY285B-05) ELISA according to manufacturer's instructions. For analysis, the TNFα, IL-6 and IFNγ levels in pg/mL was determined and plotted using GraphPad prism software (Version 10.10.1: GraphPad Software Inc). As shown in FIGS. 18A-18C, TNFα, IL-6 and IFNγ started to increase in the Mosunetuzumab treatment groups from 1 nM to 30 nM treatment, while the cytokines level in the NK cell engager's treatment groups are still at basic or lower level compared to Mosunetuzumab, indicating that the NK cell engager antibodies have a safe profile.

The effect of killing of Nalm-6 (low CD79b expressed malignant B cell line) mediated by NK cell engagers was evaluated. Malignant B cell line Nalm-6 were stained with CFSE first, then spiked into fresh PBMC (Donor 718 from Stanford blood center) (E:T=10:1) in 96-well-round-bottom plate, further incubated with serial diluted bispecific antibodies (1 to 3 dilution from 30 nM), after 48 hrs incubation, the cells were stained with CDI 9+B and then stained with cytox-blue for live dead staining, then processed on Cytek flowcytometry. Percent of cell killing was calculated as (live tumor cell number in nontreatment group−live tumor cell number in treatment group)/live tumor cell number in nontreatment group×100. Mosunetuzumab (Mosun, Tchor Bio) was used as the bench-mark positive control. The adjusted E:T=0.35:1, counted by the total of NK cells to the total of B (CD19+) cells. As shown in FIG. 19, at 48 hrs, the killing rate of Nalm-6 cells increased to 80% by bispecific antibodies. The IC50 of the two clones (ch44G2/ch6-1 (LC) Fc-optimized and ch44G2/ch6-1 (HC) Fc-optimized) were about 0.1 nM to 0.2 nM at 48 hrs for Nalm-6 cell killing.

### Example 18: The Numbers of NK Cells and Monocytes During Target Cell Killing by NK Cell Engagers

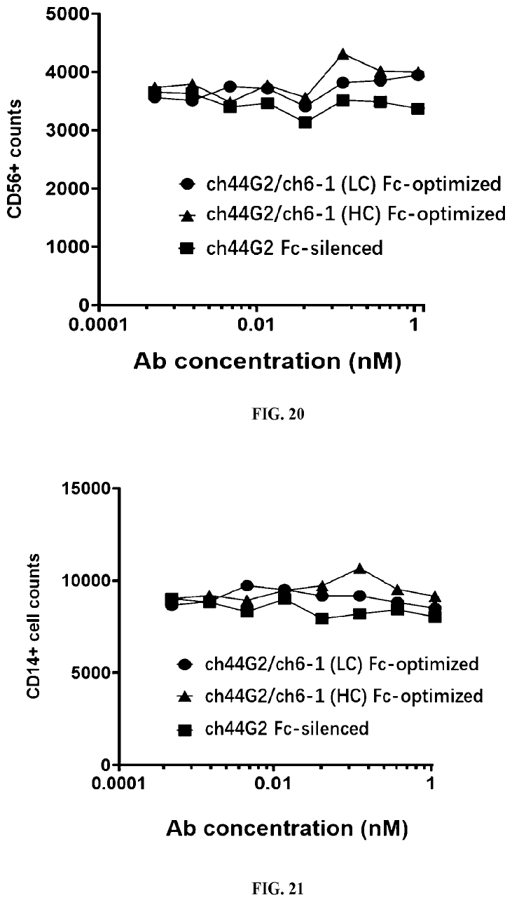

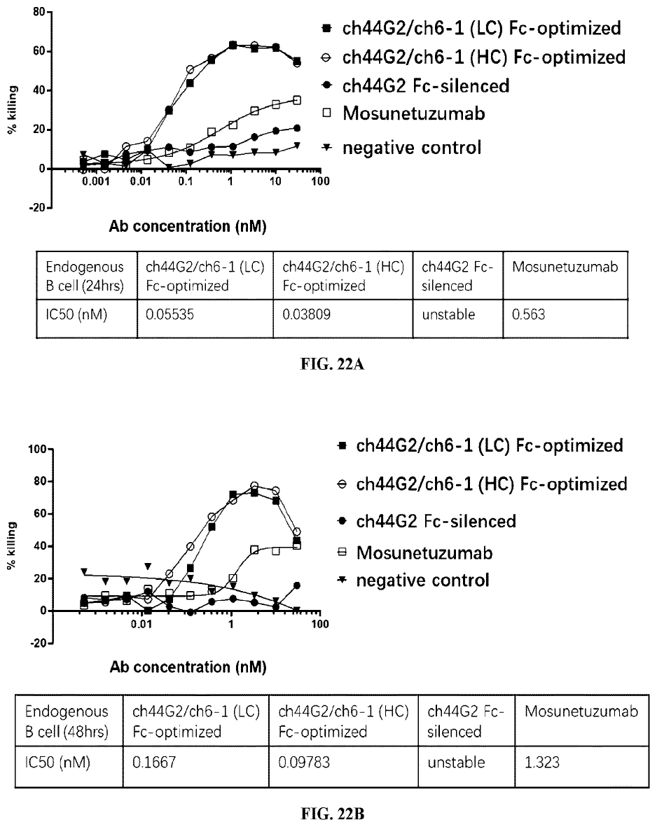

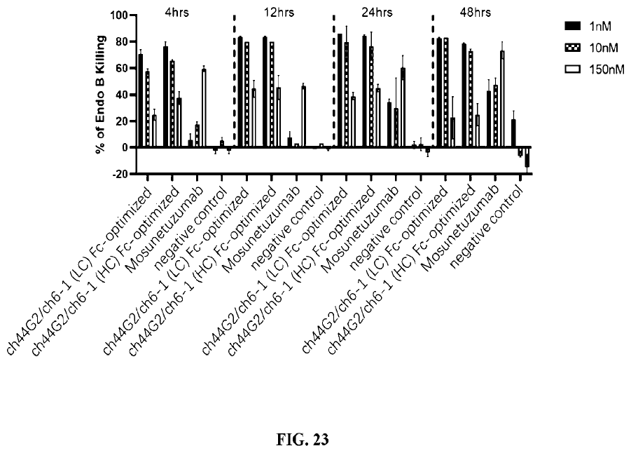

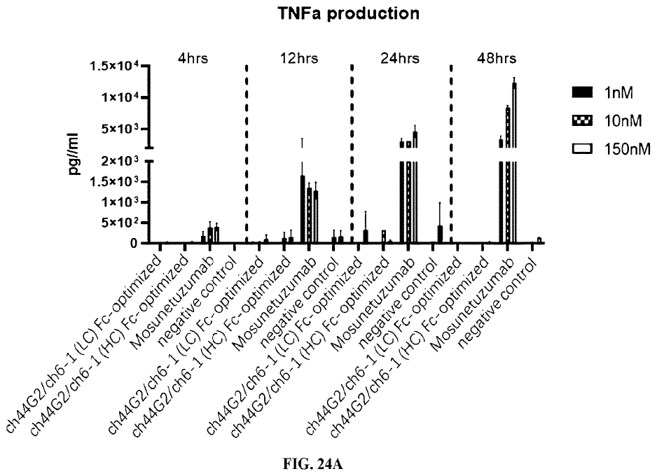

To find out whether the activated NK cells further cause self-killing to reduce its numbers, we analyzed CD69+NK cells and CD14+ monocytes to test their activation status. The NK cell engagers CD79b/NKp46 bispecific antibodies (ch44G2/ch6-1 (LC) Fc-optimized and ch44G2/ch6-1 (HC) Fc-optimized) and negative control (ch44G2 Fc-silenced) were serially diluted. Malignant B cells Nalm-6 were stained with CFSE first, then spiked into PBMC (E:T=10:1) in 96-well-round-bottom plate, further incubated with our serial diluted NK cell engagers (1 to 3 dilution from 30 nM), after 24 hrs and 48 hrs incubation, the plate were spin down, cells were stained with CD56 for NK cells, CD69 for activation marker, CD14 for Monocytes. FIG. 20 and FIG. 21 show that there were no significant changes of NK cell counts as well as Monocyte counts.

### Example 19: NK Cell Engagers with Optimized Fc Mediate Endogenous B Cell Killing by PBMC

The effect of killing of endogenous B cell mediated by NK cell engagers bispecific antibody CD79b/NKp46 was evaluated. Endogenous B cell were stained with CFSE first, then spiked into fresh PBMC (Donor 718 from Stanford blood center) (E:T=10:1) in 96-well-round-bottom plate, further incubated with serial diluted bispecific antibodies (1 to 3 dilution from 30 nM), after 48 hrs incubation, the cells were stained with CD19+B and then stained with cytox-blue for live dead staining, then processed on Cytek flow cytometry, percent of cell killing was calculated as (live tumor cell number in nontreatment group−live tumor cell number in treatment group)/live tumor cell number in nontreatment group×100. Mosunetuzumab (Mosun, Ichor Bio) was used as the bench-mark positive control. The adjusted E:T=0.35:1, counted by the total of NK cells to the total of B (CD19+) cells. As shown in FIGS. 22A-22B, it showed potent endogenous B cell killing effect at 24 hrs (600%) and 48 hrs (80%) with very low concentration in vitro.

To test NK cell engagers mediated B cells depletion, healthy human PBMC were used as a testing system. Healthy Human PBMC were isolated from donated healthy human blood and suspended in fresh medium+10% FBS. 15 μl of serially diluted testing NK cell engagers was added into designated well of a U-bottom 96 well plate. 2×10⁵ PBMC in 135 μl complete culture medium were added into each well respectively. The mixture was incubated at 37° C.+5% CO₂ for 48 hours. Cells were collected by spinning down at 1500 rpm for 5 minutes. Supernatant from each well was kept for ELISA assay. Cells pellet were resuspended in FACS buffer and stained/analyzed with Cytek cytometer. B cells were gated by L/D dye-CD19+ markers. the living B cells were counted. The efficacy of B cells depletion was calculated by the formula: the depletion efficacy=(B cells count in control group-B cells count in treated group)/B cells count in control group×100. The result indicated that NK cell engagers demonstrated rapid and potent effect on B cells depletion. As shown in FIG. 23, the significant B cells depletion could be observed at 4 hours after incubation of PBMC with NK cell engagers at 1 nM concentration and this effect lasted through test time. Compared with bench control, NK cell engagers showed superior efficiency in B cells depletion.

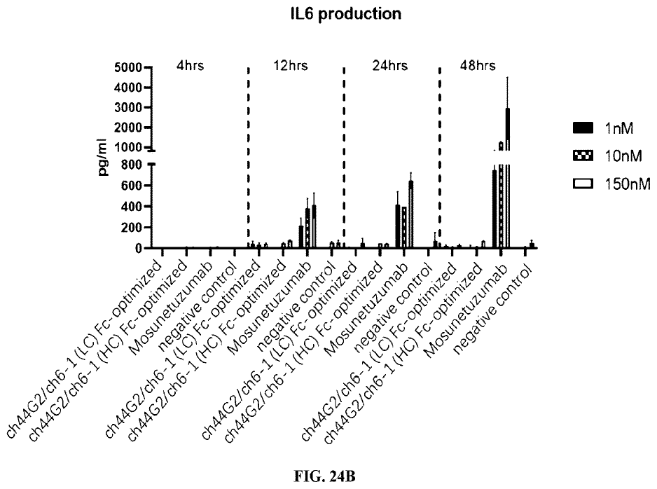

To explore the status of cytokines production induced by NK cell engagers while executing their killing effect, the supernatant from the incubation of PBMC with NK cell engagers at 48 hours was collected and cytokines were assayed by ELISA (1L6 (invitrogen Kit Cat #88-7066-88) and TNFα (invitrogen Kit Cat #88-346-88)). High binding ELISA plate were coated with capture antibodies overnight. Next day, following 3× wash with washing buffer (PBST+0.05% Tween 20), 100 μl blocking buffer (1% BSA PBST) was added into each well and plate was kept at room temperature for one hour. After washing three times with wash buffer (PBST), 50 μl sample (5 times diluted supernatant)/well (50 ul 1% FBS PBS in blank well) was added into designated well. The plate was sealed with plastic sealing film and incubated at RT for 2 hours. After incubation, the plate was washed 3 times with wash buffer. Then, 100 μl detection Abs was added into each well. The plate was sealed with plastic sealing film and incubated at RT for one more hour. After 3 times wash with wash buffer, 100 μl SP-avidin HRP was added into each well and the plate was incubated at RT for another 30 minutes, after 6 times wash with wash buffer, 100 μl TMB solution was added into each well and the plate was incubated at RT for 15 minutes. Then, 100 μl stop solution was added into each well. OD at 450 nm was read in spectrometer. As shown in FIGS. 24A-24B, NK cell engagers (ch44G2/ch6-1 (LC) Fc-optimized and ch44G2/ch6-1 (HC) Fc-optimized) induced minor production of TNFα and IL6 in the testing system, inconsiderable compared with bench control.

### Example 20: NK Cell Engagers Mediated Target Cancer B Cell Killing by Monocytes and NK Cells

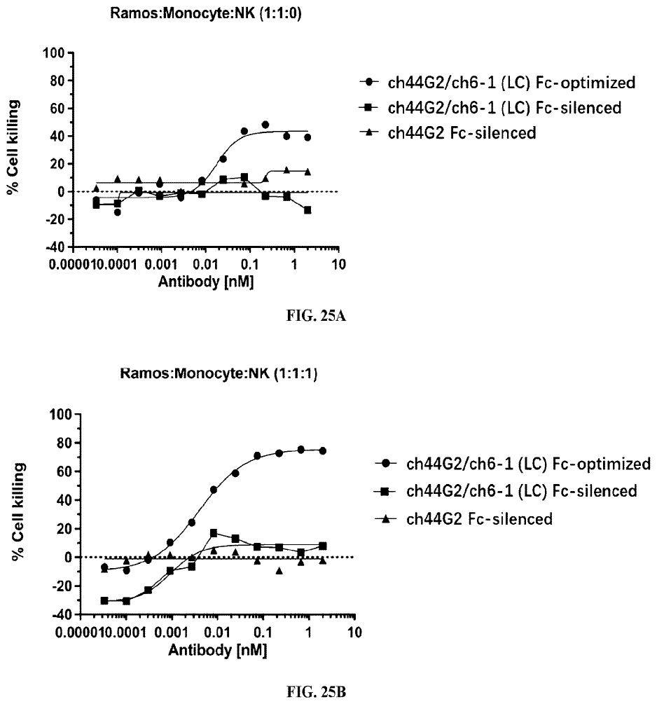

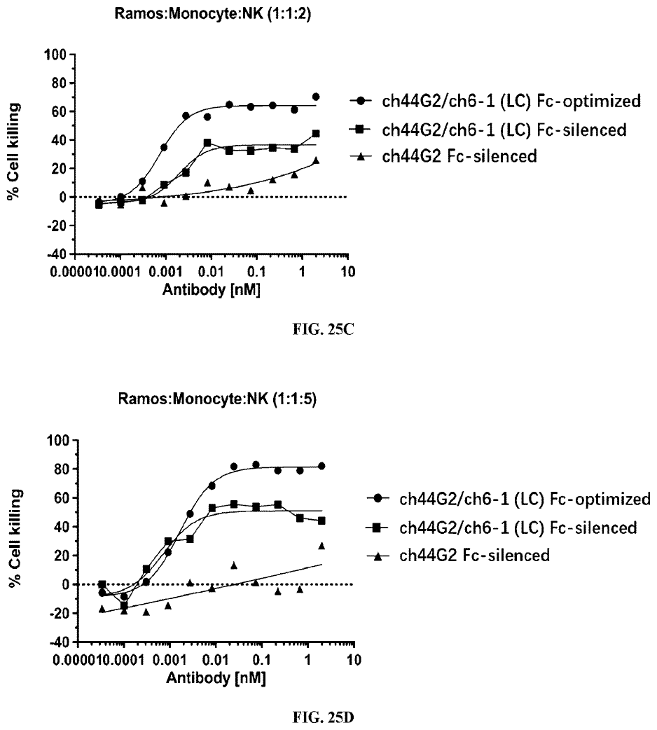

The ability of NK cell engagers CD79b/NKp46 antibodies to engage both monocytes and NK cells for a potent killing of target Ramos cells were assessed. CD14+ monocytes and CD56+NK cells (purified using Stemcell Technologies kits #17955 and #19058 from human PBMC donor #827) were purified using negative selection. Ramos cells were labelled with CFSE (Thermofisher, #C34554). About 20,000 monocytes cells were incubated with 20,000 CFSE+ Ramos (E:T ratio of 1:1) in the presence of serially diluted antibodies in complete RPMI media (with 10% heat inactivated FBS and 5% penicillin/streptomycin). Additionally, CD56+NK cells were titrated into the assays in ratios that are described in the data figures. The assay was carried out for 24 hours at 37° C. After incubation, the cells were re-suspended in 100 μl of FACS buffer with Sytox blue live/dead stain (Thermofisher, #S34857) and analyzed with a Cytek cytometer. The monocyte and NK cells mediated killing were determined by counting absolute number of CFSE+ Ramos cells by collecting a fixed volume of data for all treatment conditions. Percentage target cell killing was calculated as (Absolute number of CFSE+ Ramos cell number in nontreatment group−Absolute number of CFSE+ SYTOX−Ramos cell number in treatment group)/Absolute number of CFSE+ Ramos cell number in nontreatment group×100. As shown in Table 3 and FIGS. 25A-25D, both monocytes and NK cells can be engaged concurrently by CD79b/NKp46 antibodies for a potent killing of target cancer cell Ramos.

TABLE 3
NK Cell Engagers Mediated Target Cancer
B Cell Killing by Monocytes and NK Cells

| | Ratio (Ramos:Monocyte:NK) | | | |
|---|---|---|---|---|
| | 1:1:0 | 1:1:1 | 1:1:2 | 1:1:5 |
| antibodies | (IC50) | (IC50) | (IC50) | (IC50) |
| ch44G2/ch6-1 (LC) | 17.4 pM | 4.0 pM | 0.733 pM | 1.65 pM |
| Fc-optimized | | | | |
| ch44G2/ch6-1 (LC) | — | 0.948 pM | 1.933 pM | 0.711 pM |
| Fc-silenced | | | | |
| ch44G2 Fc-silenced | — | — | — | — |

### Example 21: Binding Abilities of Humanized NK Cell Engagers to NKp46 Expression Cells

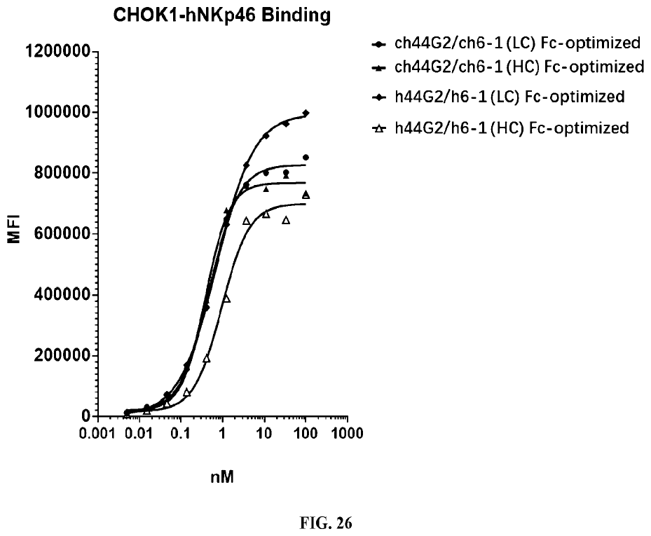

Binding abilities of NK cell engagers to human NKp46 overexpression cell line, CHOK1-hNKp46 cells were evaluated. 2 chimeric antibodies (ch44G2/ch6-1 (LC) Fc-optimized and ch44G2/ch6-1 (HC) Fc-optimized) and 2 humanized antibodies (h44G2/h6-1 (LC) Fc-optimized, and h44G2/h6-1 (HC) Fc-optimized) were serially diluted. About 100,000 CHOK1-hNKp46 cells were incubated with serially diluted CD79b/NKp46 bispecific antibodies in FACS buffer (PBS+0.5% BSA) for 60 mins on ice. After incubation cells were washed twice and detected with anti-human IgG Fc PE secondary antibody (Invitrogen, #12-4998-82). For analysis, the mean fluorescence intensity (MFI of PE) of each antibody was determined and plotted using GraphPad prism software (Version 7; GraphPad Software Inc). FIG. 26 shows that all four CD79b/NKp46 bispecific antibodies can bind CHOK1-hNKp46 (hNKp46+) cells with similar affinity.

### Example 22: Binding Abilities of Humanized NK Cell Engagers to CD79b Expression Cells

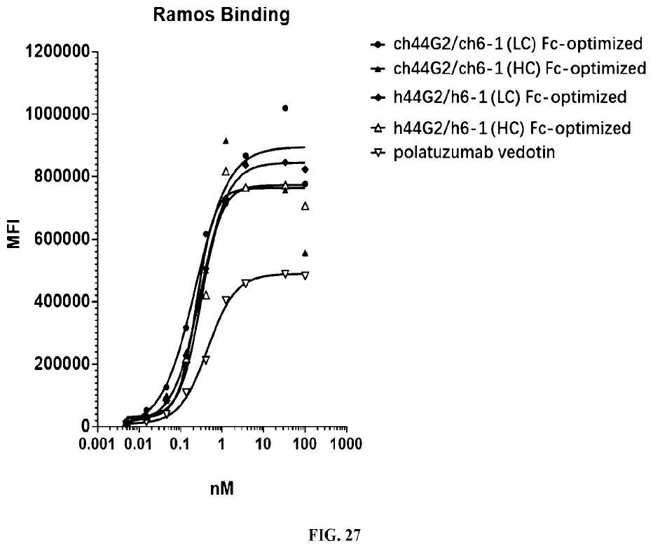

Binding abilities of NK cell engagers to target Ramos cells were evaluated. 2 chimeric antibodies (ch44G2/ch6-1 (LC) Fc-optimized and ch44G2/ch6-1 (HC) Fc-optimized), 2 humanized antibodies (h44G2/h6-1 (LC) Fc-optimized and h44G2/h6-1 (HC) Fc-optimized) and commercial Polatuzumab Vedotin (Polivy) were serially diluted. About 100,000 Ramos cells were incubated with serially diluted CD79bxNKp46 bispecific antibodies in FACS buffer (PBS+0.5% BSA) for 60 mins on ice. After incubation cells were washed twice and detected with anti-human IgG Fc PE secondary antibody (Invitrogen, #12-4998-82). For analysis, the mean fluorescence intensity (MFI of PE) of each antibody was determined and plotted using GraphPad prism software (Version 7; GraphPad Software Inc). FIG. 27 shows that all four CD79b/NKp46 bispecific antibodies can bind Ramos (CD79+) cells with similar affinity, all four CD79b/NKp46 bispecific antibodies showed better affinity than commercial Polatuzumab Vedotin.

### Example 23: Humanized NK Cell Engagers Mediate Target Cancer B Cell Killing by PBMC

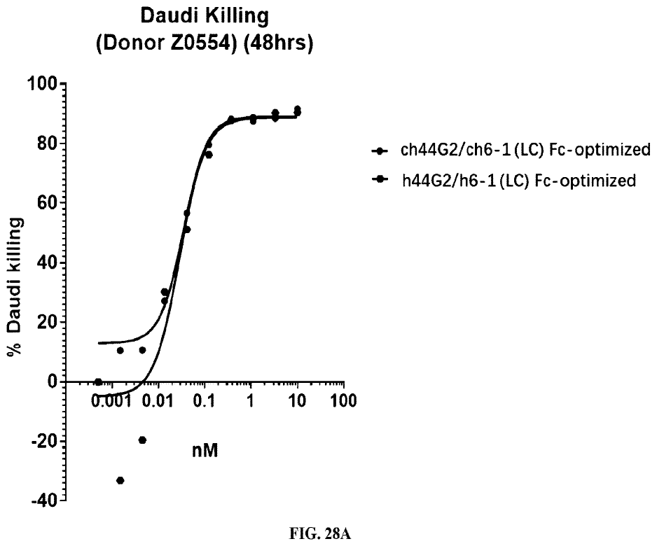

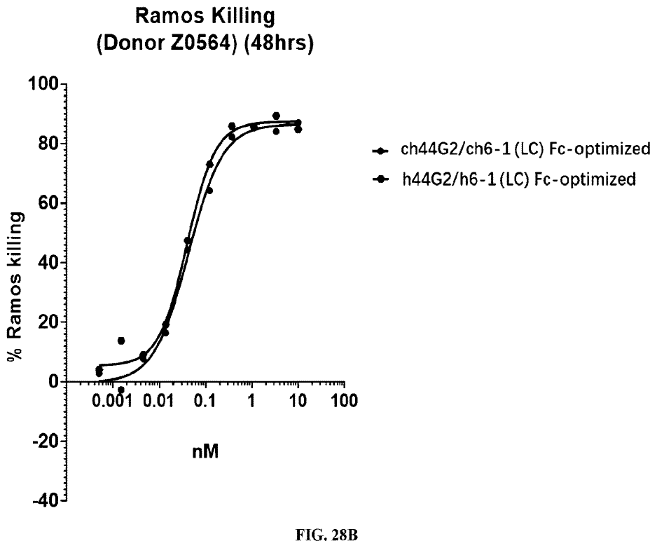

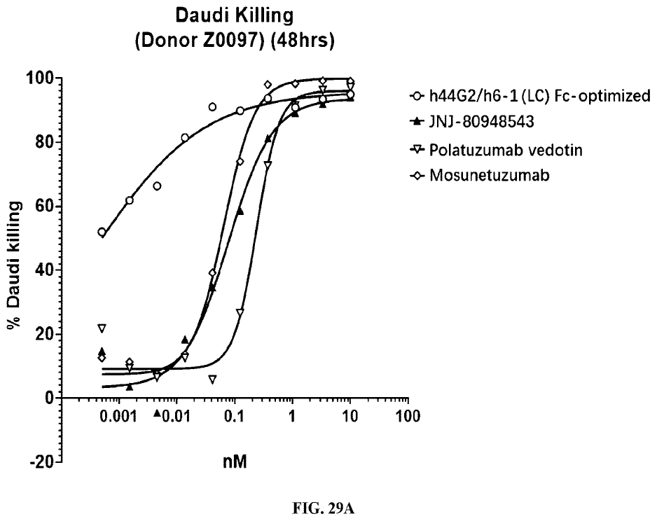

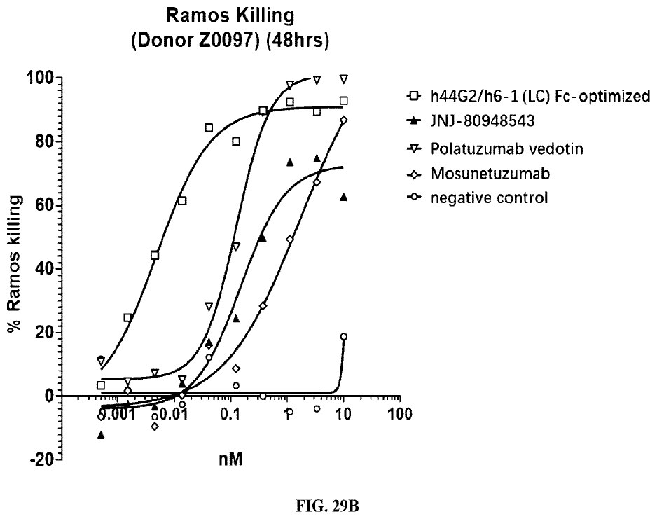

The ability of humanized NK cell engagers to kill malignant B cell line Daudi and Ramos cell in PBMCs was evaluated. The NK cell engagers (ch44G2/ch6-1 (LC) Fc-optimized and h44G2/h6-1 (LC) Fc-optimized) were serially diluted. Daudi and Ramos as target cells were stained with CFSE first, then mixed with fresh PBMCs (E:T=10; 1) in 96-well-round-bottom plate, further incubated with serial diluted CD79b/NKp46 bispecific antibodies (1 to 3 dilution from 10 nM) for 48 hrs, then the plate were spin down, cells were stained with live dead staining, then processed on CytoFLEX flowcytometry. Percent of cell killing was calculated as (live tumor cell number in nontreatment group−live tumor cell number in treatment group)/live tumor cell number in nontreatment group×100. Data of two donors were shown in FIGS. 28A-28B. The maximum killing percentage was about 90% for three NK cell engagers, humanized NK cell engagers (h44G2/h6-1 (LC) Fc-optimized) show comparable efficacy to chimeric antibody (ch44G2/ch6-1 (LC) Fc-optimized) for both Daudi cell and Ramos cell.

The cancer cell killing activity of humanized NK cell engagers with JNJ-80948543 (CD79b-CD20xCD3 antibody), commercial Polatuzumab Vedotin (Polivy) and commercial Mosunetuzumab (Mosun) were compared. The ability of humanized NK cell engagers to kill malignant B cell line Daudi cell and Ramos cell in PBMCs was evaluated. The NK cell engagers (h44G2/h6-1 (LC) Fc-optimized), commercial Polatuzumab Vedotin (Polivy), JNJ-80948543 (CD79bxCD20xCD3 antibody) and commercial Mosunetuzumab (Mosun) were serially diluted. Daudi cell and Ramos cell as target cells were stained with CFSE first, then mixed with fresh PBMCs (E:T=10:1) in 96-well-round-bottom plate, further incubated with serial diluted CD79b/NKp46 bispecific antibodies, JNJ-80948543, commercial Polatuzumab Vedotin and commercial Mosunetuzumab (1 to 3 dilution from 10 nM) for 48 hrs, then the plate were spin down, cells were stained with live dead staining, then processed on CytoFLEX flowcytometry, percent of cell killing was calculated as (live tumor cell number in nontreatment group−live tumor cell number in treatment group)/live tumor cell number in nontreatment group×100. Data was shown in FIGS. 29A-29B and Table 4. The maximum killing percentage was about 90% for humanized NK cell engagers in both Daudi and Ramos cancer cells killing. Significantly, humanized CD79bxNKp46 antibodies showed lower EC50, suggesting that humanized CD79b/NKp46 antibodies had superior killing activity against both Daudi and Ramos cancer cells compared to JNJ-80948543, commercial Polatuzumab Vedotin (Polivy) and commercial Mosunetuzumab.

TABLE 4
Humanized NK cell Engagers Mediate Target
Cancer B Cell Killing by PBMC

| | h44G2/h6-1 | | | |
|---|---|---|---|---|
| | (LC) Fc- | JNJ- | Polatuzumab | |
| | optimized | 80948543 | Vedotin | Mosunetuzumab |
| Daudi | 0.0012 | 0.0765 | 0.2363 | 0.0638 |
| (EC50 nM) | | | | |
| Ramos | 0.0076 | 0.1492 | 0.1249 | 1.691 |
| (EC50 nM) | | | | |

### Example 24: Plasma Spiked NK Cell Engagers Mediated Target Cancer B Cell Killing by PBMC

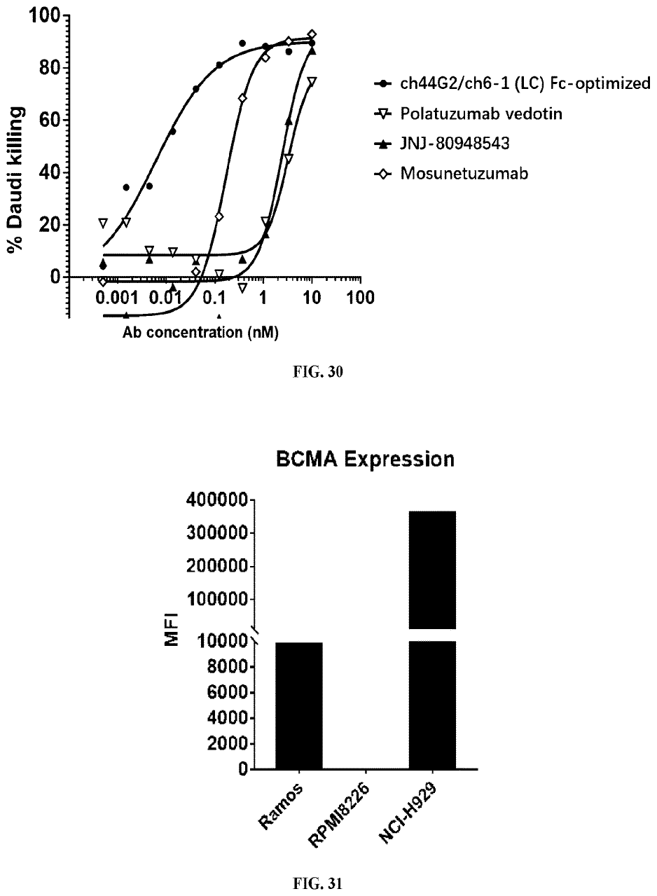

The cancer cell killing activity of NK cell engagers with commercial Polatuzumab Vedotin (Polivy), JNJ-80948543 (CD79b/CD20/CD3 antibody) and commercial Mosunetuzumab (Mosun) with plasma spiked were compared. The ability of NK cell engagers to kill malignant B cell line Daudi cell in PBMCs with plasma was evaluated. The NK cell engager (ch44G2/ch6-1 (LC) Fc-optimized), commercial Polatuzumab Vedotin (Polivy), JNJ-80948543 and commercial Mosunetuzumab (Mosun) were serially diluted. Daudi cell as target cells were stained with CFSE first, then mixed with fresh PBMCs (E:T=10:1) in 96-well-round-bottom plate, then spiked with fresh plasma (Half of the total volume) in 96-well-round-bottom plate, further incubated with serial diluted CD79b/NKp46 bispecific antibodies, commercial Polatuzumab Vedotin Polivy), JNJ-80948543 and commercial Mosunetuzumab (Mosun) (1 to 3 dilution from 10 nM) for 48 hrs, then the plate were spin down, cells were stained with live dead staining, then processed on CytoFLEX flowcytometry, percent of cell killing was calculated as (live tumor cell number in nontreatment group−live tumor cell number in treatment group)/live tumor cell number in nontreatment group×100. Data was shown in FIG. 30 and Table 5. The maximum killing percentage was about 85%-90% for NK cell engager, JNJ-80948543 and commercial Mosunetuzumab (Mosun), while about 70% for commercial Polatuzumab Vedotin (Polivy).

Significantly, even at low concentration (e.g., 0.1 nM), CD79b/NKp46 bispecific antibody (about 80%) show potently killing activity against Daudi cell, compared to commercial Polatuzumab Vedotin (Polivy), JNJ-80948543 and commercial Mosunetuzumab (Mosun) (all three lower than 30%).

TABLE 5
Plasma Spiked NK cell Engagers mediated
Target Cancer B Cell Killing by PBMC

| | ch44G2/ch6-1 | | | |
|---|---|---|---|---|
| | (LC) Fc- | Polatuzumab | JNJ- | |
| | optimized | Vedotin | 80948543 | Mosunetuzumab |
| Daudi | 0.0061 | 3.211 | 2.409 | 0.1703 |
| (EC50 | | | | |
| nM) | | | | |

### Example 25: BCMA/NKp46 Bispecific Antibody Mediate Target Multiple Myeloma Cell Killing by PBMC

To estimate BCMA antigen expression levels on multiple myeloma tumor cell surface, cell lines were stained at a saturating concentration of anti-BCMA antibody on ice for 30 min. Cells were then washed with PBS and resuspended in FACS buffer for flow cytometry analysis using CytoFLEX LX. Results were analyzed and graphed using GraphPad prism software (Version 7; GraphPad Software Inc.). The quantification of BCMA antigen density on multiple myeloma tumor cell surface are shown in Table 6 and FIG. 31.

TABLE 6
Quantification of BCMA antigen density on multiple
myeloma tumor cell and normal B cell surface

| Cell line | Ramos | RPMI8226 | NCI-H929 |
|---|---|---|---|
| MFI(PE) | 9815 | 44586 | 363189 |

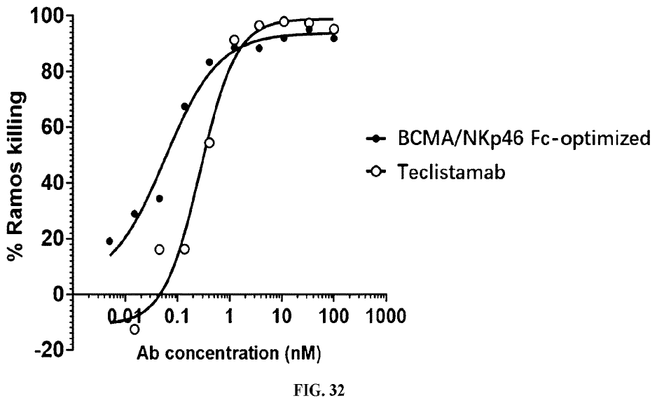

To estimate multiple myeloma cell killing efficacy in peripheral blood mononuclear cells (PBMCs) with less NK effector cells. Multiple myeloma cells, and fresh prepared PBMC were incubated with the NK engagers to test their functions in PBMC. The NK cell engagers BCMA/NKp46 bispecific antibodies (BCMA/NKp46 Fc-optimized) and positive control (Teclistamab: BCMA/CD3 antibody) were serially diluted. Tumor cells with low BCMA copies (Ramos) were stained with CFSE first, then spiked into PBMC (E:T=10:1) in 96-well-round-bottom plate, further incubated with serial diluted BCMA/NKp46 or Teclistamab bispecific antibodies (1 to 3 dilution from 100 nM), after 48 hrs incubation, the plate were spin down, cells were stained with live dead staining, then processed on CytoFLEX flow cytometry, percent of cell killing was calculated as (live tumor cell number in nontreatment group−live tumor cell number in treatment group)/live tumor cell number in nontreatment group×100. As shown in FIG. 32, the NK engagers showed potent Ramos cell killing effect at very low concentration (less than 1 nM) in vitro, the maximum specific tumor cell killing was around 90% for Ramos at 48 hrs. The adjusted BCMA/NKp46 bispecific antibody E:T=1:1 for Ramos cell killing; For Teclistamab (BCMA/CD3 antibody), the adjusted E:T=8:1. Counted by NK cells to total Ramos (BCMA+) cells, indicating NK cell engagers can promote ADCC efficiently at very low E:T ratio of NK cells to target cells.

### Example 26: EGFR/NKp46 Bispecific Antibody Mediate Target Cell Killing by PBMC

To estimate target cell killing efficacy in peripheral blood mononuclear cells (PBMCs) with NK effector cells. DLD-1 cells, and fresh prepared PBMC were incubated with the NK engagers to test their functions in PBMC. The NK cell engagers EGFR/NKp46 bispecific antibody (EGFR/NKp46 Fc-optimized) and positive control (Cetuximab) were serially diluted. DLD-1 cells were stained with CFSE first, then spiked into PBMC (E:T=5:1) in 96-well-round-bottom plate, further incubated with serial diluted EGFR/NKp46 or Cetuximab (1 to 3 dilution from 100 nM), after 48 hrs incubation, the plate were spin down, cells were stained with live dead staining, then processed on CytoFLEX flow cytometry, percent of cell killing was calculated as (live tumor cell number in nontreatment group−live tumor cell number in treatment group)/live tumor cell number in nontreatment group×100. The result showed that the NK engagers showed potent DLD-1 cell killing effect at very low concentration (less than 1 nM) in vitro.

### Example 27: EpCAM/NKp46 Bispecific Antibody Mediated Target Cell Killing by PBMC

To estimate target cell killing efficacy in peripheral blood mononuclear cells (PBMCs) with NK effector cells. DLD-1 cells, and fresh prepared PBMC were incubated with the NK engagers to test their functions in PBMC. The NK cell engagers EpCAM/NKp46 bispecific antibody (EpCAM/NKp46 Fc-optimized) and positive control (Catumaxomab: EpCAM/CD3 antibody) were serially diluted. DLD-1 cells were stained with CFSE first, then spiked into PBMC (E:T=5:1) in 96-well-round-bottom plate, further incubated with serial diluted EpCAM/NKp46 or Catumaxomab bispecific antibodies (1 to 3 dilution from 100 nM), after 48 hrs incubation, the plate were spin down, cells were stained with live dead staining, then processed on CytoFLEX flow cytometry, percent of cell killing was calculated as (live tumor cell number in nontreatment group−live tumor cell number in treatment group)/live tumor cell number in nontreatment group×100. The result showed that the NK engagers showed potent DLD-1 cell killing effect at very low concentration (less than 1 nM) in vitro.

### Example 28: Other TAA/NKp46 Bispecific Antibody Mediated Target Cell Killing by PBMC

To estimate target cell killing efficacy in peripheral blood mononuclear cells (PBMCs) with NK effector cells. DLL3/NKp46 bispecific antibody, PSMA/NKp46 bispecific antibody, STEAP1/NKp46 bispecific antibody, MUC1/NKp46 bispecific antibody, c-MET/NKp46 bispecific antibody, LRRC15/NKp46 bispecific antibody, CCR8/NKp46 bispecific antibody and OX40/NKp46 bispecific antibody were constructed.

The target cells corresponding to these antibodies, and fresh prepared PBMC were incubated with the NK engagers to test their functions in PBMC. The NK cell engagers were serially diluted. Target cells were stained with CFSE first, then spiked into PBMC (E:T=5:1) in 96-well-round-bottom plate, further incubated with serial diluted antibodies (1 to 3 dilution from 100 nM), after 48 hrs incubation, the plate were spin down, cells were stained with live dead staining, then processed on CytoFLEX flow cytometry, percent of cell killing was calculated as (live tumor cell number in nontreatment group−live tumor cell number in treatment group)/live tumor cell number in nontreatment group×100. The result showed that the NK engagers showed potent target cell killing effect at very low concentration (less than 1 nM) in vitro.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An antibody or antigen-binding fragment thereof that binds to NKp46, comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 5, 7, 9, respectively, and the selected VL CDRs 1, 2, 3 amino acid sequences are set forth in SEQ ID NOs: 10, 11, 12, respectively;
(2) the selected VH CDRs 1, 2, 3 amino acid sequences are set forth in SEQ TD NOs: 6, 8, 9, respectively, and the selected VL CDRs 1, 2, 3 amino acid sequences are set forth in SEQ ID NOs: 10, 11, 12, respectively;
(3) the selected VH CDRs 1, 2, 3 amino acid sequences are set forth in SEQ ID NOs: 15, 17, 19, respectively, and the selected VL CDRs 1, 2, 3 amino acid sequences are set forth in SEQ ID NOs: 20, 21, 22, respectively;
(4) the selected VH CDRs 1, 2, 3 amino acid sequences are set forth in SEQ ID NOs: 16, 18, 19, respectively, and the selected VL CDRs 1, 2, 3 amino acid sequences are set forth in SEQ ID NOs: 20, 21, 22, respectively;
(5) the selected VH CDRs 1, 2, 3 amino acid sequences are set forth in SEQ ID NOs: 25, 27, 29, respectively, and the selected VL CDRs 1, 2, 3 amino acid sequences are set forth in SEQ ID NOs: 30, 31, 32, respectively;
(6) the selected VH CDRs 1, 2, 3 amino acid sequences are set forth in SEQ ID NOs: 26, 28, 29, respectively, and the selected VL CDRs 1, 2, 3 amino acid sequences are set forth in SEQ ID NOs: 30, 31, 32, respectively.

2. The antibody or antigen-binding fragment thereof of claim 1, wherein the VH comprises CDRs 1, 2, 3 with the amino acid sequences set forth in SEQ ID NOs: 5, 7, and 9, respectively, and the VL comprises CDRs 1, 2, 3 with the amino acid sequences set forth in SEQ ID NOs: 10, 11, and 12, respectively, according to Kabat definition.

3. The antibody or antigen-binding fragment thereof of claim 1, wherein the VH comprises CDRs 1, 2, 3 with the amino acid sequences set forth in SEQ ID NOs: 6, 8, and 9, respectively, and the VL comprises CDRs 1, 2, 3 with the amino acid sequences set forth in SEQ ID NOs: 10, 11, and 12, respectively, according to Chothia definition.

4. The antibody or antigen-binding fragment thereof of claim 1, wherein the VH comprises CDRs 1, 2, 3 with the amino acid sequences set forth in SEQ ID NOs: 15, 17, and 19, respectively, and the VL comprises CDRs 1, 2, 3 with the amino acid sequences set forth in SEQ ID NOs: 20, 21, and 22, respectively, according to Kabat definition.

5. The antibody or antigen-binding fragment thereof of claim 1, wherein the VH comprises CDRs 1, 2, 3 with the amino acid sequences set forth in SEQ ID NOs: 16, 18, and 19, respectively, and the VL comprises CDRs 1, 2, 3 with the amino acid sequences set forth in SEQ ID NOs: 20, 21, and 22, respectively, according to Chothia definition.

6. The antibody or antigen-binding fragment thereof of claim 1, wherein the VH comprises CDRs 1, 2, 3 with the amino acid sequences set forth in SEQ ID NOs: 25, 27 and 29, respectively, and the VL comprises CDRs 1, 2, 3 with the amino acid sequences set forth in SEQ ID NOs: 30, 31 and 32, respectively, according to Kabat definition.

7. The antibody or antigen-binding fragment thereof of claim 1, wherein the VH comprises CDRs 1, 2, 3 with the amino acid sequences set forth in SEQ ID NOs: 26, 28 and 29, respectively, and the VL comprises CDRs 1, 2, 3 with the amino acid sequences set forth in SEQ ID NOs: 30, 31 and 32, respectively, according to Chothia definition.

8. The antibody or antigen-binding fragment thereof of any one of claims 1-7, wherein the antibody or antigen-binding fragment specifically binds to human NKp46 or monkey NKp46.

9. The antibody or antigen-binding fragment thereof of any one of claims 1-8, wherein the antibody or antigen-binding fragment is a human or humanized antibody or antigen-binding fragment thereof.

10. The antibody or antigen-binding fragment thereof of any one of claims 1-9, wherein the antibody or antigen-binding fragment is a single-chain variable fragment (scFv) or a multi-specific antibody (e.g., a bispecific antibody).

11. A nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 5, 7, and 9, respectively, and wherein the VH, when paired with a light chain variable region (VL) comprising the amino acid sequence set forth in SEQ ID NO: 4 binds to NKp46;
(2) an immunoglobulin light chain or a fragment thereof comprising a VL comprising CDRs 1, 2, and 3 comprising the amino acid sequences set forth in SEQ ID NOs: 10, 11 and 12, respectively, and wherein the VL, when paired with a VH comprising the amino acid sequence set forth in SEQ ID NO: 3 binds to NKp46;
(3) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 6, 8 and 9, respectively, and wherein the VH, when paired with a light chain variable region (VL) comprising the amino acid sequence set forth in SEQ ID NO: 4 binds to NKp46;
(4) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 15, 17 and 19, respectively, and wherein the VH, when paired with a light chain variable region (VL) comprising the amino acid sequence set forth in SEQ ID NO: 14 binds to NKp46;
(5) an immunoglobulin light chain or a fragment thereof comprising a VL comprising CDRs 1, 2, and 3 comprising the amino acid sequences set forth in SEQ ID NOs: 20, 21 and 22, respectively, and wherein the VL, when paired with a VH comprising the amino acid sequence set forth in SEQ ID NO: 13 binds to NKp46;
(6) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 16, 18 and 19, respectively, and wherein the VH, when paired with a light chain variable region (VL) comprising the amino acid sequence set forth in SEQ ID NO: 14 binds to NKp46;
(7) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 25, 27 and 29, respectively, and wherein the VH, when paired with a light chain variable region (VL) comprising the amino acid sequence set forth in SEQ ID NO: 24 binds to NKp46;
(8) an immunoglobulin light chain or a fragment thereof comprising a VL comprising CDRs 1, 2, and 3 comprising the amino acid sequences set forth in SEQ ID NOs: 30, 31 and 32, respectively, and wherein the VL, when paired with a VH comprising the amino acid sequence set forth in SEQ ID NO: 23 binds to NKp46;
(9) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 26, 28 and 29, respectively, and wherein the VH, when paired with a light chain variable region (VL) comprising the amino acid sequence set forth in SEQ ID NO: 24 binds to NKp46;
(10) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 5, 7, and 9, respectively, and wherein the VH, when paired with a light chain variable region (VL) comprising the amino acid sequence set forth in SEQ ID NO: 36 binds to NKp46;
(11) an immunoglobulin light chain or a fragment thereof comprising a VL comprising CDRs 1, 2, and 3 comprising the amino acid sequences set forth in SEQ ID NOs: 10, 11 and 12, respectively, and wherein the VL, when paired with a VH comprising the amino acid sequence set forth in SEQ ID NO: 35 binds to NKp46;
(12) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 6, 8 and 9, respectively, and wherein the VH, when paired with a light chain variable region (VL) comprising the amino acid sequence set forth in SEQ ID NO: 36 binds to NKp46;
(13) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 15, 17 and 19, respectively, and wherein the VH, when paired with a light chain variable region (VL) comprising the amino acid sequence set forth in SEQ ID NO: 38 binds to NKp46;
(14) an immunoglobulin light chain or a fragment thereof comprising a VL comprising CDRs 1, 2, and 3 comprising the amino acid sequences set forth in SEQ ID NOs: 20, 21 and 22, respectively, and wherein the VL, when paired with a VH comprising the amino acid sequence set forth in SEQ ID NO: 37 binds to NKp46;
(15) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 16, 18 and 19, respectively, and wherein the VH, when paired with a light chain variable region (VL) comprising the amino acid sequence set forth in SEQ ID NO: 38 binds to NKp46;
(16) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 25, 27 and 29, respectively, and wherein the VH, when paired with a light chain variable region (VL) comprising the amino acid sequence set forth in SEQ ID NO: 34 binds to NKp46;
(17) an immunoglobulin light chain or a fragment thereof comprising a VL comprising CDRs 1, 2, and 3 comprising the amino acid sequences set forth in SEQ ID NOs: 30, 31 and 32, respectively, and wherein the VL, when paired with a VH comprising the amino acid sequence set forth in SEQ ID NO: 33 binds to NKp46;
(18) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 26, 28 and 29, respectively, and wherein the VH, when paired with a light chain variable region (VL) comprising the amino acid sequence set forth in SEQ ID NO: 34 binds to NKp46.

12. The nucleic acid of claim 11, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 5, 7, and 9, respectively, or SEQ ID NOs: 6, 8 and 9, respectively.

13. The nucleic acid of claim 11, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 10, 11 and 12, respectively.

14. The nucleic acid of claim 11, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 15, 17 and 19, respectively, or SEQ ID NOs: 16, 18 and 19, respectively.

15. The nucleic acid of claim 11, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 20, 21 and 22, respectively.

16. The nucleic acid of claim 11, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 25, 27 and 29, respectively, or SEQ ID NOs: 26, 28 and 29, respectively.

17. The nucleic acid of claim 11, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 30, 31 and 32, respectively.

18. The nucleic acid of any one of claims 11-17, wherein the VH when paired with a VL specifically binds to human NKp46 or monkey NKp46, or the VL when paired with a VH specifically binds to human NKp46 or monkey NKp46.

19. The nucleic acid of any one of claims 11-18, wherein the immunoglobulin heavy chain or the fragment thereof is a human or humanized immunoglobulin heavy chain or a fragment thereof (e.g., a human IgG1 heavy chain or a fragment thereof), and the immunoglobulin light chain or the fragment thereof is a human or humanized immunoglobulin light chain or a fragment thereof.

20. The nucleic acid of any one of claims 11-19, wherein the nucleic acid encodes a single-chain variable fragment (scFv), a multi-specific antibody (e.g., a bispecific antibody) or a chimeric antigen receptor (CAR).

21. The nucleic acid of any one of claims 11-20, wherein the nucleic acid is cDNA.

22. A vector comprising one or more of the nucleic acids of any one of claims 11-21.

23. A vector comprising two of the nucleic acids of any one of claims 11-21, wherein the vector encodes the VH region and the VL region that together bind to NKp46.

24. A pair of vectors, wherein each vector comprises one of the nucleic acids of any one of claims 11-21, wherein together the pair of vectors encodes the VH region and the VL region that together bind to NKp46.

25. A cell comprising the vector of claim 22 or 23, or the pair of vectors of claim 24.

26. The cell of claim 25, wherein the cell is a CHO cell.

27. A cell comprising one or more of the nucleic acids of any one of claims 11-21.

28. A cell comprising two of the nucleic acids of any one of claims 11-21.

29. The cell of claim 28, wherein the two nucleic acids together encode the VH region and the VL region that together bind to NKp46.

30. A method of producing an antibody or an antigen-binding fragment thereof, the method comprising (a) culturing the cell of any one of claims 25-29 under conditions sufficient for the cell to produce the antibody or the antigen-binding fragment; and
(b) collecting the antibody or the antigen-binding fragment produced by the cell.

31. An antibody or antigen-binding fragment thereof that binds to NKp46 comprising a heavy chain variable region (VH) comprising an amino acid sequence that is at least 80% identical to a selected VH sequence, and a light chain variable region (VL) comprising an amino acid sequence that is at least 80% identical to a selected VL sequence, wherein the selected VH sequence and the selected VL sequence are one of the following:
(1) the selected VH sequence is SEQ ID NO: 3, and the selected VL sequence is SEQ ID NO: 4;
(2) the selected VH sequence is SEQ ID NO: 13, and the selected VL sequence is SEQ ID NO: 14;
(3) the selected VH sequence is SEQ ID NO: 23, and the selected VL sequence is SEQ ID NO: 24;
(4) the selected VH sequence is SEQ ID NO: 33, and the selected VL sequence is SEQ ID NO: 34;
(5) the selected VH sequence is SEQ ID NO: 35, and the selected VL sequence is SEQ ID NO: 36; and
(6) the selected VH sequence is SEQ ID NO: 37, and the selected VL sequence is SEQ ID NO: 38.

32. The antibody or antigen-binding fragment thereof of claim 31, wherein the VH comprises the sequence of SEQ ID NO: 3 and the VL comprises the sequence of SEQ ID NO: 4.

33. The antibody or antigen-binding fragment thereof of claim 31, wherein the VH comprises the sequence of SEQ ID NO: 13 and the VL comprises the sequence of SEQ ID NO: 14.

34. The antibody or antigen-binding fragment thereof of claim 31, wherein the VH comprises the sequence of SEQ ID NO: 23 and the VL comprises the sequence of SEQ ID NO: 24.

35. The antibody or antigen-binding fragment thereof of claim 31, wherein the VH comprises the sequence of SEQ ID NO: 33 and the VL comprises the sequence of SEQ ID NO: 34.

36. The antibody or antigen-binding fragment thereof of claim 31, wherein the VH comprises the sequence of SEQ ID NO: 35 and the VL comprises the sequence of SEQ ID NO: 36.

37. The antibody or antigen-binding fragment thereof of claim 31, wherein the VH comprises the sequence of SEQ ID NO: 37 and the VL comprises the sequence of SEQ ID NO: 38.

38. The antibody or antigen-binding fragment thereof of any one of claims 31-37, wherein the antibody or antigen-binding fragment specifically binds to human NKp46 or monkey NKp46.

39. The antibody or antigen-binding fragment thereof of any one of claims 31-38, wherein the antibody or antigen-binding fragment is a human or humanized antibody or antigen-binding fragment thereof.

40. The antibody or antigen-binding fragment thereof of any one of claims 31-39, wherein the antibody or antigen-binding fragment is a single-chain variable fragment (scFv) or a multi-specific antibody (e.g., a bispecific antibody).

41. An antibody or antigen-binding fragment thereof that cross-competes with the antibody or antigen-binding fragment thereof of any one of claims 1-10 and 31-40.

42. An antibody or antigen-binding fragment thereof that binds to NKp46 comprising
a heavy chain variable region (VH) comprising VH CDR1, VH CDR2, and VH CDR3 that are identical to VH CDR1, VH CDR2, and VH CDR3 of a selected VH sequence; and
a light chain variable region (VL) comprising VL CDR1, VL CDR2, and VL CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 3, and the selected VL sequence is SEQ ID NO: 4;
(2) the selected VH sequence is SEQ ID NO: 13, and the selected VL sequence is SEQ ID NO: 14;
(3) the selected VH sequence is SEQ ID NO: 23, and the selected VL sequence is SEQ ID NO: 24,
(4) the selected VH sequence is SEQ ID NO: 33, and the selected VL sequence is SEQ ID NO: 34,
(5) the selected VH sequence is SEQ ID NO: 35, and the selected VL sequence is SEQ ID NO: 36, and
(6) the selected VH sequence is SEQ ID NO: 37, and the selected VL sequence is SEQ ID NO: 38.

43. A method of treating a subject having cancer or an autoimmune disease, the method comprising administering a therapeutically effective amount of a composition comprising the antibody or antigen-binding fragment thereof of any one of claims 1-10 and 31-42 to the subject.

44. The method of claim 43, wherein the antibody or antigen-binding fragment thereof is a bispecific antibody that targets both an NK cell and a tumor cell expressing a tumor antigen.

45. The method of claim 43 or 44, further comprising administering a therapeutically effective amount of an anti-OX40 antibody, an anti-PD1 antibody, an anti-PDL1 antibody, an anti-PDL2 antibody, an anti-LAG-3 antibody, an anti-TIGIT antibody, an anti-CTLA-4 antibody, an anti-GITR antibody, an anti-TIM-3 antibody, an anti-4-1BB antibody, and/or an anti-CD40 antibody, to the subject.

46. A method of decreasing the rate of tumor growth, the method comprising contacting a tumor cell with an effective amount of a composition comprising an antibody or antigen-binding fragment thereof of any one of claims 1-10 and 31-42.

47. A method of killing a tumor cell, the method comprising contacting a tumor cell with an effective amount of a composition comprising the antibody or antigen-binding fragment thereof of any one of claims 1-10 and 31-42.

48. A pharmaceutical composition comprising the antibody or antigen-binding fragment thereof of any one of claims 1-10 and 31-42, and a pharmaceutically acceptable carrier.

49. An antibody or antigen-binding fragment thereof, comprising:
i) a first antigen-binding domain that specifically binds to a first antigen, wherein the first antigen is a tumor associated antigen (TAA);
ii) a second antigen-binding domain that specifically binds to a second antigen, wherein the second antigen is an NK cell surface antigen;
iii) an Fc region.

50. The antibody or antigen-binding fragment thereof of claim 49, wherein the first antigen-binding domain comprises a first heavy chain variable region (VH1) and a first light chain variable region (VL1); and the second antigen-binding domain comprises a second heavy chain variable region (VH2) and a second light chain variable region (VL2).

51. The antibody or antigen-binding fragment thereof of claim 49, wherein the second antigen-binding domain is a single-chain fragment variable (scFv) domain comprising a light chain variable domain (VL2) and a heavy chain variable domain (VH2) linked by a first linker.

52. The antibody or antigen-binding fragment thereof of claim 51, wherein the second antigen-binding domain is connected to the C-terminus of the light chain of the first antigen-binding domain through a second linker.

53. The antibody or antigen-binding fragment thereof of claim 51, wherein the second antigen-binding domain is linked to the C-terminus of the Fc region.

54. The antibody or antigen-binding fragment thereof of any one of claims 49-53, wherein the VH1 is linked to a CHI domain, and the VL1 is linked to a CL domain.

55. The antibody or antigen-binding fragment thereof of any one of claims 49-54, wherein the Fc region is an Fc region of human IgG1, IgG2, IgG3, or IgG4.

56. The antibody or antigen-binding fragment thereof of claim 55, wherein the Fc region is an Fc region of human IgG1.

57. The antibody or antigen-binding fragment thereof of claim 55 or 56, wherein the antibody or antigen-binding fragment thereof is a bispecific antibody or antigen-binding fragment thereof.

58. The antibody or antigen-binding fragment thereof of any one of claims 53-57, wherein the Fc region comprises one or more of the following (all numbering is according to EU numbering):
a Glutamic acid (E) at position 332;
a Leucine (L) at position 330;
an Alanine (A) at position 236; and/or
an Aspartic acid (D) at position 293.

59. The antibody or antigen-binding fragment thereof of any one of claims 53-57, wherein the Fc region comprises one of the following (all numbering is according to EU numbering):
i) an Alanine (A) at position 236, a Leucine (L) at position 330, and a Glutamic acid (E) at position 332;
ii) an Alanine (A) at position 236, an Aspartic acid (D) at position 293, a Leucine (L) at position 330, and a Glutamic acid (E) at position 332;
iii) an Alanine (A) at position 236;
iv) an Alanine (A) at position 236, an Aspartic acid (D) at position 293, and a Glutamic acid (E) at position 332;
v) an Aspartic acid (D) at position 293 and a Glutamic acid (E) at position 332; and
vi) an Aspartic acid (D) at position 293, a Leucine (L) at position 330, and a Glutamic acid (E) at position 332.

60. The antibody or antigen-binding fragment thereof of any one of claims 53-57, wherein the Fc region is afucosylated.

61. The antibody or antigen-binding fragment thereof of claim 59, wherein the Fc region is an Fc region of human IgG1, wherein the Fc region comprises an Alanine (A) at position 236, a Leucine (L) at position 330, and a Glutamic acid (E) at position 332, wherein the Fc region comprises an amino acid sequence that is at least 80% identical to SEQ ID NO: 43 (the numbering is according to EU numbering).

62. The antibody or antigen-binding fragment thereof of any one of claims 49-61, wherein the NK cell surface antigen is selected from NKp46, NKp30, CD16, NKG2D and NKG2A.

63. The antibody or antigen-binding fragment thereof of claim 62, wherein the NK cell surface antigen is NKp46.

64. The antibody or antigen-binding fragment thereof of any one of claims 49-63, wherein the tumor associated antigen (TAA) is CD79b, EGFR, EpCAM, B-cell maturation antigen (BCMA), DLL3, PSMA, STEAP1, MUC1, c-MET, LRRC15, CCR8, OX40, perforin, granzyme B, Fas ligand (FasL), CD1d, Membrane glycolipids, globotriaosyl-ceramide (Gb3Cer/CD77), gangliosides (GD2, GD3, and GM2), CD34, CD45, human leukocyte antigen-DR (HLA-DR), CD123, CD38, CLL1, CD105, CD71, SSC, MAGE, MUC16, CD19, WT-1, B7H3, TEMS, CD22, LI-CAM, ROR-1, CEA, 4-1BB, ETA, 5T4, adenocarcinoma antigen, alpha-fetoprotein (AFP), BAFF, B-lymphoma cell, C242 antigen, CA-125, carbonic anhydrase 9 (CA-IX), CCR4, CD133, CD152, CD20, CD125, CD200, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, IL-5, IL-13, IL-6, IL-15, insulin-like growth factor I receptor, integrin a5b1, integrin anb3, MORAb-009, MS4A1, mucin CanAg, N-glycolylneuraminic acid, NPC-1C, PD-1, PD-L1, PDGF-R a, PDL192, phosphatidylserine, prostatic carcinoma cells, RANKL, RON, SCH 900105, SDC1, SLAMF7, TAG-72, tenascin C, TGF beta 2, TGF-b, TRAIL-R1, TRAIL-R2, tumor antigen CTAA16.88, VEGF-A, VEGFR-1, VEGFR2, or vimentin.

65. The antibody or antigen-binding fragment thereof of any one of claims 49-64, wherein the first antigen is CD79b; and the second antigen is NKp46.

66. The antibody or antigen-binding fragment thereof of claim 65, wherein
the first heavy chain variable region (VH1) comprises complementarity determining regions (CDRs) 1, 2, and 3, wherein the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and
the first light chain variable region (VL1) comprises CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence,
wherein the selected VH1 CDRs 1, 2, and 3 amino acid sequences, and the selected VL1 CDRs 1, 2, and 3 amino acid sequences are one of the following:
(1) the selected VH1 CDRs 1, 2, 3 amino acid sequences are set forth in SEQ ID NOs: 49, 51 and 53, respectively, and the selected VL1 CDRs 1, 2, 3 amino acid sequences are set forth in SEQ ID NOs: 54, 55 and 56, respectively; and
(2) the selected VH1 CDRs 1, 2, 3 amino acid sequences are set forth in SEQ ID NOs: 50, 52 and 53, respectively, and the selected VL1 CDRs 1, 2, 3 amino acid sequences are set forth in SEQ ID NOs: 54, 55 and 56, respectively, and
(3) the selected VH1 CDRs 1, 2, 3 amino acid sequences and the selected VL1 CDRs 1, 2, 3 amino acid sequences are selected from Polatuzumab, PRV-3279 and SHR-A1912.

67. The antibody or antigen-binding fragment thereof of claim 65 or 66, wherein
the second heavy chain variable region (VH2) comprises CDRs 1, 2, and 3, wherein the VH2 CDR1 region comprises an amino acid sequence that is at least 80% identical to a selected VH2 CDR1 amino acid sequence, the VH2 CDR2 region comprises an amino acid sequence that is at least 80% identical to a selected VH2 CDR2 amino acid sequence, and the VH2 CDR3 region comprises an amino acid sequence that is at least 80% identical to a selected VH2 CDR3 amino acid sequence; and
the second light chain variable region (VL2) comprises CDRs 1, 2, and 3, wherein the VL2 CDR1 region comprises an amino acid sequence that is at least 80% identical to a selected VL2 CDR1 amino acid sequence, the VL2 CDR2 region comprises an amino acid sequence that is at least 80% identical to a selected VL2 CDR2 amino acid sequence, and the VL2 CDR3 region comprises an amino acid sequence that is at least 80% identical to a selected VL2 CDR3 amino acid sequence,
wherein the selected VH2 CDRs 1, 2, and 3 amino acid sequences, and the selected VL2 CDRs 1, 2, and 3 amino acid sequences are one of the following:
(1) the selected VH2 CDRs 1, 2, 3 amino acid sequences are set forth in SEQ ID NOs: 5, 7, and 9, respectively, and the selected VL2 CDRs 1, 2, 3 amino acid sequences are set forth in SEQ ID NOs: 10, 11, and 12, respectively;
(2) the selected VH2 CDRs 1, 2, 3 amino acid sequences are set forth in SEQ ID NOs: 6, 8, and 9, respectively, and the selected VL2 CDRs 1, 2, 3 amino acid sequences are set forth in SEQ ID NOs: 10, 11 and 12, respectively;
(3) the selected VH2 CDRs 1, 2, 3 amino acid sequences are set forth in SEQ ID NOs: 15, 17, and 19, respectively, and the selected VL2 CDRs 1, 2, 3 amino acid sequences are set forth in SEQ ID NOs: 20, 21, and 22, respectively;
(4) the selected VH2 CDRs 1, 2, 3 amino acid sequences are set forth in SEQ ID NOs: 16, 18, and 19, respectively, and the selected VL2 CDRs 1, 2, 3 amino acid sequences are set forth in SEQ ID NOs: 20, 21 and 22, respectively;
(5) the selected VH2 CDRs 1, 2, 3 amino acid sequences are set forth in SEQ ID NOs: 25, 27, and 29, respectively, and the selected VL2 CDRs 1, 2, 3 amino acid sequences are set forth in SEQ ID NOs: 30, 31, and 32, respectively;
(6) the selected VH2 CDRs 1, 2, 3 amino acid sequences are set forth in SEQ ID NOs: 26, 28, and 29, respectively, and the selected VL2 CDRs 1, 2, 3 amino acid sequences are set forth in SEQ ID NOs: 30, 31 and 32, respectively.

68. The antibody or antigen-binding fragment thereof of any one of claims 65-67, wherein
(1) the selected VH1 CDRs 1, 2, 3 amino acid sequences are set forth in SEQ ID NOs: 49, 51, and 53, respectively, and the selected VL1 CDRs 1, 2, 3 amino acid sequences are set forth in SEQ ID NOs: 54, 55, and 56, respectively; and the selected VH2 CDRs 1, 2, 3 amino acid sequences are set forth in SEQ ID NOs: 5, 7, and 9, respectively, and the selected VL2 CDRs 1, 2, 3 amino acid sequences are set forth in SEQ ID NOs: 10, 11, and 12, respectively;
(2) the selected VH1 CDRs 1, 2, 3 amino acid sequences are set forth in SEQ ID NOs: 50, 52, and 53, respectively, and the selected VL1 CDRs 1, 2, 3 amino acid sequences are set forth in SEQ ID NOs: 54, 55, and 56, respectively; and the selected VH2 CDRs 1, 2, 3 amino acid sequences are set forth in SEQ ID NOs: 6, 8, and 9, respectively, and the selected VL2 CDRs 1, 2, 3 amino acid sequences are set forth in SEQ ID NOs: 10, 11 and 12, respectively;
(3) the selected VH1 CDRs 1, 2, 3 amino acid sequences are set forth in SEQ ID NOs: 49, 51, and 53, respectively, and the selected VL1 CDRs 1, 2, 3 amino acid sequences are set forth in SEQ ID NOs: 54, 55, and 56, respectively; and the selected VH2 CDRs 1, 2, 3 amino acid sequences are set forth in SEQ ID NOs: 15, 17, and 19, respectively, and the selected VL2 CDRs 1, 2, 3 amino acid sequences are set forth in SEQ ID NOs: 20, 21, and 22, respectively;
(4) the selected VH1 CDRs 1, 2, 3 amino acid sequences are set forth in SEQ ID NOs: 50, 52, and 53, respectively, and the selected VL1 CDRs 1, 2, 3 amino acid sequences are set forth in SEQ ID NOs: 54, 55, and 56, respectively; and the selected VH2 CDRs 1, 2, 3 amino acid sequences are set forth in SEQ ID NOs: 16, 18, and 19, respectively, and the selected VL2 CDRs 1, 2, 3 amino acid sequences are set forth in SEQ ID NOs: 20, 21 and 22, respectively;
(5) the selected VH1 CDRs 1, 2, 3 amino acid sequences are set forth in SEQ ID NOs: 49, 51, and 53, respectively, and the selected VL1 CDRs 1, 2, 3 amino acid sequences are set forth in SEQ ID NOs: 54, 55, and 56, respectively; and the selected VH2 CDRs 1, 2, 3 amino acid sequences are set forth in SEQ ID NOs: 25, 27, and 29, respectively, and the selected VL2 CDRs 1, 2, 3 amino acid sequences are set forth in SEQ ID NOs: 30, 31, and 32, respectively; or
(6) the selected VH1 CDRs 1, 2, 3 amino acid sequences are set forth in SEQ ID NOs: 50, 52, and 53, respectively, and the selected VL1 CDRs 1, 2, 3 amino acid sequences are set forth in SEQ ID NOs: 54, 55, and 56, respectively; and the selected VH2 CDRs 1, 2, 3 amino acid sequences are set forth in SEQ ID NOs: 26, 28, and 29, respectively, and the selected VL2 CDRs 1, 2, 3 amino acid sequences are set forth in SEQ ID NOs: 30, 31 and 32, respectively.

69. The antibody or antigen-binding fragment thereof of any one of claims 49-64, wherein the first antigen is BCMA; and the second antigen is NKp46.

70. The antibody or antigen-binding fragment thereof of claim 69, wherein
the first heavy chain variable region (VH1) comprises complementarity determining regions (CDRs) 1, 2, and 3, wherein the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and
the first light chain variable region (VL1) comprises CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence,
wherein the selected VH1 CDRs 1, 2, and 3 amino acid sequences, and the selected VL1 CDRs 1, 2, and 3 amino acid sequences are as following:
(1) the selected VH1 CDRs 1, 2, 3 amino acid sequences are set forth in SEQ ID NOs: 88, 89 and 90, respectively, and the selected VL1 CDRs 1, 2, 3 amino acid sequences are set forth in SEQ ID NOs: 91, 92 and 93, respectively;
(2) the selected VH1 CDRs 1, 2, 3 amino acid sequences and the selected VL1 CDRs 1, 2, 3 amino acid sequences are the corresponding VH CDRs 1, 2, 3 and VL CDRs 1, 2, 3 of Zevorcabtagene Autoleucel, Elranatamab, Teclistamab, Equecabtagene Autoleuce, Ciltacabtagene autoleucel, Idecabtagene Vicleucel, Belantamab, Linvoseltamab, Alnuctamab, TNB-383B or SEA-BCMA.

71. The antibody or antigen-binding fragment thereof of claim 69 or 70, wherein
the second heavy chain variable region (VH2) comprises CDRs 1, 2, and 3, wherein the VH2 CDR1 region comprises an amino acid sequence that is at least 80% identical to a selected VH2 CDR1 amino acid sequence, the VH2 CDR2 region comprises an amino acid sequence that is at least 80% identical to a selected VH2 CDR2 amino acid sequence, and the VH2 CDR3 region comprises an amino acid sequence that is at least 80% identical to a selected VH2 CDR3 amino acid sequence; and
the second light chain variable region (VL2) comprises CDRs 1, 2, and 3, wherein the VL2 CDR1 region comprises an amino acid sequence that is at least 80% identical to a selected VL2 CDR1 amino acid sequence, the VL2 CDR2 region comprises an amino acid sequence that is at least 80% identical to a selected VL2 CDR2 amino acid sequence, and the VL2 CDR3 region comprises an amino acid sequence that is at least 80% identical to a selected VL2 CDR3 amino acid sequence,
wherein the selected VH2 CDRs 1, 2, and 3 amino acid sequences, and the selected VL2 CDRs 1, 2, and 3 amino acid sequences are as following:
the selected VH2 CDRs 1, 2, 3 amino acid sequences are set forth in SEQ ID NOs: 25, 27 and 29, respectively, and the selected VL2 CDRs 1, 2, 3 amino acid sequences are set forth in SEQ ID NOs: 30, 31, and 32, respectively.

72. The antibody or antigen-binding fragment thereof of any one of claims 69-71, wherein
the selected VH1 CDRs 1, 2, 3 amino acid sequences are set forth in SEQ ID NOs: 88, 89 and 90, respectively, and the selected VL1 CDRs 1, 2, 3 amino acid sequences are set forth in SEQ ID NOs: 91, 92 and 93, respectively; and the selected VH2 CDRs 1, 2, 3 amino acid sequences are set forth in SEQ ID NOs: 25, 27 and 29, respectively, and the selected VL2 CDRs 1, 2, 3 amino acid sequences are set forth in SEQ ID NOs: 30, 31, and 32, respectively.

73. The antibody or antigen-binding fragment thereof of any one of claims 49-64, wherein the first antigen is EGFR; and the second antigen is NKp46.

74. The antibody or antigen-binding fragment thereof of claim 73, wherein
the first heavy chain variable region (VH1) comprises complementarity determining regions (CDRs) 1, 2, and 3, wherein the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and
the first light chain variable region (VL1) comprises CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence,
wherein the selected VH1 CDRs 1, 2, and 3 amino acid sequences, and the selected VL1 CDRs 1, 2, and 3 amino acid sequences are as following:
(1) the selected VH1 CDRs 1, 2, 3 amino acid sequences are set forth in SEQ ID NOs: 94, 95 and 96, respectively, and the selected VL1 CDRs 1, 2, 3 amino acid sequences are set forth in SEQ ID NOs: 97, 98 and 99, respectively;
(2) the selected VH1 CDRs 1, 2, 3 amino acid sequences and the selected VL1 CDRs 1, 2, 3 amino acid sequences are the corresponding VH CDRs 1, 2, 3 and VL CDRs 1, 2, 3 of Amivantamab, Cetuximab, Necitumumab, Panitumumab, Nimotuzumab, BL-B01D1, Demupitamab, Depatuxizumab, Futuximab, Modotuximab, JMT-101, MRG-003, Pimurutamab, QL1203, SI-BOO1 and Ametumumab.

75. The antibody or antigen-binding fragment thereof of claim 73 or 74, wherein
the second heavy chain variable region (VH2) comprises CDRs 1, 2, and 3, wherein the VH2 CDR1 region comprises an amino acid sequence that is at least 80% identical to a selected VH2 CDR1 amino acid sequence, the VH2 CDR2 region comprises an amino acid sequence that is at least 80% identical to a selected VH2 CDR2 amino acid sequence, and the VH2 CDR3 region comprises an amino acid sequence that is at least 80% identical to a selected VH2 CDR3 amino acid sequence; and
the second light chain variable region (VL2) comprises CDRs 1, 2, and 3, wherein the VL2 CDR1 region comprises an amino acid sequence that is at least 80% identical to a selected VL2 CDR1 amino acid sequence, the VL2 CDR2 region comprises an amino acid sequence that is at least 80% identical to a selected VL2 CDR2 amino acid sequence, and the VL2 CDR3 region comprises an amino acid sequence that is at least 80% identical to a selected VL2 CDR3 amino acid sequence,
wherein the selected VH2 CDRs 1, 2, and 3 amino acid sequences, and the selected VL2 CDRs 1, 2, and 3 amino acid sequences are as following:
the selected VH2 CDRs 1, 2, 3 amino acid sequences are set forth in SEQ ID NOs: 25, 27 and 29, respectively, and the selected VL2 CDRs 1, 2, 3 amino acid sequences are set forth in SEQ ID NOs: 30, 31, and 32, respectively.

76. The antibody or antigen-binding fragment thereof of any one of claims 73-75, wherein
the selected VH1 CDRs 1, 2, 3 amino acid sequences are set forth in SEQ ID NOs: 94, 95 and 96, respectively, and the selected VL1 CDRs 1, 2, 3 amino acid sequences are set forth in SEQ ID NOs: 97, 98 and 99, respectively; and the selected VH2 CDRs 1, 2, 3 amino acid sequences are set forth in SEQ ID NOs: 25, 27 and 29, respectively, and the selected VL2 CDRs 1, 2, 3 amino acid sequences are set forth in SEQ ID NOs: 30, 31, and 32, respectively.

77. The antibody or antigen-binding fragment thereof of any one of claims 49-64, wherein the first antigen is EpCAM; and the second antigen is NKp46.

78. The antibody or antigen-binding fragment thereof of claim 77, wherein
the first heavy chain variable region (VH1) comprises complementarity determining regions (CDRs) 1, 2, and 3, wherein the VH1 CDR1 region comprises an amino acid sequence that is at least 80% identical to a selected VH1 CDR1 amino acid sequence, the VH1 CDR2 region comprises an amino acid sequence that is at least 80% identical to a selected VH1 CDR2 amino acid sequence, and the VH1 CDR3 region comprises an amino acid sequence that is at least 80% identical to a selected VH1 CDR3 amino acid sequence; and
the first light chain variable region (VL1) comprises CDRs 1, 2, and 3, wherein the VL1 CDR1 region comprises an amino acid sequence that is at least 80% identical to a selected VL1 CDR1 amino acid sequence, the VL1 CDR2 region comprises an amino acid sequence that is at least 80% identical to a selected VL1 CDR2 amino acid sequence, and the VL1 CDR3 region comprises an amino acid sequence that is at least 80% identical to a selected VL1 CDR3 amino acid sequence,
wherein the selected VH1 CDRs 1, 2, and 3 amino acid sequences, and the selected VL1 CDRs 1, 2, and 3 amino acid sequences are as following:
(1) the selected VH1 CDRs 1, 2, 3 amino acid sequences are set forth in SEQ ID NOs: 100, 101 and 102, respectively, and the selected VL1 CDRs 1, 2, 3 amino acid sequences are set forth in SEQ ID NOs: 103, 104 and 105, respectively;
(2) the selected VH1 CDRs 1, 2, 3 amino acid sequences and the selected VL1 CDRs 1, 2, 3 amino acid sequences are the corresponding VH CDRs 1, 2, 3 and VL CDRs 1, 2, 3 of Catumaxomab, M-701, Adecatumumab, Edrecolomab, Tefibazumab, VB1-008 or VB1-050.

79. The antibody or antigen-binding fragment thereof of claim 77 or 78, wherein
the second heavy chain variable region (VH2) comprises CDRs 1, 2, and 3, wherein the VH2 CDR1 region comprises an amino acid sequence that is at least 80% identical to a selected VH2 CDR1 amino acid sequence, the VH2 CDR2 region comprises an amino acid sequence that is at least 80% identical to a selected VH2 CDR2 amino acid sequence, and the VH2 CDR3 region comprises an amino acid sequence that is at least 80% identical to a selected VH2 CDR3 amino acid sequence; and
the second light chain variable region (VL2) comprises CDRs 1, 2, and 3, wherein the VL2 CDR1 region comprises an amino acid sequence that is at least 80% identical to a selected VL2 CDR1 amino acid sequence, the VL2 CDR2 region comprises an amino acid sequence that is at least 80% identical to a selected VL2 CDR2 amino acid sequence, and the VL2 CDR3 region comprises an amino acid sequence that is at least 80% identical to a selected VL2 CDR3 amino acid sequence,
wherein the selected VH2 CDRs 1, 2, and 3 amino acid sequences, and the selected VL2 CDRs 1, 2, and 3 amino acid sequences are as following:
the selected VH2 CDRs 1, 2, 3 amino acid sequences are set forth in SEQ ID NOs: 25, 27 and 29, respectively, and the selected VL2 CDRs 1, 2, 3 amino acid sequences are set forth in SEQ ID NOs: 30, 31, and 32, respectively.

80. The antibody or antigen-binding fragment thereof of any one of claims 77-79, wherein the selected VH1 CDRs 1, 2, 3 amino acid sequences are set forth in SEQ ID NOs: 100, 101 and 102, respectively, and the selected VL1 CDRs 1, 2, 3 amino acid sequences are set forth in SEQ ID NOs: 103, 104 and 105, respectively; and the selected VH2 CDRs 1, 2, 3 amino acid sequences are set forth in SEQ ID NOs: 25, 27 and 29, respectively, and the selected VL2 CDRs 1, 2, 3 amino acid sequences are set forth in SEQ ID NOs: 30, 31, and 32, respectively.

81. The antibody or antigen-binding fragment thereof of any one of claims 49-64, wherein the first antigen is DLL3; and the second antigen is NKp46.

82. The antibody or antigen-binding fragment thereof of claim 81, wherein the CDRS of DLL3 antigen-binding fragment are the corresponding VH CDRs 1, 2, 3 and VL CDRs 1, 2, 3 of Rovalpituzumab tesirine, Tarlatamab, HPN328 or BI 764532.

83. The antibody or antigen-binding fragment thereof of any one of claims 49-64, wherein the first antigen is PSMA; and the second antigen is NKp46.

84. The antibody or antigen-binding fragment thereof of claim 83, wherein the CDRs of PSMA antigen-binding fragment are the corresponding VH CDRs 1, 2, 3 and VL CDRs 1, 2, 3 of HPN-424, LAVA-1207, REGN-5678, REGN4336, Acapatamab or AMG-340.

85. The antibody or antigen-binding fragment thereof of any one of claims 49-64, wherein the first antigen is STEAP1; and the second antigen is NKp46.

86. The antibody or antigen-binding fragment thereof of claim 85, wherein the CDRs of STEAP1 antigen-binding fragment are the corresponding VH CDRs 1, 2, 3 and VL CDRs 1, 2, 3 of Xaluritamig, ABBV-969 or Vandortuzumab vedotin.

87. The antibody or antigen-binding fragment thereof of any one of claims 49-64, wherein the first antigen is MUC1; and the second antigen is NKp46.

88. The antibody or antigen-binding fragment thereof of claim 87, wherein the CDRs of MUC1 antigen-binding fragment are the corresponding VH CDRs 1, 2, 3 and VL CDRs 1, 2, 3 of MAB-AR20.5 or DS-3939.

89. The antibody or antigen-binding fragment thereof of any one of claims 49-64, wherein the first antigen is c-MET; and the second antigen is NKp46.

90. The antibody or antigen-binding fragment thereof of claim 89, wherein the CDRs of c-MET antigen-binding fragment are the corresponding VH CDRs 1, 2, 3 and VL CDRs 1, 2, 3 of Amivantamab-VMJM, Telisotuzumab vedotin, ABBV-400, Bafisontamab, CKD-702, Emibetuzumab, RC-108, MCLA-129, REGN-5093 or REGN5093-M114.

91. The antibody or antigen-binding fragment thereof of any one of claims 49-64, wherein the first antigen is LRRC15; and the second antigen is NKp46.

92. The antibody or antigen-binding fragment thereof of claim 91, wherein the CDRs of LRRC15 antigen-binding fragment are the corresponding VH CDRs 1, 2, 3 and VL CDRs 1, 2, 3 of QL-315, RAD-502 or Samrotamab.

93. The antibody or antigen-binding fragment thereof of any one of claims 49-64, wherein the first antigen is CCR8; and the second antigen is NKp46.

94. The antibody or antigen-binding fragment thereof of claim 93, wherein the CDRs of CCR8 antigen-binding fragment are the corresponding VH CDRs 1, 2, 3 and VL CDRs 1, 2, 3 of BMS-986340, LM-108, S-531011, AMG-355, BAY3375968, BGB-A3055, CM369, HBM-1022, PSB-114 or SRF-114.

95. The antibody or antigen-binding fragment thereof of any one of claims 49-64, wherein the first antigen is OX40; and the second antigen is NKp46.

96. The antibody or antigen-binding fragment thereof of claim 93, wherein the CDRs of OX40 antigen-binding fragment are the corresponding VH CDRs 1, 2, 3 and VL CDRs 1, 2, 3 of Amlitelimab, Rocatinlimab, INBRX-106, BGB-A445, INCAGN-1949, Ivuxolimab, MEDI-6469, BAT-6026, IMG-007, ABBV-368 or BMS-986178.

97. The antibody or antigen-binding fragment thereof of any one of claims 49-96, wherein the Fc region has increased complement-dependent cytotoxicity (CDC), increased antibody-dependent cellular cytotoxicity (ADCC) or increased half-life.

98. An antibody or antigen-binding fragment thereof that cross-competes with the antibody or antigen-binding fragment thereof of any one of claims 49-97.

99. A method of treating a subject having cancer, the method comprising administering a therapeutically effective amount of a composition comprising the antibody or antigen-binding fragment thereof of any one of claims 49-98 to the subject.

100. The method of claim 99, wherein the cancer is a solid tumor or a blood tumor.

101. The method of claim 100, wherein the cancer is breast cancer, lung cancer, stomach cancer, colorectal cancer, prostate cancer, ovarian cancer, colon cancer, esophageal cancer, tracheal cancer, gastric cancer bladder cancer, uterine cancer, rectal cancer, cancer of the small intestine, pancreatic cancer and liver cancer.

102. The method of claim 100, wherein the cancer is multiple myeloma, B-cell lymphoma, diffuse Large B-Cell Lymphoma, acute B-cell leukemia, chronic lymphocytic leukemia, B-cell prelymphocytic leukemia, spleen with villous lymphocytes Lymphoma, hairy cell leukemia, follicular lymphoma, and mantle cell lymphoma

103. The method of any one of claims 99-102, wherein the subject is further treated with an effective amount of an anti-4-1BB antibody, an anti-OX40 antibody, an anti-PD-1 antibody, an anti-CTLA4 antibody, an anti-CD40 antibody, or an anti-PD-L1 antibody.

104. A method of decreasing the rate of tumor growth, the method comprising contacting a tumor cell with an effective amount of a composition comprising an antibody or antigen-binding fragment thereof of any one of claims 49-98.

105. A method of killing a tumor cell, the method comprising contacting a tumor cell with an effective amount of a composition comprising the antibody or antigen-binding fragment thereof of any one of claims 49-98.

106. A method of increasing immune response in a subject, the method comprising administering to the subject an effective amount of a composition comprising the antibody or antigen-binding fragment thereof of any one of claims 49-98.

107. A method of treating a subject having autoimmune disease, the method comprising administering a therapeutically effective amount of a composition comprising the antibody or antigen-binding fragment thereof of any one of claims 49-98 to the subject.

108. The method of claim 107, wherein the autoimmune disease is selected from rheumatoid arthritis, psoriasis, multiple sclerosis, immune thrombocytopenic purpura, myasthenia gravis, neuromyelitis optica, IgG4-related diseases, systemic Lupus Erythematosus, lupus nephritis, giant cell arteritis, takayasu disease, cold agglutinin disease, warm autoimmune hemolytic anemia, and anti-neutrophil cytoplasmic antibody (ANCA) associated vasculitides including for example, tranulomatosis with polyangiitis (GPA) (Wegener's Granulomatosis) or Microscopic Polyangiitis (MPA).

109. The method of claim 108, wherein the autoimmune disease is multiple sclerosis, systemic lupus erythematosus or rheumatoid arthritis.

110. A pharmaceutical composition comprising the antibody or antigen-binding fragment thereof of any one of claims 49-98, and a pharmaceutically acceptable carrier.


SEQUENCE LISTING (111 sequences)

SEQ ID NO: 1 - protein, 233 aa, synthetic construct
QQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPDTWGTYLLTTETGLQKDHALWDHTAQN

SEQ ID NO: 2 - protein, 236 aa, synthetic construct
PKQTLPKPIIRAESTYMVPKEKQATLCCQGSYGAVEYQLHFEGSLFAVERPKPPERINGVKFHIPDMNSRKAGRYSCIYRVGELWSERSDLLDLVVTEMYDTPTLSVHPGPEVTSGEKVTFYCRLDTATSMFLLLKEGRSRDVQRSYGKVQAEFPMGPVTTAHRGSYRCFGSYNNYAWSFPSEPVKLLVTGDIENTSLAPTDPTFPDSWDTCLLTRETGLQKDLALWDHTAQNLLR

SEQ ID NO: 3 - protein, 116 aa, synthetic construct
QSLEESGGRLVTPGTPLTLTCTVSGFSPSSYYMSWVRQAPGKGLEYIGIIFSGGSTWYASWAKGRVTISRTSTTVDLKMTSLTTEDTATYFCARGDAVGSGYWGLWGPGTLVTVSS

SEQ ID NO: 4 - protein, 108 aa, synthetic construct
AFELTQTPSSVEAAVGGTVTINCQASQDIYIYLAWYQQKPGQPPKLLIYSASTLASGVSSRFTGSRSGTQFTLTISDLEPADAATYYCQSYDGTSNTGFGGGTEVVVR

SEQ ID NO: 5 - protein, 5 aa, synthetic construct
SYYMS

SEQ ID NO: 6 - protein, 7 aa, synthetic construct
GFSPSSY

SEQ ID NO: 7 - protein, 16 aa, synthetic construct
IIFSGGSTWYASWAKG

SEQ ID NO: 8 - protein, 5 aa, synthetic construct
FSGGS

SEQ ID NO: 9 - protein, 11 aa, synthetic construct
GDAVGSGYWGL

SEQ ID NO: 10 - protein, 11 aa, synthetic construct
QASQDIYIYLA

SEQ ID NO: 11 - protein, 7 aa, synthetic construct
SASTLAS

SEQ ID NO: 12 - protein, 10 aa, synthetic construct
QSYDGTSNTG

SEQ ID NO: 13 - protein, 116 aa, synthetic construct
QSVEESGGRLVTPGTPLTLTCTVSGFSLSSYHMSWVRQAPGKGLEWIGYILANTDSYYANWAKGRFTISKTSTTVDLKITSPTTEDTATYFCARYDNHDAAYSTLWGPGTLVTVSS

SEQ ID NO: 14 - protein, 110 aa, synthetic construct
AYDMTQTPASVEVAVGGTVTIKCQASQSISTALAWYQQKPGQPPKLLIYKASTLASGVSSRFKGSGSGTQFTLTISGVEPADAATYYCQQGYSRSNVDNVFGGGTEVVVK

SEQ ID NO: 15 - protein, 5 aa, synthetic construct
SYHMS

SEQ ID NO: 16 - protein, 7 aa, synthetic construct
GFSLSSY

SEQ ID NO: 17 - protein, 16 aa, synthetic construct
YILANTDSYYANWAKG

SEQ ID NO: 18 - protein, 5 aa, synthetic construct
LANTD

SEQ ID NO: 19 - protein, 11 aa, synthetic construct
YDNHDAAYSTL

SEQ ID NO: 20 - protein, 11 aa, synthetic construct
QASQSISTALA

SEQ ID NO: 21 - protein, 7 aa, synthetic construct
KASTLAS

SEQ ID NO: 22 - protein, 12 aa, synthetic construct
QQGYSRSNVDNV

SEQ ID NO: 23 - protein, 116 aa, synthetic construct
QSLEESGGGLVQPEGSLTLTCTASGFSFSSSAWICWVRQAPGKGLEWIGCINTVSVNAYYASWAKGRFTISKTSSTTVTLQVTSLTAADTATHFCARNPYNSLRLWGPGTLVTVSS

SEQ ID NO: 24 - protein, 111 aa, synthetic construct
ADVVMTQTPASVSEPVGGTITINCQASENIYSSLAWYQQKPGQPPKLLMYDVSDLASGVPSRFSGSGSGTEFALTISDVQPDDAATYFCLGGYPSNNYRMAFGGGTEVVVK

SEQ ID NO: 25 - protein, 6 aa, synthetic construct
SSAWIC

SEQ ID NO: 26 - protein, 8 aa, synthetic construct
GFSFSSSA

SEQ ID NO: 27 - protein, 17 aa, synthetic construct
CINTVSVNAYYASWAKG

SEQ ID NO: 28 - protein, 6 aa, synthetic construct
NTVSVN

SEQ ID NO: 29 - protein, 8 aa, synthetic construct
NPYNSLRL

SEQ ID NO: 30 - protein, 11 aa, synthetic construct
QASENIYSSLA

SEQ ID NO: 31 - protein, 7 aa, synthetic construct
DVSDLAS

SEQ ID NO: 32 - protein, 12 aa, synthetic construct
LGGYPSNNYRMA

SEQ ID NO: 33 - protein, 118 aa, synthetic construct
EQQLLESGGGLVQPGGSLRLSCAASGFSFSSSAWICWVRQAPGKCLEWIGCINTVSVNAYYASWAKGRFTISKDSSKNTVYLQMNSLRAEDTAVYFCARNPYNSLRLWGPGTLVTVSS

SEQ ID NO: 34 - protein, 110 aa, synthetic construct
DVQMTQSPSTLSASVGDRVTITCQASENIYSSLAWYQQKPGKPPKLLMYDVSDLASGVPSRFSGSGSGTEFTLTISSLQPDDFATYFCLGGYPSNNYRMAFGCGTKVEIK

SEQ ID NO: 35 - protein, 119 aa, synthetic construct
EQQLLESGGGLVQPGGSLRLSCAASGFSPSSYYMSWVRQAPGKCLEWIGIIFSGGSTWYASWAKGRFTISKDSSKNTVYLQMNSLRAEDTAVYFCARGDAVGSGYWGLWGPGTLVTVSS

SEQ ID NO: 36 - protein, 108 aa, synthetic construct
DVQMTQSPSTLSASVGDRVTITCQASQDIYIYLAWYQQKPGKPPKLLMYSASTLASGVPSRFSGSGSGTEFTLTISSLQPDDFATYFCQSYDGTSNTGFGCGTKVEIK

SEQ ID NO: 37 - protein, 119 aa, synthetic construct
EQQLLESGGGLVQPGGSLRLSCAASGFSLSSYHMSWVRQAPGKCLEWIGYILANTDSYYANWAKGRFTISKDSSKNTVYLQMNSLRAEDTAVYFCARYDNHDAAYSTLWGPGTLVTVSS

SEQ ID NO: 38 - protein, 110 aa, synthetic construct
DVQMTQSPSTLSASVGDRVTITCQASQSISTALAWYQQKPGKPPKLLMYKASTLASGVPSRFSGSGSGTEFTLTISSLQPDDFATYFCQQGYSRSNVDNVFGCGTKVEIK

SEQ ID NO: 39 - protein, 120 aa, synthetic construct
STGSEVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSS

SEQ ID NO: 40 - protein, 107 aa, synthetic construct
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELK

SEQ ID NO: 41 - protein, 330 aa, synthetic construct
ASTKGPSVFPLAPSSKSTSGGTAALGCLVKDYFPEPVTVSWSSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 330 aa, synthetic construct
ASTKGPSVFPLAPSSKSTSGGTAALGCLVKDYFPEPVTVSWSSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 107 aa, synthetic construct [sequence omitted]

SEQ ID NO: 45 - protein, 120 aa, synthetic construct
QSVRETGGGLVQPGGSLKLSCKASGFTINIDYWISWVRQAPGKGLEWIGIIDPGDGTTYFAIWVNGRFTISFDNTQNTVDLQMNSLTAADTATYFCVREFSASAYQYDLWGPGTLVTVSS

SEQ ID NO: 46 - protein, 110 aa, synthetic construct
DLVMTQTPSSVSAAVGGTVSINCRASEDIENYLAWYQQKPGQPPKLLIYRASTLSSGVSSRFSGSGSGTEFTLTISGVQPDDAATYYCQQGYRNSNVDNPFGGGTEMVVK

SEQ ID NO: 47 - protein, 121 aa, synthetic construct
EQQLVETGGGLIQPGGSLRLSCAASGFTINIDYWISWVRQAPGKGLEWIGIIDPGDGTTYFAIWVSGRFTISFDNSKNTVYLQMNSLRAEDTAVYFCVREFSASAYQYDLWGPGTLVTVSS

SEQ ID NO: 48 - protein, 110 aa, synthetic construct
ELVMTQSPATLSVSPGERATLSCRASEDIENYLAWYQQKPGQPPRLLIYRASTLSSGVPARFSGSGSGTEFTLTISSLQSEDFAVYYCQQGYRNSNVDNPFGGGTKVEIK

SEQ ID NO: 49 - protein, 6 aa, synthetic construct
IDYWIS

SEQ ID NO: 50 - protein, 8 aa, synthetic construct
GFTINIDY

SEQ ID NO: 51 - protein, 17 aa, synthetic construct
IIDPGDGTTYFAIWVNG

SEQ ID NO: 52 - protein, 6 aa, synthetic construct
DPGDGT

SEQ ID NO: 53 - protein, 11 aa, synthetic construct
EFSASAYQYDL

SEQ ID NO: 54 - protein, 11 aa, synthetic construct
RASEDIENYLA

SEQ ID NO: 55 - protein, 7 aa, synthetic construct
RASTLSS

SEQ ID NO: 56 - protein, 12 aa, synthetic construct
QQGYRNSNVDNP

SEQ ID NO: 57 - protein, 450 aa, synthetic construct
QSVRETGGGLVQPGGSLKLSCKASGFTINIDYWISWVRQAPGKGLEWIGIIDPGDGTTYFAIWVNGRFTISFDNTQNTVDLQMNSLTAADTATYFCVREFSASAYQYDLWGPGTLVTVSSASTKGPSVFPLAPSSKSTSGGTAALGCLVKDYFPEPVTVSWSSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 217 aa, synthetic construct
DLVMTQTPSSVSAAVGGTVSINCRASEDIENYLAWYQQKPGQPPKLLIYRASTLSSGVSSRFSGSGSGTEFTLTISGVQPDDAATYYCQQGYRNSNVDNPFGGGTEMVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 450 aa, synthetic construct; identical to SEQ ID NO: 57

SEQ ID NO: 60 - protein, 481 aa, synthetic construct
DLVMTQTPSSVSAAVGGTVSINCRASEDIENYLAWYQQKPGQPPKLLIYRASTLSSGVSSRFSGSGSGTEFTLTISGVQPDDAATYYCQQGYRNSNVDNPFGGGTEMVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSAFELTQTPSSVEAAVGGTVTINCQASQDIYIYLAWYQQKPGQPPKLLIYSASTLASGVSSRFTGSRSGTQFTLTISDLEPADAATYYCQSYDGTSNTGFGGGTEVVVRGGGGSGGGGSGGGGSGGGGSQSLEESGGRLVTPGTPLTLTCTVSGFSPSSYYMSWVRQAPGKGLEYIGIIFSGGSTWYASWAKGRVTISRTSTTVDLKMTSLTTEDTATYFCARGDAVGSGYWGLWGPGTLVTVSS

SEQ ID NO: 61 - protein, 450 aa, synthetic construct; identical to SEQ ID NO: 57

SEQ ID NO: 62 - protein, 483 aa, synthetic construct
DLVMTQTPSSVSAAVGGTVSINCRASEDIENYLAWYQQKPGQPPKLLIYRASTLSSGVSSRFSGSGSGTEFTLTISGVQPDDAATYYCQQGYRNSNVDNPFGGGTEMVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSAYDMTQTPASVEVAVGGTVTIKCQASQSISTALAWYQQKPGQPPKLLIYKASTLASGVSSRFKGSGSGTQFTLTISGVEPADAATYYCQQGYSRSNVDNVFGGGTEVVVKGGGGSGGGGSGGGGSGGGGSQSVEESGGRLVTPGTPLTLTCTVSGFSLSSYHMSWVRQAPGKGLEWIGYILANTDSYYANWAKGRFTISKTSTTVDLKITSPTTEDTATYFCARYDNHDAAYSTLWGPGTLVTVSS

SEQ ID NO: 63 - protein, 450 aa, synthetic construct; identical to SEQ ID NO: 57

SEQ ID NO: 64 - protein, 484 aa, synthetic construct
DLVMTQTPSSVSAAVGGTVSINCRASEDIENYLAWYQQKPGQPPKLLIYRASTLSSGVSSRFSGSGSGTEFTLTISGVQPDDAATYYCQQGYRNSNVDNPFGGGTEMVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSADVVMTQTPASVSEPVGGTITINCQASENIYSSLAWYQQKPGQPPKLLMYDVSDLASGVPSRFSGSGSGTEFALTISDVQPDDAATYFCLGGYPSNNYRMAFGGGTEVVVKGGGGSGGGGSGGGGSGGGGSQSLEESGGGLVQPEGSLTLTCTASGFSFSSSAWICWVRQAPGKGLEWIGCINTVSVNAYYASWAKGRFTISKTSSTTVTLQVTSLTAADTATHFCARNPYNSLRLWGPGTLVTVSS

SEQ ID NO: 65 - protein, 450 aa, synthetic construct
QSVRETGGGLVQPGGSLKLSCKASGFTINIDYWISWVRQAPGKGLEWIGIIDPGDGTTYFAIWVNGRFTISFDNTQNTVDLQMNSLTAADTATYFCVREFSASAYQYDLWGPGTLVTVSSASTKGPSVFPLAPSSKSTSGGTAALGCLVKDYFPEPVTVSWSSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 481 aa, synthetic construct; identical to SEQ ID NO: 60

SEQ ID NO: 67 - protein, 450 aa, synthetic construct; identical to SEQ ID NO: 65

SEQ ID NO: 68 - protein, 483 aa, synthetic construct; identical to SEQ ID NO: 62

SEQ ID NO: 69 - protein, 450 aa, synthetic construct; identical to SEQ ID NO: 65

SEQ ID NO: 70 - protein, 484 aa, synthetic construct; identical to SEQ ID NO: 64

SEQ ID NO: 71 - protein, 450 aa, synthetic construct
QSVRETGGGLVQPGGSLKLSCKASGFTINIDYWISWVRQAPGKGLEWIGIIDPGDGTTYFAIWVNGRFTISFDNTQNTVDLQMNSLTAADTATYFCVREFSASAYQYDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 217 aa, synthetic construct; identical to SEQ ID NO: 58

SEQ ID NO: 73 - protein, 450 aa, synthetic construct; identical to SEQ ID NO: 71

SEQ ID NO: 74 - protein, 484 aa, synthetic construct; identical to SEQ ID NO: 64

SEQ ID NO: 75 - protein, 712 aa, synthetic construct
QSVRETGGGLVQPGGSLKLSCKASGFTINIDYWISWVRQAPGKGLEWIGIIDPGDGTTYFAIWVNGRFTISFDNTQNTVDLQMNSLTAADTATYFCVREFSASAYQYDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSADVVMTQTPASVSEPVGGTITINCQASENIYSSLAWYQQKPGQPPKLLMYDVSDLASGVPSRFSGSGSGTEFALTISDVQPDDAATYFCLGGYPSNNYRMAFGGGTEVVVKGGGGSGGGGSGGGGSGGGGSQSLEESGGGLVQPEGSLTLTCTASGFSFSSSAWICWVRQAPGKGLEWIGCINTVSVNAYYASWAKGRFTISKTSSTTVTLQVTSLTAADTATHFCARNPYNSLRLWGPGTLVTVSS

SEQ ID NO: 76 - protein, 217 aa, synthetic construct; identical to SEQ ID NO: 58

SEQ ID NO: 77 - protein, 451 aa, synthetic construct
EQQLVETGGGLIQPGGSLRLSCAASGFTINIDYWISWVRQAPGKGLEWIGIIDPGDGTTYFAIWVSGRFTISFDNSKNTVYLQMNSLRAEDTAVYFCVREFSASAYQYDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 480 aa, synthetic construct
ELVMTQSPATLSVSPGERATLSCRASEDIENYLAWYQQKPGQPPRLLIYRASTLSSGVPARFSGSGSGTEFTLTISSLQSEDFAVYYCQQGYRNSNVDNPFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSDVQMTQSPSTLSASVGDRVTITCQASENIYSSLAWYQQKPGKPPKLLMYDVSDLASGVPSRFSGSGSGTEFTLTISSLQPDDFATYFCLGGYPSNNYRMAFGCGTKVEIKGGGGSGGGGSGGGGSGGGGSEQQLLESGGGLVQPGGSLRLSCAASGFSFSSSAWIVWVRQAPGKCLEWIGVINTVSVNAYYASWAKGRFTISKDSSKNTVYLQMNSLRAEDTAVYFCARNPYNSLRLWGPGTLVTVSS

SEQ ID NO: 79 - protein, 713 aa, synthetic construct
EQQLVETGGGLIQPGGSLRLSCAASGFTINIDYWISWVRQAPGKGLEWIGIIDPGDGTTYFAIWVSGRFTISFDNSKNTVYLQMNSLRAEDTAVYFCVREFSASAYQYDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDVQMTQSPSTLSASVGDRVTITCQASENIYSSLAWYQQKPGKPPKLLMYDVSDLASGVPSRFSGSGSGTEFTLTISSLQPDDFATYFCLGGYPSNNYRMAFGCGTKVEIKGGGGSGGGGSGGGGSGGGGSEQQLLESGGGLVQPGGSLRLSCAASGFSFSSSAWIVWVRQAPGKCLEWIGVINTVSVNAYYASWAKGRFTISKDSSKNTVYLQMNSLRAEDTAVYFCARNPYNSLRLWGPGTLVTVSS

SEQ ID NO: 80 - protein, 217 aa, synthetic construct
ELVMTQSPATLSVSPGERATLSCRASEDIENYLAWYQQKPGQPPRLLIYRASTLSSGVPARFSGSGSGTEFTLTISSLQSEDFAVYYCQQGYRNSNVDNPFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 451 aa, synthetic construct
EVQLLESGGGLVQPGGSLRLSCAASGFTFSNYDMAWVRQAPGKGLEWVSSISTRGDITSYRDSVKGRFTISRDNSKNTLYLQMNSLRAEDTAVYYCARQDYYTDYMGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 477 aa, synthetic construct
AIQMTQSPSSLSASVGDRVTITCRASEDIYNGLAWYQQKPGKAPKLLIYGASSLQDGVPSRFSGSGSGTEFTLTISSLQPEDEATYYCAGPHKYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSDVQMTQSPSTLSASVGDRVTITCQASENIYSSLAWYQQKPGKPPKLLMYDVSDLASGVPSRFSGSGSGTEFTLTISSLQPDDFATYFCLGGYPSNNYRMAFGCGTKVEIKGGGGSGGGGSGGGGSGGGGSEQQLLESGGGLVQPGGSLRLSCAASGFSFSSSAWIVWVRQAPGKCLEWIGVINTVSVNAYYASWAKGRFTISKDSSKNTVYLQMNSLRAEDTAVYFCARNPYNSLRLWGPGTLVTVSS

SEQ ID NO: 83 - protein, 450 aa, synthetic construct
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCARNPISIPAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 478 aa, synthetic construct
QPVLTQPPSVSVAPGKTARITCGGNNIGSKSVHWYQQKPGQAPVLVIYYDSDRPSGIPERFSGSNSGNTATLTISRVEAGDEADYYCQVWDTSSDHVLFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSDVQMTQSPSTLSASVGDRVTITCQASENIYSSLAWYQQKPGKPPKLLMYDVSDLASGVPSRFSGSGSGTEFTLTISSLQPDDFATYFCLGGYPSNNYRMAFGCGTKVEIKGGGGSGGGGSGGGGSGGGGSEQQLLESGGGLVQPGGSLRLSCAASGFSFSSSAWIVWVRQAPGKCLEWIGVINTVSVNAYYASWAKGRFTISKDSSKNTVYLQMNSLRAEDTAVYFCARNPYNSLRLWGPGTLVTVSS

SEQ ID NO: 85 - protein, 446 aa, synthetic construct
QVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARDGPWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 477 aa, synthetic construct
NIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSDVQMTQSPSTLSASVGDRVTITCQASENIYSSLAWYQQKPGKPPKLLMYDVSDLASGVPSRFSGSGSGTEFTLTISSLQPDDFATYFCLGGYPSNNYRMAFGCGTKVEIKGGGGSGGGGSGGGGSGGGGSEQQLLESGGGLVQPGGSLRLSCAASGFSFSSSAWIVWVRQAPGKCLEWIGVINTVSVNAYYASWAKGRFTISKDSSKNTVYLQMNSLRAEDTAVYFCARNPYNSLRLWGPGTLVTVSS

SEQ ID NO: 87 - protein, 20 aa, synthetic construct
GGGGSGGGGSGGGGSGGGGS

SEQ ID NO: 88 - protein, 5 aa, synthetic construct
NYDMA

SEQ ID NO: 89 - protein, 17 aa, synthetic construct
SISTRGDITSYRDSVKG

SEQ ID NO: 90 - protein, 12 aa, synthetic construct
QDYYTDYMGFAY

SEQ ID NO: 91 - protein, 11 aa, synthetic construct
RASEDIYNGLA

SEQ ID NO: 92 - protein, 7 aa, synthetic construct
GASSLQD

SEQ ID NO: 93 - protein, 9 aa, synthetic construct
AGPHKYPLT

SEQ ID NO: 94 - protein, 7 aa, synthetic construct
SGSYYWS

SEQ ID NO: 95 - protein, 16 aa, synthetic construct
YIYYSGSTNYNPSLKS

SEQ ID NO: 96 - protein, 10 aa, synthetic construct
NPISIPAFDI

SEQ ID NO: 97 - protein, 11 aa, synthetic construct
GGNNIGSKSVH

SEQ ID NO: 98 - protein, 7 aa, synthetic construct
YDSDRPS

SEQ ID NO: 99 - protein, 11 aa, synthetic construct
QVWDTSSDHVL

SEQ ID NO: 100 - protein, 5 aa, synthetic construct
NYLIE

SEQ ID NO: 101 - protein, 17 aa, synthetic construct
VINPGSGGTNYNEKFKG

SEQ ID NO: 102 - protein, 7 aa, synthetic construct
DGPWFAY

SEQ ID NO: 103 - protein, 11 aa, synthetic construct
KASENVVTYVS

SEQ ID NO: 104 - protein, 7 aa, synthetic construct
GASNRYT

SEQ ID NO: 105 - protein, 9 aa, synthetic construct
GQGYSYPYT

SEQ ID NO: 106 - protein, 121 aa, synthetic construct
EVQLLESGGGLVQPGGSLRLSCAASGFTFSNYDMAWVRQAPGKGLEWVSSISTRGDITSYRDSVKGRFTISRDNSKNTLYLQMNSLRAEDTAVYYCARQDYYTDYMGFAYWGQGTLVTVSS

SEQ ID NO: 107 - protein, 107 aa, synthetic construct
AIQMTQSPSSLSASVGDRVTITCRASEDIYNGLAWYQQKPGKAPKLLIYGASSLQDGVPSRFSGSGSGTEFTLTISSLQPEDEATYYCAGPHKYPLTFGGGTKVEIK

SEQ ID NO: 108 - protein, 120 aa, synthetic construct
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCARNPISIPAFDIWGQGTMVTVSS

SEQ ID NO: 109 - protein, 108 aa, synthetic construct
QPVLTQPPSVSVAPGKTARITCGGNNIGSKSVHWYQQKPGQAPVLVIYYDSDRPSGIPERFSGSNSGNTATLTISRVEAGDEADYYCQVWDTSSDHVLFGGGTKLTVL

SEQ ID NO: 110 - protein, 116 aa, synthetic construct
QVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARDGPWFAYWGQGTLVTVSA

SEQ ID NO: 111 - protein, 107 aa, synthetic construct
NIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIK